(19) United States
(12) Patent Application Publication
(10) Pub. No.: US 2025/0250357 A1
(43) Pub. Date: Aug. 7, 2025

(54) ANTI-MSLN ANTIBODY AND APPLICATION THEREOF

(71) Applicant: NANJING ZAIMING PHARMACEUTICAL CO., LTD., Nanjing (CN)

(72) Inventors: Cuiqing YANG, Shanghai (CN); Xiaohui SHAO, Shanghai (CN); Jingli LIAO, Shanghai (CN); Yayuan FU, Shanghai (CN); Zhuoxiao CAO, Shanghai (CN); Renhong TANG, Shanghai (CN); Jinsheng REN, Nanjing (CN)

(21) Appl. No.: 18/561,582

(22) PCT Filed: May 19, 2022
(86) PCT No.: PCT/CN2022/093759

§ 371 Date: Nov. 16, 2023

(30) Foreign Application Priority Data
May 19, 2021 (CN) 202110543185.0
Nov. 9, 2021 (CN) 202111321146.2

(51) Int. Cl. C07K 16/30; G01N 33/68

(52) U.S. Cl. CPC C07K 16/30; G01N 33/6854; C07K 2317/24; C07K 2317/33; C07K 2317/34; C07K 2317/92

(57) ABSTRACT

An anti-MSLN antibody, a preparation method therefor, and an application thereof. The MSLN antibody has a high affinity with MSLN protein, and is applicable to the preparation of drugs for the treatment of tumors, etc.

### TECHNICAL FIELD

The present invention relates to the field of antibodies, in particular to an anti-MSLN antibody and use thereof.

### BACKGROUND

Mesothelin (MSLN) is a differentiation antigen present on normal mesothelial cells, and can be expressed in the mesothelial cells of the normal pleurae, pericardia and peritonea. Although expression is limited in normal tissues, MSLN has been found to be expressed in 90% of epithelioid malignant pleural mesothelioma cells, 69% of lung adenocarcinoma cells, 60% of breast cancer cells, 46% of esophageal cancer cells, pancreatic tumor cells, and ovarian cancer cells (Morello A et al., Cancer Discov. 2016; 6 (2): 133-146; Baldo P et al., Onco Targets Ther. 2017; 10:5337-5353; Argani P et al., Clin Cancer Res. 2001; 7 (12): 3862-3868; Hassan R et al., Clin Cancer Res. 2004; 10 (12 Pt 1): 3937-3942). Therefore, MSLN is likely to be an important target for cancer therapy.

The MSLN gene which is located in chromosome 16 p13.3 has a total length of 8 kb, with a cDNA length of 2138 bp, has an 1884-bp open reading frame, contains 17 exons, and encodes 628 amino acids. The MSLN gene encodes a precursor protein of 71 kDa. The MSLN precursor protein is anchored to the cell membrane by glycophosphatidylinositol (GPI), and can be hydrolyzed by furin into two portions: an N-terminal soluble protein with a molecular weight of 31 kDa (known as megakaryocyte-potentiating factor (MPF)) and a cell surface glycoprotein with a molecular weight of 40 kDa (i.e., mature MSLN) (Chang K et al., Proc Natl Acad Sci USA. 1996; 93 (1): 136-140; Manzanares M Á et al., Hepatol Commun. 2017; 2 (2): 155-172).

The biological function of mesothelin has not yet been fully elucidated. Researchers studied mice with the MSLN gene knocked out and found that the mice showed no abnormalities in development, reproduction, and blood cell count, indicating that it did not affect the normal growth and development of the mice. (Bera T K et al., Mol Cell Biol. 2000; 20 (8): 2902-2906).

The abnormal expression of MSLN plays an important role in the proliferation, differentiation, adhesion, and drug resistance of tumor cells. The overexpression of MSLN can activate NF-κB (nuclear factor kappa-light-chain-enhancer of activated B cells), MAPK (mitogen-activated protein kinase), and PI3K (phosphoinositide 3-kinases) signaling pathways to induce apoptosis or promote cell proliferation, migration and metastasis by inducing the activation and expression of MMP7 (matrix metalloproteinase-7) and MMP9 (matrix metalloproteinase-9). Studies have shown that MSLN can block taxol-induced apoptosis of tumor cells and increase the tolerance of cancer cells to drugs by simultaneously activating PI3K/AKT (protein kinase B, PKB) and MAPK/ERK (extracellular regulated protein kinase) signaling pathways (Bharadwaj U et al., Mol cancer. 2011; 10:106; Cheng W F et al., Br J Cancer. 2009; 100 (7): 1144-1153).

Drug development approaches targeting MSLN include immunotoxins, vaccines, chimeric monoclonal antibodies, ADCs (antibody-drug conjugates), and CAR-Ts (chimeric antigen receptors T-cells). Antibody drugs mainly mediate apoptosis of tumor cells or inhibit proliferation of tumor cells through antibody neutralization, antibody-dependent cell-mediated cytotoxicity (ADCC), antibody-dependent cell-mediated phagocytosis (ADCP), binding of antibodies to effector molecules (toxins or inhibitors), and the like, so as to target and kill tumor cells.

Amatuximab (MORAb-009) is a chimeric, high-affinity antibody consisting of a single-chain variable fragment of an anti-MSLN antibody SS1 with a human IgG1/K constant region, can prevent adhesion of MSLN-expressing tumor cells to CA125, and can kill tumor cells by ADCC effect (Hassan R et al., Cancer Immun. 2007; 7:20).

Anetumab Ravtansine (BAY94-9343) is an antibody conjugate drug composed of a fully human anti-MSLN antibody (MF-T) and a maytansine derivative DM4 (tubulin polymerase inhibitor) which are linked by a reductive disulfide linker (Grosso F et al., Future Oncol. 2012; 8 (3): 293-305). BAY94-9343 binds to tumor cells and can be internalized to enter lysosomes to release DM4 to kill tumor cells.

MSLN CAR-T cell therapy has shown encouraging results in mouse transplantation models of mesothelioma, ovarian cancer, and lung cancer, and the University of Pennsylvania and the Memorial Sloan Kettering Cancer Center are conducting clinical trials for indications such as pancreatic, ovarian, and pleural tumors (Beatty G L et al., Cancer Immunol Res. 2015 February; 3 (2): 217; Adusumilli PS presented in 11th Annual PEGS Europe Summit, Lisbon).

### SUMMARY

The present invention provides an anti-MSLN antibody, a nucleic acid for encoding the antibody, a method for preparing the antibody, a pharmaceutical composition comprising the antibody, and related use of the pharmaceutical composition in treating a tumor.

In a first aspect, the present invention provides an antibody or an antigen-binding fragment specifically binding to MSLN, wherein the antibody or the antigen-binding fragment comprises: (a) an HCDR1, an HCDR2 and an HCDR3 of a VH set forth in any one of SEQ ID NOs: 594-596, 604-607, 615-618, 626-631, 569-571, 581-584, 593, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, or 156, or a sequence having at least 70% identity to or having 1, 2, 3 or more amino acid insertions, deletions and/or substitutions compared with the HCDR1, the HCDR2 and/or the HCDR3, wherein the substitutions are preferably conservative amino acid substitutions; and/or (b) an LCDR1, an LCDR2 and an LCDR3 of a VL set forth in any one of SEQ ID NOs: 589-592, 601-603, 611-614, 624-625, 565-568, 577-580, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, or 157, or a sequence having at least 70% identity to or having 1, 2, 3 or more amino acid insertions, deletions and/or substitutions compared with the LCDR1, the LCDR2 and/or the LCDR3, wherein the substitutions are preferably conservative amino acid substitutions.

Preferably, the HCDR1-3 and/or the LCDR1-3 are determined according to the Kabat numbering scheme, and more preferably, the HCDR1-3 and/or the LCDR1-3 comprise sequences set forth in Table 23.

In some embodiments, preferably, the HCDR1-3 are selected from any sequence combination of the following VH1-VH77 or sequence combinations having at least 70% identity to or having 1, 2, 3 or more amino acid insertions, deletions and/or substitutions compared with the sequence combination, wherein the substitutions are preferably conservative amino acid substitutions:

| | SEQ ID NO: | | | | SEQ ID NO: | | |
|---|---|---|---|---|---|---|---|
| No. | HCDR1 | HCDR2 | HCDR3 | No. | HCDR1 | HCDR2 | HCDR3 |
| VH1 | 160 | 161 | 162 | VH36 | 373 | 374 | 375 |
| VH2 | 166 | 167 | 168 | VH37 | 379 | 380 | 381 |
| VH3 | 172 | 173 | 174 | VH38 | 385 | 386 | 387 |
| VH4 | 178 | 179 | 180 | VH39 | 391 | 392 | 393 |
| VH5 | 184 | 185 | 186 | VH40 | 394 | 395 | 396 |
| VH6 | 190 | 191 | 192 | VH41 | 400 | 401 | 402 |
| VH7 | 196 | 197 | 198 | VH42 | 403 | 404 | 405 |
| VH8 | 202 | 203 | 204 | VH43 | 409 | 410 | 411 |
| VH9 | 208 | 209 | 210 | VH44 | 415 | 416 | 417 |
| VH10 | 214 | 215 | 216 | VH45 | 421 | 422 | 423 |
| VH11 | 220 | 221 | 222 | VH46 | 424 | 425 | 426 |
| VH12 | 226 | 227 | 228 | VH47 | 430 | 431 | 432 |
| VH13 | 232 | 233 | 234 | VH48 | 436 | 437 | 438 |
| VH14 | 238 | 239 | 240 | VH49 | 442 | 443 | 444 |
| VH15 | 244 | 245 | 246 | VH50 | 448 | 449 | 450 |
| VH16 | 250 | 251 | 252 | VH51 | 454 | 455 | 456 |
| VH17 | 256 | 257 | 258 | VH52 | 460 | 461 | 462 |
| VH18 | 262 | 263 | 264 | VH53 | 466 | 467 | 468 |
| VH19 | 268 | 269 | 270 | VH54 | 472 | 473 | 474 |
| VH20 | 277 | 278 | 279 | VH55 | 478 | 479 | 480 |
| VH21 | 283 | 284 | 285 | VH56 | 481 | 482 | 483 |
| VH22 | 289 | 290 | 291 | VH57 | 487 | 488 | 489 |
| VH23 | 295 | 296 | 297 | VH58 | 493 | 494 | 495 |
| VH24 | 301 | 302 | 303 | VH59 | 499 | 500 | 501 |
| VH25 | 307 | 308 | 309 | VH60 | 505 | 506 | 507 |
| VH26 | 313 | 314 | 315 | VH61 | 511 | 512 | 513 |
| VH27 | 319 | 320 | 321 | VH62 | 517 | 518 | 519 |
| VH28 | 325 | 326 | 327 | VH63 | 523 | 524 | 525 |
| VH29 | 331 | 332 | 333 | VH64 | 526 | 527 | 528 |
| VH30 | 337 | 338 | 339 | VH65 | 532 | 533 | 534 |
| VH31 | 343 | 344 | 345 | VH66 | 535 | 536 | 537 |
| VH32 | 349 | 350 | 351 | VH67 | 541 | 542 | 543 |
| VH33 | 355 | 356 | 357 | VH68 | 547 | 548 | 549 |
| VH34 | 361 | 362 | 363 | VH69 | 553 | 554 | 555 |
| VH35 | 367 | 368 | 369 | VH70 | 559 | 560 | 561 |
| VH71 | 232 | 587 | 234 | VH72 | 232 | 588 | 234 |
| VH73 | 415 | 599 | 417 | VH74 | 415 | 600 | 417 |
| VH75 | 403 | 609 | 405 | VH76 | 403 | 610 | 405 |
| VH77 | 283 | 632 | 285 | | | | |

the LCDR1-3 are selected from any sequence combination of the following VL1-VL67 or sequence combinations having at least 70% identity to or having 1, 2, 3 or more amino acid insertions, deletions and/or substitutions compared with the sequence combination, wherein the substitutions are preferably conservative amino acid substitutions:

| | SEQ ID NO. | | |
|---|---|---|---|
| No. | LCDR1 | LCDR2 | LCDR3 |
| VL1 | 163 | 164 | 165 |
| VL2 | 169 | 170 | 171 |
| VL3 | 175 | 176 | 177 |
| VL4 | 181 | 182 | 183 |
| VL5 | 187 | 188 | 189 |
| VL6 | 193 | 194 | 195 |
| VL7 | 199 | 200 | 201 |
| VL8 | 205 | 206 | 207 |
| VL9 | 211 | 212 | 213 |
| VL10 | 217 | 218 | 219 |
| VL11 | 223 | 224 | 225 |
| VL12 | 229 | 230 | 231 |
| VL13 | 235 | 236 | 237 |
| VL14 | 241 | 242 | 243 |
| VL15 | 247 | 248 | 249 |
| VL16 | 253 | 254 | 255 |
| VL17 | 259 | 260 | 261 |
| VL18 | 265 | 266 | 267 |
| VL19 | 271 | 272 | 273 |
| VL20 | 274 | 275 | 276 |
| VL21 | 280 | 281 | 282 |
| VL22 | 286 | 287 | 288 |
| VL23 | 292 | 293 | 294 |
| VL24 | 298 | 299 | 300 |
| VL25 | 304 | 305 | 306 |
| VL26 | 310 | 311 | 312 |
| VL27 | 316 | 317 | 318 |
| VL28 | 322 | 323 | 324 |
| VL29 | 328 | 329 | 330 |
| VL30 | 334 | 335 | 336 |
| VL31 | 340 | 341 | 342 |
| VL32 | 346 | 347 | 348 |
| VL33 | 352 | 353 | 354 |
| VL34 | 358 | 359 | 360 |
| VL35 | 364 | 365 | 366 |
| VL36 | 370 | 371 | 372 |
| VL37 | 376 | 377 | 378 |
| VL38 | 382 | 383 | 384 |
| VL39 | 388 | 389 | 390 |
| VL40 | 397 | 398 | 399 |
| VL41 | 406 | 407 | 408 |
| VL42 | 412 | 413 | 414 |
| VL43 | 418 | 419 | 420 |
| VL44 | 427 | 428 | 429 |
| VL45 | 433 | 434 | 435 |
| VL46 | 439 | 440 | 441 |
| VL47 | 445 | 446 | 447 |
| VL48 | 451 | 452 | 453 |
| VL49 | 457 | 458 | 459 |
| VL50 | 463 | 464 | 465 |
| VL51 | 469 | 470 | 471 |
| VL52 | 475 | 476 | 477 |
| VL53 | 484 | 485 | 486 |
| VL54 | 490 | 491 | 492 |
| VL55 | 496 | 497 | 498 |
| VL56 | 502 | 503 | 504 |
| VL57 | 508 | 509 | 510 |
| VL58 | 514 | 515 | 516 |
| VL59 | 520 | 521 | 522 |
| VL60 | 529 | 530 | 531 |
| VL61 | 538 | 539 | 540 |
| VL62 | 544 | 545 | 546 |
| VL63 | 550 | 551 | 552 |
| VL64 | 556 | 557 | 558 |
| VL65 | 562 | 563 | 564 |
| VL66 | 622 | 317 | 318 |
| VL67 | 623 | 317 | 318 |

In some embodiments, preferably, the antibody or the antigen-binding fragment comprises a sequence combination selected from: VH1+VL1, VH2+VL2, VH3+VL3, VH4+VL4, VH5+VL5, VH6+VL6, VH7+VL7, VH8+VL8, VH9+VL9, VH10+VL10, VH11+VL11, VH12+VL12, VH13+VL13, VH14+VL14, VH15+VL15, VH16+VL16, VH17+VL17, VH18+VL18, VH19+VL19, VH19+VL20, VH20+VL21, VH21+VL22, VH22+VL23, VH23+VL24, VH24+VL25, VH25+VL26, VH26+VL27, VH27+VL28, VH28+VL29, VH29+VL30, VH30+VL31, VH31+VL32, VH32+VL33, VH33+VL34, VH34+VL35, VH35+VL36, VH36+VL37, VH37+VL38, VH38+VL39, VH39+VL38, VH40+VL40, VH41+VL38, VH42+VL41, VH43+VL42, VH44+VL43, VH45+VL38, VH46+VL44, VH47+VL45, VH48+VL46, VH49+VL47, VH50+VL48, VH51+VL49, VH52+VL50, VH53+VL51, VH54+VL52, VH55+VL14, VH56+VL53, VH57+VL54, VH58+VL55, VH59+VL56, VH60+VL57, VH61+VL58, VH62+VL59, VH63+VL14, VH64+VL60, VH65+VL60, VH66+VL61, VH67+VL62, VH68+VL63, VH69+VL64, VH70+VL65, VH72+VL13, VH73+VL43, VH74+VL43, VH75+VL41, VH76+VL41, VH26+VL66, or VH77+VL22, and a sequence combination having at least 70% identity to or having 1, 2, 3 or more amino acid insertions, deletions and/or substitutions compared with a sequence of the sequence combination, wherein the substitutions are preferably conservative amino acid substitutions.

In some embodiments, preferably, the antibody or the antigen-binding fragment comprises: (1) a sequence having at least 80%, 85%, 90%, 91%, 92%, 93%, 94%, 95%, 96%, 97%, 98%, 99%, or 100% identity to or having at most 20, 19, 18, 17, 16, 15, 14, 13, 12, 11, 10, 9, 8, 7, 6, 5, 4, 3, 2, or 1 mutation compared with a VH set forth in any one of SEQ ID NOs: 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 569-571, 581-584, 593-596, 604-607, 615-618, or 626-631, wherein the mutation may be selected from an insertion, a deletion and/or a substitution, and the substitution is preferably a conservative amino acid substitution; and/or (2) a sequence having at least 80, 85%, 90%, 91%, 92%, 93%, 94%, 95%, 96%, 97%, 98%, 99%, or 100% identity to or having at most 20, 19, 18, 17, 16, 15, 14, 13, 12, 11, 10, 9, 8, 7, 6, 5, 4, 3, 2, or 1 mutation compared with a VL set forth in any one of SEQ ID NOs: 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 565-568, 577-580, 589-592, 601-603, 611-614, or 624-625, wherein the mutation may be selected from an insertion, a deletion and/or a substitution, and the substitution is preferably a conservative amino acid substitution.

In some embodiments, preferably, the antibody or the antigen-binding moiety comprises: (1) a sequence set forth in SEQ ID NO: 16 and a sequence set forth in SEQ ID NO: 17; (2) a sequence set forth in SEQ ID NO: 18 and a sequence set forth in SEQ ID NO: 19; (3) a sequence set forth in SEQ ID NO: 20 and a sequence set forth in SEQ ID NO: 21; (4) a sequence set forth in SEQ ID NO: 22 and a sequence set forth in SEQ ID NO: 23; (5) a sequence set forth in SEQ ID NO: 24 and a sequence set forth in SEQ ID NO: 25; (6) a sequence set forth in SEQ ID NO: 26 and a sequence set forth in SEQ ID NO: 27; (7) a sequence set forth in SEQ ID NO: 28 and a sequence set forth in SEQ ID NO: 29; (8) a sequence set forth in SEQ ID NO: 30 and a sequence set forth in SEQ ID NO: 31; (9) a sequence set forth in SEQ ID NO: 32 and a sequence set forth in SEQ ID NO: 33; (10) a sequence set forth in SEQ ID NO: 34 and a sequence set forth in SEQ ID NO: 35; (11) a sequence set forth in SEQ ID NO: 36 and a sequence set forth in SEQ ID NO: 37; (12) a sequence set forth in SEQ ID NO: 38 and a sequence set forth in SEQ ID NO: 39; (13) a sequence set forth in SEQ ID NO: 40 and a sequence set forth in SEQ ID NO: 41; (14) a sequence set forth in SEQ ID NO: 42 and a sequence set forth in SEQ ID NO: 43; (15) a sequence set forth in SEQ ID NO: 44 and a sequence set forth in SEQ ID NO: 45; (16) a sequence set forth in SEQ ID NO: 46 and a sequence set forth in SEQ ID NO: 47; (17) a sequence set forth in SEQ ID NO: 48 and a sequence set forth in SEQ ID NO: 49; (18) a sequence set forth in SEQ ID NO: 50 and a sequence set forth in SEQ ID NO: 51; (19) a sequence set forth in SEQ ID NO: 52 and a sequence set forth in SEQ ID NO: 53; (20) a sequence set forth in SEQ ID NO: 54 and a sequence set forth in SEQ ID NO: 55; (21) a sequence set forth in SEQ ID NO: 56 and a sequence set forth in SEQ ID NO: 57; (22) a sequence set forth in SEQ ID NO: 58 and a sequence set forth in SEQ ID NO: 59; (23) a sequence set forth in SEQ ID NO: 60 and a sequence set forth in SEQ ID NO: 61; (24) a sequence set forth in SEQ ID NO: 62 and a sequence set forth in SEQ ID NO: 63; (25) a sequence set forth in SEQ ID NO: 64 and a sequence set forth in SEQ ID NO: 65; (26) a sequence set forth in SEQ ID NO: 66 and a sequence set forth in SEQ ID NO: 67; (27) a sequence set forth in SEQ ID NO: 68 and a sequence set forth in SEQ ID NO: 69; (28) a sequence set forth in SEQ ID NO: 70 and a sequence set forth in SEQ ID NO: 71; (29) a sequence set forth in SEQ ID NO: 72 and a sequence set forth in SEQ ID NO: 73; (30) a sequence set forth in SEQ ID NO: 74 and a sequence set forth in SEQ ID NO: 75; (31) a sequence set forth in SEQ ID NO: 76 and a sequence set forth in SEQ ID NO: 77; (32) a sequence set forth in SEQ ID NO: 78 and a sequence set forth in SEQ ID NO: 79; (33) a sequence set forth in SEQ ID NO: 80 and a sequence set forth in SEQ ID NO: 81; (34) a sequence set forth in SEQ ID NO: 82 and a sequence set forth in SEQ ID NO: 83; (35) a sequence set forth in SEQ ID NO: 84 and a sequence set forth in SEQ ID NO: 85; (36) a sequence set forth in SEQ ID NO: 86 and a sequence set forth in SEQ ID NO: 87; (37) a sequence set forth in SEQ ID NO: 88 and a sequence set forth in SEQ ID NO: 89; (38) a sequence set forth in SEQ ID NO: 90 and a sequence set forth in SEQ ID NO: 91; (39) a sequence set forth in SEQ ID NO: 92 and a sequence set forth in SEQ ID NO: 93; (40) a sequence set forth in SEQ ID NO: 94 and a sequence set forth in SEQ ID NO: 95; (41) a sequence set forth in SEQ ID NO: 96 and a sequence set forth in SEQ ID NO: 97; (42) a sequence set forth in SEQ ID NO: 98 and a sequence set forth in SEQ ID NO: 99; (43) a sequence set forth in SEQ ID NO: 100 and a sequence set forth in SEQ ID NO: 101; (44) a sequence set forth in SEQ ID NO: 102 and a sequence set forth in SEQ ID NO: 103; (45) a sequence set forth in SEQ ID NO: 104 and a sequence set forth in SEQ ID NO: 105; (46) a sequence set forth in SEQ ID NO: 106 and a sequence set forth in SEQ ID NO: 107; (47) a sequence set forth in SEQ ID NO: 108 and a sequence set forth in SEQ ID NO: 109; (48) a sequence set forth in SEQ ID NO: 110 and a sequence set forth in SEQ ID NO: 111; (49) a sequence set forth in SEQ ID NO: 112 and a sequence set forth in SEQ ID NO: 113; (50) a sequence set forth in SEQ ID NO: 114 and a sequence set forth in SEQ ID NO: 115; (51) a sequence set forth in SEQ ID NO: 116 and a sequence set forth in SEQ ID NO: 117; (52) a sequence set forth in SEQ ID NO: 118 and a sequence set forth in SEQ ID NO: 119; (53) a sequence set forth in SEQ ID NO: 120 and a sequence set forth in SEQ ID NO: 121; (54) a sequence set forth in SEQ ID NO: 122 and a sequence set forth in SEQ ID NO: 123; (55) a sequence set forth in SEQ ID NO: 124 and a sequence set forth in SEQ ID NO: 125; (56) a sequence set forth in SEQ ID NO: 126 and a sequence set forth in SEQ ID NO: 127; (57) a sequence set forth in SEQ ID NO: 128 and a sequence set forth in SEQ ID NO: 129; (58) a sequence set forth in SEQ ID NO: 130 and a sequence set forth in SEQ ID NO: 131; (59) a sequence set forth in SEQ ID NO: 132 and a sequence set forth in SEQ ID NO: 133; (60) a sequence set forth in SEQ ID NO: 134 and a sequence set forth in SEQ ID NO: 135; (61) a sequence set forth in SEQ ID NO: 136 and a sequence set forth in SEQ ID NO: 137; (62) a sequence set forth in SEQ ID NO: 138 and a sequence set forth in SEQ ID NO: 139; (63) a sequence set forth in SEQ ID NO: 140 and a sequence set forth in SEQ ID NO: 141; (64) a sequence set forth in SEQ ID NO: 142 and a sequence set forth in SEQ ID NO: 143; (65) a sequence set forth in SEQ ID NO: 144 and a sequence set forth in SEQ ID NO: 145; (66) a sequence set forth in SEQ ID NO: 146 and a sequence set forth in SEQ ID NO: 147; (67) a sequence set forth in SEQ ID NO: 148 and a sequence set forth in SEQ ID NO: 149; (68) a sequence set forth in SEQ ID NO: 150 and a sequence set forth in SEQ ID NO: 151; (69) a sequence set forth in SEQ ID NO: 152 and a sequence set forth in SEQ ID NO: 153; (70) a sequence set forth in SEQ ID NO: 154 and a sequence set forth in SEQ ID NO: 155; (71) a sequence set forth in SEQ ID NO: 156 and a sequence set forth in SEQ ID NO: 157; (72) a sequence set forth in SEQ ID NO: 569 and a sequence set forth in SEQ ID NO: 565; (73) a sequence set forth in SEQ ID NO: 569 and a sequence set forth in SEQ ID NO: 566; (74) a sequence set forth in SEQ ID NO: 570 and a sequence set forth in SEQ ID NO: 565; (75) a sequence set forth in SEQ ID NO: 570 and a sequence set forth in SEQ ID NO: 566; (76) a sequence set forth in SEQ ID NO: 570 and a sequence set forth in SEQ ID NO: 568; (77) a sequence set forth in SEQ ID NO: 571 and a sequence set forth in SEQ ID NO: 565; (78) a sequence set forth in SEQ ID NO: 581 and a sequence set forth in SEQ ID NO: 577; (79) a sequence set forth in SEQ ID NO: 581 and a sequence set forth in SEQ ID NO: 580; (80) a sequence set forth in SEQ ID NO: 582 and a sequence set forth in SEQ ID NO: 577; (81) a sequence set forth in SEQ ID NO: 582 and a sequence set forth in SEQ ID NO: 580; (82) a sequence set forth in SEQ ID NO: 584 and a sequence set forth in SEQ ID NO: 577; (83) a sequence set forth in SEQ ID NO: 584 and a sequence set forth in SEQ ID NO: 580; (84) a sequence set forth in SEQ ID NO: 593 and a sequence set forth in SEQ ID NO: 589; (85) a sequence set forth in SEQ ID NO: 593 and a sequence set forth in SEQ ID NO: 590; (86) a sequence set forth in SEQ ID NO: 593 and a sequence set forth in SEQ ID NO: 591; (87) a sequence set forth in SEQ ID NO: 593 and a sequence set forth in SEQ ID NO: 592; (88) a sequence set forth in SEQ ID NO: 594 and a sequence set forth in SEQ ID NO: 589; (89) a sequence set forth in SEQ ID NO: 594 and a sequence set forth in SEQ ID NO: 590; (90) a sequence set forth in SEQ ID NO: 594 and a sequence set forth in SEQ ID NO: 591; (91) a sequence set forth in SEQ ID NO: 594 and a sequence set forth in SEQ ID NO: 592; (92) a sequence set forth in SEQ ID NO: 595 and a sequence set forth in SEQ ID NO: 589; (93) a sequence set forth in SEQ ID NO: 595 and a sequence set forth in SEQ ID NO: 590; (94) a sequence set forth in SEQ ID NO: 595 and a sequence set forth in SEQ ID NO: 591; (95) a sequence set forth in SEQ ID NO: 595 and a sequence set forth in SEQ ID NO: 592; (96) a sequence set forth in SEQ ID NO: 596 and a sequence set forth in SEQ ID NO: 589; (97) a sequence set forth in SEQ ID NO: 596 and a sequence set forth in SEQ ID NO: 590; (98) a sequence set forth in SEQ ID NO: 596 and a sequence set forth in SEQ ID NO: 591; (99) a sequence set forth in SEQ ID NO: 596 and a sequence set forth in SEQ ID NO: 592; (100) a sequence set forth in SEQ ID NO: 604 and a sequence set forth in SEQ ID NO: 601; (101) a sequence set forth in SEQ ID NO: 604 and a sequence set forth in SEQ ID NO: 602; (102) a sequence set forth in SEQ ID NO: 604 and a sequence set forth in SEQ ID NO: 603; (103) a sequence set forth in SEQ ID NO: 605 and a sequence set forth in SEQ ID NO: 601; (104) a sequence set forth in SEQ ID NO: 605 and a sequence set forth in SEQ ID NO: 602; (105) a sequence set forth in SEQ ID NO: 605 and a sequence set forth in SEQ ID NO: 603; (106) a sequence set forth in SEQ ID NO: 606 and a sequence set forth in SEQ ID NO: 601; (107) a sequence set forth in SEQ ID NO: 606 and a sequence set forth in SEQ ID NO: 602; (108) a sequence set forth in SEQ ID NO: 606 and a sequence set forth in SEQ ID NO: 603; (109) a sequence set forth in SEQ ID NO: 607 and a sequence set forth in SEQ ID NO: 601; (110) a sequence set forth in SEQ ID NO: 607 and a sequence set forth in SEQ ID NO: 602; (111) a sequence set forth in SEQ ID NO: 607 and a sequence set forth in SEQ ID NO: 603; (112) a sequence set forth in SEQ ID NO: 611 and a sequence set forth in SEQ ID NO: 615; (113) a sequence set forth in SEQ ID NO: 611 and a sequence set forth in SEQ ID NO: 616; (114) a sequence set forth in SEQ ID NO: 611 and a sequence set forth in SEQ ID NO: 617; (115) a sequence set forth in SEQ ID NO: 611 and a sequence set forth in SEQ ID NO: 618; (116) a sequence set forth in SEQ ID NO: 612 and a sequence set forth in SEQ ID NO: 615; (117) a sequence set forth in SEQ ID NO: 612 and a sequence set forth in SEQ ID NO: 616; (118) a sequence set forth in SEQ ID NO: 612 and a sequence set forth in SEQ ID NO: 617; (119) a sequence set forth in SEQ ID NO: 612 and a sequence set forth in SEQ ID NO: 618; (120) a sequence set forth in SEQ ID NO: 613 and a sequence set forth in SEQ ID NO: 616; (121) a sequence set forth in SEQ ID NO: 624 and a sequence set forth in SEQ ID NO: 631; (122) a sequence set forth in SEQ ID NO: 625 and a sequence set forth in SEQ ID NO: 629; or (123) sequences having at least 70%, 75%, 80%, 85%, 90%, 95%, 96%, 97%, 98%, 99%, or more identity to the sequences set forth in (1) to (122) above.

Further, in some embodiments, the antibody or the antigen-binding fragment comprises or does not comprise an antibody heavy chain constant region and/or light chain variable region; optionally, the antibody heavy chain constant region may be selected from human, alpaca, mouse, rat, rabbit, or sheep; optionally, the antibody heavy chain constant region may be selected from IgG, IgM, IgA, IgE, or IgD, and the IgG may be selected from IgG1, IgG2, IgG3, or IgG4; optionally, the heavy chain constant region may be selected from an Fc region, a CH3 region, a heavy chain constant region without a CH1 fragment, or an intact heavy chain constant region; preferably, the heavy chain constant region has an amino acid sequence set forth in SEQ ID NO: 158; preferably, the light chain constant region has an amino acid sequence set forth in SEQ ID NO: 159.

Further, in some embodiments, the antibody or the antigen-binding fragment specifically binds to a human MSLN protein; preferably, the antibody or the antigen-binding fragment binds to human MSLN with a dissociation constant (KD) of not greater than 8.00E-7 M.

Further, in some embodiments, the antibody or the antigen-binding fragment is: (1) a chimeric antibody or a fragment thereof; (2) a humanized antibody or a fragment thereof; or (3) a fully human antibody or a fragment thereof.

Further, in some embodiments, the antibody or the antigen-binding fragment is selected from a monoclonal antibody, a polyclonal antibody, a natural antibody, an engineered antibody, a monospecific antibody, a multispecific antibody (e.g., a bispecific antibody), a monovalent antibody, a multivalent antibody, an intact antibody, a fragment of an intact antibody, a naked antibody, a conjugated antibody, a chimeric antibody, a humanized antibody, a fully human antibody, a Fab, a Fab′, a Fab′-SH, an F(ab′)₂, an Fd, an Fv, an scFv, a diabody, or a single domain antibody.

Further, in some embodiments, the antibody or the antigen-binding fragment is further conjugated to a therapeutic agent or a tracer; preferably, the therapeutic agent is selected from a drug, a toxin, a radioisotope, a chemotherapeutic agent, or an immunomodulator, and the tracer is selected from a radiocontrast medium, a paramagnetic ion, a metal, a fluorescent label, a chemiluminescent label, an ultrasound contrast agent, and a photosensitizer.

In a second aspect, the present invention provides a multispecific molecule, wherein the multispecific molecule comprises the antibody or the antigen-binding fragment according to the first aspect; preferably, the multispecific molecule further comprises an antibody or an antigen-binding fragment specifically binding to an antigen other than MSLN or binding to an epitope of MSLN different from that of the antibody or the antigen-binding fragment according to the first aspect.

In some embodiments, preferably, the antigen other than MSLN is an antigen on the surface of a T cell, a B cell, a natural killer cell, a dendritic cell, a macrophage, a monocyte, or a neutrophil; preferably, the antigen other than MSLN is selected from: CD3, CD3γ, CD3δ, CD3ε, CD3ζ, CD16, CD16A, CD32B, PD-1, PD-2, PD-L1, VEGF, NKG2D, CD19, CD20, CD40, CD47, 4-1BB, CD137, EGFR, EGFRVIII, TNF-alpha, CD33, HER2, HER3, HAS, CD5, CD27, EphA2, EpCAM, MUC1, MUC16, CEA, Claudin18.2, folate receptor, Claudin6, WT1, NY-ESO-1, MAGE3, ASGPR1, or CDH16.

In some embodiments, preferably, the multispecific molecule is a tandem scFv, a bifunctional antibody (Db), a single chain bifunctional antibody (scDb), a dual affinity retargeting (DART) antibody, an F(ab′)₂, a dual variable domain (DVD) antibody, a knobs-into-holes (KiH) antibody, a dock-and-lock (DNL) antibody, a chemically cross-linked antibody, a heteropoly antibody, or a heteroconjugate antibody.

In a third aspect, the present invention provides a chimeric antigen receptor (CAR), wherein the chimeric antigen receptor at least comprises an extracellular antigen-binding domain, a transmembrane domain, and an intracellular signaling domain; the extracellular antigen-binding domain comprises the antibody or the antigen-binding fragment according to the first aspect.

In a fourth aspect, the present invention provides an immune effector cell, wherein the immune effector cell expresses the chimeric antigen receptor according to the third aspect or comprises a nucleic acid fragment encoding the chimeric antigen receptor according to the third aspect; preferably, the immune effector cell is selected from a T cell, a natural killer (NK) cell, a natural killer T (NKT) cell, a double negative T (DNT) cell, a monocyte, a macrophage, a dendritic cell, or a mast cell; the T cell is preferably selected from a cytotoxic T cell, a regulatory T cell, or a helper T cell; preferably, the immune effector cell is an auto-immune effector cell or an allogeneic immune effector cell.

In a fifth aspect, the present invention provides an isolated nucleic acid fragment, wherein the nucleic acid fragment encodes the antibody or the antigen-binding fragment according to the first aspect, the multispecific molecule according to the second aspect, or the chimeric antigen receptor according to the third aspect.

In a sixth aspect, the present invention provides a vector, wherein the vector comprises the nucleic acid fragment according to the fifth aspect.

In a seventh aspect, the present invention provides a host cell, wherein the host cell comprises the vector according to the sixth aspect; preferably, the cell is a prokaryotic cell or a eukaryotic cell, e.g., a bacteria (E. coli), a fungus (yeast), an insect cell, or a mammalian cell (a CHO cell line or a 293T cell line).

In an eighth aspect, the present invention further provides a method for preparing an antibody or an antigen-binding fragment or a multispecific molecule, wherein the method comprises: culturing the cell according to the seventh aspect described above, and isolating an antibody or an antigen-binding fragment expressed by the cell or a multispecific molecule expressed by the cell in a suitable condition.

In a ninth aspect, the present invention further provides a method for preparing an immune effector cell, wherein the method comprises introducing a nucleic acid fragment encoding the CAR according to the third aspect into the immune effector cell; optionally, the method further comprises initiating expression of the CAR according to the third aspect in the immune effector cell.

In a tenth aspect, the present invention further provides a pharmaceutical composition, wherein the pharmaceutical composition comprises the antibody or the antigen-binding fragment according to the first aspect, the multispecific antibody according to the second aspect, the immune effector cell according to the fourth aspect, the nucleic acid fragment according to the fifth aspect, the vector according to the sixth aspect, or a product prepared by the method according to the eighth or ninth aspect; optionally, the pharmaceutical composition further comprises a pharmaceutically acceptable carrier, diluent or adjuvant; optionally, the pharmaceutical composition further comprises an additional antineoplastic agent.

In an eleventh aspect, the present invention further provides use of the antibody or the antigen-binding fragment according to the first aspect, the multispecific antibody according to the second aspect, the immune effector cell according to the fourth aspect, the nucleic acid fragment according to the fifth aspect, the vector according to the sixth aspect, a product prepared by the method according to the eighth or ninth aspect, or the pharmaceutical composition according to the tenth aspect in preparing a medicament for preventing and/or treating a tumor, wherein the tumor is preferably mesothelioma, lung cancer, breast cancer, esophageal cancer, pancreatic cancer, ovarian cancer, or pleural cancer, more preferably epithelioid malignant pleural mesothelioma or lung adenocarcinoma.

In a twelfth aspect, the present invention provides a method for preventing and/or treating a tumor, comprising: administering to a patient in need thereof an effective amount of the antibody or the antigen-binding fragment according to the first aspect, the multispecific antibody according to the second aspect, the immune effector cell according to the fourth aspect, the nucleic acid fragment according to the fifth aspect, the vector according to the sixth aspect, a product prepared by the method according to the eighth or ninth aspect, or the pharmaceutical composition according to the tenth aspect, wherein the tumor is preferably mesothelioma, lung cancer, breast cancer, esophageal cancer, pancreatic cancer, ovarian cancer, or pleural cancer, more preferably epithelioid malignant pleural mesothelioma or lung adenocarcinoma.

In a thirteenth aspect, the present invention provides a kit, comprising the antibody or the antigen-binding fragment according to the first aspect, the multispecific antibody according to the second aspect, the immune effector cell according to the fourth aspect, the nucleic acid fragment according to the fifth aspect, the vector according to the sixth aspect, a product prepared by the method according to the eighth or ninth aspect, or the pharmaceutical composition according to the tenth aspect.

In a fourteenth aspect, the present invention provides a method for detecting MSLN expression, comprising: contacting a sample to be tested with the antibody or the antigen-binding fragment according to the first aspect in a condition allowing formation of a complex by the antibody or the antigen-binding fragment according to the first aspect and MSLN.

In a fifteenth aspect, the present invention provides a method for inhibiting the proliferation or migration of a cell expressing MSLN in vitro, comprising: contacting the cell with the antibody or the antigen-binding fragment according to the first aspect in a condition allowing formation of a complex by the antibody or the antigen-binding fragment according to the first aspect and MSLN.

### TERMINOLOGY AND DEFINITIONS

Unless otherwise defined herein, scientific and technical terms used in correlation with the present invention shall have the meanings that are commonly understood by those skilled in the art.

Furthermore, unless otherwise stated herein, terms used in the singular form herein shall include the plural form, and vice versa. More specifically, as used in this description and the appended claims, unless otherwise clearly indicated, the singular forms “a”, “an”, and “the” include referents in the plural form.

The terms “including”, “comprising”, and “having” herein are used interchangeably and are intended to indicate the inclusion of a solution, implying that there may be elements other than those listed in the solution. Meanwhile, it should be understood that the descriptions “including”, “comprising”, and “having” as used herein also provide the solution of “consisting of . . . ”. Illustratively, “a composition, comprising A and B” should be understood as the following technical solution: a composition consisting of A and B, and a composition containing other components in addition to A and B, all fall within the scope of the aforementioned “a composition”.

The term “and/or” as used herein includes the meanings of “and”, “or”, and “all or any other combination of elements linked by the term”.

The term “MSLN” herein refers to mesothelin (MSLN), which is a differentiation antigen present on normal mesothelial cells, and may be expressed in the mesothelial cells of the normal pleurae, pericardia and peritonea. Although the expression is limited in normal tissues, MSLN has been found to be highly expressed in epithelioid malignant pleural mesothelioma cells, lung adenocarcinoma cells, breast cancer cells, esophageal cancer cells, pancreatic tumor cells, ovarian cancer cells, etc. The term “MSLN” includes MSLN proteins of any human and non-human animal species, and specifically includes human MSLN as well as MSLN of non-human mammals.

The term “specific binding” herein means that an antigen-binding molecule (e.g., an antibody) specifically binds to an antigen and substantially identical antigens, generally with high affinity, but does not bind to unrelated antigens with high affinity. Affinity is generally reflected in an equilibrium dissociation constant (KD), where a low KD indicates a high affinity. In the case of antibodies, high affinity generally means having a KD of about 1×10⁻⁶ M or less, about 1×10⁻⁷ M or less, about 1×10⁻⁸ M or less, about 1×10⁻⁹ M or less, about 1×10⁻¹⁰ M or less, 1×10⁻¹¹ M or less, or 1×10⁻¹² M or less. KD is calculated as follows: KD=Kd/Ka, where Kd represents the dissociation rate and Ka represents the association rate. The equilibrium dissociation constant KD can be measured by methods well known in the art, such as surface plasmon resonance (e.g., Biacore) or equilibrium dialysis. Illustratively, KD can be obtained by the method as described in Example 5 herein.

The term “antigen-binding molecule” herein is used in its broadest sense and refers to a molecule that specifically binds to an antigen. Illustratively, the antigen-binding molecule includes, but is not limited to, an antibody or an antibody mimetic. “Antibody mimetic” refers to an organic compound or a binding domain that is capable of specifically binding to an antigen, but is not structurally related to an antibody. Illustratively, the antibody mimetic includes, but is not limited to, affibody, affitin, affilin, a designed ankyrin repeat protein (DARPin), a nucleic acid aptamer, and a Kunitz domain peptide.

The term “antibody” herein is used in its broadest sense and refers to a polypeptide or a combination of polypeptides that comprises sufficient sequence from an immunoglobulin heavy chain variable region and/or sufficient sequence from an immunoglobulin light chain variable region to be capable of specifically binding to an antigen. “Antibody” herein encompasses various forms and various structures as long as they exhibit the desired antigen-binding activity. “Antibody” herein includes alternative protein scaffolds or artificial scaffolds having grafted complementarity determining regions (CDRs) or CDR derivatives. Such scaffolds include antibody-derived scaffolds comprising mutations introduced to, for example, stabilize the three-dimensional structure of the antibody, and fully synthetic scaffolds comprising, for example, biocompatible polymers. See, e.g., Korndorfer et al., 2003, Proteins: Structure, Function, and Bioinformatics, 53 (1): 121-129 (2003); and Roque et al., Biotechnol. Prog. 20:639-654 (2004). Such scaffolds may also include non-antibody derived scaffolds, such as scaffold proteins known in the art to be useful for grafting CDRs, including, but not limited to tenascin, fibronectin, peptide aptamers, and the like.

The term “antibody” herein includes a typical “four-chain antibody”, which is an immunoglobulin consisting of two heavy chains (HCs) and two light chains (LCs). The heavy chain refers to a polypeptide chain consisting of, from the N-terminus to the C-terminus, a heavy chain variable region (VH), a heavy chain constant region CH1 domain, a hinge region (HR), a heavy chain constant region CH2 domain, and a heavy chain constant region CH3 domain; moreover, when the full-length antibody is of IgE isoform, the heavy chain optionally further comprises a heavy chain constant region CH4 domain. The light chain is a polypeptide chain consisting of, from the N-terminus to the C-terminus, a light chain variable region (VL) and a light chain constant region (CL). The heavy chains are connected to each other and to the light chains through disulfide bonds to form a Y-shaped structure. The heavy chain constant regions of immunoglobulins differ in their amino acid composition and arrangement, and thus in their antigenicity. Accordingly, “immunoglobulin” herein may be divided into five classes, or isoforms of immunoglobulins, i.e., IgM, IgD, IgG, IgA, and IgE, with their corresponding heavy chains being u, 8, Y, a, and & chains, respectively. The Ig of the same class may also be divided into different subclasses according to the differences in the amino acid composition of the hinge regions and the number and location of disulfide bonds in the heavy chains. For example, IgG may be divided into IgG1, IgG2, IgG3, and IgG4, and IgA may be divided into IgA1 and IgA2. Light chains are divided into K or A chains according to the differences in the constant regions. Each of the five classes of Ig may have a k chain or a 2 chain.

“Antibody” herein may be derived from any animal, including, but not limited to, human and non-human animals, wherein the non-human animals may be selected from primates, mammals, rodents, and vertebrates, such as Camelidae species, Lama glama, Lama guanicoe, Vicugna pacos, sheep, rabbits, mice, rats, or Chondrichthyes (e.g., shark).

“Antibody” herein includes but is not limited to, monoclonal antibodies, polyclonal antibodies, monospecific antibodies, multispecific antibodies (e.g., bispecific antibodies), monovalent antibodies, multivalent antibodies, intact antibodies, fragments of an intact antibody, naked antibodies, conjugated antibodies, chimeric antibodies, humanized antibodies, or fully human antibodies.

The term “monoclonal antibody” herein refers to an antibody obtained from a population of substantially homogeneous antibodies, that is, the individual antibodies constituting the population are identical and/or bind to the same epitope, except for possible variants (e.g., containing naturally occurring mutations or arising during the production of the formulation, such variants typically being present in minor amounts). In contrast to polyclonal antibody formulations that generally comprise different antibodies directed against different determinants (epitopes), each monoclonal antibody in a monoclonal antibody formulation is directed against a single determinant on the antigen. The modifier “monoclonal” herein is not to be construed as requiring the production of the antibody or the antigen-binding molecule by any particular method. For example, monoclonal antibodies can be prepared by a variety of techniques, including (but not limited to) a hybridoma technique, a recombinant DNA method, a phage library display technique, methods that utilize transgenic animals containing all or part of human immunoglobulin loci, and other methods known in the art.

The term “natural antibody” herein refers to an antibody that is made and paired by the immune system of a multicellular organism. The term “engineered antibody” herein refers to a non-natural antibody obtained by genetic engineering, antibody engineering, and the like. Illustratively, “engineered antibody” includes humanized antibodies, small molecule antibodies (e.g., scFv and the like), bispecific antibodies, and the like.

The term “monospecific” herein means having one or more binding sites, each of which binds to the same epitope of the same antigen.

The term “multispecific antibody” herein means having at least two antigen-binding sites, each of which binds to a different epitope of the same antigen or a different epitope of a different antigen. Thus, the terms such as “bispecific”, “trispecific”, and “tetraspecific” refer to the number of different epitopes to which an antibody/antigen-binding molecule can bind.

The term “valent” herein refers to the presence of a specified number of binding sites in an antibody/antigen-binding molecule. Thus, the terms “monovalent”, “divalent”, “tetravalent”, and “hexavalent” refer to the presence of one binding site, two binding sites, four binding sites, and six binding sites, respectively, in an antibody/antigen-binding molecule.

“Full-length antibody”, “complete antibody”, and “intact antibody” herein are used interchangeably and refer to an antibody having a structure substantially similar to that of a natural antibody.

“Antigen-binding fragment” and “antibody fragment” herein are used interchangeably and do not have the entire structure of an intact antibody, but comprise only a portion of the intact antibody or a variant of the portion, wherein the portion or the variant of the portion has the ability to bind to an antigen. “Antigen-binding fragment” or “antibody fragment” herein includes but is not limited to, a Fab, a Fab′, a Fab′-SH, an F(ab′)₂, an Fd, an Fv, an scFv, a diabody, and a single domain antibody.

An intact antibody is digested by papain to produce two identical antigen-binding fragments, called “Fab” fragments, each of which contains a heavy chain variable domain and a light chain variable domain, as well as a light chain constant domain and a first heavy chain constant domain (CH1). Thus, the term “Fab fragment” herein refers to an antibody fragment comprising a light chain fragment comprising the VL domain and the constant domain (CL) of a light chain, and the VH domain and the first constant domain (CH1) of a heavy chain. A Fab′ fragment differs from the Fab fragment by the addition of a few residues (including one or more cysteines from an antibody hinge region) at the carboxyl terminus of the heavy chain CH1 domain. Fab′-SH is a Fab′ fragment in which the cysteine residue in the constant domain carries a free thiol group. Pepsin treatment produces an F(ab′)₂ fragment having two antigen-binding sites (two Fab fragments) and a portion of the Fc region.

The term “Fd” herein refers to an antibody consisting of VH and CH1 domains. The term “Fv” herein refers to an antibody fragment consisting of VL and VH domains of a single arm. An Fv fragment is generally considered to be the smallest antibody fragment that can form an intact antigen-binding site. It is generally believed that the six CDRs provide antigen-binding specificity to the antibody. However, even one variable region (e.g., an Fd fragment, which contains only three CDRs specific to an antigen) is capable of recognizing and binding to an antigen, although its affinity may be lower that of than an intact binding site.

The term “scFv” (single-chain variable fragment) herein refers to a single polypeptide chain comprising VL and VH domains, wherein the VL and VH are linked through a linker (see, e.g., Bird et al., Science 242:423-426 (1988); Huston et al., Proc. Natl. Acad. Sci. USA 85:5879-5883 (1988); and Pluckthun, The Pharmacology of Monoclonal Antibodies, Vol. 113, Roseburg and Moore Ed., Springer-Verlag, New York, pp 269-315 (1994)). Such scFv molecules may have a general structure: NH2-VL-linker-VH-COOH or NH2-VH-linker-VL-COOH. An appropriate linker in the prior art consists of GGGGS amino acid sequence repeats or a variant thereof. For example, a linker having the amino acid sequence (GGGGS)₄ can be used, and variants thereof can also be used (Holliger et al. (1993), Proc. Natl. Acad. Sci. USA 90:6444-6448). Other linkers that can be used in the present invention are described in Alfthan et al. (1995), Protein Eng. 8:725-731; Choi et al. (2001), Eur. J. Immunol. 31:94-106; Hu et al. (1996), Cancer Res. 56:3055-3061; Kipriyanov et al. (1999), J. Mol. Biol. 293:41-56; and Roovers et al. (2001), Cancer Immunol. In some cases, there may also be disulfide bonds between the VH and VL of the scFv, forming a disulfide-linked Fv (dsFv).

The term “diabody” herein refers to an antibody having VH and VL domains that are expressed on a single polypeptide chain, but using a linker that is too short to allow the pairing of the two domains on the same chain, thereby forcing the domains to pair with the complementary domains of the other chain and generating two antigen-binding sites (see, e.g., Holliger P. et al., Proc. Natl. Acad. Sci. USA 90:6444-6448 (1993), and Poljak R. J. et al., Structure 2:1121-1123 (1994))

The term “naked antibody” herein refers to an antibody that is not conjugated to a therapeutic agent or tracer. The term “conjugated antibody” herein refers to an antibody that is conjugated to a therapeutic agent or tracer.

The term “chimeric antibody” herein refers to an antibody in which a portion of the light chain or/and heavy chain is derived from one antibody (which may be derived from a particular species or belong to a particular antibody class or subclass) and another portion of the light chain or/and heavy chain is derived from another antibody (which may be derived from the same or a different species or belong to the same or a different antibody class or subclass), but which nevertheless retains binding activity to a target antigen (U.S. Pat. No. 4,816,567 to Cabilly et al.; Morrison et al., Proc. Natl. Acad. Sci. USA, 81:6851 6855 (1984)). For example, the term “chimeric antibody” can include an antibody (e.g., a human-murine chimeric antibody) in which the heavy and light chain variable regions of the antibody are derived from a first antibody (e.g., a murine antibody) and the heavy and light chain constant regions of the antibody are derived from a second antibody (e.g., a human antibody).

The term “humanized antibody” herein refers to a genetically engineered non-human antibody that has an amino acid sequence modified to increase homology to the sequence of a human antibody. Generally, all or part of the CDRs of a humanized antibody is derived from a non-human antibody (donor antibody), and all or part of the non-CDRs (e.g., variable region FRs and/or constant regions) is derived from a human immunoglobulin (receptor antibody). The humanized antibody generally retains or partially retains the desired properties of the donor antibody, including, but not limited to, antigen specificity, affinity, reactivity, the ability to increase the activity of immune cells, the ability to enhance immune response, and the like.

The term “fully human antibody” herein refers to an antibody having variable regions in which both the FRs and CDRs are derived from human germline immunoglobulin sequences. Furthermore, if the antibody comprises constant regions, the constant regions are also derived from human germline immunoglobulin sequences. The fully human antibody herein may include amino acid residues that are not encoded by human germline immunoglobulin sequences (e.g., mutations introduced by random or site-specific mutagenesis in vitro or by somatic mutations in vivo). However, “fully human antibody” herein does not include antibodies in which CDR sequences derived from the germline of another mammalian species (e.g., mouse) have been grafted onto human framework sequences.

The term “variable region” herein refers to a region of a heavy or light chain of an antibody involved in the binding of the antibody to an antigen. “Heavy chain variable region” is used interchangeably with “VH” and “HCVR”, and “light chain variable region” is used interchangeably with “VL” and “LCVR”. Heavy and light chain variable domains (VH and VL, respectively) of natural antibodies generally have similar structures, each of which contains four conservative framework regions (FRs) and three hypervariable regions (HVRs). See, e.g., Kindt et al., Kuby Immunology, 6th ed., W. H. Freeman and Co., p. 91 (2007). A single VH or VL domain may be sufficient to provide antigen-binding specificity. The terms “complementarity determining region” and “CDR” herein are used interchangeably and generally refer to a hypervariable region (HVR) of a heavy chain variable region (VH) or a light chain variable region (VL), which is also known as the complementarity determining region because it is precisely complementary to an epitope in a spatial structure, wherein the heavy chain variable chain CDR may be abbreviated as HCDR and the light chain variable chain CDR may be abbreviated as LCDR. The terms “framework region” or “FR” are used interchangeably and refer to those amino acid residues of an antibody heavy chain variable region or light chain variable region, other than CDRs. Generally, a typical antibody variable region consists of 4 FRs and 3 CDRs in the following order: FR1-CDR1-FR2-CDR2-FR3-CDR3-FR4.

For further description of the CDRs, see Kabat et al., J. Biol. Chem., 252:6609-6616 (1977); Kabat et al., United States Department of Health and Human Services, Sequences of proteins of immunological interest (1991); Chothia et al., J. Mol. Biol. 196:901-917 (1987); Al-Lazikani B. et al., J. Mol. Biol., 273:927-948 (1997); MacCallum et al., J. Mol. Biol. 262:732-745 (1996); Abhinandan and Martin, Mol. Immunol., 45:3832-3839 (2008); Lefranc M. P. et al., Dev. Comp. Immunol., 27:55-77 (2003); and Honegger and Pluckthun, J. Mol. Biol., 309:657-670 (2001). “CDR” herein may be labeled and defined in a manner well known in the art, including, but not limited to, Kabat numbering scheme, Chothia numbering scheme, or IMGT numbering scheme; the tool sites used include, but are not limited to, AbRSA site (website: cao.labshare.cn/AbRSA/cdrs.php), ab Ysis site (website: abysis.org/abysis/sequence_input/key_annotation/key_annotation.cgi), and IMGT site (website: imgt.org/3Dstructure-DB/cgi/DomainGapAlign.cgi#results). The CDR herein includes overlaps and subsets of amino acid residues defined in different ways.

The term “Kabat numbering scheme” herein generally refers to the immunoglobulin alignment and numbering scheme proposed by Elvin A. Kabat (see, e.g., Kabat et al., Sequences of Proteins of Immunological Interest, 5th Ed. Public Health Service, National Institutes of Health, Bethesda, Md., 1991).

The term “heavy chain constant region” herein refers to the carboxyl-terminal portion of an antibody heavy chain that is not directly involved in the binding of the antibody to an antigen, but exhibits effector functions, such as interaction with an Fc receptor, which has a more conservative amino acid sequence relative to the variable domain of the antibody. The “heavy chain constant region” at least comprises: a CH1 domain, a hinge region, a CH2 domain, a CH3 domain, or a variant or fragment thereof. The “heavy chain constant region” includes a “full-length heavy chain constant region” having a structure substantially similar to that of a natural antibody constant region, and a “heavy chain constant region fragment” including only “a portion of the full-length heavy chain constant region”. Illustratively, a typical “full-length antibody heavy chain constant region” consists of the CH1 domain-hinge region-CH2 domain-CH3 domain. When the antibody is IgE, it further comprises a CH4 domain; and when the antibody is a heavy chain antibody, it does not comprise a CH1 domain. Illustratively, a typical “heavy chain constant region fragment” may be selected from CH1, Fc, or CH3 domains.

The term “light chain constant region” herein refers to the carboxyl-terminal portion of an antibody light chain that is not directly involved in the binding of the antibody to an antigen, wherein the light chain constant region may be selected from a constant k domain and a constant 2 domain.

The term “Fc” herein refers to the carboxyl-terminal portion of an antibody that is formed by the hydrolysis of an intact antibody by papain, which typically comprises the CH3 and CH2 domains of the antibody. The Fc region includes, for example, an Fc region of native sequence, a recombinant Fc region, and a variant Fc region. Although the boundaries of the Fc region of an immunoglobulin heavy chain may vary slightly, the Fc region of a human IgG heavy chain is generally defined as extending from an amino acid residue at position Cys226, or from Pro230, to the carboxyl terminus thereof. The C-terminal lysine of the Fc region (residue 447 according to the Kabat numbering scheme) may be removed, for example, during production or purification of the antibody, or by recombinant engineering of the nucleic acids encoding the heavy chain of the antibody, and thus, the Fc region may or may not include Lys447.

The term “conservative amino acid” herein generally refers to amino acids that belong to the same class or have similar characteristics (e.g., charge, side chain size, hydrophobicity, hydrophilicity, backbone conformation, and rigidity). Illustratively, the amino acids in each of the following groups are conservative amino acid residues of each other, and substitutions of amino acid residues within the groups are conservative amino acid substitutions:

Illustratively, the following six groups are examples of amino acids that are considered to be conservative replacements of each other: 1) alanine (A), serine(S), and threonine (T); 2) aspartic acid (D) and glutamic acid (E); 3) asparagine (N) and glutamine (Q); 4) arginine (R), lysine (K), and histidine (H); 5) isoleucine (I), leucine (L), methionine (M), and valine (V); and 6) phenylalanine (F), tyrosine (Y), and tryptophan (W).

The term “identity” herein can be obtained by calculating as follows: to determine the percent “identity” of two amino acid sequences or two nucleic acid sequences, the sequences are aligned for optimal comparison purposes (e.g., for optimal alignment, gaps can be introduced in one or both of the first and second amino acid sequences or nucleic acid sequences, or non-homologous sequences can be discarded for comparison). Amino acid residues or nucleotides at corresponding amino acid positions or nucleotide positions are then compared. When a position in the first sequence is occupied by the same amino acid residue or nucleotide at the corresponding position in the second sequence, the molecules are identical at this position.

The percent identity between two sequences varies with the identical positions shared by the sequences, taking into account the number of gaps that need to be introduced and the length of each gap for optimal alignment of the two sequences.

A mathematical algorithm can be used to compare two sequences and calculate the percent identity between the sequences. For example, the percent identity between two amino acid sequences is determined with the Needlema and Wunsch algorithm ((1970) J. Mol. Biol., 48:444-453; available at website: gcg.com) which have been integrated into the GAP program of the GCG software package, using the Blosum 62 matrix or PAM250 matrix and gap weight of 16, 14, 12, 10, 8, 6, or 4 and length weight of 1, 2, 3, 4, 5, or 6. For another example, the percent identity between two nucleotide sequences is determined with the GAP program of the GCG software package (available at website: gcg.com), using the NWSgapdna.CMP matrix and gap weight of 40, 50, 60, 70, or 80 and length weight of 1, 2, 3, 4, 5, or 6. A particularly preferred parameter set (and one that should be used unless otherwise stated) is a Blossum62 scoring matrix with a gap penalty of 12, a gap extension penalty of 4, and a frameshift gap penalty of 5.

The percent identity between two amino acid sequences or nucleotide sequences can also be determined with a PAM120 weighted remainder table, a gap length penalty of 12, and a gap penalty of 4, using the E. Meyers and W. Miller algorithm ((1989) CABIOS, 4:11-17) which has been incorporated into the ALIGN program (version 2.0).

Additionally or alternatively, the nucleic acid sequences and protein sequences described herein can be further used as “query sequences” to perform searches against public databases to, e.g., identify other family member sequences or related sequences. For example, such searches can be performed using the NBLAST and XBLAST programs (version 2.0) of Altschul et al., (1990) J. Mol. Biol., 215:403-10. BLAST nucleotide searches can be performed using the NBLAST program, with a score of 100 and a word length of 12, to obtain nucleotide sequences homologous to the nucleic acid (SEQ ID NO: 1) molecule of the present invention. BLAST protein searches can be performed using the XBLAST program, with a score of 50 and a word length of 3, to obtain amino acid sequences homologous to the protein molecule of the present invention. To obtain gapped alignment results for the purpose of comparison, gapped BLAST can be used as described in Altschul et al. (1997) Nucleic Acids Res. 25:3389-3402. When using the BLAST and gapped BLAST programs, the default parameters of the respective programs (e.g., XBLAST and NBLAST) can be used. See website: nebi.nlm.nih.gov.

The term “chimeric antigen receptor (CAR)” herein refers to an artificial cell surface receptor engineered to be expressed on an immune effector cell and specifically bound to an antigen, which comprises at least (1) an extracellular antigen-binding domain, e.g., a variable heavy or light chain of an antibody, (2) a transmembrane domain that anchors the CAR into the immune effector cell, and (3) an intracellular signaling domain. The CAR is capable of redirecting T cells and other immune effector cells to a selected target, e.g., a cancer cell, in a non-MHC-restricted manner using the extracellular antigen-binding domain.

The term “nucleic acid” herein includes any compound and/or substance that comprises a polymer of nucleotides. Each nucleotide consists of a base, in particular a purine or pyrimidine base (i.e., cytosine (C), guanine (G), adenine (A), thymine (T), or uracil (U)), a sugar (i.e., deoxyribose or ribose), and a phosphate group. Generally, a nucleic acid molecule is described as a sequence of bases, whereby the bases represent the primary structure (linear structure) of the nucleic acid molecule. The sequence of bases is generally expressed as 5′ to 3′. In this context, the term “nucleic acid molecule” encompasses deoxyribonucleic acid (DNA), including, e.g., complementary DNA (cDNA) and genomic DNA; ribonucleic acid (RNA), in particular in the synthetic form of messenger RNA (mRNA), DNA or RNA; and polymers comprising a mixture of two or more of these molecules. The nucleic acid molecule may be linear or cyclic. Furthermore, the term “nucleic acid molecule” includes both sense and antisense strands, as well as single- and double-stranded forms. Moreover, the nucleic acid molecules described herein may contain naturally occurring or non-naturally occurring nucleotides. Examples of non-naturally occurring nucleotides include modified nucleotide bases having derived sugar or phosphate backbone linkages or chemically modified residues. The nucleic acid molecule also encompasses DNA and RNA molecules suitable for use as vectors for direct expression of the antibodies of the present invention in vitro and/or in vivo, e.g., in a host or patient. Such DNA (e.g., cDNA) or RNA (e.g., mRNA) vectors may be unmodified or modified. For example, mRNA may be chemically modified to enhance the stability of the RNA vector and/or the expression of the encoded molecule, so that the mRNA can be injected into a subject to produce antibodies in vivo (see, e.g., Stadler et al., Nature Medicine 2017, published online, Jun. 12, 2017, doi: 10.1038/nm.4356 or EP 2 101 823 B1). “Isolated” nucleic acid herein refers to a nucleic acid molecule that has been separated from components of its natural environment. The isolated nucleic acid includes a nucleic acid molecule contained in a cell that generally contains the nucleic acid molecule, but the nucleic acid molecule is present extrachromosomally or at a chromosomal location different from its natural chromosomal location.

The term “vector” herein refers to a nucleic acid molecule capable of amplifying another nucleic acid to which it has been linked. The term includes vectors that serve as self-replicating nucleic acid structures as well as vectors integrated into the genome of a host cell into which they have been introduced. Certain vectors are capable of directing the expression of nucleic acids to which they are operably linked. Such vectors are called “expression vectors” herein.

The term “host cell” herein refers to a cell into which an exogenous nucleic acid has been introduced, including the progeny of such a cell. Host cells include “transformants” and “transformed cells”, which include primary transformed cells and progenies derived therefrom, regardless of the number of passages. Progenies may not be exactly the same as parent cells in terms of nucleic acid content, and may contain mutations. Mutant progenies having the same function or biological activity that are screened or selected from the primary transformed cells are included herein.

The term “pharmaceutical composition” herein refers to a formulation that exists in a form allowing the biological activity of the active ingredient contained therein to be effective, and does not contain additional ingredients having unacceptable toxicity to a subject to which the pharmaceutical composition is administered.

The term “treatment” herein refers to surgical or therapeutic treatment for the purpose of preventing or slowing (reducing) the progression of an undesired physiological or pathological change, e.g., a cancer, in a subject being treated. Beneficial or desired clinical results include, but are not limited to, alleviation of symptoms, decrease of severity of disease, stabilization (i.e., not worsening) of state of disease, delay or slowing of disease progression, amelioration or palliation of state of disease, and remission (whether partial or total), whether detectable or undetectable. Subjects in need of treatment include those already with a disorder or disease, as well as those who are susceptible to a disorder or disease or those who intend to prevent a disorder or disease. When referring to terms such as slowing, alleviation, decrease, palliation, and remission, their meanings also include elimination, disappearance, nonoccurrence, etc.

The term “subject” herein refers to an organism that receives treatment for a particular disease or disorder described herein. Examples of subjects and patients include mammals, such as human, primates (e.g., monkey), or non-primate mammals, that receive treatment for a disease or disorder.

The term “effective amount” herein refers to an amount of a therapeutic agent that is effective in preventing or alleviating symptoms of a disease or the progression of the disease when administered to a cell, tissue or subject alone or in combination with another therapeutic agent. “Effective amount” also refers to an amount of a compound that is sufficient to alleviate symptoms, e.g., to treat, cure, prevent, or alleviate related medical disorders, or to increase the rates at which such disorders are treated, cured, prevented, or alleviated. When the active ingredient is administered alone to an individual, a therapeutically effective dose refers to the amount of the ingredient alone. When a combination is used, a therapeutically effective dose refers to the combined amounts of the active ingredients that produce the therapeutic effect, whether administered in combination, sequentially, or simultaneously.

The term “cancer” herein refers to or describes a physiological condition in mammals that is typically characterized by unregulated cell growth. Included in this definition are benign and malignant cancers. The term “tumor” or “neoplasm” herein refers to all neoplastic cell growth and proliferation, whether malignant or benign, and all pre-cancerous and cancerous cells and tissues. The terms “cancer” and “tumor” are not mutually exclusive when referred to herein.

The term “EC50” herein refers to the half maximum effective concentration, which includes the antibody concentration that induces a halfway response between the baseline and maximum after a specified exposure time. EC50 essentially represents the antibody concentration at which 50% of the maximum effect is observed, and can be measured by methods known in the art.

### BRIEF DESCRIPTION OF THE DRAWINGS

FIG. 1 shows the purity of human MSLN protein as assayed by SDS-PAGE.

In FIG. 2, A shows the binding activity of human MSLN-R3-rFc protein to control antibodies as assayed by ELISA; B shows the binding activity of human MSLN-FL-his protein to control antibodies as assayed by ELISA; C shows the binding activity of human MSLN-R1-his protein to control antibodies as assayed by ELISA; D shows the binding activity of human MSLN-R2-his protein to control antibodies as assayed by ELISA; and E shows the binding activity of human MSLN-R3-his protein to control antibodies as assayed by ELISA.

FIG. 3 shows the binding activity of control antibodies to MSLN proteins as assayed by ELISA.

In FIG. 4, A shows assay results for the expression level of MSLN in Hela cells using a control antibody Tab 106 by FACS; B shows assay results for the expression level of MSLN in Hela cells using a control antibody Tab 131 by FACS; and C shows assay results for the expression level of MSLN in Hela cells using a control antibody Tab 142 by FACS.

In FIG. 5, A shows assay results for the expression level of MSLN in OVCAR3 cells using a control antibody Tab 106 by FACS; B shows assay results for the expression level of MSLN in OVCAR3 cells using a control antibody Tab131 by FACS; and C shows assay results for the expression level of MSLN in OVCAR3 cells using a control antibody Tab 142 by FACS.

In FIG. 6, A shows screening results of CHO-K1-hMSLN-2C8 cells transfected with human MSLN protein using a control antibody Tab020 by FACS; B shows screening results of CHO-K1-hMSLN-2D11 cells transfected with human MSLN protein using a control antibody Tab020 by FACS; and C shows screening results of CHO-K1-hMSLN-2C5 cells transfected with human MSLN protein using a control antibody Tab020 by FACS.

FIG. 7 shows assay results for the expression level in HEK293T-monkey MSLN cells using an NB149 antiserum by FACS.

In FIG. 8, A shows screening results of HEK293T-hMSLN-B8 cells transfected with human MSLN protein using a control antibody Tab020 by FACS; B shows screening results of HEK293T-hMSLN-2A4 cells transfected with human MSLN protein using a control antibody Tab020 by FACS; and C shows screening results of HEK293T-hMSLN-2A7 cells transfected with human MSLN protein using a control antibody Tab020 by FACS.

FIG. 9 shows screening results of HEK293T cells transfected with human MSLN-R3 protein using a control antibody Tab 106 by FACS.

In FIG. 10, A shows assay results for binding reactions of control antibodies with human tumor cells (OVCAR3 tumor cells) by FACS; B shows assay results for binding reactions of control antibodies with CHO-K1-hMSLN-2C8 recombinant cells by FACS; and C shows assay results for binding reactions of control antibodies with HEK293T-monkey MSLN recombinant cells by FACS.

In FIG. 11, A shows assay results for serum antibody titers of mice after immunization using human MSLN-FL-his, MSLN-R3-his, and MSLN-R3-3 proteins; and B and C show assay results for serum antibody titers of mice after immunization using human MSLN-FL-his proteins.

In FIGS. 12, A and B show assay results for serum antibody titers of mice after immunization using human MSLN-R1-his proteins.

In FIGS. 13, A and B show assay results for serum antibody titers of mice after immunization using human MSLN-R2-his proteins.

In FIGS. 14, A and B show assay results for serum antibody titers of mice after immunization using human MSLN-R3-his proteins.

In FIGS. 15, A and B show assay results for serum antibody titers of mice after immunization using human MSLN-R3-3 proteins.

In FIGS. 16, A, B, and C show assay results for serum antibody titers of mice after immunization using different cells.

In FIG. 17, A shows assay results for serum antibody titers of mice after immunization using OVCAR3 cells; B shows assay results for serum antibody titers of mice after immunization using HEK293T-hMSLN cells; C shows assay results for serum antibody titers of mice after immunization using HEK293T-monkey MSLN cells; and D shows assay results for serum antibody titers of mice after immunization using HEK293T cells.

In FIGS. 18, A and B show assay results for serum antibody titers of mice after immunization using different cells.

In FIG. 19, A shows assay results for binding reactions of F1 chimeric antibodies with human MSLN-FL-his protein by ELISA; B shows assay results for binding reactions of F1 chimeric antibodies with human MSLN-R1-his protein by ELISA; C shows assay results for binding reactions of F1 chimeric antibodies with human MSLN-R2-his protein by ELISA; D shows assay results for binding reactions of F1 chimeric antibodies with human MSLN-R3-his protein by ELISA; and E shows assay results for binding reactions of F1 chimeric antibodies with human MSLN-R3-3 by ELISA.

In FIG. 20, A shows assay results for binding reactions of F2 chimeric antibodies with human MSLN-FL-his protein by ELISA; B shows assay results for binding reactions of F2 chimeric antibodies with human MSLN-R1-his protein by ELISA; C shows assay results for binding reactions of F2 chimeric antibodies with human MSLN-R2-his protein by ELISA; D shows assay results for binding reactions of F2 chimeric antibodies with human MSLN-R3-his protein by ELISA; and E shows assay results for binding reactions of F2 chimeric antibodies with human MSLN-R3-3 by ELISA.

In FIG. 21, A shows assay results for binding reactions of F3 chimeric antibodies with human MSLN-FL-his protein by ELISA; B shows assay results for binding reactions of F3 chimeric antibodies with human MSLN-R1-his protein by ELISA; C shows assay results for binding reactions of F3 chimeric antibodies with human MSLN-R2-his protein by ELISA; D shows assay results for binding reactions of F3 chimeric antibodies with human MSLN-R3-his protein by ELISA; and E shows assay results for binding reactions of F3 chimeric antibodies with human MSLN-R3-3 by ELISA.

In FIG. 22, A shows assay results for binding reactions of F4, 5 and 6 chimeric antibodies with human MSLN-FL-his protein by ELISA; B shows assay results for binding reactions of F4, 5 and 6 chimeric antibodies with human MSLN-R1-his protein by ELISA; C shows assay results for binding reactions of F4, 5 and 6 chimeric antibodies with human MSLN-R2-his protein by ELISA; D shows assay results for binding reactions of F4, 5 and 6 chimeric antibodies with human MSLN-R3-his protein by ELISA; and E shows assay results for binding reactions of F4, 5 and 6 chimeric antibodies with human MSLN-R3-3 by ELISA.

In FIGS. 23, A and B show assay results for binding reactions of F7 chimeric antibodies with human MSLN-FL-his protein by ELISA; C and D show assay results for binding reactions of F7 chimeric antibodies with human MSLN-R1-his protein by ELISA; E and F show assay results for binding reactions of F7 chimeric antibodies with human MSLN-R2-his protein by ELISA; and G and H show assay results for binding reactions of F7 chimeric antibodies with human MSLN-R3-his protein by ELISA.

In FIGS. 24, A and B show assay results for binding reactions of F8 chimeric antibodies with human MSLN-FL-his protein by ELISA; C and D show assay results for binding reactions of F8 chimeric antibodies with human MSLN-R1-his protein by ELISA; E and F show assay results for binding reactions of F8 chimeric antibodies with human MSLN-R2-his protein by ELISA; and G and H show assay results for binding reactions of F8 chimeric antibodies with human MSLN-R3-his protein by ELISA.

In FIG. 25, A shows assay results for binding reactions of F1 chimeric antibodies with OVCAR3 cells by FACS; B shows assay results for binding reactions of F1 chimeric antibodies with HEK293T-hMSLN-B8 cells by FACS; C shows assay results for binding reactions of F1 chimeric antibodies with HEK293T-hMSLN-R3 cells by FACS; D shows assay results for binding reactions of F1 chimeric antibodies with HEK293T-monkey MSLN cells by FACS; E shows assay results for binding reactions of F1 chimeric antibodies with A431 cells by FACS; and F shows assay results for binding reactions of F1 chimeric antibodies with 293T cells by FACS.

In FIG. 26, A shows assay results for binding reactions of F2 chimeric antibodies with OVCAR3 cells by FACS; B shows assay results for binding reactions of F2 chimeric antibodies with HEK293T-hMSLN-B8 cells by FACS; C shows assay results for binding reactions of F2 chimeric antibodies with HEK293T-hMSLN-R3 cells by FACS; D shows assay results for binding reactions of F2 chimeric antibodies with HEK293T-monkey MSLN cells by FACS; E shows assay results for binding reactions of F2 chimeric antibodies with A431 cells by FACS; and F shows assay results for binding reactions of F2 chimeric antibodies with 293T cells by FACS.

In FIG. 27, A shows assay results for binding reactions of F3 chimeric antibodies with OVCAR3 cells by FACS; B shows assay results for binding reactions of F3 chimeric antibodies with HEK293T-hMSLN-B8 cells by FACS; C shows assay results for binding reactions of F3 chimeric antibodies with HEK293T-hMSLN-R3 cells by FACS; D shows assay results for binding reactions of F3 chimeric antibodies with HEK293T-monkey MSLN cells by FACS; E shows assay results for binding reactions of F3 chimeric antibodies with A431 cells by FACS; and F shows assay results for binding reactions of F3 chimeric antibodies with 293T cells by FACS.

In FIG. 28, A shows assay results for binding reactions of F4, F5, and F6 chimeric antibodies with OVCAR3 cells by FACS; B shows assay results for binding reactions of F4, F5, and F6 chimeric antibodies with HEK293T-hMSLN-B8 cells by FACS; C shows assay results for binding reactions of F4, F5, and F6 chimeric antibodies with HEK293T-hMSLN-R3 cells by FACS; D shows assay results for binding reactions of F4, F5, and F6 chimeric antibodies with HEK293T-monkey MSLN cells by FACS; E shows assay results for binding reactions of F4, F5, and F6 chimeric antibodies with 293T cells by FACS; and F shows assay results for binding reactions of F4, F5, and F6 chimeric antibodies with A431 cells by FACS.

In FIGS. 29, A and B show assay results for binding reactions of F7 chimeric antibodies with OVCAR3 cells by FACS; C and D show assay results for binding reactions of F7 chimeric antibodies with HEK293T-hMSLN-R3 cells by FACS; E and F show assay results for binding reactions of F7 chimeric antibodies with HEK293T-monkey MSLN cells by FACS; G and H show assay results for binding reactions of F7 chimeric antibodies with 293T cells by FACS; and I and J show assay results for binding reactions of F7 chimeric antibodies with A431 cells by FACS.

In FIGS. 30, A and B show assay results for binding reactions of F8 chimeric antibodies with OVCAR3 cells by FACS; C and D show assay results for binding reactions of F8 chimeric antibodies with HEK293T-hMSLN-R3 cells by FACS; E and F show assay results for binding reactions of F8 chimeric antibodies with HEK293T-monkey MSLN cells by FACS; G and H show assay results for binding reactions of F8 chimeric antibodies with 293T cells by FACS; and I and J show assay results for binding reactions of F8 chimeric antibodies with A431 cells by FACS.

FIGS. 31-37 show inhibition rates of chimeric antibodies as assayed by competitive ELISA.

In FIG. 38, A-H show assay results for binding reactions of humanized antibodies with human MSLN full-length protein by ELISA.

In FIG. 39, A-F show assay results for binding reactions of humanized antibodies with human MSLN-R3 protein by ELISA.

In FIG. 40A and FIG. 40B, A-J show assay results for binding reactions of humanized antibodies with HEK293T-hMSLN-R3 cells by FACS.

In FIG. 41A and FIG. 41B, A-J show assay results for binding reactions of humanized antibodies with HEK293T null cells by FACS.

In FIG. 42A and FIG. 42B, A-J show assay results for binding reactions of humanized antibodies with OVCAR3 cells by FACS.

In FIG. 43A and FIG. 43B, A-J show assay results for binding reactions of humanized antibodies with A431 cells by FACS.

In FIG. 44A and FIG. 44B, A-J show assay results for binding reactions of humanized antibodies with HEK293T-monkey MSLN cells by FACS.

### DETAILED DESCRIPTION

The present invention will be further described with reference to specific examples, and the advantages and features of the present invention will become more apparent with the description. Experimental procedures without specified conditions in the examples are conducted according to conventional conditions or conditions recommended by the manufacturers. Reagents or instruments without specified manufacturers used herein are conventional products that are commercially available.

The examples are exemplary only and do not limit the scope of the present invention in any way. It will be understood by those skilled in the art that various changes or substitutions in form and details may be made to the technical solutions of the present invention without departing from the spirit and scope of the present invention, and that these changes and substitutions shall fall within the scope of the present invention.

### EXAMPLE 1. PREPARATION OF CONTROL ANTIBODIES, IDENTIFICATION OF ENDOGENOUS CELLS, AND PREPARATION OF OVER-EXPRESSION CELL STRAIN

### 1.1. Preparation of Control Antibodies

YP218, YP3, and YP223 sequences were from the patent US2015252118A1, m912 sequence was from the patent WO2009120769A1, and Amatuximab sequence was from the patent US20140127237A1. VH and VL sequences of clone YP218 recognizing the epitope of human MSLN R3 and clone YP3 recognizing the conformational epitope of human MSLN were recombined into human IgG1 CH and CL expression vectors; VH and VL sequences of clone YP223 recognizing the epitope of human MSLN R2 were recombined into rabbit IgG1 CH and CL expression vectors; and VH and VL of clones m912 and YP218 recognizing the epitope of human MSLN R3 were linked by three GGGGS linkers and then recombined into a human IgG1 Fc expression vector to give a recombinant plasmid. Both the plasmid construction and the expression and purification of antibodies were completed by Biointron Biological Inc.

Amatuximab, a YP218 human IgG1 antibody, a YP223 rabbit IgG1 antibody, a YP3 human IgG1 antibody, a YP218 scFv-human IgG1 Fc (hFc) antibody, and an m912 scFv-human IgG1 Fc (hFc) antibody were designated as Tab 142 (Amatuximab), Tab106 (YP218, hIgG1), Tab020 (YP223, rabbitIgG1), Tab107 (YP3, hIgG1), Tab108 (YP218, scFv-hIgG1 Fc), and Tab 131 (m912, scFv-hIgG1 Fc), respectively.

TABLE 1
Sequence information of control antibodies

| Sequence name | Sequence No. | Amino acid sequence |
|---|---|---|
| YP223 VH | SEQ ID NO: 1 | QEQLEESGGDLVQPEGSLTLTCKASGLDFSSSYWICWVRQAPGK |
| | | GLEWIGCRHTFTANTWSASWVNGRFTISRSTSLGTVDLKMTSLT |
| | | AADTATYFCARDESNNDGWDFKLWGPGTLVTVSS |
| YP223 VL | SEQ ID NO: 2 | AYDMTQTPASVSAAVGGTVTIKCQASQSISNYLAWYQQKPGQPP |
| | | KLLIYQASTLAPGVSSRFKGSGSGTEFTLTISGVECADAATYYCQ |
| | | QGYTSSNVENVFGGGTGVVV |
| YP218 VH | SEQ ID NO: 3 | QQQLEESGGGLVKPEGSLTLTCKASGFDLGFYFYACWVRQAPGK |
| | | GLEWIACIYTAGSGSTYYASWAKGRFTISKASSTTVTLQMTSLAA |
| | | ADTATYFCARSTANTRSTYYLNLWGPGTLVTVSS |
| YP218 VL | SEQ ID NO: 4 | DVVMTQTPASVSEPVGGTVTIKCQASQRISSYLSWYQQKPGQRP |
| | | KLLIFGASTLASGVPSRFKGSGSGTEYTLTISDLECADAATYYCQ |
| | | SYAYFDSNNWHAFGGGTEVVV |
| YP3 VH | SEQ ID NO: 5 | QEQLVESGGGLVQPGASLTLTCTASGIDFSRYYMCWVRQAPGKG |
| | | LEGIACIYIGGSGSTYYASWAKGRFTISKASSTTVTLQMTSLTAAD |
| | | TATYFCARGTNLNYIFRLWGPGTLVTVSS |
| YP3 VL | SEQ ID NO: 6 | DVVMTQTPSPVSAAVGGTVTIKCQASQSINNGLAWYQQKPGQP |
| | | PRLLIYSASNLESGVPSRFKGSGSGTEFTLTISDLECDDAATYYCQ |
| | | CIWDGNSYVNAFGGGTEVVV |
| m912 scFv | SEQ ID NO: 7 | QVQLQESGPGLVKPSETLSLTCTVSGGSVSSGSYYWSWIRQPPG |
| | | KGLEWIGYIYYSGSTNYNPSLKSRVTISVDTSKNQFSLKLSSVTA |
| | | ADTAVYYCAREGKNGAFDIWGQGTMVTVSSGGGGSGGGGSGG |
| | | GGSDIQMTQSPSSLSASVGDRVTITCRASQSISSYLNWYQQKPGK |
| | | APKLLIYAASSLQSGVPSGFSGSGSGTDFTLTISSLQPEDFATYYC |
| | | QQSYSTPLTFGGGTKVEIK |
| YP218 scFv | SEQ ID NO: 8 | QQQLEESGGGLVKPEGSLTLTCKASGFDLGFYFYACWVRQAPGK |
| | | GLEWIACIYTAGSGSTYYASWAKGRFTISKASSTTVTLQMTSLAA |
| | | ADTATYFCARSTANTRSTYYLNLWGPGTLVTVSSGGGGSGGGGS |
| | | GGGGSDVVMTQTPASVSEPVGGTVTIKCQASQRISSYLSWYQQK |
| | | PGQRPKLLIFGASTLASGVPSRFKGSGSGTEYTLTISDLECADAAT |
| | | YYCQSYAYFDSNNWHAFGGGTEVVV |
| Amatuxmab VH- | SEQ ID NO: 9 | QVQLQQSGPELEKPGASVKISCKASGYSFTGYTMNWVKQSHGK |
| CH | | SLEWIGLITPYNGASSYNQKFRGKATLTVDKSSSTAYMDLLSLTS |
| | | EDSAVYFCARGGYDGRGFDYWGSGTPVTVSSASTKGPSVFPLAP |
| | | SSKSTSGGTAALGCLVKDYFPEPVTVSWNSGALTSGVHTFPAVLQSSG |
| | | LYSLSSVVTVPSSSLGTQTYICNVNHKPSNTKVDKKVEPKSCDKTHTC |
| | | PPCPAPELLGGPSVFLFPPKPKDTLMISRTPEVTCVVVDVSHEDPEV |
| | | KFNWYVDGVEVHNAKTKPREEQYNSTYRVVSVLTVLHQDWLNGKE |
| | | YKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVD |
| | | KSRWQQGNVFSCSVMHEALHNHYTQKSLSLSPGK |
| Amatuximab VL- | SEQ ID NO: 10 | DIELTQSPAIMSASPGEKVTMTCSASSSVSYMHWYQQKSGTSPK |
| CL | | RWIYDTSKLASGVPGRFSGSGSGNSYSLTISSVEAEDDATYYCQQ |
| | | WSKHPLTFGSGTKVEIKRTVAAPSVFIFPPSDEQLKSGTASVVCLLN |
| | | NFYPREAKVQWKVDNALQSGNSQESVTEQDSKDSTYSLSSTLTLSKA |
| | | DYEKHKVYACEVTHQGLSSPVTKSFNRGEC |
| hFc | SEQ ID NO: 11 | EPKSADKTHTCPPCPAPELLGGPSVFLFPPKPKDTLMISRTPEVTC |
| | | VVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSREEMTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPGK |
| CH-hIgG1 | SEQ ID NO: 12 | ASTKGPSVFPLAPSSKSTSGGTAALGCLVKDYFPEPVTVSWNSG |
| (human IgG1 | | ALTSGVHTFPAVLQSSGLYSLSSVVTVPSSSLGTQTYICNVNHKPS |
| heavy chain | | NTKVDKKVEPKSCDKTHTCPPCPAPELLGGPSVFLFPPKPKDTL |
| constant region) | | MISRTPEVTCVVVDVSHEDPEVKFNWYVDGVEVHNAKTKPREE |
| | | QYNSTYRVVSVLTVLHQDWLNGKEYKCKVSNKALPAPIEKTISK |
| | | AKGQPREPQVYTLPPSREEMTKNQVSLTCLVKGFYPSDIAVEWE |
| | | SNGQPENNYKTTPPVLDSDGSFFLYSKLTVDKSRWQQGNVFSCS |
| | | VMHEALHNHYTQKSLSLSPGK |
| CL-hIgG1 | SEQ ID NO: 13 | RTVAAPSVFIFPPSDEQLKSGTASVVCLLNNFYPREAKVQWKVD |
| (human IgG1 | | NALQSGNSQESVTEQDSKDSTYSLSSTLTLSKADYEKHKVYACE |
| light chain | | VTHQGLSSPVTKSFNRGEC |
| constant region) | | |
| CH-rabbitIgG1 | SEQ ID NO: 14 | GQPKAPSVFPLAPCCGDTPSSTVTLGCLVKGYLPEPVTVTWNSG |
| (rabbit IgG1 | | TLTNGVRTFPSVRQSSGLYSLSSVVSVTSSSQPVTCNVAHPATNT |
| heavy chain | | KVDKTVAPSTCSKPTCPPPELLGGPSVFIFPPKPKDTLMISRTPEV |
| constant region) | | TCVVVDVSQDDPEVQFTWYINNEQVRTARPPLREQQFNSTIRVV |
| | | STLPIAHQDWLRGKEFKCKVHNKALPAPIEKTISKARGQPLEPKV |
| | | YTMGPPREELSSRSVSLTCMINGFYPSDISVEWEKNGKAEDNYK |
| | | TTPAVLDSDGSYFLYSKLSVPTSEWQRGDVFTCSVMHEALHNHY |
| | | TQKSISRSPGK |
| CL-rabbitIgG1 | SEQ ID NO: 15 | GDPVAPTVLIFPPAADQVATGTVTIVCVANKYFPDVTVTWEVDG |
| (rabbit IgG1 light | | TTQTTGIENSKTPQNSADCTYNLSSTLTLTSTQYNSHKEYTCKVT |
| chain constant | | QGTTSVVQSFNRGDC |
| region) | | |

### 1.2. Preparation of Human MSLN-R3-rFc, MSLN-FL-his, MSLN-R1-his, MSLN-R2-his, and MSLN-R3-his

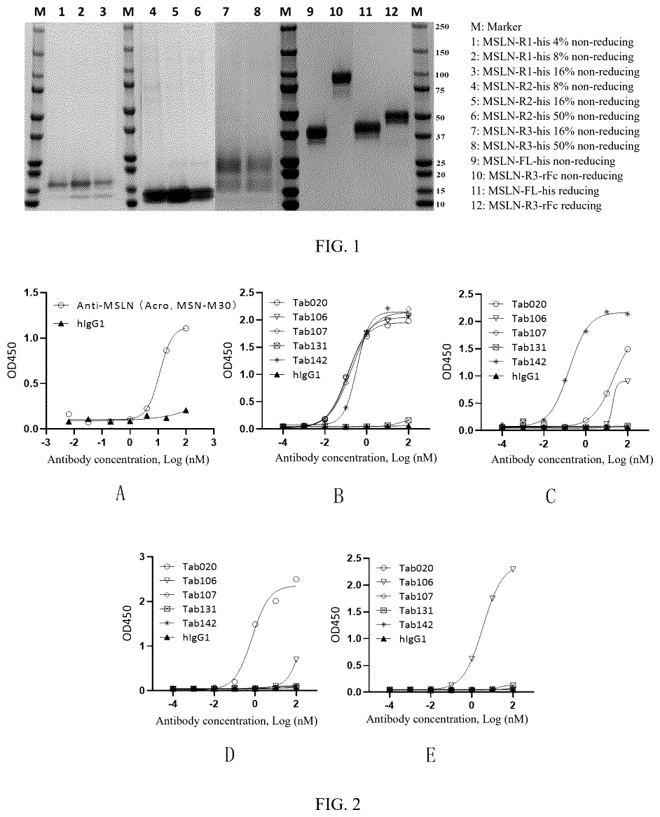

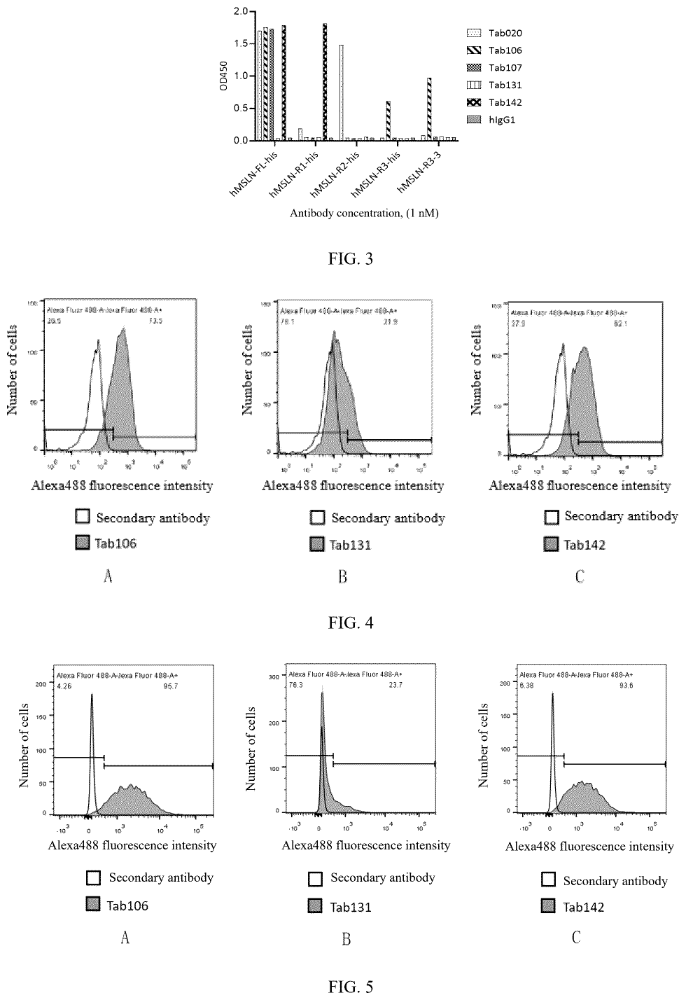

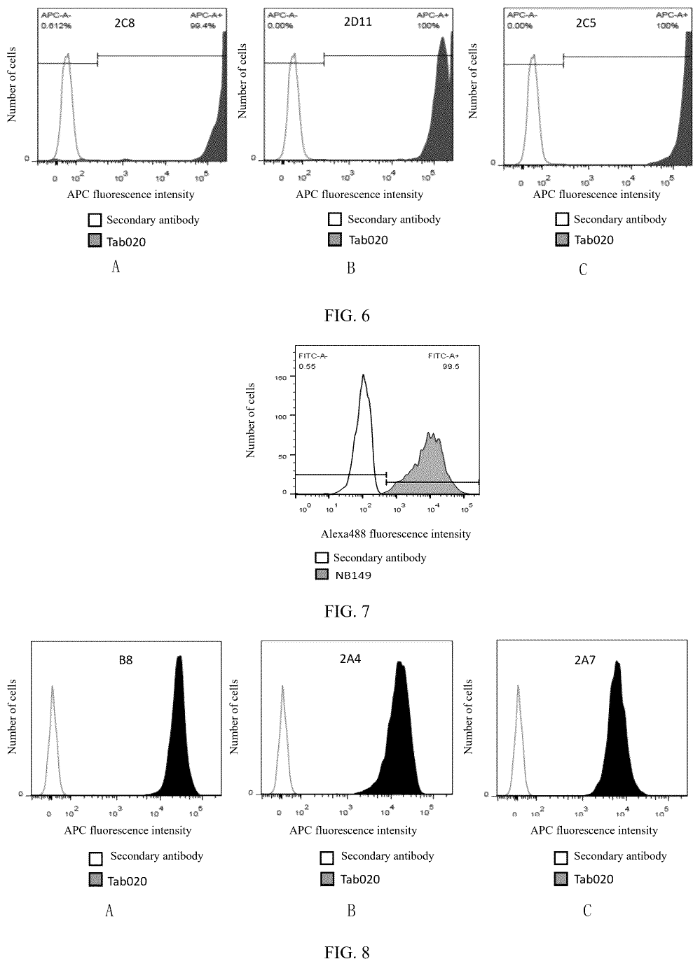

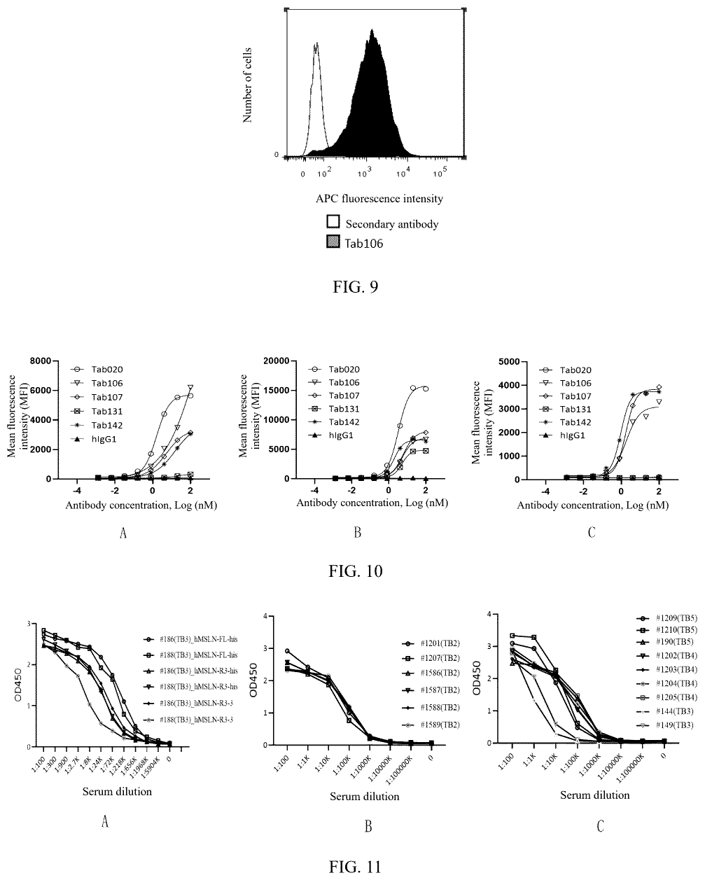

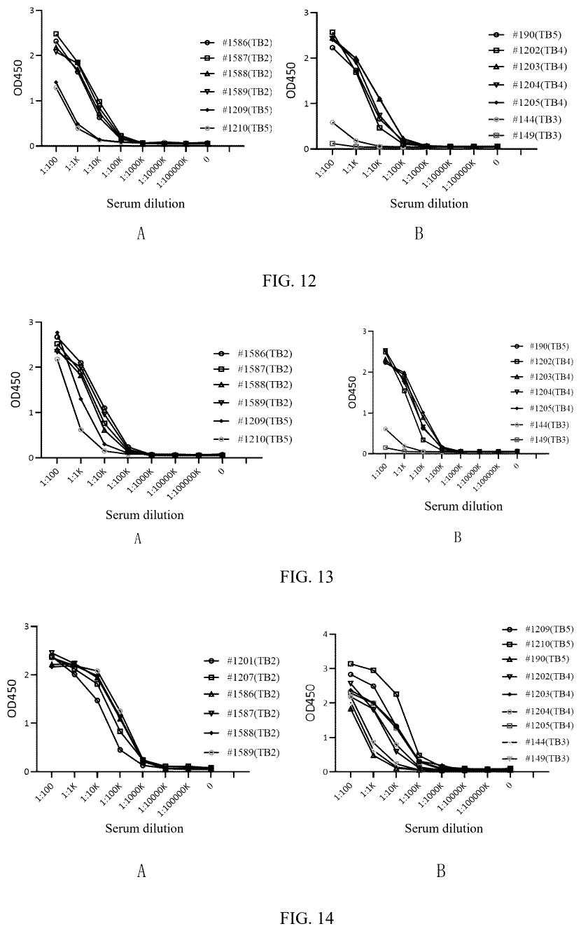

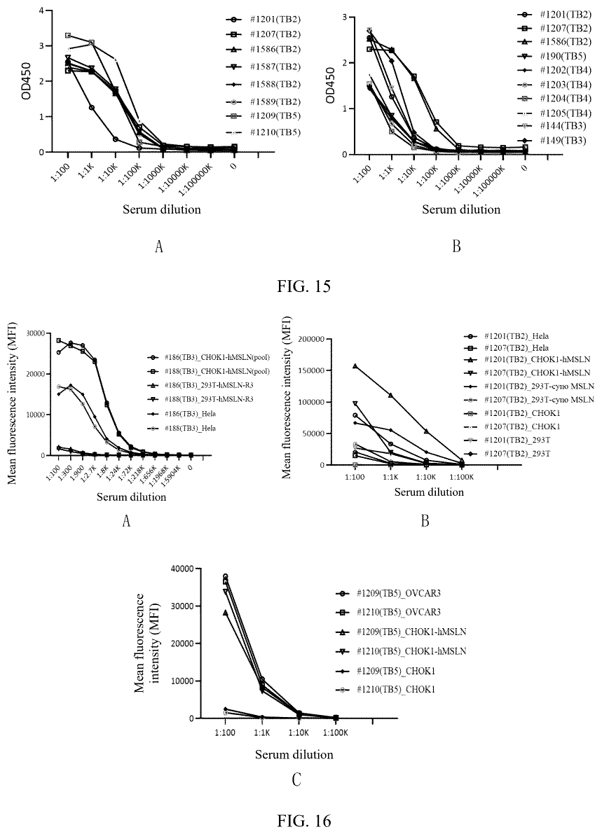

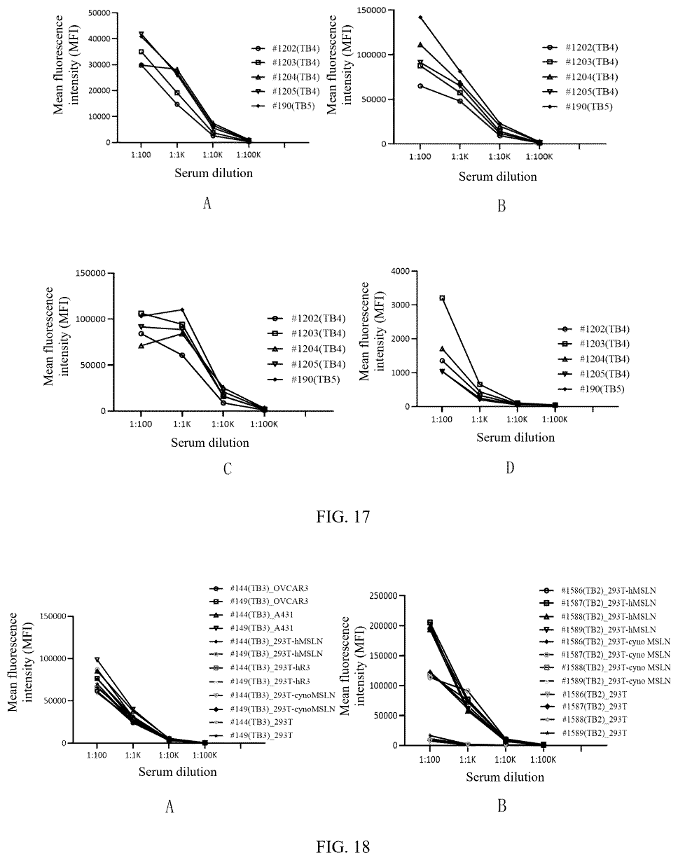

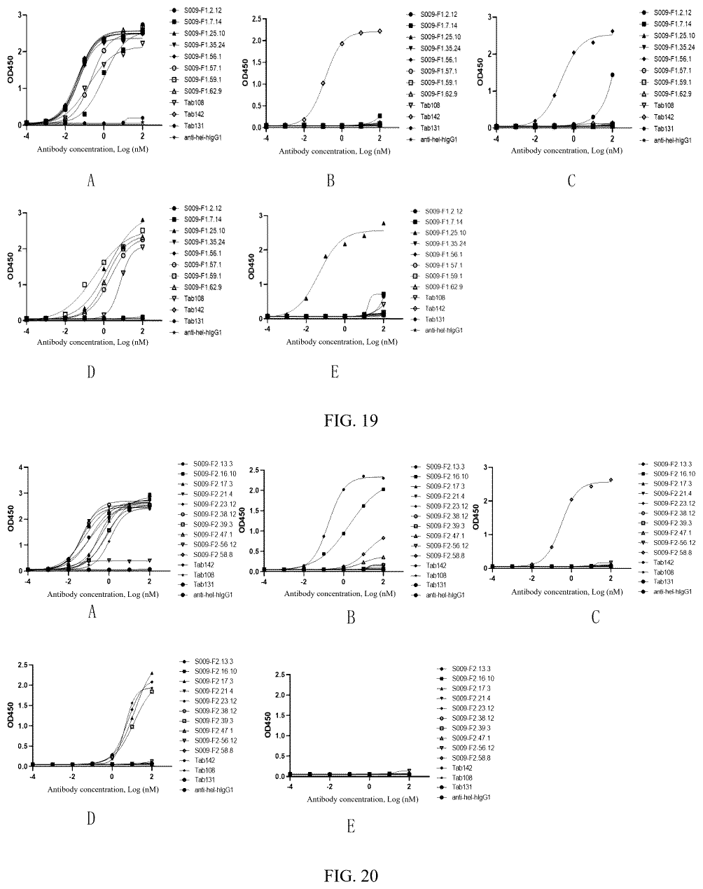

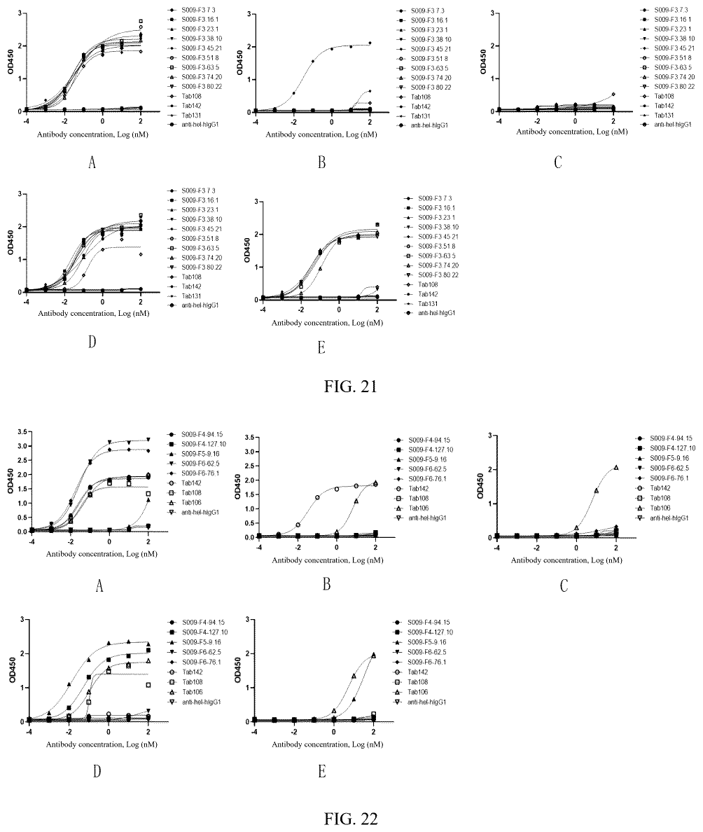

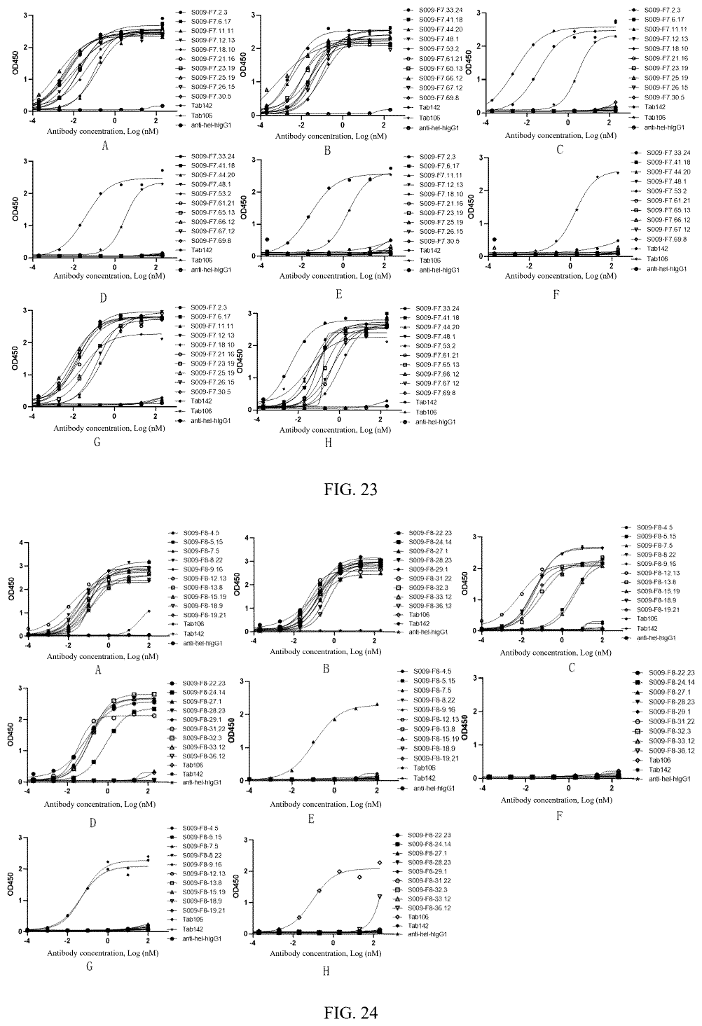

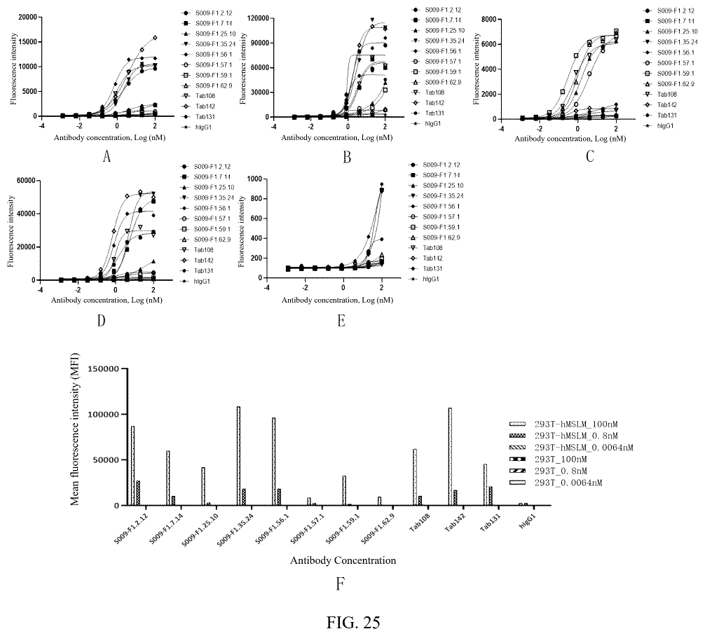

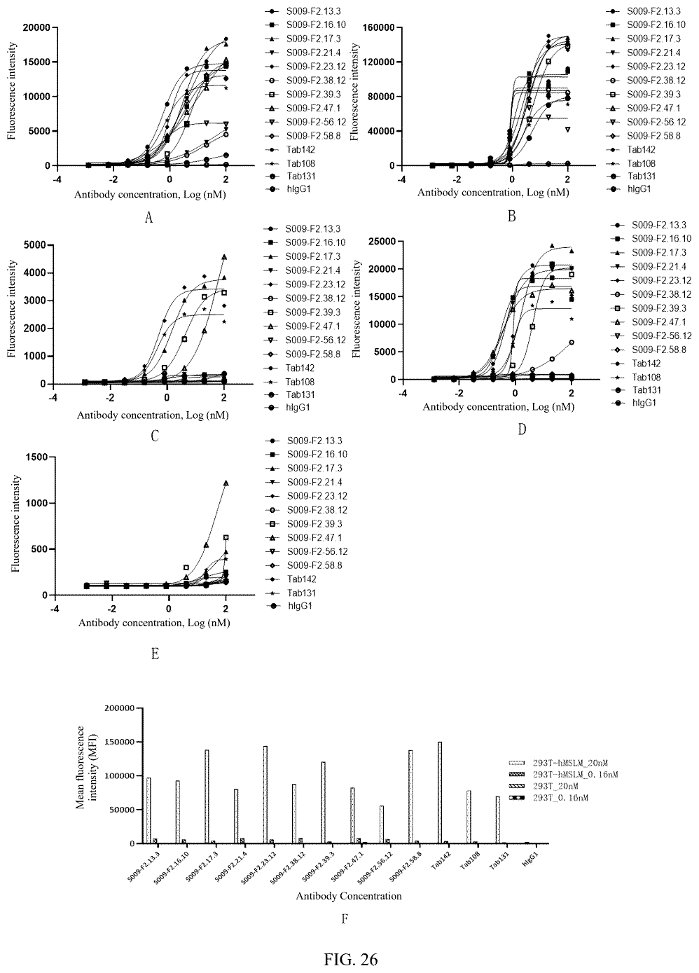

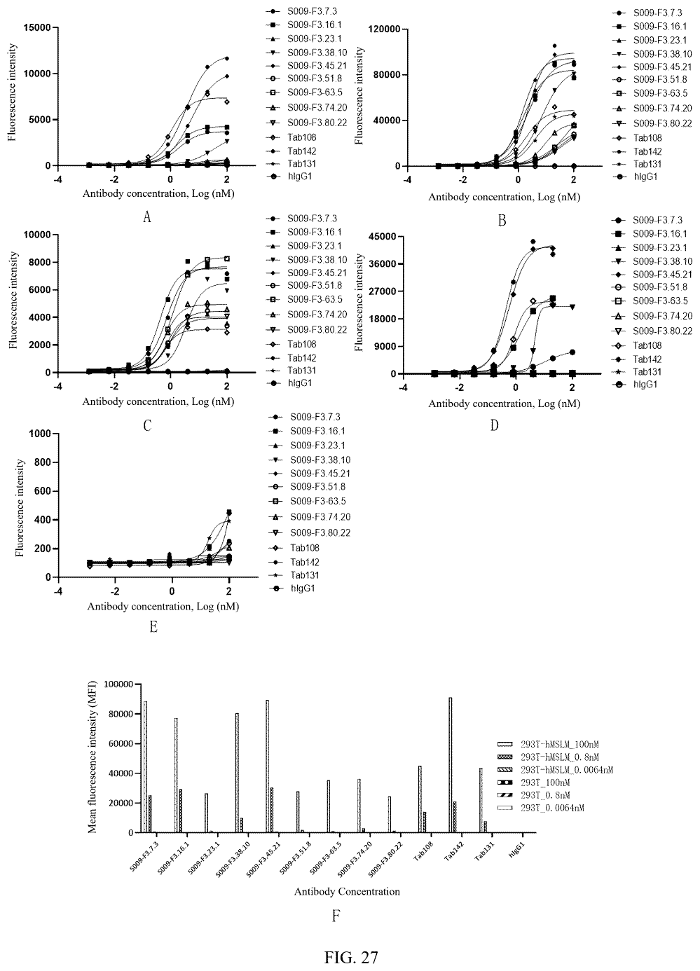

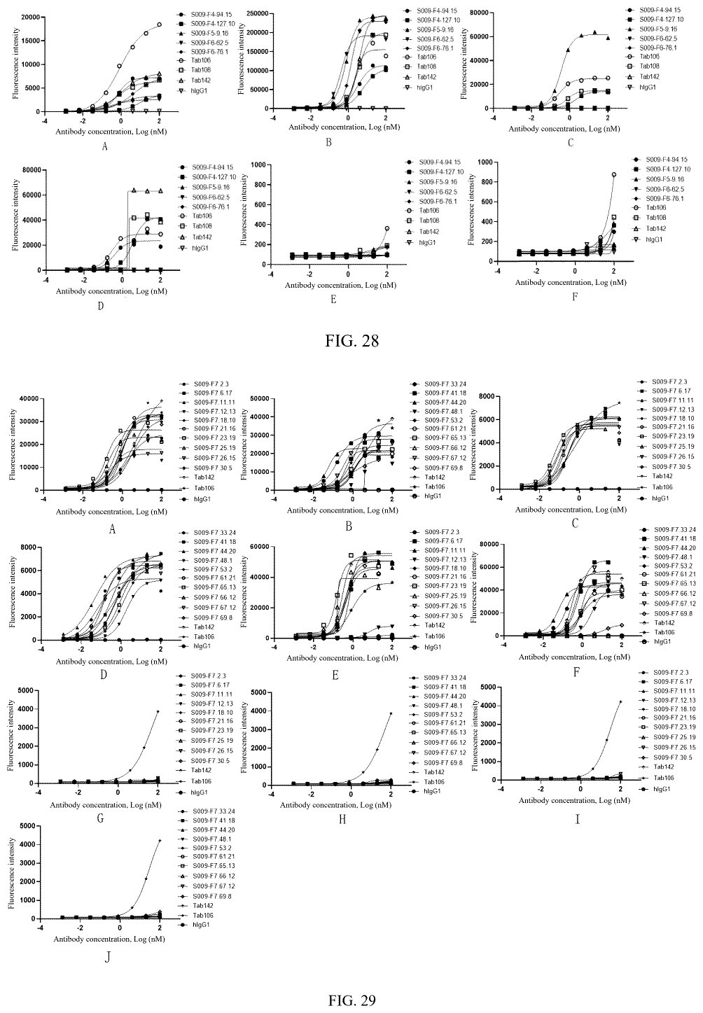

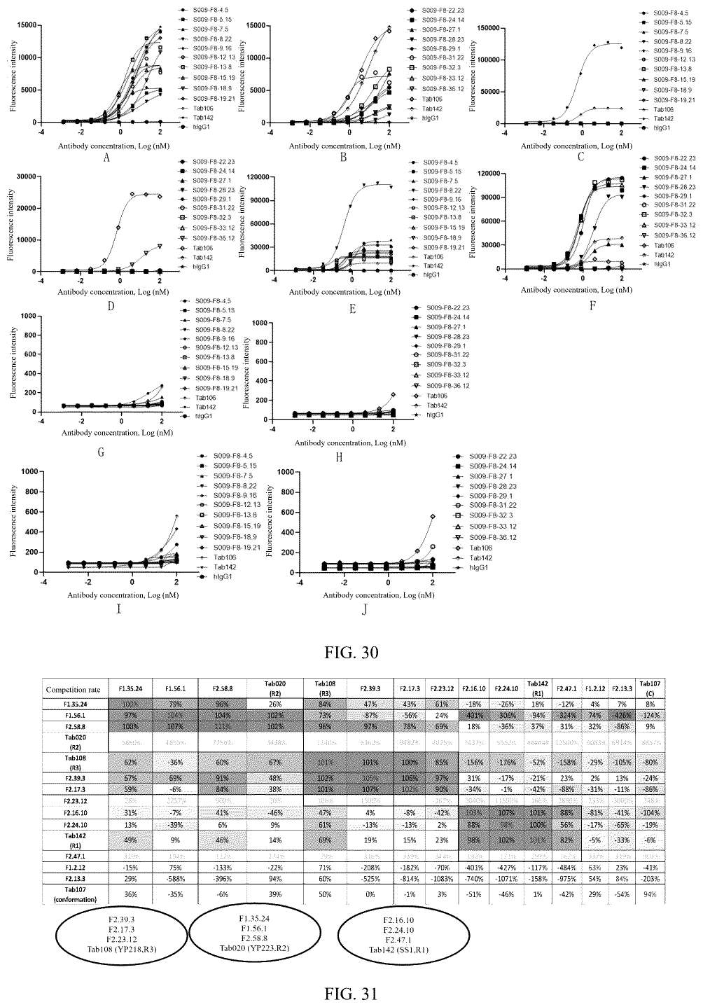

The MSLN protein has 3 extracellular IgG-like domains, wherein Region1 (R1) is located at the most distal membrane end and Region3 (R3) is located at the most proximal membrane end, with the antigen-binding epitope of Amatuximab located at R1 and YP218 at R3. Nucleotide sequences encoding the extracellular domain amino acid sequences Glu296-Gly580 (MSLN-FL), Glu296-Thr390 (MSLN-R1), Ser391-Asn486 (MSLN-R2), and Met487-Ser598 (MSLN-R3) of a human MSLN protein (NCBI: AAH09272.1) were separately cloned into a pTT5 vector (implemented by General Biol (Anhui) Co., Ltd), and plasmids were prepared according to an established standard molecular biology method. For details, see Sambrook, J., Fritsch, E. F., and Maniatis, T. (1989). Molecular Cloning: A Laboratory Manual, Second Edition (Plainview, New York: Cold Spring Harbor Laboratory Press). HEK293E cells (purchased from Suzhou Yiyan Biotech Co., Ltd.) were transiently transfected (PEI, Polysciences, Cat. No. 24765-1) and expanded at 37° C. using FreeStyle™ 293 (Thermofisher scientific, Cat. No. 12338018). After 6 days, the cell culture medium was collected and centrifuged to remove cell components to give a culture supernatant containing the extracellular domain of the human MSLN protein. The culture supernatant was loaded onto a nickel ion affinity chromatography column HisTrap™ Excel (GE Healthcare, Cat. No. GE17-3712-06), and meanwhile, the changes in UV absorption value (A280 nm) were monitored using an ultraviolet (UV) detector. After loading, the nickel ion affinity chromatography column was washed with 20 mM PB and 0.5 M NaCl (pH 7.4) until the UV absorption value returned to the baseline, and then gradient elution (2%, 4%, 8%, 16%, 50%, and 100%) was performed with buffer A (20 mM PB, 0.5 M NaCl (pH 7.4)) and buffer B (20 mM PB, 0.5 M NaCl, 500 mM imidazole). A His-tagged human MSLN protein eluted from the nickel ion affinity chromatography column was collected. The culture supernatant was loaded onto a protein A chromatography column (the protein A filler AT Protein A Diamond and the chromatography column BXK16/26 were both purchased from Bestchrom). The protein A chromatography column was washed with phosphate-buffered saline (PBS, pH 7.4) and 20 mM PB, 1 M NaCl (pH 7.2) in sequence, and was finally eluted with citric acid buffer (pH 3.4). A rabbit Fc (rFc)-tagged human MSLN protein eluted from the protein A chromatography column was collected. Dialysis was performed with phosphate-buffered saline (PBS, pH 7.4) at 4° C. overnight in a refrigerator. The dialyzed protein was subjected to 0.22 μM sterile filtration, subpackaged, and stored at −80° C., giving a purified human MSLN extracellular domain protein. The target bands of the sample as assayed by SDS-PAGE reducing gel and non-reducing gel are shown in FIG. 1.

Human MSLN protein (NCBI: AAH09272.1):
MALPTARPLLGSCGTPALGSLLFLLFSLGWVQPSRTLAGETGQEAAPLD
GVLANPPNISSLSPRQLLGFPCAEVSGLSTERVRELAVALAQKNVKLST
EQLRCLAHRLSEPPEDLDALPLDLLLFLNPDAFSGPQACTRFFSRITKA
NVDLLPRGAPERQRLLPAALACWGVRGSLLSEADVRALGGLACDLPGRF
VAESAEVLLPRLVSCPGPLDQDQQEAARAALQGGGPPYGPPSTWSVSTM
DALRGLLPVLGQPIIRSIPQGIVAAWRQRSSRDPSWRQPERTILRPRFR
REVEKTACPSGKKAPEIDESLIFYKKWELEACVDAALLATQMDRVNAIP
FTYEQLDVLKHKLDELYPQGYPESVIQHLGYLFLKMSPEDIRKWNVTSL
ETLKALLEVNKGHEMSPQVATLIDRFVKGRGQLDKDTLDTLTAFYPGYL
CSLSPEELSSVPPSSIWAVRPQDLDTCDPRQLDVLYPKARLAFQNMNGS
EYFVKIQSFLGGAPTEDLKALSQQNVSMDLATFMKLRTDAVLPLTVAEV
QKLLGPHVEGLKAEERHRPVRDWILRQRQDDLDTLGLGLQGGIPNGYLV
LDLSMQEALSGTPCLLGPGPVLTVLALLLASTLA.

The prepared human MSLN proteins described above were assayed by ELISA using positive control antibodies recognizing different epitopes, and the assay results are shown in FIG. 2 and Tables 2-6. The human MSLN-R3-rFc, MSLN-FL-his, MSLN-R1-his, MSLN-R2-his, and MSLN-R3-his proteins had binding activity to the anti-human MSLN antibody (purchased from Acro, Cat. No. MSN-M30) or the control antibodies, and were consistent with the binding epitopes of Tab 142 (Amatuximab), Tab106 (YP218), Tab020 (YP223), and Tab107 (YP3) reported in the product specifications or literature, indicating that the aforementioned proteins with binding activity have been prepared.

TABLE 2
Assay results for binding reactions of human MSLN-R3-rFc
protein with antibodies by ELISA
OD450 nm

Antibody

| Concentration (nM) | anti-hMSLN | hIgG1 |
|---|---|---|
| 100 | 1.11 | 0.21 |
| 20 | 0.86 | 0.12 |
| 4 | 0.23 | 0.15 |
| 1 | 0.11 | 0.09 |
| 0.2 | 0.08 | 0.08 |
| 0.032 | 0.07 | 0.11 |
| 0.0064 | 0.16 | 0.08 |
| 0 | 0.07 | 0.08 |

TABLE 3
Assay results for binding reactions of human MSLN-FL-his
protein with antibodies by ELISA
OD450 nm

| Concentration | Antibody | | | | | |
|---|---|---|---|---|---|---|
| (nM) | Tab020 | Tab106 | Tab107 | Tab131 | Tab142 | hIgG1 |
| 100 | 1.98 | 2.08 | 2.20 | 0.16 | 2.08 | 0.06 |
| 10 | 1.90 | 1.99 | 2.00 | 0.07 | 2.21 | 0.05 |
| 1 | 1.70 | 1.76 | 1.74 | 0.05 | 1.79 | 0.05 |
| 0.1 | 0.95 | 0.92 | 0.85 | 0.04 | 0.39 | 0.05 |
| 0.01 | 0.18 | 0.16 | 0.15 | 0.04 | 0.15 | 0.05 |
| 0.001 | 0.06 | 0.05 | 0.06 | 0.05 | 0.05 | 0.05 |
| 0.0001 | 0.05 | 0.04 | 0.05 | 0.05 | 0.05 | 0.04 |
| 0 | 0.05 | 0.05 | 0.05 | 0.05 | 0.05 | 0.05 |

TABLE 4
Assay results for binding reactions of human MSLN-R1-his
protein with antibodies by ELISA
OD450 nm

| Concentration | Antibody | | | | | |
|---|---|---|---|---|---|---|
| (nM) | Tab020 | Tab106 | Tab107 | Tab131 | Tab142 | hIgG1 |
| 100 | 1.49 | 0.90 | 0.08 | 0.09 | 2.14 | 0.06 |
| 10 | 0.68 | 0.12 | 0.06 | 0.05 | 2.18 | 0.05 |
| 1 | 0.19 | 0.06 | 0.05 | 0.06 | 1.82 | 0.05 |
| 0.1 | 0.07 | 0.05 | 0.05 | 0.05 | 0.92 | 0.05 |
| 0.01 | 0.05 | 0.04 | 0.06 | 0.11 | 0.19 | 0.05 |
| 0.001 | 0.10 | 0.17 | 0.05 | 0.14 | 0.07 | 0.05 |
| 0.0001 | 0.05 | 0.04 | 0.04 | 0.05 | 0.11 | 0.05 |
| 0 | 0.06 | 0.05 | 0.05 | 0.05 | 0.05 | 0.05 |

TABLE 5
Assay results for binding reactions of human MSLN-R2-his
protein with antibodies by ELISA
OD450 nm

| Concentration | Antibody | | | | | |
|---|---|---|---|---|---|---|
| (nM) | Tab020 | Tab106 | Tab107 | Tab131 | Tab142 | hIgG1 |
| 100 | 2.50 | 0.70 | 0.09 | 0.10 | 0.11 | 0.06 |
| 10 | 2.01 | 0.11 | 0.05 | 0.05 | 0.09 | 0.05 |
| 1 | 1.49 | 0.05 | 0.05 | 0.04 | 0.07 | 0.05 |
| 0.1 | 0.20 | 0.05 | 0.04 | 0.05 | 0.05 | 0.05 |
| 0.01 | 0.07 | 0.04 | 0.04 | 0.04 | 0.04 | 0.05 |
| 0.001 | 0.05 | 0.04 | 0.05 | 0.04 | 0.05 | 0.05 |
| 0.0001 | 0.05 | 0.04 | 0.04 | 0.04 | 0.05 | 0.05 |
| 0 | 0.05 | 0.05 | 0.05 | 0.05 | 0.05 | 0.04 |

TABLE 6
Assay results for binding reactions of human MSLN-R3-his
protein with antibodies by ELISA
OD450 nm

| Concentration | Antibody | | | | | |
|---|---|---|---|---|---|---|
| (nM) | Tab020 | Tab106 | Tab107 | Tab131 | Tab142 | hIgG1 |
| 100 | 0.05 | 2.29 | 0.09 | 0.12 | 0.05 | 0.05 |
| 10 | 0.05 | 1.75 | 0.05 | 0.05 | 0.05 | 0.05 |
| 1 | 0.05 | 0.62 | 0.05 | 0.04 | 0.04 | 0.05 |
| 0.1 | 0.05 | 0.12 | 0.05 | 0.04 | 0.05 | 0.05 |
| 0.01 | 0.05 | 0.06 | 0.04 | 0.03 | 0.04 | 0.05 |
| 0.001 | 0.05 | 0.05 | 0.05 | 0.05 | 0.05 | 0.04 |
| 0.0001 | 0.05 | 0.05 | 0.05 | 0.05 | 0.05 | 0.04 |
| 0 | 0.05 | 0.05 | 0.05 | 0.05 | 0.05 | 0.05 |

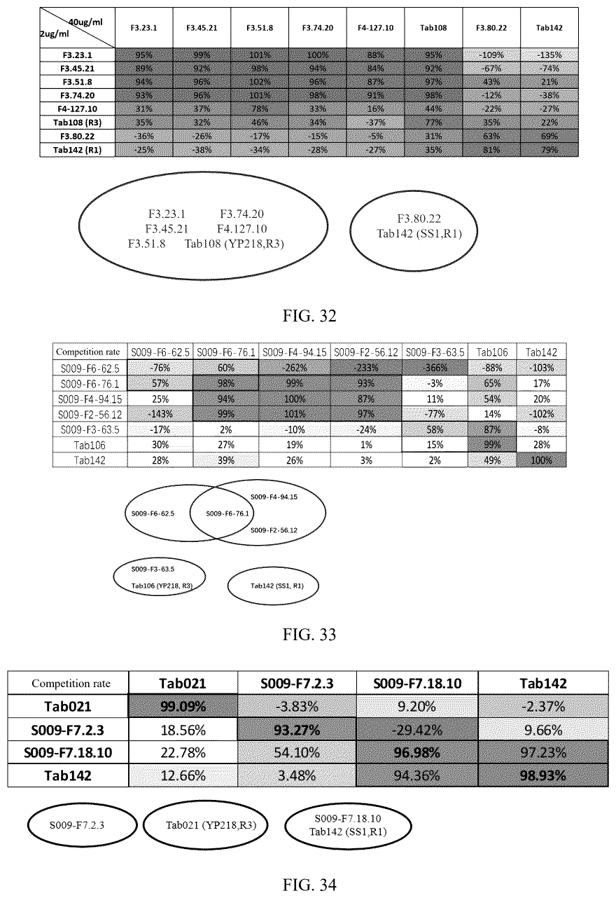

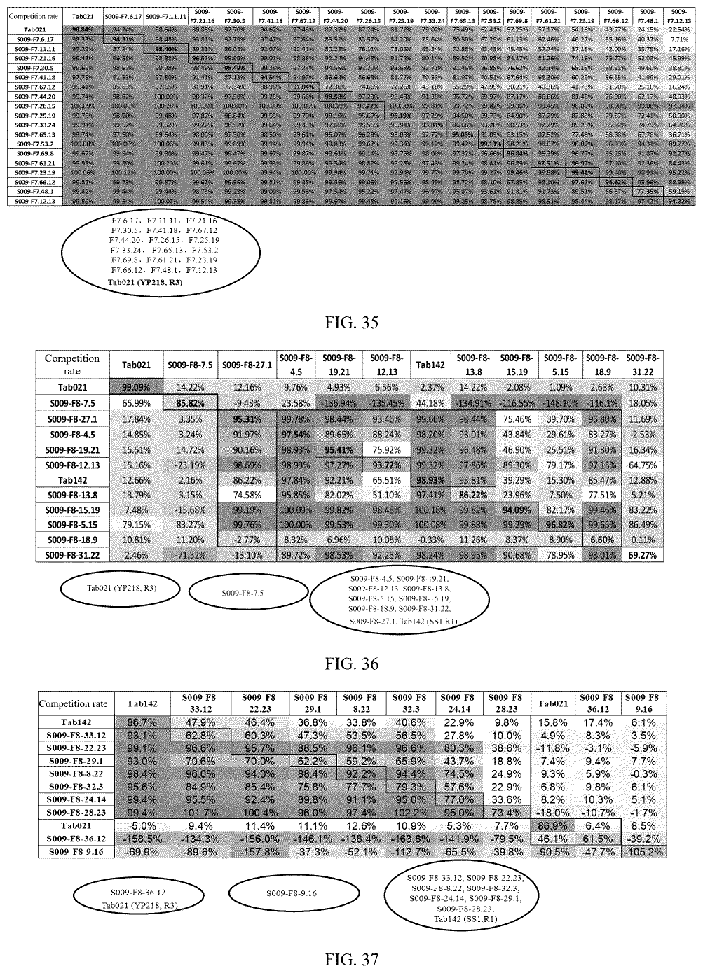

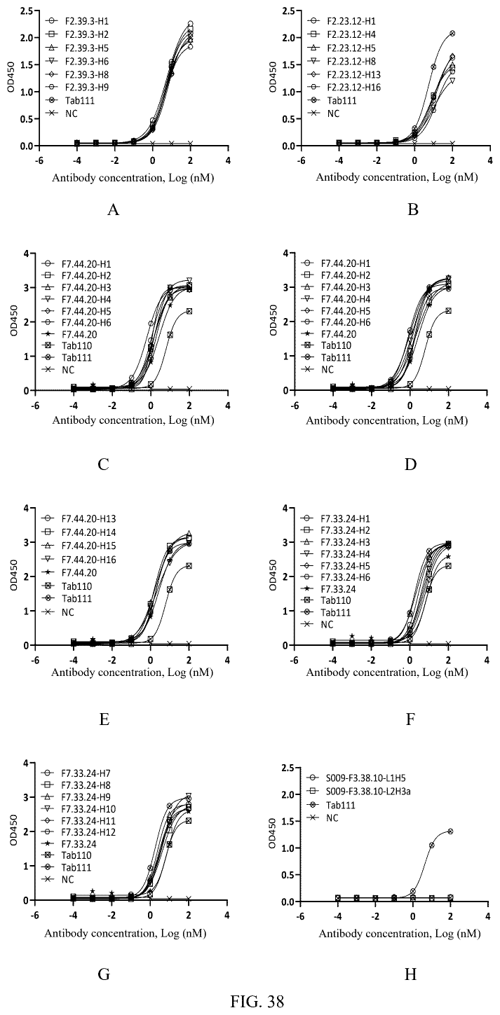

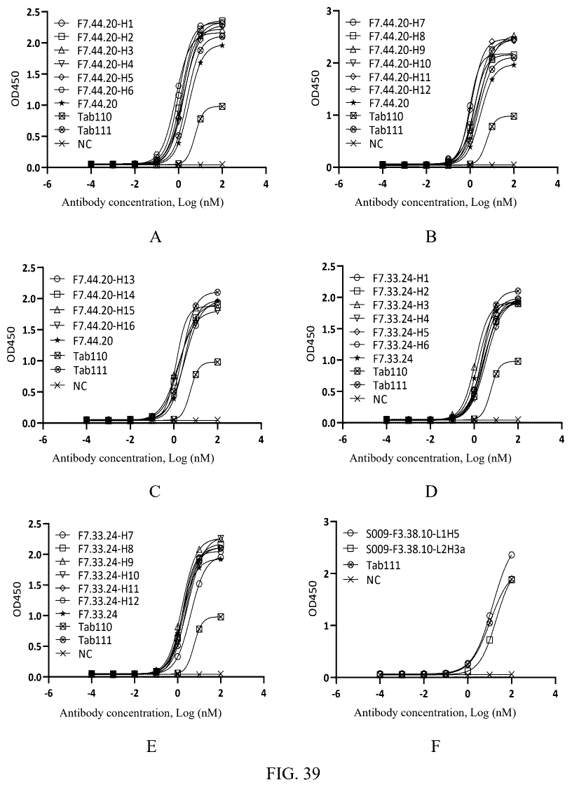

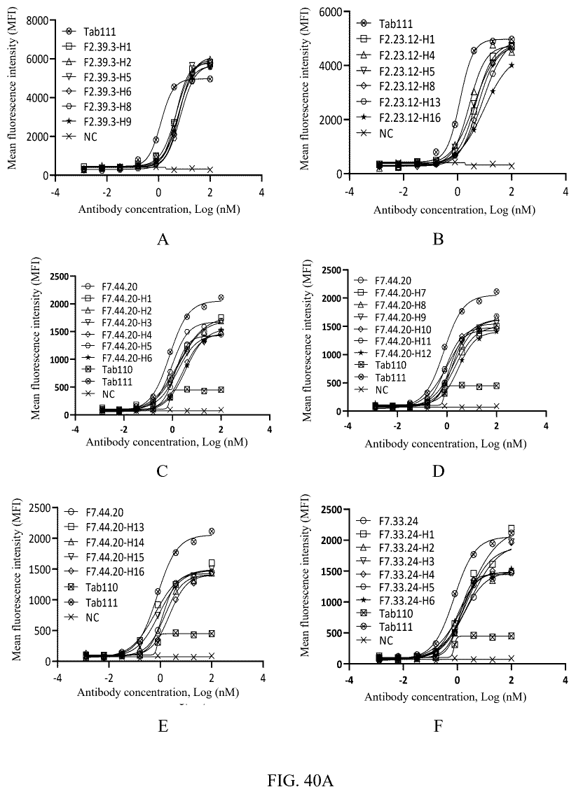

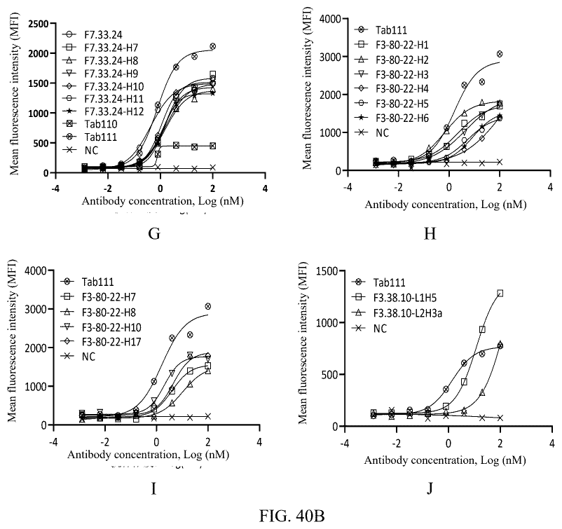

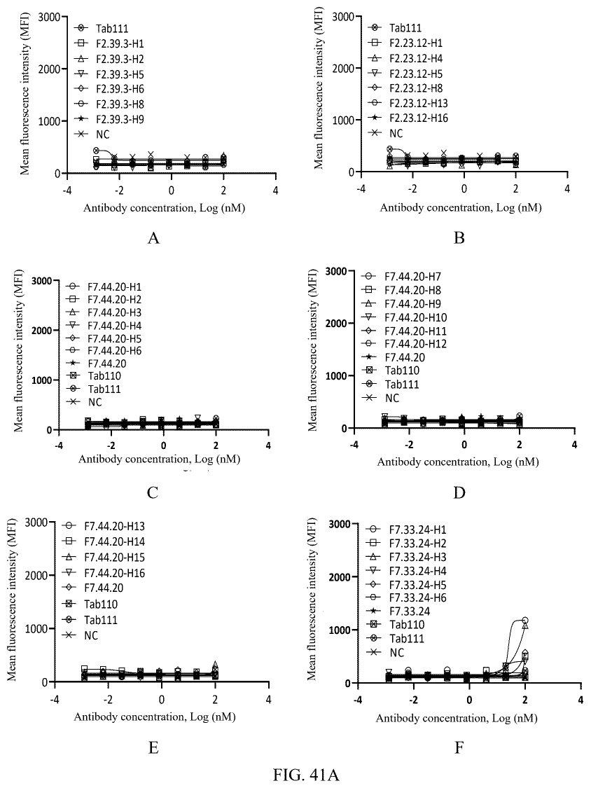

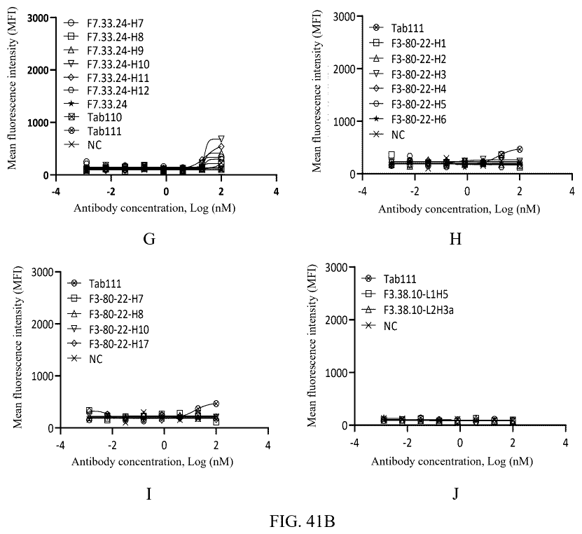

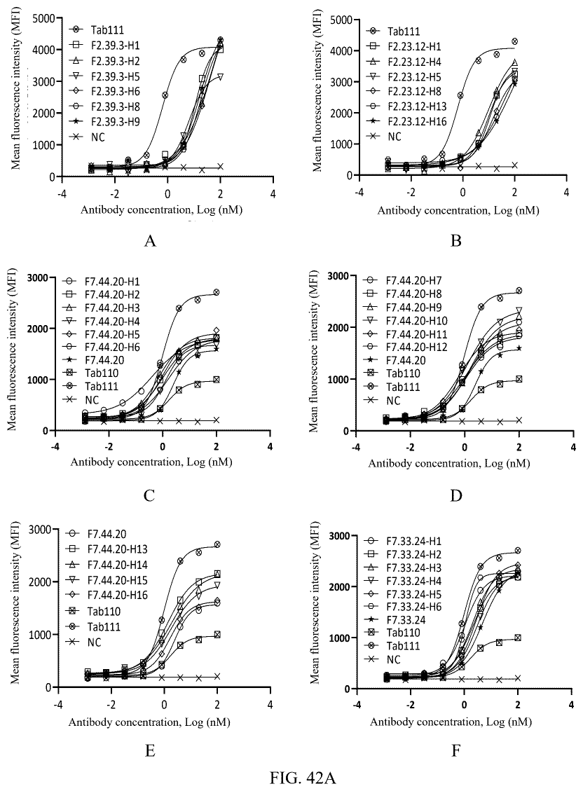

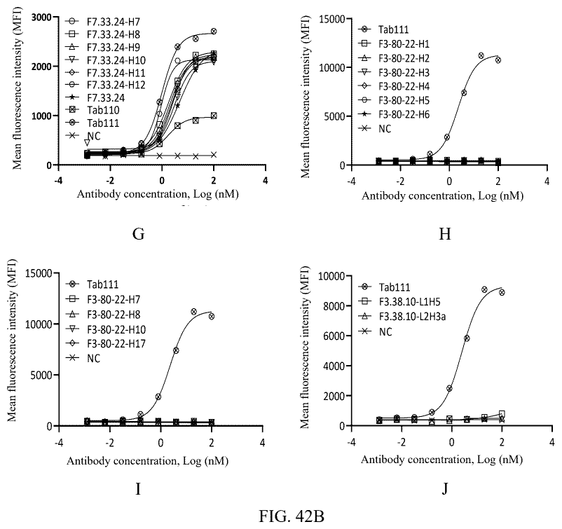

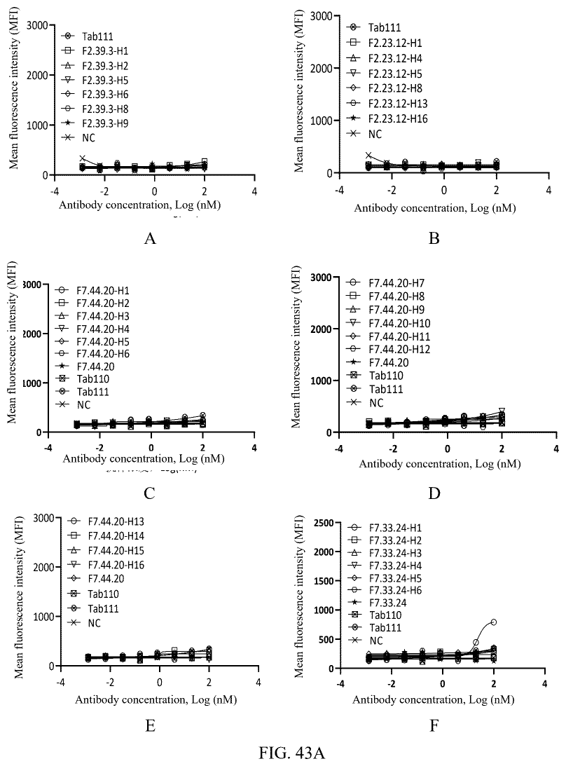

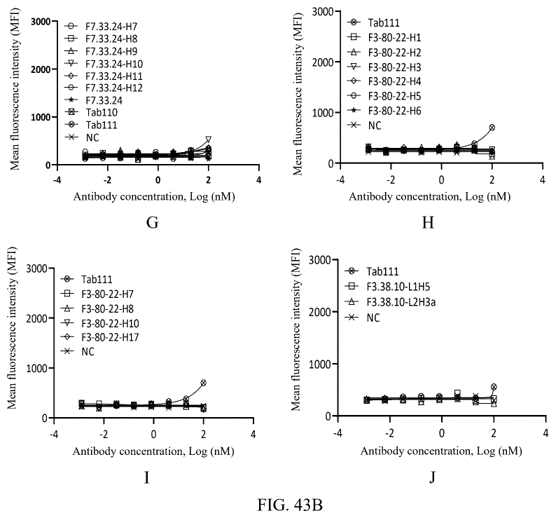

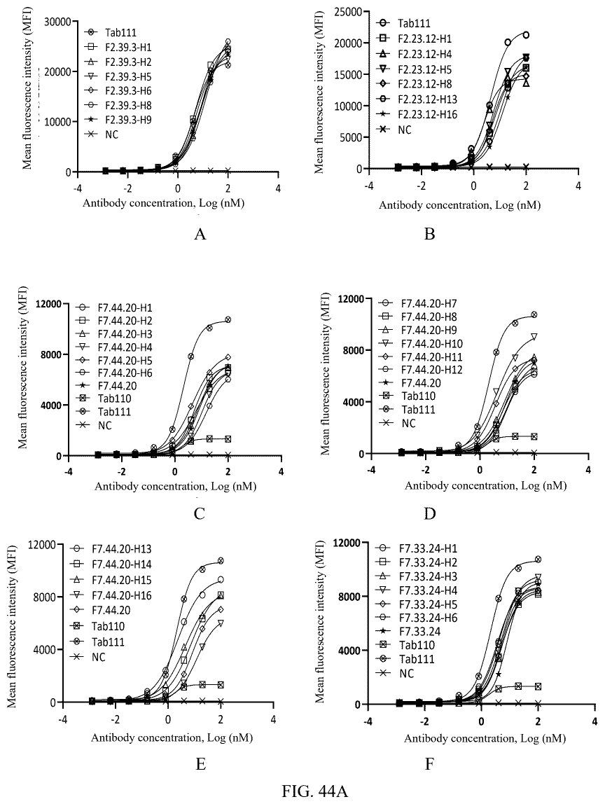

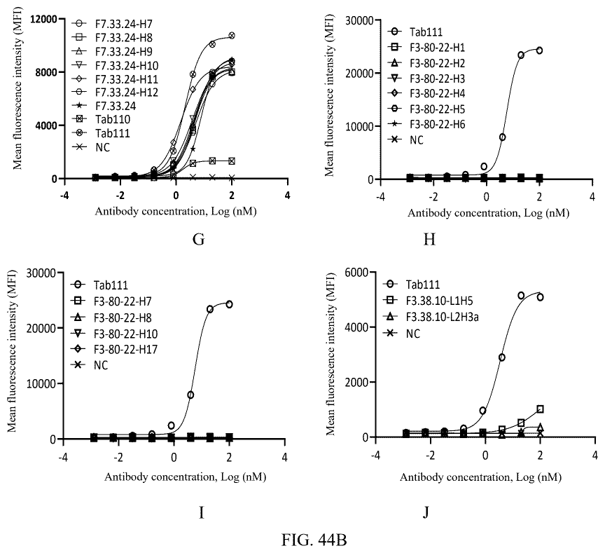

The binding activity of the control antibodies to the human MSLN-FL-His protein, MSLN-R1-His protein, MSLN-R2-His protein, MSLN-R3-His protein, and MSLN-R3-3 polypeptide (R3-3 is a smaller epitope in R3, purchased from GL Biochem, Cat. No. 406676) are shown in Table 7 and FIG. 3, and the results show that Tab020 (YP223), Tab142 (Amatuximab), Tab 106 (YP218), and Tab107 (YP3) antibodies had good binding activity to the human MSLN-FL-His protein and that Tab 131 (m912 scFv-hFc) had almost no binding activity to the human MSLN-FL-his protein under the same experimental conditions.

TABLE 7
Assay results for binding reactions of control antibodies
with human MSLN-FL-his protein by ELISA
OD450 nm

| | Antibody (1 nM) | | | | | |
|---|---|---|---|---|---|---|
| Antigen | Tab020 | Tab106 | Tab107 | Tab131 | Tab142 | hIgG1 |
| hMSLN-FL-his | 1.70 | 1.76 | 1.74 | 0.05 | 1.79 | 0.05 |
| hMSLN-R1-his | 0.19 | 0.06 | 0.05 | 0.06 | 1.82 | 0.05 |
| hMSLN-R2-his | 1.49 | 0.05 | 0.05 | 0.04 | 0.07 | 0.05 |
| hMSLN-R3-his | 0.05 | 0.62 | 0.05 | 0.04 | 0.04 | 0.05 |
| hMSLN-R3-3 | 0.09 | 0.98 | 0.07 | 0.07 | 0.06 | 0.06 |

### 1.3. Identification of Cell Strains Endogenously Expressing Human MSLN Protein

Cells endogenously expressing a human MSLN protein were expanded to the logarithmic growth phase in a T-75 cell culture flask, the medium supernatant was discarded by centrifugation, and the cell pellet was washed twice with PBS. 20 nM Tab 106, Tab131 and Tab 142 antibodies were used as primary antibodies, and an FITC-labeled secondary antibody (purchased from Invitrogen, Cat. No. A18830) was assayed and analyzed by FACS (FACS Canto™, purchased from BD). The results are shown in Table 8, FIG. 4, and FIG. 5, indicating that the cells endogenously expressing the human MSLN protein had binding activity to all of Tab 106, Tab 131, and Tab 142.

TABLE 8
Expression level of MSLN in tumor cells as assayed by FACS

| | Endogenous- | Mean Fluorescence Intensity | | | |
|---|---|---|---|---|---|
| | expression | Secondary | | | |
| No. | cell line | antibody control | Tab106 | Tab131 | Tab142 |
| 1 | Hela | 71 | 647 | 219 | 533 |
| 2 | OVCAR3 | 75 | 3484 | 211 | 2096 |

### 1.4. Preparation of CHO-K1 Recombinant Cell Strains Expressing Human MSLN Full-Length Protein

A nucleotide sequence encoding a full-length amino acid sequence of human MSLN (NCBI: AAH09272.1) was cloned into a pcDNA3.1 vector, and a plasmid was prepared (implemented by General Biol (Anhui) Co., Ltd). After plasmid transfection (Lipofectamine® 3000 Transfection Kit, purchased from Invitrogen, Cat. No. L3000-015) of a CHO-K1 cell line (purchased from Shanghai Institutes for Biological Sciences, Chinese Academy of Sciences), the cells were selectively cultured in a DMEM/F12 medium containing 10 μg/mL puromycin and 10% (w/w) fetal bovine serum for 2 weeks, and positive monoclonal cells were sorted onto a 96-well plate on a flow cytometer FACSAriaII (purchased from BD Biosciences) using a rabbit anti-human MSLN antibody (Tab020) and a goat anti-rabbit IgG Fab antibody (cell signaling, Cat. No. 4414S) and cultured under the conditions of 37° C. and 5% (v/v) CO₂. After about 2 weeks, some of the monoclonal wells were selected for amplification. The amplified clones were screened by flow cytometry. Monoclonal cell lines with better growth and higher fluorescence intensity were selected for further expansion and cryopreserved in liquid nitrogen.

The specific selection results are shown in Table 9 and FIG. 6, with samples only incubated with the secondary antibody as controls. Table 9 indicates that a series of CHO-K1 monoclonal cell lines with positive expression of human MSLN have been prepared. In FIG. 6, the abscissa represents the fluorescence intensity of the cells, and the ordinate represents the number of the cells. The results indicate that 2C8, 2D11, and 2C5 are recombinant CHO-K1 cell strains highly expressing human MSLN protein.

TABLE 9
Assay results of CHO-K1 recombinant cell lines expressing
human MSLN full-length protein by FACS

| | | Mean fluorescence intensity of cells | |
|---|---|---|---|
| | Clone No. of stably | Secondary antibody | Tab020 |
| No. | transfected cell line | control | antibody |
| 1 | CHO-K1-hMSLN-2C8 | 37 | 233609 |
| 2 | CHO-K1-hMSLN-2D11 | 37 | 217642 |
| 3 | CHO-K1-hMSLN-2C5 | 37 | 205726 |

### 1.5. Preparation of Recombinant HEK293T Cell Strain Expressing Monkey MSLN Protein

A nucleotide sequence encoding a full-length amino acid sequence of monkey MSLN (NCBI: XP_028696439.1) was cloned into a pcDNA3.1 vector, and a plasmid was prepared. After the plasmid transfection (Lipofectamine® 3000 Transfection Kit, purchased from Invitrogen, Cat. No. L3000-015) of an HEK293T cell line (purchased from ATCC), the cells were selectively cultured in a DMEM/F12 medium containing 10 μg/mL puromycin and 10% (w/w) fetal bovine serum for 2 weeks, subcloned in a 96-well culture plate by a limiting dilution method, and cultured under the conditions of 37° C. and 5% (v/v) CO₂. After about 2 weeks, some of the polyclonal wells were selected for amplification in a 6-well plate. The amplified clones were assayed and analyzed by an FACS flow cytometer using an NB149 antiserum, and the cell strains with better growth and higher fluorescence intensity were selected for further expansion and cryopreserved in liquid nitrogen. The results for the expression level are shown in Table 10 and FIG. 7, showing that HEK293T-monkey-MSLN screened with puromycin under pressure has a relatively single positive peak and thus can be used for assaying the cross activity of antibodies to the monkey MSLN protein by FACS.

Full-length amino acid sequence of monkey MSLN (NCBI: XP_028696439.1):

MALPMARPLSGSCGTPAVGSLLFLLFSLGWVQPSRVLAGETRQALCPQE
AAPLDGILTNAPDIASLSPRQLLGFTCVEVSGLSTELVQELAVALGQKN
VKLSAEQLRCLAHRLSEPPEDLDALPLDLLLFLNPDAFSGPQACTHFFS
RVAKANVDLLPRGAPERQRLLPAALTCWGVRGSLLSEADVRALGGLACD
LPGCFVAESAEVVLPRLVRCLGPLDQDQQEAARAALQRGGPPYGPPSTW
SISTLDDLQSLLPVLGQPVIHSIPKGILAAWRQRSSRDPSWQQPEQTVL
RPRFRRDVERTTCPPEKEVHEIDESLIFYKKRELEACVDAALLAAQMDR
VDAIPFTYEQLDVLKHKLDELYPQGYPESVIRHLGHLFLKMSPEDIRKW
NVTSLETLKALLKVSKGHEMSAQVATLIDRVVVGRGQLDKDTVDTLTAF
CPGCLCSLSPERLSSVPPSVIGAVRPQDLDTCGPRQLDVLYPKARLAFQ
NMSGSEYFVKIRPFLGGAPTEDVKALSQQNVSMDLATFMKLRREAVLPL
TVAEVQKLLGPHVEGLKVEEQHSPVRDWILKQRQDDLDTLGLGLQGGIP
NGYLILDLSVREALSGTPCLLGPGPVLTVLALLLASTLA.

TABLE 10
Assay results of HEK293T recombinant cell line expressing
monkey MSLN full-length protein by FACS

| | | Mean fluorescence intensity of cells | |
|---|---|---|---|
| | Clone No. of stably | Secondary antibody | NB149 |
| No. | transfected cell line | control | antiserum |
| 1 | HEK293T-monkey MSLN | 105 | 12423 |

### 1.6. Preparation of Recombinant HEK293T Cell Strains Expressing Human MSLN Protein

A nucleotide sequence encoding a full-length amino acid sequence of human MSLN (NCBI: AAH09272.1) was cloned into a pcDNA3.1 vector, and a plasmid was prepared. After plasmid transfection (Lipofectamine® 3000 Transfection Kit, purchased from Invitrogen, Cat. No. L3000-015) of an HEK293T cell line (purchased from ATCC), the cells were selectively cultured in a DMEM medium containing 5 μg/mL puromycin and 10% (w/w) fetal bovine serum for 2 weeks, and positive monoclonal cells were sorted onto a 96-well plate on a flow cytometer FACSAriaII (purchased from BD Biosciences) using a rabbit anti-human MSLN antibody (Tab020) and a goat anti-rabbit IgG Fab antibody (cell signaling, Cat. No. 4414S) and cultured under the conditions of 37° C. and 5% (v/v) CO₂. After about 2 weeks, some of the monoclonal wells were selected for amplification. The amplified clones were assayed and analyzed by an FACS flow cytometer using a Tab020 antibody, and the cell strains with better growth and higher fluorescence intensity were selected for further expansion and cryopreserved in liquid nitrogen. The results for the expression level are shown in Table 11 and FIG. 8, showing that HEK293T-human MSLN screened with puromycin under pressure has a single positive peak and that B8, 2A4, and 2A7 are recombinant HEK293T cell strains highly expressing the human MSLN protein and thus can be used for assaying the binding activity of antibodies to the human MSLN protein by FACS.

TABLE 11
Assay results of HEK293T recombinant cell lines
expressing human MSLN full-length protein by FACS

| | | Mean fluorescence intensity of cells | |
|---|---|---|---|
| | Clone No. of stably | Secondary antibody | Tab020 |
| No. | transfected cell line | control | antibody |
| 1 | HEK293T-hMSLN-B8 | 1 | 24400 |
| 2 | HEK293T-hMSLN-2A4 | 1 | 15400 |
| 3 | HEK293T-hMSLN-2A7 | 1 | 6581 |

### 1.7. Preparation of Recombinant HEK293T Cell Strain Expressing Human MSLN-R3 Protein

A nucleotide sequence encoding the amino acid sequence of human MSLN-R3 (NCBI: Met487-Ser606 of AAH09272.1) was cloned into a pcDNA3.1 vector, and a plasmid was prepared. After plasmid transfection (Lipofectamine® 3000 Transfection Kit, purchased from Invitrogen, Cat. No. L3000-015) of an HEK293T cell line (purchased from ATCC), the cells were selectively cultured in a DMEM medium containing 5 μg/mL puromycin and 10% (w/w) fetal bovine serum for 2 weeks, and positive monoclonal cells were sorted onto a 96-well plate on a flow cytometer FACSAriaII (purchased from BD Biosciences) using an anti-human MSLN-R3 antibody (Tab 106) and a goat anti-human IgG H+L antibody (Jackson, Cat. No. 109605088) and cultured under the conditions of 37° C. and 5% (v/v) CO₂. After about 2 weeks, some of the monoclonal wells were selected for amplification. The amplified clones were assayed and analyzed by an FACS flow cytometer using a Tab 106 antibody, and the cell strains with better growth and higher fluorescence intensity were selected for further expansion and cryopreserved in liquid nitrogen. The results for the expression level are shown in Table 12 and FIG. 9, showing that HEK293T-human MSLN-R3 screened with puromycin under pressure has a relatively single positive peak and thus can be used for assaying the binding activity of antibodies to the human MSLN-R3 protein by FACS.

TABLE 12
Assay results of HEK293T recombinant cell line
expressing human MSLN-R3protein by FACS

| | | Mean fluorescence intensity of cells | |
|---|---|---|---|
| | Clone No. of stably | Secondary antibody | Tab106 |
| No. | transfected cell line | control | antibody |
| 1 | HEK293T-hMSLN-R3 | 100 | 1689 |

### 1.8. Assay on Binding of Recombinant Cell Lines to Control Antibodies

The binding activity of the control antibodies to the cells expressing human MSLN or monkey MSLN are shown in Tables 13-15 and FIG. 10, and the IgG subtype control is human IgG1. Tab 142, Tab020, Tab 106, and Tab 107 had good binding activity to OVCAR3 tumor cells expressing the human MSLN protein and CHO-K1-hMSLN-2C8 recombinant cells, and the binding activity of Tab131 was relatively weak. Tab 142, Tab 106, and Tab 107 had binding activity to HEK293T-monkey MSLN recombinant cells, and the cross-binding activity of Tab020 and Tab 131 to monkey MSLN was hardly detected under the same experimental conditions.

TABLE 13
Assay results for binding reactions of control
antibodies with OVCAR3 tumor cells by FACS

| Concentration | Antibody | | | | | |
|---|---|---|---|---|---|---|
| (nM) | Tab020 | Tab106 | Tab107 | Tab131 | Tab142 | hIgG1 |
| 100 | 5643 | 6202 | 3154 | 328 | 3053 | 93 |
| 20 | 5534 | 3484 | 2636 | 211 | 2096 | 78 |
| 4 | 4407 | 2082 | 1520 | 113 | 1009 | 74 |
| 0.8 | 1716 | 858 | 619 | 82 | 389 | 76 |
| 0.16 | 533 | 308 | 229 | 76 | 155 | 74 |
| 0.032 | 222 | 140 | 111 | 75 | 99 | 75 |
| 0.0064 | 113 | 91 | 80 | 71 | 81 | 74 |
| 0.00128 | 91 | 111 | 76 | 75 | 79 | 75 |

TABLE 14
Assay results for binding reactions of control antibodies
with CHO-K1-hMSLN-2C8 recombinant cells by FACS

| Concentration | Antibody | | | | | |
|---|---|---|---|---|---|---|
| (nM) | Tab020 | Tab106 | Tab107 | Tab131 | Tab142 | hIgG1 |
| 100 | 15258 | 6716 | 7862 | 4764 | 6315 | 72 |
| 20 | 15440 | 6269 | 6930 | 4650 | 6819 | 156 |
| 4 | 9001 | 2785 | 2960 | 1949 | 5157 | 110 |
| 0.8 | 2290 | 812 | 767 | 559 | 1310 | 80 |
| 0.16 | 593 | 270 | 264 | 200 | 410 | 59 |
| 0.032 | 206 | 119 | 120 | 154 | 152 | 59 |
| 0.0064 | 96 | 77 | 70 | 118 | 99 | 65 |
| 0.00128 | 75 | 68 | 63 | 62 | 71 | 116 |

TABLE 15
Assay results for binding reactions of control antibodies
with HEK293T-monkey MSLN recombinant cells by FACS

| Concentration | Antibody | | | | | |
|---|---|---|---|---|---|---|
| (nM) | Tab020 | Tab106 | Tab107 | Tab131 | Tab142 | hIgG1 |
| 100 | 126 | 3292 | 3930 | 87 | 3724 | 127 |
| 20 | 100 | 2665 | 3649 | 85 | 3654 | 84 |
| 4 | 90 | 2441 | 3140 | 86 | 3609 | 85 |
| 0.8 | 88 | 860 | 918 | 87 | 1678 | 87 |
| 0.16 | 86 | 313 | 288 | 83 | 503 | 85 |
| 0.032 | 85 | 142 | 131 | 106 | 192 | 125 |
| 0.0064 | 85 | 100 | 96 | 85 | 115 | 85 |
| 0.00128 | 85 | 90 | 119 | 93 | 98 | 84 |

### EXAMPLE 2. PREPARATION OF ANTI-HUMAN MSLN HYBRIDOMA MONOCLONAL ANTIBODIES

### 2.1. Immunization of Animals

Anti-human MSLN monoclonal antibodies were produced by immunization of mice.

The laboratory animals were 6- to 8-week-old female BALB/c AnNCrl mice (purchased from Vital River) or SJL/JorllcoCrl mice (purchased from Shanghai SLAC Laboratory Animal Co., Ltd.) for experiments, which were housed in an SPF environment. The purchased mice were housed in a laboratory environment for 1 week, in 12/12 hour light/dark cycles adjustment, at a temperature of 20-25° C., with humidity at 40%-60%. The acclimatized mice were immunized according to the following scheme. The immune antigens were as follows: (1) protein immunogens: human MSLN-FL-hFc protein, MSLN-FL-his protein, MSLN-R3-rFc protein (self-prepared); and (2) 293T cells transfected with different MSLN proteins as immunogens: 293T-hMSLN, 293T-hMSLN R3, and 293T-hMSLN R3/mMSLN R1-2 (self-prepared). 6 groups of mice were immunized with the above antigens individually or in combination (see Table 16 for the immunization scheme). For initial immunization, 0.1 mL of immunogen was emulsified with TiterMax (purchased from Sigma, Cat. No. T2684) and then injected subcutaneously and intraperitoneally, that is, 50 μg of immunogenic protein or 5E6 cells were injected into each mouse. For booster immunization, 0.1 mL of immunogen was injected subcutaneously and intraperitoneally using Imject Alum Adjuvant (purchased from Thermofisher scientific, Cat. No. 77161), that is, 25 μg of immunogen was injected into each mouse. The frequency of immunization was once a week. Blood was collected before fusion, and the antibody titer in mouse serum was assayed by ELISA and FACS. The results are shown in FIGS. 11-18, indicating that the post-immunization sera of the mice immunized with the above proteins and cells showed antigen-antibody reactions with different degrees of binding activity to the immunogens, wherein the blank control was 1% (w/w) BSA. The data in the table are OD_{450 nm} and MFI values.

TABLE 16
Mouse immunization groups and immunization scheme

| | | | | Immunization | | | Mice |
|---|---|---|---|---|---|---|---|
| | | Mouse | | dose | Immunization | | selected for |
| Group | Immunogen | strain | Mouse No. | (μg/animal) | method | Adjuvant | fusion |
| 1 | MSLN-FL-hFc; | Balb/c | #186-189 | 50/25 | IP/SC/FP | Titer Max | #186, #188, |
| | (MSLN-R3-rFc) | | | | | (only for PI); | #190 |
| | | | | | | Alum + CpG | |
| 2 | MSLN-FL-his | SJL | #1201-1205 | 50/25 | IP/SC/FP | Titer Max | #1201, |
| | | | | | | (only for PI); | #1202, |
| | | | | | | Alum + CpG | #1203, |
| | | | | | | | #1204, |
| | | | | | | | #1205 |
| 3 | MSLN-R3-rFc; | SJL | #1206-1210 | 100/50 | IP/SC/FP | Titer Max | #1207, |
| | 293T-hMSLN | | | | | (only for PI); | #1209, |
| | | | | | | Alum + CpG | #1210 |
| 4 | MSLN-R3-rFc; | SJL | #1586-1590 | 100/50 (25) | IP/SC/FP | Titer Max | #1586, |
| | MSLN-FL-hFc | | | | | (only for PI); | #1587, |
| | | | | | | Alum + CpG | #1588, |
| | | | | | | | #1589 |
| 5 | 293T-hMSLN | SJL | #141-145 | 0.5~1E7 | IP | N/A | #144 |
| | R3 | | | | | | |
| 6 | 293T-hMSLN | SJL | #146-150 | 0.5~1E7 | IP | N/A | #149 |
| | R3/mMSLNR1-2 | | | | | | |

### 2.2. Splenocyte Fusion and Hybridoma Screening

ACK Lysing Buffer (purchased from Gibco, Cat. No. A1049201) was added to lyse red blood cells mixed in splenocytes to obtain a splenocyte suspension. The cells were washed 3 times by centrifugation at 1000 rpm in a DMEM (purchased from Gibco, Cat. No. 11995-073) basal medium and then mixed with mouse myeloma cells SP2/0 (purchased from ATCC, CRL-1581) at a ratio of 2:1 in number of viable cells. The mixture was subjected to cell fusion using BTX ECM2001+ efficient electrofusion (see METHODS IN ENZYMOLOGY, VOL. 220). The fused cells were diluted into a DMEM medium containing 20% fetal bovine serum (ExCell Bio, Cat. No. FSD500) and 1×HAT (purchased from Sigma, Cat. No. H0262), wherein the percentage was a percentage by mass. Then the cells were added to a 96-well cell culture plate at 2×10⁴ cells/200 μL/well, and the plate was put into an incubator at 37° C. with 5% CO₂, wherein the percentage was a percentage by volume. After 14 days, cell fusion plate supernatants were screened by ELISA, and ELISA-positive clones were amplified into a 24-well plate and expanded in a DMEM medium containing 10% (w/w) HT (purchased from Sigma, Cat. No. H0137) fetal bovine serum under the conditions of 37° C. and 5% (v/v) CO₂. After 3 days of culture, the expanded culture medium in the 24-well plate was centrifuged, and the supernatant was collected and then analyzed for antibody subtypes. The binding activity to human MSLN proteins and human MSLN-positive cells was determined by ELISA and FACS (see Example 1.2 and Example 1.3 for the assays of binding activity, respectively).

Based on screening results from the 24-well plate, hybridoma cells from the positive groups in ELISA and FACS assays were selected as eligible positive clones, and the eligible hybridoma cells were selected, subcloned into a 96-well plate by a limiting dilution method, and cultured in a DMEM medium containing 10% (w/w) FBS (purchased from Gibco) under the conditions of 37° C. and 5% (v/v) CO₂. After 10 days of subcloning, primary screening was performed by ELISA and FACS, and a single positive monoclone was selected and amplified into a 24-well plate for further culture.

Based on assay results for the sample from the 24-well plate, the optimal clone was selected, expanded in a DMEM medium containing 10% (w/w) FBS (purchased from Gibco) under the conditions of 37° C. and 5% (v/v) CO₂, and then cryopreserved in liquid nitrogen to give the hybridoma cell of the present invention.

### EXAMPLE 3. AMINO ACID SEQUENCING FOR LIGHT AND HEAVY CHAIN VARIABLE REGIONS OF HYBRIDOMA-POSITIVE CLONES

Hybridoma cells in the logarithmic growth phase were collected, fully lysed with Trizol (Invitrogen, Cat No. 15596-018), and then stored at −80° C. for sequencing. Suzhou Genewiz Biological Technology Co., Ltd. was entrusted to complete the amino acid sequencing of light and heavy chain variable regions of hybridoma-positive clones for the samples. Sequencing results were analyzed using MOE software, and then an evolutionary tree was constructed based on the amino acid sequences of the variable region-encoded protein; after the sequences close to each other on the evolutionary tree were eliminated based on sequence similarity, 71 clones were obtained by screening, including 8 clones in F1 series (S009-F1.2.12, S009-F1.7.14, S009-F1.25.10, S009-F1.35.24, S009-F1.56.1, S009-F1.57.1, S009-F1.59.1, and S009-F1.62.9, see SEQ ID NOs: 16-31 in Table 17), 10 clones in F2 series (S009-F2.13.3, S009-F2.16.10, S009-F2.17.3, S009-F2.21.4, S009-F2.23.12, S009-F2.38.12, S009-F2.39.3, S009-F2.47.1, S009-F2-56.12, and S009-F2.58.8, see SEQ ID NOs: 32-51 in Table 18), 9 clones in F3 series (S009-F3.7.3, S009-F3.16.1, S009-F3.23.1, S009-F3.38.10, S009-F3.45.21, S009-F3.51.8, S009-F3-63.5, S009-F3.74.20, and S009-F3.80.22, see SEQ ID NOs: 52-69 in Table 19), 2 clones in F4 series (S009-F4-94.15 and S009-F4-127.10, see SEQ ID NOs: 70-73 in Table 20), 1 clone in F5 series (S009-F5-9.16, see SEQ ID NOs: 74-75 in Table 20), 2 clones in F6 series (S009-F6-62.5 and S009-F6-76.1, see SEQ ID NOs: 76-79 in Table 20), 20 clones in F7 series (S009-F7.2.3, S009-F7.6.17, S009-F7.11.11, S009-F7.12.13, S009-F7.18.10, S009-F7.21.16, S009-F7.23.19, S009-F7.25.19, S009-F7.26.15, S009-F7.30.5, S009-F7.33.24, S009-F7.41.18, S009-F7.44.20, S009-F7.48.1, S009-F7.53.2, S009-F7.61.21, S009-F7.65.13, S009-F7.66.12, S009-F7.67.12, and S009-F7.69.8, see SEQ ID NOs: 80-119 in Table 21), and 19 clones in F8 series (S009-F8-4.5, S009-F8-5.15, S009-F8-7.5, S009-F8-8.22, S009-F8-9.16, S009-F8-12.13, S009-F8-13.8, S009-F8-15.19, S009-F8-18.9, S009-F8-19.21, S009-F8-22.23, S009-F8-24.14, S009-F8-27.1, S009-F8-28.23, S009-F8-29.1, S009-F8-31.22, S009-F8-32.3, S009-F8-33.12, and S009-F8-36.12, see SEQ ID NOs: 120-157 in Table 22).

TABLE 17
Amino acid sequence information of light and heavy chain variable regions of
MSLN F1 hybridoma-positive clones

| Sequence name | Sequence No. | Amino acid sequence |
|---|---|---|
| S009-F1.2.12 | SEQ ID NO: 16 | QVQLKQTGPGLVQPSQSLSITCTVSGFSLTNYAVHWVRQSPG |
| heavy chain | | KGLEWLGVIWSGGATDYNTVFISRLSISKDNSKSQVFFKVNS |
| variable region | | LQVDDTAIYYCARTGSGYAMDYWGQGTSVTVSS |
| S009-F1.2.12 | SEQ ID NO: 17 | DIVLTQSPATLSVTPGDRVSLSCRTSHNVNTYLHWYQQKSHE |
| light chain | | SPRLLIKYASQSISEIPSRFSGSGSGTNFTLSINSVETEDFGMYF |
| variable region | | CHQTNRWPLTFGAGTKLELK |
| S009-F1.7.14 | SEQ ID NO: 18 | DVQLQESGPGLVKPSQSLSLTCTVTAYSITSDYAWNWIRQFPG |
| heavy chain | | NKLEWMGCIRYSGGTTYNPSLKSRISITRDTSKNQFFLQLNSV |
| variable region | | TTEDTATYHCARSRQLGDAGFDYWGQGTTLTVSS |
| S009-F1.7.14 | SEQ ID NO: 19 | QIVLTQSPAIMSASPGEKVTISCSASSSVSYMYWYQQKPGSSP |
| light chain | | KPWISRTSNLASGVPARFSGSGSGTSYSLTISSMEAEDAATYY |
| variable region | | CQQYHSYPPTLGAGTKLELK |
| S009-F1.25.10 | SEQ ID NO: 20 | EVQLQQSGPELVKPGASLKISCKASGYSFTDYTMNWVKQSH |
| heavy chain | | GKNLEWIGLFNPYNGGISYNQKFKGKATLTVDKSSNTAYMEL |
| variable region | | LSLTSDDSAVYFCARDGRGGFYAMDYWGQGTSVTVSS |
| S009-F1.25.10 | SEQ ID NO: 21 | DIQMTQTTSSLSASLGDRVTISCRASQDISIYLNWYQQKPDGP |
| light chain | | VKLLIYYTSRLHSGVPSRFSGSGSGTDFSLTISNLEQEDIATYF |
| variable region | | CQQGYTLPPWTFGGGTKLEIK |
| S009-F1.35.24 | SEQ ID NO: 22 | EVQLQQSEPELVKPGASVRISCKASGYSFTDYYMHWVKQSP |
| heavy chain | | ENRLEWIGEINPSTGGTSYNPKFKDKATLTVDKSSSTAYMQL |
| variable region | | KSLTSEESAVYYCTRYHYYGSSSYVMDYWGQGTSVTVSS |
| S009-F1.35.24 | SEQ ID NO: 23 | QIVLTQSPAILSASPGERVTMTCSASSSVYHMHWFQQKSGTSP |
| light chain | | KRWVYDTSKLASGVPARFSGSGSGTSYSLTISSMEAEDAATY |
| variable region | | YCQHWRTNPLTFGAGTKLELK |
| S009-F1.56.1 | SEQ ID NO: 24 | EVQLQQSGAEVVKPGASVKLSCTASGFNIRHTYMHWVKQRP |
| heavy chain | | EQGLEWIGRIDPANGNTEYDPKFQGKATITADTSSNTAYLQLS |
| variable region | | SLTSEDTAVYYCARDGWYIDVWGAGTTVTVSS |
| S009-F1.56.1 | SEQ ID NO: 25 | DIVMSQSPSSLAVSVGEKVTMSCKSSQSLLYSNNQKNYLAWY |
| light chain | | QQKPGQSPKLLIYWASTRESGVPDRFTGSGSGTDFTLTISSVK |
| variable region | | AEDLAVYYCQRYYSYPWTFGGGTKLEIK |
| S009-F1.57.1 | SEQ ID NO: 26 | EAQLQQSGAVFVKPGASVKLSCTASGFSSKDIFLHWVKQRPE |
| heavy chain | | QGLEWIGRIDPASDNTIYDPKFQDRATITSDRSSNTAYLQFSSL |
| variable region | | TSEDTAVYFCLYFDFWGQGTTLTVSS |
| S009-F1.57.1 | SEQ ID NO: 27 | DIVMTQATPSISVTPGESVSISCRSSESLLHSNGNTYLYWFLQR |
| light chain | | PGQSPQLLIYRMSNLASGVPDRFSVSGSGTAFTLRISRVEAED |
| variable region | | VGVYYCMQHLEYPLTFGAGTKLELK |
| S009-F1.59.1 | SEQ ID NO: 28 | EVKLEGSGGGLVQPGGSMMLSCVATGFTFSSYWMNWVRQSP |
| heavy chain | | DRGLEWVAEIRLKSNGFAAFYAESVKGRFTISRDDSKSSVYL |
| variable region | | QMNNLRTEDTGIYYCTYFVHWGQGILVTVSE |
| S009-F1.59.1 | SEQ ID NO: 29 | DIVLTQSPTSLAVSLGQRATISCRTSESVEYFGTILIQWYQQKP |
| light chain | | GQPPKLLIFGASNVESGVPARFSGSGSGTDFSLNIHPVEEEDIA |
| variable region | | MYFCQQNRKVPYTFGGGTTLELK |
| S009-F1.62.9 | SEQ ID NO: 30 | EVQLQQSGADLVKPGASVKLSCTVFGLNFKDTFMHWVRQRP |
| heavy chain | | EQGLEWIGRIDPANDNSIYGPKFQDKATITADTSSNTAYLHLSS |
| variable region | | LTSEDTAVYYCLYFDNWGHGTTLTVSS |
| S009-F1.62.9 | SEQ ID NO: 31 | DIVMTQAASSEPVTPGESVSISCRSDKSLLHSNGHNYLYWFL |
| light chain | | QRPGQSPHLLIYRMSNLASGVPDRFSGSGSGTAFTLRISRVEA |
| variable region | | EDVGVYYCMQHLEYPLTFGAGTKLELK |

TABLE 18
Amino acid sequence information of light and heavy chain variable regions of
MSLN F2 hybridoma-positive clones

| Sequence name | Sequence No. | Amino acid sequence |
|---|---|---|
| S009-F2.13.3 | SEQ ID NO: 32 | QVQLQQSGAELMKPGASVKLSCKATGYTFTGYWIEWVK |
| heavy chain | | QRPGHGLEWIGEILPGSGSTNYNEKFKGKATFTADTSSNT |
| variable region | | AYMQLSSLTTEDSAIYYCARAGAWFAYWGQGTLVTVSA |
| S009-F2.13.3 light | SEQ ID NO: 33 | QIVLTQSPAIMSASLGERVTMTCTASSSVSSSYLHWYQQK |
| chain variable | | PGSSPKLWTYSTSNLASGVPARFSGSGSGTSYSLTISSMEA |
| region | | EDAATYYCHQYHRSPWTFGGGTKLEIK |
| S009-F2.16.10 | SEQ ID NO: 34 | QVQLQQPGAELIKPGASVKLSCKASGYTFTSYWMHWVK |
| heavy chain | | QRPGQGLEWIGIIHPNIGSTNYNERFKSKATLTVDKSSSTA |
| variable region | | YMQLSSLTSEDSAVYYCARRSSNYGDWYFDVWGTGTTV |
| | | TVSS |
| S009-F2.16.10 | SEQ ID NO: 35 | DIVMTQSHRFMSTSVGDRVSITCKASQDVGTSVAWYQQK |
| light chain variable | | PGQSPKLLIYWASTQHTGVPDRFTGSGSGTDFTLTINNVQ |
| region | | SEDLLDYFCQQYSSYPLTFGAGTKLELK |
| S009-F2.17.3 | SEQ ID NO: 36 | QVQLQQPGTELVTPGASVKLSCKASGYSFTSYWMHWVK |
| heavy chain | | QRPGQGLEWIGNINPSNGDTFYNEKFKNKATLTVDKSSST |
| variable region | | AYMQLSSLTSEDSAVYYCARGWLRDYWGQGTTLTVSS |
| S009-F2.17.3 light | SEQ ID NO: 37 | DIVMTQSPSSLAVSVGQKVTMSCKSSQSLLNSSSQKNYLA |
| chain variable | | WYQQKPGQSPKLLVYFASTKDSGVPDRFIGSGSGTDFTLT |
| region | | INSVQAEDLADYFCQQHYTTPYTFGGGTKLEIK |
| S009-F2.21.4 | SEQ ID NO: 38 | EVQLQQSGPELVKPGASVKIPCKASGYTFTDYNMDWVK |
| heavy chain | | QSHGKSLEWIGDINPNNGGSIYNQRFKGKATLTVDKSSST |
| variable region | | AYMELRSLTSEDSAVYYCARRAYYSTGYFDVWGTGTTVT |
| | | VSS |
| S009-F2.21.4 light | SEQ ID NO: 39 | QIVLTQSPALMSASPGEKVTITCSASSSISYMHWFQQKPGT |
| chain variable | | SPKLWIYSTSTLASGVPARFSGSGSGTSYSLTISRMEAEDT |
| region | | ATYYCQQRSSYPPTFGGGTKLEIK |
| S009-F2.23.12 | SEQ ID NO: 40 | QVQLQQPGTELVKPGASVKLSCKASGYTFTNYWMHWV |
| heavy chain | | KQRPGQGLEWIGNINPSNGGPYYNERFRSKATLTVDKSSS |
| variable region | | TAYMQLSSLTSEDSAVYYCARPYYGSSYGYFDYWGQGTT |
| | | LTVSS |
| S009-F2.23.12 | SEQ ID NO: 41 | DIQMTQSPSSLSASLGGKVTITCKASQDINKYIAWYQHKP |
| light chain variable | | GKGPRLLIHYTSELQPGIPSRFSGNGSGRDYSFSISNLEPED |
| region | | IATYYCLQYANPLRTFGGGTKLEIK |
| S009-F2.38.12 | SEQ ID NO: 42 | QVQLQQPGTELVKPGASVKLSCKASGYTFTSYWMHWVK |
| heavy chain | | QRPGQGLEWIGNINPSNGGTNYNEKIKNKATLTVDKSSST |
| variable region | | AYMQLSSLTSEDSAVYYCARWNYYGNYPFDYWGQGTTL |
| | | TVSS |
| S009-F2.38.12 | SEQ ID NO: 43 | DIVMTQSQKFMSTTVGDRVSITCKASQNVGTAVAWYQQK |
| light chain variable | | PGQSPKLLIYSASNRYTGVPDRFTGSGSGTDFTLTISNMQS |
| region | | EDLADYFCQQYSSYPLTFGAGTKLELK |
| S009-F2.39.3 | SEQ ID NO: 44 | QVQLQQPGTELVKPGASVKLSCKASGYTFTNYWMHWV |
| heavy chain | | KQRPGQGLEWIGNINPSSGDSYYNERFMSKAKMTVDKSS |
| variable region | | STAYMQLSSLTSEDSAVYYCARSGGLWLAFWGPGTLVTV |
| | | SA |
| S009-F2.39.3 light | SEQ ID NO: 45 | DIVMTQSPSSLAMSVGQKVTMSCKSSQTLLNSVSQNNYL |
| chain variable | | AWYQQKPGQSPTLLVYFASTRESGVPDRFIGGGSGTDFTL |
| region | | TISSVQAEDLADYFCQQHYRTPYTFGGGTNLEIK |
| S009-F2.47.1 | SEQ ID NO: 46 | QVQLQQPGAELVKPGASVKLSCKASGYTFTSYWMHWV |
| heavy chain | | KQRPGQGLEWIGMIHPNSGSTNYNEKFKSKATLTVDKSSS |
| variable region | | TAYMQLSSLTSEDSAVYYCARPVVPYWYFDVWGTGTTV |
| | | TVSS |
| S009-F2.47.1 light | SEQ ID NO: 47 | DIVMTQSQKFMSTTVGDRVSITCKASQNVGTAVAWYQQK |
| chain variable | | PGQSPKLLIYSASNRYTGVPDRFTGSGSGTDFTLTISNMQS |
| region | | EDLADYFCQQSSSYPLTFGAGTKLELK |
| S009-F2-56.12 | SEQ ID NO: 48 | DVQLQESGPGMVKPSQSLSLTCTVTGYSITSGYDWHWIR |
| heavy chain | | HFPGNKLEWMGYISYSGSTNYNPSLKSRISITHDTSKNHF |
| variable region | | FLKLKSVTTEDTATYYCARGTGPDYWGQGTTLTVSS |
| S009-F2-56.12 | SEQ ID NO: 49 | DIVMTQSQKFMSTSVGDRVSITCKASQNVRTAVAWYQQK |
| light chain variable | | PGQSPKALIHLPSNRHTGVPDRFTGSGSGTDFTLTISNVQS |
| region | | EDLADYFCLQHWNYPLTFGGGTKLEIK |
| S009-F2.58.8 | SEQ ID NO: 50 | QVQLLQSGAELAKPGASVKLSCKASGYTFTSYWVHWVK |
| heavy chain | | QRPGQGLEWIGYINPNSGYTKYNQKFKDKATLTADKSSS |
| variable region | | TAYMQLSSLTYEDSAVYYCADHYYGSSRDYFDYWGQGT |
| | | TLTVSS |
| S009-F2.58.8 light | SEQ ID NO: 51 | QIVLTQSPAIMSASPGEKVTMTCSASSSVSYMYWYQQRP |
| chain variable | | GSSPRLLIYDTSNLASGVPVRFSGSGSGTSYSLTISRMEAE |
| region | | DAATYYCQQWNSYPYTFGGGTKLEIK |

TABLE 19
Amino acid sequence information of light and heavy chain variable regions of
MSLN F3 hybridoma-positive clones

| Sequence name | Sequence No. | Amino acid sequence |
|---|---|---|
| S009-F3.7.3 heavy | SEQ ID NO: 52 | EVNFEESGGGLVQPGGSMKLSCVASGFTFSNYWMNWV |
| chain variable | | RQSPEKGLEWVAQIRLKSDNYATHYAESVKGRFTISRDD |
| region | | FKSSVYLQMNNLRAEDTGIYYCTVGTGGYWGQGTTLT |
| | | VSS |
| S009-F3.7.3 light | SEQ ID NO: 53 | DIVLTQSPASLAMSLGKRATISCRASESVSIVGTNVIHWF |
| chain variable | | QQKPGQPPKLLIYHASNLETGVPARFSGSGSGTDFTLTID |
| region | | PVEEDDVAIYHCLQSRKIPWTFGGGTKLEIK |
| S009-F3.16.1 | SEQ ID NO: 54 | EVKFEESGGGLVQPGGSMKLSCVASGFTFSNYWMNWV |
| heavy chain | | RQSPEKGLEWVAQIRLKSDNYATHYAESVKGRFSISRDD |
| variable region | | SKSSVYLQMNNLRAEDTGIYYCSVGTGGYWGQGTTLT |
| | | VSS |
| S009-F3.16.1 light | SEQ ID NO: 55 | DIVLTQSPASLALSPGKRATISCRASESVSIIGTDVIHWFQ |
| chain variable | | QKPGQPPKLLIYHASNLETGVPARFSGSGSRTDFTLTIDP |
| region | | VEEDDVAIYYCLQSRKIPWTFGGGTKLEIK |
| S009-F3.23.1 | SEQ ID NO: 56 | QVQLQQSGADLVRPGASVTLSCKASGYTFTDYEMHWV |
| heavy chain | | KQTPVHGLEWIGAIDPETGDTVYNQNFKGKAILTADKSS |
| variable region | | STAYMELRSLTSEDSAVYYCTRYGYDWGWGQGTTLTVS |
| | | S |
| S009-F3.23.1 light | SEQ ID NO: 57 | DVVMTQTPLTLSVTIGQPASISCKSSQSLLYSNGKTYLN |
| chain variable | | WLLQRPGQSPKRLIYLVSKLDSGVPDRFTGSASGTDFTL |
| region | | KISRVEADDLGVYYCVQGTHFPWTFGGGTKLEIK |
| S009-F3.38.10 | SEQ ID NO: 58 | EVQLQQSGPVLVKPGASVKMSCEASGYIFTDYYMNWV |
| heavy chain | | KKSHGKSLEWIGVINPKNGVISHNQKFKGKATLTVDKSS |
| variable region | | NTAYMELSSLTSEDSAVYYCANYGSRFYAMDYWGQGTS |
| | | VTVSS |
| S009-F3.38.10 | SEQ ID NO: 59 | DVVMTQTPLSLPVSLGDQASISCRSSQSLIHSDGNTYLQ |
| light chain variable | | WYLQKSGQSPKLLIYKVSNRFSGVPDRFSGSGSGTDFTL |
| region | | KISRVETEDLGVYFCSQTTHVPFTFGSGTKLEIK |
| S009-F3.45.21 | SEQ ID NO: 60 | DVQLQESGPGLVKPSQSLSLTCSVTGDSITSGYYWNWIR |
| heavy chain | | QFPGNKLEWMGFIRFDGTNNYNPSLKNRISITRDTSKNQ |
| variable region | | FFLKLNSVTTEDTATYYCAREGSYAPAWFAYWGQGTLVT |
| | | VSA |
| S009-F3.45.21 | SEQ ID NO: 61 | QIVLTQSPAIMSASPGEKVTMTCSASSSLSYMYWYQQKP |
| light chain variable | | GSSPRLLIYDTSNLASGVPVRFSGSGSGTSYSLTISRMEA |
| region | | EDAATYYCQQWSSYPPTFGGGTKLEIK |
| S009-F3.51.8 light | SEQ ID NO: 63 | DVVMTQTPLTLSVTVGQPASISCKSSQSLLYSDGKTYLN |
| chain variable | | WLLQRPGQSPKRLIYLVSKLDSGVPDRFTASGSGTDFTL |
| region | | KISRVEAEDLGVYYCWQGTHFPWTFGGGTKLEIK |
| S009-F3-63.5 | SEQ ID NO: 64 | QVQLQQSGAELVRPGASVTLSCKASGYTFTDYEMHWV |
| heavy chain | | KQTPVHGLEWIGGIDPETGDTAYNQQFKGKAILTADRSS |
| variable region | | STAYMELRSLTSEDSAVYYCTNYASSREDYWGQGTTLTV |
| | | SS |
| S009-F3-63.5 light | SEQ ID NO: 65 | DVVMTQTPLTLSVTIGQPASISCKSSQSLLYSNGKTYLSW |
| chain variable | | LLQRPGQSPKRLIYQVSKLDSGVPDRFTGSGSGTEFTLKI |
| region | | STVEAEDLGVYYCVQITHFPQTFGGGTKLEIK |
| S009-F3.74.20 | SEQ ID NO: 66 | RVQLQQSGAELVRPGASVTLSCKASGYTFTDSEMHWVK |
| heavy chain | | QTPVHGLEWIGAIDPEIDGTAYNQNFRDKAILTADKSSST |
| variable region | | AYMELRSLTSEDSAVYYCTTYFGSGYGYFDVWGTGTTV |
| | | TVSS |
| S009-F3.74.20 | SEQ ID NO: 67 | DVVMTQTPLSLSVSLGDQASISCRSSQSLVYSNGNTYLH |
| light chain variable | | WFLQKPGQSPKLLIYKVSNRFSGVPDRFSGSGSGTDFTL |
| region | | KINRVEAEDLGVYFCSQSTHIPFTFGAGTKLELK |
| S009-F3.80.22 | SEQ ID NO: 68 | QVQLQQSGAELVRPGASVTLSCKASGYTFTDYEIHWVK |
| heavy chain | | QTPVHGLEWIGAFDPEIGGSAYNQKFKDRATLTADKSSS |
| variable region | | TAYMELHSLTAEDSAVYYCTDYYGSSSGYFDVWGTGTT |
| | | VTVSS |
| S009-F3.80.22 | SEQ ID NO: 69 | DVVMTQTPLSLPVSLGDQASISCRSSQSLEHNNGNTYLH |
| light chain variable | | WFLHKPGQSPKLLIYKVSNRFSGVPDRFSGSGSGTDFTL |
| region | | KISRVEAEDLGVYFCSQSTHVPFTFGSGTKLEIK |

TABLE 20
Amino acid sequence information of light and heavy chain variable regions of
MSLN F4, F5, and F6 hybridoma-positive clones

| Sequence name | Sequence No. | Amino acid sequence |
|---|---|---|
| S009-F4-94.15 | SEQ ID NO: 70 | QVQLHQPGAELVRPGSSVKLSCKASGYTFTSYWVHWVQ |
| heavy chain | | QRPRQGLEWIGNIDPSDSEIHYNQKFKDKATLTVDKSSST |
| variable region | | AYIHLSRLTSEDSAIYYCVRGGVPWFAYWGQGTLVTVSA |
| S009-F4-94.15 | SEQ ID NO: 71 | QIVLTQSPAIMSASPGEKVTLTCSASSSVSSTYLYWFQQKP |
| light chain variable | | GSSPKVWIYSTSNLASGVPARFSGSGSGTSYSLTISSMEAE |
| region | | DAASYFCHQWISYPYTFGGGTKLEIK |
| S009-F4-127.10 | SEQ ID NO: 72 | EVQLQQSGPVLVKPGASVKMSCKASGYTFTDYYMNWV |
| heavy chain | | KQSHGKSPEWIGVINPYNDVISYNQKFKGKATLTVDKSSS |
| variable region | | TAYMELNSLTSEDSAVYYCANYGSSYYAMDYWGQGTSV |
| | | TVSS |
| S009-F4-127.10 | SEQ ID NO: 73 | DVVMTQTPLSLSVSLGDQASISCRSSQSLVHSNGNTYLQ |
| light chain variable | | WSLQKPGQSPNLLIYKVSNRFSGVPDRFSGSGSGTDFTLK |
| region | | ISRVEAEDLGVYFCSQTTHVPFTFGGGTKLEIR |
| S009-F5-9.16 | SEQ ID NO: 74 | EVQLRQSGPVLVKPGASVKMSCKASGYTFTDYFMNWVK |
| heavy chain | | PSHGKSLEWIGVINPSNGVINYNQRFKGKATLTVDKSSST |
| variable region | | AYMELNSLTSEDSAVYYCARSYDFAWFAYWGQGTLVTVS |
| | | A |
| S009-F5-9.16 light | SEQ ID NO: 75 | DVLMTQTPLSLPVSLGDQASISCRSSQNIVHSNGNTYLEW |
| chain variable | | YLQKPGQSPKLLIYTVSNRFSGVPDRFSGSGSGTDFTLKIS |
| region | | RVEAEDLGVYYCFQGSHVPYTFGGGTKLEIK |
| S009-F6-62.5 | SEQ ID NO: 76 | QVQLQQPGTELVKPGASVKLSCRASGYTFTAYWMHWVK |
| heavy chain | | QRPGQGLEWIGNINPSNGGTDFNEKFKSKATLTVDKSSST |
| variable region | | AYMQLSSLTSEDSAVYYCARGRGYFDVWGTGTTVTVSS |
| S009-F6-62.5 light | SEQ ID NO: 77 | DILLTQSPAILSVSPGERVSFSCRASKNIGTSIHWYQQRTN |
| chain variable | | GSPRLLIKYASESISGIPSRFSGSGSGTDFTLNINSVESEDIA |
| region | | DYYCQQSNSWPTLTFGAGTKLELK |
| S009-F6-76.1 | SEQ ID NO: 78 | QVQLQQSGPELVKPGASVKISCKASGYTFTDYYINWVKQ |
| heavy chain | | RPGQGLEWIGWIFPGRGSTFYYEKFKGKATLTVDKSSSTA |
| variable region | | YMLLSSLTSEDSAVYFCARRGDSAGYEDLDYWGQGTSV |
| | | TVSS |
| S009-F6-76.1 light | SEQ ID NO: 79 | NIVMTQSPKFMSMSVGERVTLSCKASENVVTYVSWYQQ |
| chain variable | | KPKQSPKLLIYGASKRYTGVPDRFTGSGSATDFTLTISSVQ |
| region | | AEDLGDYHCGQSYSYPYTFGGGTKLEIK |

TABLE 21
Amino acid sequence information of light and heavy chain variable regions of
MSLN F7 hybridoma-positive clones

| Sequence name | Sequence No. | Amino acid sequence |
|---|---|---|
| S009-F7.2.3 heavy | SEQ ID NO: 80 | ELQLVESGGDLVKPGGSLKLSCAASGFTFSSNGMSWLRQTP |
| chain variable | | DKRLEWVATISSGGRYTYYPDSVKGRFTISRDNAKNTLYLQ |
| region | | MSSLKSEDTAMYYCARRGIYDYFDCWGQGTTLTVSS |
| S009-F7.2.3 light | SEQ ID NO: 81 | DIQMTQSPASLSISVGETVTITCRASENIHSSLAWYQQKQGK |
| chain variable | | SPQLLVYAATNLADGVPSRFSGSGSGSQYSLKINSLQSEDFG |
| region | | TYYCQHFWGTPWTFGGGTKLDIK |
| S009-F7.6.17 | SEQ ID NO: 82 | QVQLQQSGAELARPGASVKMSCKASGYTFTTYTMHWVKQ |
| heavy chain | | RPGQGLEWIGYISPTGDFTKYNQKFKDKATLTTDKSSNTAF |
| variable region | | MQLSSLTSEDSAVYYCARWNGTVWFAYWGQGTLVTVSA |
| S009-F7.6.17 light | SEQ ID NO: 83 | DIVMTQSQKFMSTSVGDRVTVTCKASQALGTNVAWYQQKP |
| chain variable | | GQSPKVLIYSASYRYSGVPYRFAGGGSGTDFTLTITNVQSED |
| region | | LAEYFCQQYNNYPLTFGAGTKLELK |
| S009-F7.11.11 | SEQ ID NO: 84 | EVQLQQSGPELVKPGASVKIPCKASGYTITDYNMDWVKQS |
| heavy chain | | RGKSLEWIGDVFPYNGDSIYNQKFEGRATLTVDKYSSTAYM |
| variable region | | ELRGLTSEDTAVYFCARRKTGTGYFDVWGTGTTVTVSS |
| S009-F7.11.11 | SEQ ID NO: 85 | QLVLTQSPAIMSASPGEKVTLTCSASSSVSSTYLSWYLQKPG |
| light chain variable | | SSPKLWIYSTSNLASGVPARFSGSGSGTSYSLTISSMEAEDAA |
| region | | SYFCHQWSSYPYTFGGGTKLEIK |
| S009-F7.12.13 | SEQ ID NO: 86 | EVQLQQSGPELVKPGASVKIPCKASGYTFTDYNMDWVKQS |
| heavy chain | | HGKGLEWIGDINPNTDDTIYNQKFNGKATLTVDKSSSTAYM |
| variable region | | ELRSLTSEDTAVYYCSRRLIGTGYFDVWGTGTTVTVSS |
| S009-F7.12.13 | SEQ ID NO: 87 | QIVLTQSPAIMSASPGEKVTLTCSASSSVISTYLCWYRQKPGS |
| light chain variable | | SPELWIYSTSNLASGVPARFSGSGSGTSYSLTISSMEAEDAAS |
| region | | YFCHQWSNYPYTFGGGTKLGMK |
| S009-F7.18.10 | SEQ ID NO: 88 | QVQLQQSGAELVRPGTSVKVSCKASGYAFTNYLIEWVKQR |
| heavy chain | | PGQGLEWIGVINPGSGGTKYNEKFKGKATLTADKSSSTAYM |
| variable region | | QLSSLTSEDSAVYFCASDYDYDPFAYWGQGTLVTVSA |
| S009-F7.18.10 | SEQ ID NO: 89 | DIVMTQSHKFMSTSVGDRVSITCKASQDVSTAVAWYQRKPG |
| light chain variable | | QSPKLLIYWASTRHTGVPDRFTGSGSGTDYTLTISYMQAEDL |
| region | | ALYYCQQHYSTPYTFGGGTKLEIK |
| S009-F7.21.16 | SEQ ID NO: 90 | QVQLQQPGTELVKPGASVKLSCKASGYTFTTFWMHWVKQ |
| heavy chain | | RPGQGLEWIGGVNPTNGGTNYNEKFRTKATLTVDKSSTTAD |
| variable region | | MQLSSLTSEDSAVYYCAPHYISSRPGFAYWGQGTLVTVSG |
| S009-F7.21.16 | SEQ ID NO: 91 | DIVMTQSQKFMSTSGGDRVSVTCKASQNVGTNVAWYQQKP |
| light chain variable | | GQSPKALIYSASYRYSGVPDRFTGSGSGTDFILTISNVQSEDL |
| region | | AEYFCQQYNSYPLTFGGGTKLEIK |
| S009-F7.23.19 | SEQ ID NO: 92 | EVKLVESGGGLVQPGGSLSLSCAASGFTFTDYYMSWVRQPP |
| heavy chain | | GKALEWLGFIRSKANAYTTEYSASVKGRFTISRDNSQSILYL |
| variable region | | QMNDLRAEDSATYYCARYYYYGSGYVWYFDVWGTGTTV |
| | | TVSS |
| S009-F7.23.19 | SEQ ID NO: 93 | DIVMTQSQKFMSTSVGDRVSVTCKASQSVGTNVAWYQQKP |
| light chain variable | | GQFPKALIYSASYRYSGVPDRFTGSGSGTDFTLTIINVQSEDL |
| region | | AEYFCQQYNSYPLTFGAGTKLELK |
| S009-F7.25.19 | SEQ ID NO: 94 | QVQLQQPGTELVKPGASVKLSCKASGYTFTSYWMHWVKQ |
| heavy chain | | RPGQGLEWIGNIIPSNGGTKYNEKFKSKATLTVDKSSSTAYM |
| variable region | | QLSSLTSEDSAVYYCSPHYYGGSPGFAYWGQGTLITVSA |
| S009-F7.25.19 | SEQ ID NO: 95 | DIVMTQSQKFMSTSVGDRVSVICKASQNVGTNVAWYQQKP |
| light chain variable | | GQSPKALIYSASYRYSGVPDRFTGSGSGTDFTLTISNVQSED |
| region | | LAEYFCQQYNSYPLTFGGGTKLEIK |
| S009-F7.26.15 | SEQ ID NO: 96 | DVQLQESGPGLVKPSQTVFLTCTVTGISITTGNYRWSWIRQF |
| heavy chain | | PGNKLEWIGYIYYTGFITYNPSLTSRTTITRDTPKNQFFLEMK |
| variable region | | SLTAEDTATYYCARDDYDYDVFAYWGQGTLVTVSA |
| S009-F7.26.15 | SEQ ID NO: 97 | DIQMTQSPASLSASVGETVTITCRASGNIHNYLAWYHQKQG |
| light chain variable | | KSPQFLVYKAKTLADGVPSRFSGSGSGTQYSLKINRLQPEDF |
| region | | GSYYCQHFLSIPLTFGAGTKLELK |
| S009-F7.30.5 | SEQ ID NO: 98 | QVQLQQPKTELVKPGASVKLSCKASGYIFTTYWMHWVKQ |
| heavy chain | | RPGQGLEWIGNVNPSNGGTMYNEKFERRATLTVDKSSSTAD |
| variable region | | MQLSSLTSEDSAVYYCVPHYIGSRPGFAYWGQGTLVTVSG |
| S009-F7.30.5 light | SEQ ID NO: 99 | DIVMTQSQKFMSTSVGDRVSVTCKASQNVGTNVAWYQHKP |
| chain variable | | GQSPKALLYSASYRYSGVPDRFTGSGSGTDFTLTITNVQSED |
| region | | LAEYFCQQYNSYPLTFGGGTKLEIK |
| S009-F7.33.24 | SEQ ID NO: 100 | QVQLQQPGTELVKPGASVKLSCKASGYTFTSYWMHWVKQ |
| heavy chain | | RPGQGLEWIGNVNPSNGGSNYNEKFKNKATLTVDKSSSTAD |
| variable region | | MQLNSLTSEDSAVYYCAPHYIGSRPGFAYWGQGTLVTVSG |
| S009-F7.33.24 | SEQ ID NO: 101 | DIVMTQSQKFMSTSVGDRVSVTCKASQNVGTNIAWYQQKP |
| light chain variable | | GQSPKALIYSASYRYSGVPDRFTGSGSGTDFTLTITNVQSED |
| region | | LAEYFCQQYNSYPLTFGGGTKVEIK |
| S009-F7.41.18 | SEQ ID NO: 102 | QVQLQQPGTELVKPGASVKLSCKASDYTFTSYWMHWVKQ |
| heavy chain | | RPGQGLEWIGSINPSNGDTYYNEKFKNKATLTVDKSSSTAY |
| variable region | | MQLSSLTSDDSAVYYCARGWYFDVWGTGTTVTVSS |
| S009-F7.41.18 | SEQ ID NO: 103 | DIVMTQSPSSLTVSVGQKVTMSCKSSQSLLNSSSQKNYLAW |
| light chain variable | | YQQKPGQSPKLLVYFASTRESGVPDRFIGSGSGTDFTLIISSV |
| region | | QAEDLADYFCQQHYSTPRTFGGGTKLEII |
| S009-F7.44.20 | SEQ ID NO: 104 | EVQLQQSGPELVKPGASVKIPCKASGYTFTDYNMDWVKQS |
| heavy chain | | HGKSLEWIGDINPSTGGTIYNQKFNGKATLTEDKSASTVYM |
| variable region | | EFRSLTSDDTAVYYCARRRIGTGYFDVWGTGTTVTVSS |
| S009-F7.44.20 | SEQ ID NO: 105 | QIVLTQSPALMSASPGEKVTLTCSASSSVISSYLSWYQQKPG |
| light chain variable | | SSPKLLIYRTSNLASGVPARFSGSGSGTSYSLTISSMEAEDAA |
| region | | SYFCHQWSSFPYTFGGGTKLEIK |
| S009-F7.48.1 | SEQ ID NO: 106 | QVQLQQPGTELVKPGASVKLSCKASGYTFTSYWMHWVKQ |
| heavy chain | | RPGQGLEWIGNINPSNGGTNNNENFKSKATLTVDTSSSTAY |
| variable region | | MQLSSLTSEDSAVYYCVRNGYHGYWYFDVWGTGTTVTVS |
| | | S |
| S009-F7.48.1 light | SEQ ID NO: 107 | DIVMTQSQKFMSTSVGDRVSVTCKASQNVGTNVAWYQQQP |
| chain variable | | GQSPKALIYSASYRYSGVPDRFTGSVSGTDFTLTISNVQSED |
| region | | LAEYFCQQYNSYPLTFGGGTKLEIK |
| S009-F7.53.2 | SEQ ID NO: 108 | EVQLQQSGPELVKPGASVKIPCKTSGYTFTDYNMDWVKQS |
| heavy chain | | HGKSLEWIGDINPNTDGAIYNQKFQNKATLTVDKSSSTAYM |
| variable region | | ELRSLTSEDTAVYYCTRRKLGRKFFDYWGQGTTLTVSS |
| S009-F7.53.2 light | SEQ ID NO: 109 | QIVLTQSPAIMSASPGEQVTMTCSASSSVGYMNWYQQKPGS |
| chain variable | | SPRLLIYDTSNLASGVPVHFSGSGSGTSYSLTISRMEAAFAAT |
| region | | YYCQQWYVYPYTFGGGTKLEIK |
| S009-F7.61.21 | SEQ ID NO: 110 | DVQLQQSGPELVEPGASVRIPCKAAGHTFTDYNVDWVKQS |
| heavy chain | | HGQSLEWIGDVNPNTDGAIYNQKFEGKATLTVDTSSSTAYM |
| variable region | | ELRSLTSEDTAVYFCARRRLGQGGFDSWGQGTTLTVSS |
| S009-F7.61.21 | SEQ ID NO: 111 | QVVLTQSPAIMSASPGEKVTLTCGASSSLISKYLYWYQQKPG |
| light chain variable | | SSPKLWIYSTSNLASGVPARFSGSGSGTSYSLTISSMEAEDAA |
| region | | SYFCHQWSSYPYTFGGGTKVEIK |
| S009-F7.65.13 | SEQ ID NO: 112 | EVQLQQSGPELVKPGTSVKILCKASGDTFTAYNMDWVKQR |
| heavy chain | | HGQSLEWIGDINPNTDSTIYNQKFEGKAILTVDKSSSTAYME |
| variable region | | LRSLTSEDTAVYYCARRKLGRGYFDYWGQGTTLTVSS |
| S009-F7.65.13 | SEQ ID NO: 113 | QIVLTQSPAIMSASPGEKVTMTCSVNLSVRYIYWYQQKPGSS |
| light chain variable | | PRLLIHDTSNLASGVPVRFSGSGSGTSYSLTISRMEAEDAAT |
| region | | YYCQQWSSFPYTFGGGTKLEIK |
| S009-F7.66.12 | SEQ ID NO: 114 | QIPLQQSGPELVKPGASVRISCKASDYAFSSSWMNWVKQRP |
| heavy chain | | EKGLEWIGRIFVESGNTHYNDKFNGKATLTADKSSRTAYIQL |
| variable region | | SSLTSEDSAVYFCTRENIFYHGHSSWFAYWGQGTLVTVSA |
| S009-F7.66.12 | SEQ ID NO: 115 | DIVLTQSPASLVVSLGQRATISCRAGESVDDFGISYVHWYQQ |
| light chain variable | | KPGQPPKLLIYRAANLESGIPARFSGSGSRTDFTLTINPVETD |
| region | | DVAIYYCQQNNKDPFTFGSGTKLEIK |
| S009-F7.67.12 | SEQ ID NO: 116 | QVQLQQPGTELVKPGASVRLSCRASGYSFSSYWMHWVKQ |
| heavy chain | | RPGQGLEWLGNINPTTGDINYNEKFRNRATLAVDKSSTTAY |
| variable region | | LQLTSLTSEDSAVYFCVRHDGFLWGQGTTLTVSS |
| S009-F7.67.12 | SEQ ID NO: 117 | DIQMTQSPASLSASVGETVTITCRASENIYSYLAWYQQKQG |
| light chain variable | | KSPQLLVYYAKTLAEGVSSRFSGSGSGTQFSLKINSLQPEDF |
| region | | GTYYCQHQFGTPRTFGGGTKLEIR |
| S009-F7.69.8 | SEQ ID NO: 118 | EVHLQQSGPELVKPGASVRIPCKASGYTFTDYNMDWVKQS |
| heavy chain | | HGKSLEWIGDINPDNGGTIYTQKFKGKATLTVDKSSSTAYM |
| variable region | | ELRSLTSEDTAVYYCARRLHYYGSSGLDYWGQGTTLTVSS |
| S009-F7.69.8 light | SEQ ID NO: 119 | DIQMTQSPASLSAYVGETVTITCRASGNIHNYLAWYQQKQG |
| chain variable | | KSPQLLVYNAKTLADGVPSRFSGSGSGTQYSLKINSLQPEDF |
| region | | GSYYCQHFWSTVWTFGGGTKLEIK |

TABLE 22
Amino acid sequence information of light and heavy chain variable regions of
MSLN F8 hybridoma-positive clones

| Sequence name | Sequence No. | Amino acid sequence |
|---|---|---|
| S009-F8-4.5 heavy | SEQ ID NO: 120 | QVQLQQPGAELVKPGASVKLSCKASGYTFTIYWMHWVKQ |
| chain variable | | RPGQGLEWIGMIHPNSGNTNYNEKFKSKATLTVDKSSSTAY |
| region | | MQLSSLTSEDSAVYYCSLIHWYFDVWGTGTTVTVSS |
| S009-F8-4.5 light | SEQ ID NO: 121 | QIVLTQSPAIMSASPGEKVTMTCSASSSVSYMYWYQQKPGS |
| chain variable | | SPRLLIYDTSNLASGVPVRFSGSGSGTSYSLTISRMEAEDAAT |
| region | | YYCQQWSSYPLTFGAGTKLELK |
| S009-F8-5.15 | SEQ ID NO: 122 | QVQLQQPGTELVKPGASVKLSCKASGYTFTSYWMHWVKQ |
| heavy chain | | RPGQGLEWIGHSNPSNGGTNYNEKFKSKATLTVDKSSSTAY |
| variable region | | MQLSSLTSEDSAVYYCARWVRGSNYEYFDVWGTGTTVTVS |
| | | S |
| S009-F8-5.15 light | SEQ ID NO: 123 | DIVMTQSQKFMSTTVGDRVSIICKASQNVGSAVAWCQQKPG |
| chain variable | | QSPKLLIYSASNRYTGVPDRFTGSGSGTDFTLTISNMQSEDL |
| region | | ADYFCQQYSSYPLTFAGGTKLEIK |
| S009-F8-7.5 heavy | SEQ ID NO: 124 | DVQLQESGPGLVKPSQSLSLTCSVTGYSITSGYYWNWIRQFP |
| chain variable | | GNKLEWMAYIRYDGSNNYNPSLKNRISIIRDTSKNQFFLRLN |
| region | | SVTTEDTATYYCAKSYYGMDDWGQGISVTVSS |
| S009-F8-7.5 light | SEQ ID NO: 125 | DIQMTQSPASLSASVGETVTITCRTSGDIHNYLAWYQQKQG |
| chain variable | | KSPQLLVYKAKTLENGVPSRFTGSGSGTQFSLKIDSLQPEDF |
| region | | GTYYCQHFWTTPYTFGGGTKLEIK |
| S009-F8-8.22 | SEQ ID NO: 126 | EVQLQQSGPELVKPGASVKMSCKASGYTFTDYIIHWVKQS |
| heavy chain | | HGKSLEWIAYIFPNNGGTGYNQKFRGKATLTVNKSSSTAYM |
| variable region | | ELRSLTSEDSAVYYCARWRLRQYFDVWGTGTTVTVSS |
| S009-F8-8.22 light | SEQ ID NO: 127 | DIVMTQSQKFMSTIVGDRVSITCKASQNVGTAVAWYQQKPG |
| chain variable | | QSPKLLIYSASNRYTGVPDRFTGSGSGTDFTLTISNMQSEDL |
| region | | ADYFCQQYSSYPLTFGGGTKLEIK |
| S009-F8-9.16 | SEQ ID NO: 128 | EVHLQQSGPVLVKPGASVKMSCKASGYTFTDYFMNWVKQ |
| heavy chain | | SHGKSLEWIGVINPHNGYVNYNQKFQGRATLTVDKSSSTVY |
| variable region | | MELNSLTSEDSAVYHCARSAESAWFAYWGQRTLVTVSA |
| S009-F8-9.16 light | SEQ ID NO: 129 | DVLMTQTPLSLPVSLGDQVSISCRSSQSIVHVKGNTYLEWY |
| chain variable | | LQKPGQSPELLIYKVSNRFSGVPDRFSGGGSGTDFTLKISRV |
| region | | EPEDLGVYYCFQGSHLPYTFGGGTKLEIK |
| S009-F8-12.13 | SEQ ID NO: 130 | QVQLQQPGAELVKPGASVKLSCKASGYTFTSYWMHWVKQ |
| heavy chain | | RPGQGLEWIGMIHPNSGSTNYNEKFKSKATLTVDKSSSTAY |
| variable region | | MQLSSLTSEDSAVYYCARRTPIITLVGHWYFDVWGTGTTVT |
| | | VSS |
| S009-F8-12.13 | SEQ ID NO: 131 | DIVMTQSHKFMSTSVGDRVSITCKASQDVGTAVAWYQQKP |
| light chain variable | | GQSPKLLIYWASTRHTGVPDRFTGSGSGTDFTLTISNVQSED |
| region | | LADYFCQQYSSYPLTFGAGTKLELK |
| S009-F8-13.8 | SEQ ID NO: 132 | EFQLQQSGPELVKPGASVKISCKASGYSFTDYNMNWVKQT |
| heavy chain | | NGKSLEWIGIINPNYGTSSYNQKFKGKATLTVDQSSSTAYMQ |
| variable region | | LNSLTSEDSAVYYCARVGRDNSGFDYWGQGTTLTVSS |
| S009-F8-13.8 light | SEQ ID NO: 133 | QIVLTQSPAIMSASPGEKVTMTCSASSSVSYMYWYQQKPGS |
| chain variable | | SPRLLIYDTSNLASGVPVRFSGSGSGTSYSLTISRMEAEDAAT |
| region | | YYCQQWSSYPPTFGSGTELEIK |
| S009-F8-15.19 | SEQ ID NO: 134 | EFQLQQSGPELVKPGASVKISCKASGYSFTDYNMNWVKQS |
| heavy chain | | NGKSLEWIGIINPYYGTTSNNQNFKGKATLTVDQSSSTAYM |
| variable region | | QLNSLTSDDSAVYYCARWLRRDAMDYWGQGTSVTVSS |
| S009-F8-15.19 | SEQ ID NO: 135 | DIVMTPSHKFMSTSVGDRVSIACKASQDVGSSVAWYQQKP |
| light chain variable | | GQSPKLLIYWTSTRHTGVPDRFTGSGSGTDFTLTISNVQSED |
| region | | LADYFCQQYSSYPWTFGGGTKLEIK |
| S009-F8-18.9 | SEQ ID NO: 136 | EFQLQQSGPELVKPGASVKISCKASGYSFTDYNMNWVKQS |
| heavy chain | | NGKSLEWIGIINPNYGTTSYNQKFKGRATLTVDQSSSTAYVQ |
| variable region | | LNSLTSEDSAVYYCARGGYDNEAMDYWGQGTSVTVSS |
| S009-F8-18.9 light | SEQ ID NO: 137 | QIVLTQSPAIMSASPGEKVTMTCSASSSVSYMHWYQQKSGT |
| chain variable | | SPKRWIYDTSKLASGVPARFSGSGSGTSYSLTISSMEAEDAA |
| region | | TYYCQQWSSNPPTFGAGTKLELK |
| S009-F8-19.21 | SEQ ID NO: 138 | EFQLQQSGPELVKPGASVKISCKTSGYSFTDYNMNWVKQS |
| heavy chain | | NGKSLEWIGVINPNYGTTGYNQKFKGKATLTVDQSSSTAYM |
| variable region | | QLNSLTSEDSAVYYCARGGYDGEAMDYWGHGTSVTVSS |
| S009-F8-19.21 | SEQ ID NO: 139 | QIVLTQSPAIMSASPGEKVTMTCSASSSVNYMHWYQQKSGT |
| light chain variable | | SPKRWIYDTSKLASGVPARFSGSGSGTSYSLTISSMEAEDAA |
| region | | TYYCQQWSSYPPTFGGGTKLEIK |
| S009-F8-22.23 | SEQ ID NO: 140 | QVQLKQSGPGLVQPSQSLSITCTVSGFSLFSYGVHWVRQSP |
| heavy chain | | GKGLEWLGVIWSGGSTDYNAAFISRLSISKDNSKSQVFFKM |
| variable region | | NSLQADDTAMYYCARNGGSMITTLYYAMDYWGQGTSVTV |
| | | SS |
| S009-F8-22.23 | SEQ ID NO: 141 | DVVMTQTPLSLPVSLGDQASISCRSSQSLVHSYGNTYLHWY |
| light chain variable | | LQKPGQSPKLLIYKVSNRFSGVPDRFSGSGSGTDFTLKISRV |
| region | | EAEDLGVYFCSQSTHIPFTFGGGTKLEIK |
| S009-F8-24.14 | SEQ ID NO: 142 | EFQLQQSGPELVKPGASVKISCKASGYSFTDYNMNWVKLS |
| heavy chain | | NGKSLEWIGVIIPNYGTSSYNQKFKGKATLTVDQSSSTAYMQ |
| variable region | | LNSLTSEDSAVYYCARWDNYYGSSLDYWGQGTTLTVSS |
| S009-F8-24.14 | SEQ ID NO: 143 | DIVMTQSQKFMSTTVGDRVSITCKASQNVGTAVAWYQQKP |
| light chain variable | | GQSPKLLIYSASNRYTGVPDRFTGSGSGTDFILTISNMQSEDL |
| region | | ADYFCQQYSSYPLTFGAGTKLELK |
| S009-F8-27.1 | SEQ ID NO: 144 | EVOLVESGGGLVKPGGSLKLSCAASGFTFSSYAMSWVRQTP |
| heavy chain | | EKRLEWVATISDGGNYTYYPDNVKGRFTISRDNAKNNLYLQ |
| variable region | | MSHLKSEDTAMYYCAGDRGYPYAMDSWGQGTSVTVSS |
| S009-F8-27.1 light | SEQ ID NO: 145 | NIMMTQSPSSLAVSAGEKVTMSCKSSQSVLYSSNQKNYLA |
| chain variable | | WYQQKPGQSPKLLIYWASTRESGVPDRFTGSGSGTDFTLTIS |
| region | | NVHAEDLAVYYCHQYLSSLTFGAGTKLELK |
| S009-F8-28.23 | SEQ ID NO: 146 | EVQLVESGGDLVKPGGSLKLSCAASGFTFSSYAMSWVRQTP |
| heavy chain | | EKRLEWVATISDGGSYTYYPDNVEGRFTISRDNAKNNLYLQ |
| variable region | | MSHLKSEDTAMYYCAGDRGYSYALDYWGQGTSVTVSS |
| S009-F8-28.23 | SEQ ID NO: 147 | NIMMTQSPSSLAVSAGEKVTMSCKSSQSVLYSSNQKNYLA |
| light chain variable | | WYQQKPGQSPKLLIYWASTRESGVPDRFTGSGSGTDFTLTIS |
| region | | SVQAEDLAVYYCHQYLSSLTFGAGTKLELK |
| S009-F8-29.1 | SEQ ID NO: 148 | QVQLQQSGAELARPGASVKMSCKASGYTFISYTMHWVKQ |
| heavy chain | | RPGQGLEWIGYINPSSGYTKYNQKFKDKATLTADKSSSTAY |
| variable region | | MQLNSLTSEDSAVYYCARYYSNPDYYAMDYWGQGTSVTV |
| | | SS |
| S009-F8-29.1 light | SEQ ID NO: 149 | QIVLTQSPAIMSASPGEKVTITCSASSSVSYMHWFQQKPGTS |
| chain variable | | PKLWIYSTSNLASGVPARFSGSGSGTSYSLTISRMEAEDAAT |
| region | | YYCQQRSSYPLTFGGGTKLELK |
| S009-F8-31.22 | SEQ ID NO: 150 | EVQLVESGGDLVKPGGSLKLSCAASGFTFSTYGMSWVRQTP |
| heavy chain | | DKRLEWVATISSGGSYTYYPDSLKGRFTISRDNAKNTLYLQ |
| variable region | | MSSLKSEDTAMYYCARHEGYYYGKDYWGQGTTLTVSS |
| S009-F8-31.22 | SEQ ID NO: 151 | DIVLTQSPATLSVTPGDSVSLSCRASHSISNNLHWYQQKSHE |
| light chain variable | | SPRLLIKYASQSISGIPSKFSGSGSGTDFTLSINSVETEDFGMY |
| region | | FCQQTNSWPLTFGAGTKLELK |
| S009-F8-32.3 | SEQ ID NO: 152 | QVQLKESGPGLVAPSQSLSITCTVSGFSLSSYAISWVRQPPGK |
| heavy chain | | GLEWLGVIWTGGGTNYNSALKSRLSISKDNSKSQVFLKMN |
| variable region | | SLQTDDTARFYCARNNYGRLDYAMDYWGQGTSVTVSS |
| S009-F8-32.3 light | SEQ ID NO: 153 | DVVMTQTPLSLPVSLGDQASISCRSSQSLVHGNGNTYLHWY |
| chain variable | | LQKPGQSPKLLIYKVSNRFSGVPDRFSGSGSGTDFTLKISRV |
| region | | ETEDLGVYFCSQSTHVPFTFGSGTKLEIK |
| S009-F8-33.12 | SEQ ID NO: 154 | EVQLQQSGAELVKPGASVKLSCTASAFNIKDYYMHWVKQR |
| heavy chain | | TEQGLEWIGRIDPEDGETKYAPKFQGKATITADTSSNTAYLQ |
| variable region | | LSSLTSEDTAVYYCASGSGTGAMDYWGQGTSVTVSS |
| S009-F8-33.12 | SEQ ID NO: 155 | DIVMTQSQKFMSTSVGDRVSITCKASQNVRTAVGWYQQKP |
| light chain variable | | GQSPKALIYLASNRHTGVPDRFTGSGSGTDFTLTISNVQSED |
| region | | LADYFCLQHWNSPYTFGGGTKLVIK |
| S009-F8-36.12 | SEQ ID NO: 156 | EVQLQQSGPELVKPGASVKISCKASGYTFTDYYMNWVKQS |
| heavy chain | | HGKSLEWIGDINPNNGGTSYNQKFKGKATLTVDKSSSTAYM |
| variable region | | ELRSLTSEDSAVYYCASWGYGSSSRGYFDYWGQGTTLTVSS |
| S009-F8-36.12 | SEQ ID NO: 157 | QIVLSQSPAILSASPGEKVTMTCRASSSVSYMHWYQQKPGS |
| light chain variable | | SPKPWIYATSNLASGVPARFSGSGSGTSYSLTISRVEAEDAAT |
| region | | YYCQQWSSNPWTFGGGTKLEIK |

The sequences of heavy chain variable regions of the 71 clones were each cloned into an expression vector pcDNA3.4-B1HH1 containing a signal peptide and a heavy chain constant region of a human antibody IgG1 (sequence of the heavy chain constant region:

ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDKS
RWQQGNVFSCSVMHEALHNHYTQKSLSLSPGK, SEQ ID NO: 158) by Biointron
Biological Inc.

The sequences of light chain variable regions of the 71 clones were each cloned into an expression vector pcDNA3.4-B1HLK containing a signal peptide and a Kappa light chain constant region of a human antibody IgG1 (sequence of the light chain constant region:

RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC, SEQ ID NO: 159)

to obtain an expression vector for human-murine chimeric antibodies, and antibodies were prepared according to the method in Example 1.1.

TABLE 23
Kabat analysis of VH and VL sequences of MSLN chimeric antibodies

| Antibody | | | Antibody | | |
|---|---|---|---|---|---|
| variable region | CDR | Kabat numbering | variable region | CDR | Kabat numbering |
| F1.2.12 VH | HCDR1 | NYAVH | F7.11.11 VL | LCDR1 | SASSSVSSTYLS |
| | | (SEQ ID NO: 160) | | | (SEQ ID NO: 364) |
| | HCDR2 | VIWSGGATDYNTVFIS | | LCDR2 | STSNLAS |
| | | (SEQ ID NO: 161) | | | (SEQ ID NO: 365) |
| | HCDR3 | TGSGYAMDY | | LCDR3 | HQWSSYPYT |
| | | (SEQ ID NO: 162) | | | (SEQ ID NO: 366) |
| F1.2.12 VL | LCDR1 | RTSHNVNTYLH | F7.12.13 VH | HCDR1 | DYNMD |
| | | (SEQ ID NO: 163) | | | (SEQ ID NO: 367) |
| | LCDR2 | YASQSIS | | HCDR2 | DINPNTDDTIYNQKFNG |
| | | (SEQ ID NO: 164) | | | (SEQ ID NO: 368) |
| | LCDR3 | HQTNRWPLT | | HCDR3 | RLIGTGYFDV |
| | | (SEQ ID NO: 165) | | | (SEQ ID NO: 369) |
| F1.7.14 VH | HCDR1 | SDYAWN | F7.12.13 VL | LCDR1 | SASSSVISTYLC |
| | | (SEQ ID NO: 166) | | | (SEQ ID NO: 370) |
| | HCDR2 | CIRYSGGTTYNPSLKS | | LCDR2 | STSNLAS |
| | | (SEQ ID NO: 167) | | | (SEQ ID NO: 371) |
| | HCDR3 | SRQLGDAGFDY | | LCDR3 | HQWSNYPYT |
| | | (SEQ ID NO: 168) | | | (SEQ ID NO: 372) |
| F1.7.14 VL | LCDR1 | SASSSVSYMY | F7.18.10 VH | HCDR1 | NYLIE |
| | | (SEQ ID NO: 169) | | | (SEQ ID NO: 373) |
| | LCDR2 | RTSNLAS | | HCDR2 | VINPGSGGTKYNEKFKG |
| | | (SEQ ID NO: 170) | | | (SEQ ID NO: 374) |
| | LCDR3 | QQYHSYPPT | | HCDR3 | DYDYDPFAY |
| | | (SEQ ID NO: 171) | | | (SEQ ID NO: 375) |
| F1.25.10 VH | HCDR1 | DYTMN | F7.18.10 VL | LCDR1 | KASQDVSTAVA |
| | | (SEQ ID NO: 172) | | | (SEQ ID NO: 376) |
| | HCDR2 | LFNPYNGGISYNQKFKG | | LCDR2 | WASTRHT |
| | | (SEQ ID NO: 173) | | | (SEQ ID NO: 377) |
| | HCDR3 | DGRGGFYAMDY | | LCDR3 | QQHYSTPYT |
| | | (SEQ ID NO: 174) | | | (SEQ ID NO: 378) |
| F1.25.10 VL | LCDR1 | RASQDISIYLN | F7.21.16 VH | HCDR1 | TFWMH |
| | | (SEQ ID NO: 175) | | | (SEQ ID NO: 379) |
| | LCDR2 | YTSRLHS | | HCDR2 | GVNPTNGGTNYNEKFRT |
| | | (SEQ ID NO: 176) | | | (SEQ ID NO: 380) |
| | LCDR3 | QQGYTLPPWT | | HCDR3 | HYISSRPGFAY |
| | | (SEQ ID NO: 177) | | | (SEQ ID NO: 381) |
| F1.35.24 VH | HCDR1 | DYYMH | F7.21.16/ | LCDR1 | KASQNVGTNVA |
| | | (SEQ ID NO: 178) | F7.25.19/ | | (SEQ ID NO: 382) |
| | HCDR2 | EINPSTGGTSYNPKFKD | F7.30.5/ | LCDR2 | SASYRYS |
| | | (SEQ ID NO: 179) | F7.48.1 VL | | (SEQ ID NO: 383) |
| | HCDR3 | YHYYGSSSYVMDY | | LCDR3 | QQYNSYPLT |
| | | (SEQ ID NO: 180) | | | (SEQ ID NO: 384) |
| F1.35.24 VL | LCDR1 | SASSSVYHMH | F7.23.19 VH | HCDR1 | DYYMS |
| | | (SEQ ID NO: 181) | | | (SEQ ID NO: 385) |
| | LCDR2 | DTSKLAS | | HCDR2 | FIRSKANAYTTEYSASVKG |
| | | (SEQ ID NO: 182) | | | (SEQ ID NO: 386) |
| | LCDR3 | QHWRTNPLT | | HCDR3 | YYYYGSGYVWYFDV |
| | | (SEQ ID NO: 183) | | | (SEQ ID NO: 387) |
| F1.56.1 VH | HCDR1 | HTYMH | F7.23.19 VL | LCDR1 | KASQSVGTNVA |
| | | (SEQ ID NO: 184) | | | (SEQ ID NO: 388) |
| | HCDR2 | RIDPANGNTEYDPKFQG | | LCDR2 | SASYRYS |
| | | (SEQ ID NO: 185) | | | (SEQ ID NO: 389) |
| | HCDR3 | DGWYIDV | | LCDR3 | QQYNSYPLT |
| | | (SEQ ID NO: 186) | | | (SEQ ID NO: 390) |
| F1.56.1 VL | LCDR1 | KSSQSLLYSNNQKNYLA | F7.25.19 VH | HCDR1 | SYWMH |
| | | (SEQ ID NO: 187) | | | (SEQ ID NO: 391) |
| | LCDR2 | WASTRES | | HCDR2 | NIIPSNGGTKYNEKFKS |
| | | (SEQ ID NO: 188) | | | (SEQ ID NO: 392) |
| | LCDR3 | QRYYSYPWT | | HCDR3 | HYYGGSPGFAY |
| | | (SEQ ID NO: 189) | | | (SEQ ID NO: 393) |
| F1.57.1 VH | HCDR1 | DIFLH | F7.26.15 VH | HCDR1 | TGNYRWS |
| | | (SEQ ID NO: 190) | | | (SEQ ID NO: 394) |
| | HCDR2 | RIDPASDNTIYDPKFQD | | HCDR2 | YIYYTGFITYNPSLTS |
| | | (SEQ ID NO: 191) | | | (SEQ ID NO: 395) |
| | HCDR3 | FDF | | HCDR3 | DDYDYDVFAY |
| | | (SEQ ID NO: 192) | | | (SEQ ID NO: 396) |
| F1.57.1 VL | LCDR1 | RSSESLLHSNGNTYLY | F7.26.15 VL | LCDR1 | RASGNIHNYLA |
| | | (SEQ ID NO: 193) | | | (SEQ ID NO: 397) |
| | LCDR2 | RMSNLAS | | LCDR2 | KAKTLAD |
| | | (SEQ ID NO: 194) | | | (SEQ ID NO: 398) |
| | LCDR3 | MQHLEYPLT | | LCDR3 | QHFLSIPLT |
| | | (SEQ ID NO: 195) | | | (SEQ ID NO: 399) |
| F1.59.1 VH | HCDR1 | SYWMN | F7.30.5 VH | HCDR1 | TYWMH |
| | | (SEQ ID NO: 196) | | | (SEQ ID NO: 400) |
| | HCDR2 | EIRLKSNGFAAFYAESVKG | | HCDR2 | NVNPSNGGTMYNEKFER |
| | | (SEQ ID NO: 197) | | | (SEQ ID NO: 401) |
| | HCDR3 | FVH | | HCDR3 | HYIGSRPGFAY |
| | | (SEQ ID NO: 198) | | | (SEQ ID NO: 402) |
| F1.59.1 VL | LCDR1 | RTSESVEYFGTILIQ | F7.33.24 VH | HCDR1 | SYWMH |
| | | (SEQ ID NO: 199) | | | (SEQ ID NO: 403) |
| | LCDR2 | GASNVES | | HCDR2 | NVNPSNGGSNYNEKFKN |
| | | (SEQ ID NO: 200) | | | (SEQ ID NO: 404) |
| | LCDR3 | QQNRKVPYT | | HCDR3 | HYIGSRPGFAY |
| | | (SEQ ID NO: 201) | | | (SEQ ID NO: 405) |
| F1.62.9 VH | HCDR1 | DTFMH | F7.33.24 VL | LCDR1 | KASQNVGTNIA |
| | | (SEQ ID NO: 202) | | | (SEQ ID NO: 406) |
| | HCDR2 | RIDPANDNSIYGPKFQD | | LCDR2 | SASYRYS |
| | | (SEQ ID NO: 203) | | | (SEQ ID NO: 407) |
| | HCDR3 | FDN | | LCDR3 | QQYNSYPLT |
| | | (SEQ ID NO: 204) | | | (SEQ ID NO: 408) |
| F1.62.9 VL | LCDR1 | RSDKSLLHSNGHNYLY | F7.41.18 VH | HCDR1 | SYWMH |
| | | (SEQ ID NO: 205) | | | (SEQ ID NO: 409) |
| | LCDR2 | RMSNLAS | | HCDR2 | SINPSNGDTYYNEKFKN |
| | | (SEQ ID NO: 206) | | | (SEQ ID NO: 410) |
| | LCDR3 | MQHLEYPLT | | HCDR3 | GWYFDV |
| | | (SEQ ID NO: 207) | | | (SEQ ID NO: 411) |
| F2.13.3 VH | HCDR1 | GYWIE | F7.41.18 VL | LCDR1 | KSSQSLLNSSSQKNYLA |
| | | (SEQ ID NO: 208) | | | (SEQ ID NO: 412) |
| | HCDR2 | EILPGSGSTNYNEKFKG | | LCDR2 | FASTRES |
| | | (SEQ ID NO: 209) | | | (SEQ ID NO: 413) |
| | HCDR3 | AGAWFAY | | LCDR3 | QQHYSTPRT |
| | | (SEQ ID NO: 210) | | | (SEQ ID NO: 414) |
| F2.13.3 VL | LCDR1 | TASSSVSSSYLH | F7.44.20 VH | HCDR1 | DYNMD |
| | | (SEQ ID NO: 211) | | | (SEQ ID NO: 415) |
| | LCDR2 | STSNLAS | | HCDR2 | DINPSTGGTIYNQKFNG |
| | | (SEQ ID NO: 212) | | | (SEQ ID NO: 416) |
| | LCDR3 | HQYHRSPWT | | HCDR3 | RRIGTGYFDV |
| | | (SEQ ID NO: 213) | | | (SEQ ID NO: 417) |
| F2.16.10 VH | HCDR1 | SYWMH | F7.44.20 VL | LCDR1 | SASSSVISSYLS |
| | | (SEQ ID NO: 214) | | | SEQ ID NO: 418) |
| | HCDR2 | IIHPNIGSTNYNERFKS | | LCDR2 | RTSNLAS |
| | | (SEQ ID NO: 215) | | | (SEQ ID NO: 419) |
| | HCDR3 | RSSNYGDWYFDV | | LCDR3 | HQWSSFPYT |
| | | (SEQ ID NO: 216) | | | (SEQ ID NO: 420) |
| F2.16.10 VL | LCDR1 | KASQDVGTSVA | F7.48.1 VH | HCDR1 | SYWMH |
| | | (SEQ ID NO: 217) | | | (SEQ ID NO: 421) |
| | LCDR2 | WASTQHT | | HCDR2 | NINPSNGGTNNNENFKS |
| | | (SEQ ID NO: 218) | | | (SEQ ID NO: 422) |
| | LCDR3 | QQYSSYPLT | | HCDR3 | NGYHGYWYFDV |
| | | (SEQ ID NO: 219) | | | (SEQ ID NO: 423) |
| F2.17.3 VH | HCDR1 | SYWMH | F7.53.2 VH | HCDR1 | DYNMD |
| | | (SEQ ID NO: 220) | | | (SEQ ID NO: 424) |
| | HCDR2 | NINPSNGDTFYNEKFKN | | HCDR2 | DINPNTDGAIYNQKFQN |
| | | (SEQ ID NO: 221) | | | (SEQ ID NO: 425) |
| | HCDR3 | GWLRDY | | HCDR3 | RKLGRKFFDY |
| | | (SEQ ID NO: 222) | | | (SEQ ID NO: 426) |
| F2.17.3 VL | LCDR1 | KSSQSLLNSSSQKNYLA | F7.53.2 VL | LCDR1 | SASSSVGYMN |
| | | (SEQ ID NO: 223) | | | (SEQ ID NO: 427) |
| | LCDR2 | FASTKDS | | LCDR2 | DTSNLAS |
| | | (SEQ ID NO: 224) | | | (SEQ ID NO: 428) |
| | LCDR3 | QQHYTTPYT | | LCDR3 | QQWYVYPYT |
| | | (SEQ ID NO: 225) | | | (SEQ ID NO: 429) |
| F2.21.4 VH | HCDR1 | DYNMD | F7.61.21 VH | HCDR1 | DYNVD |
| | | (SEQ ID NO: 226) | | | (SEQ ID NO: 430) |
| | HCDR2 | DINPNNGGSIYNQRFKG | | HCDR2 | DVNPNTDGAIYNQKFEG |
| | | (SEQ ID NO: 227) | | | (SEQ ID NO: 431) |
| | HCDR3 | RAYYSTGYFDV | | HCDR3 | RRLGQGGFDS |
| | | (SEQ ID NO: 228) | | | (SEQ ID NO: 432) |
| F2.21.4 VL | LCDR1 | SASSSISYMH | F7.61.21 VL | LCDR1 | GASSSLISKYLY |
| | | (SEQ ID NO: 229) | | | (SEQ ID NO: 433) |
| | LCDR2 | STSTLAS | | LCDR2 | STSNLAS |
| | | (SEQ ID NO: 230) | | | (SEQ ID NO: 434) |
| | LCDR3 | QQRSSYPPT | | LCDR3 | HQWSSYPYT |
| | | (SEQ ID NO: 231) | | | (SEQ ID NO: 435) |
| F2.23.12 VH | HCDR1 | NYWMH | F7.65.13 VH | HCDR1 | AYNMD |
| | | (SEQ ID NO: 232) | | | (SEQ ID NO: 436) |
| | HCDR2 | NINPSNGGPYYNERFRS | | HCDR2 | DINPNTDSTIYNQKFEG |
| | | (SEQ ID NO: 233) | | | (SEQ ID NO: 437) |
| | HCDR3 | PYYGSSYGYFDY | | HCDR3 | RKLGRGYFDY |
| | | (SEQ ID NO: 234) | | | (SEQ ID NO: 438) |
| F2.23.12 VL | LCDR1 | KASQDINKYIA | F7.65.13 VL | LCDR1 | SVNLSVRYIY |
| | | (SEQ ID NO: 235) | | | (SEQ ID NO: 439) |
| | LCDR2 | YTSELQP | | LCDR2 | DTSNLAS |
| | | (SEQ ID NO: 236) | | | (SEQ ID NO: 440) |
| | LCDR3 | LQYANPLRT | | LCDR3 | QQWSSFPYT |
| | | (SEQ ID NO: 237) | | | (SEQ ID NO: 441) |
| F2.38.12 VH | HCDR1 | SYWMH | F7.66.12 VH | HCDR1 | SSWMN |
| | | (SEQ ID NO: 238) | | | (SEQ ID NO: 442) |
| | HCDR2 | NINPSNGGTNYNEKIKN | | HCDR2 | RIFVESGNTHYNDKFNG |
| | | (SEQ ID NO: 239) | | | (SEQ ID NO: 443) |
| | HCDR3 | WNYYGNYPFDY | | HCDR3 | ENIFYHGHSSWFAY |
| | | (SEQ ID NO: 240) | | | (SEQ ID NO: 444) |
| F2.38.12/ | LCDR1 | KASQNVGTAVA | F7.66.12 VL | LCDR1 | RAGESVDDFGISYVH |
| F8-8.22/ | | (SEQ ID NO: 241) | | | (SEQ ID NO: 445) |
| F8-24.14 VL | LCDR2 | SASNRYT | | LCDR2 | RAANLES |
| | | (SEQ ID NO: 242) | | | (SEQ ID NO: 446) |
| | LCDR3 | QQYSSYPLT | | LCDR3 | QQNNKDPFT |
| | | (SEQ ID NO: 243) | | | (SEQ ID NO: 447) |
| F2.39.3 VH | HCDR1 | NYWMH | F7.67.12 VH | HCDR1 | SYWMH |
| | | (SEQ ID NO: 244) | | | (SEQ ID NO: 448) |
| | HCDR2 | NINPSSGDSYYNERFMS | | HCDR2 | NINPTTGDINYNEKFRN |
| | | (SEQ ID NO: 245) | | | (SEQ ID NO: 449) |
| | HCDR3 | SGGLWLAF | | HCDR3 | HDGFL |
| | | (SEQ ID NO: 246) | | | (SEQ ID NO: 450) |
| F2.39.3 VL | LCDR1 | KSSQTLLNSVSQNNYLA | F7.67.12 VL | LCDR1 | RASENIYSYLA |
| | | (SEQ ID NO: 247) | | | (SEQ ID NO: 451) |
| | LCDR2 | FASTRES | | LCDR2 | YAKTLAE |
| | | (SEQ ID NO: 248) | | | (SEQ ID NO: 452) |
| | LCDR3 | QQHYRTPYT | | LCDR3 | QHQFGTPRT |
| | | (SEQ ID NO: 249) | | | (SEQ ID NO: 453) |
| F2.47.1 VH | HCDR1 | SYWMH | F7.69.8 VH | HCDR1 | DYNMD |
| | | (SEQ ID NO: 250) | | | (SEQ ID NO: 454) |
| | HCDR2 | MIHPNSGSTNYNEKFKS | | HCDR2 | DINPDNGGTIYTQKFKG |
| | | (SEQ ID NO: 251) | | | (SEQ ID NO: 455) |
| | HCDR3 | PVVPYWYFDV | | HCDR3 | RLHYYGSSGLDY |
| | | (SEQ ID NO: 252) | | | (SEQ ID NO: 456) |
| F2.47.1 VL | LCDR1 | KASQNVGTAVA | F7.69.8 VL | LCDR1 | RASGNIHNYLA |
| | | (SEQ ID NO: 253) | | | (SEQ ID NO: 457) |
| | LCDR2 | SASNRYT | | LCDR2 | NAKTLAD |
| | | (SEQ ID NO: 254) | | | (SEQ ID NO: 458) |
| | LCDR3 | QQSSSYPLT | | LCDR3 | QHFWSTVWT |
| | | (SEQ ID NO: 255) | | | (SEQ ID NO: 459) |
| F2-56.12 VH | HCDR1 | SGYDWH | F8-4.5 VH | HCDR1 | IYWMH |
| | | (SEQ ID NO: 256) | | | (SEQ ID NO: 460) |
| | HCDR2 | YISYSGSTNYNPSLKS | | HCDR2 | MIHPNSGNTNYNEKFKS |
| | | (SEQ ID NO: 257) | | | (SEQ ID NO: 461) |
| | HCDR3 | GTGPDY | | HCDR3 | IHWYFDV |
| | | (SEQ ID NO: 258) | | | (SEQ ID NO: 462) |
| F2-56.12 VL | LCDR1 | KASQNVRTAVA | F8-4.5 VL | LCDR1 | SASSSVSYMY |
| | | (SEQ ID NO: 259) | | | (SEQ ID NO: 463) |
| | LCDR2 | LPSNRHT | | LCDR2 | DTSNLAS |
| | | (SEQ ID NO: 260) | | | (SEQ ID NO: 464) |
| | LCDR3 | LQHWNYPLT | | LCDR3 | QQWSSYPLT |
| | | (SEQ ID NO: 261) | | | (SEQ ID NO: 465) |
| F2.58.8 VH | HCDR1 | SYWVH | F8-5.15 VH | HCDR1 | SYWMH |
| | | (SEQ ID NO: 262) | | | (SEQ ID NO: 466) |
| | HCDR2 | YINPNSGYTKYNQKFKD | | HCDR2 | HSNPSNGGTNYNEKFKS |
| | | (SEQ ID NO: 263) | | | (SEQ ID NO: 467) |
| | HCDR3 | HYYGSSRDYFDY | | HCDR3 | WVRGSNYEYFDV |
| | | (SEQ ID NO: 264) | | | (SEQ ID NO: 468) |
| F2.58.8 VL | LCDR1 | SASSSVSYMY | F8-5.15 VL | LCDR1 | KASQNVGSAVA |
| | | (SEQ ID NO: 265) | | | (SEQ ID NO: 469) |
| | LCDR2 | DTSNLAS | | LCDR2 | SASNRYT |
| | | (SEQ ID NO: 266) | | | (SEQ ID NO: 470) |
| | LCDR3 | QQWNSYPYT | | LCDR3 | QQYSSYPLT |
| | | (SEQ ID NO: 267) | | | (SEQ ID NO: 471) |
| F3.7.3/ | HCDR1 | NYWMN | F8-7.5 VH | HCDR1 | SGYYWN |
| F3.16.1 VH | | (SEQ ID NO: 268) | | | (SEQ ID NO: 472) |
| | HCDR2 | QIRLKSDNYATHYAESVK | | HCDR2 | YIRYDGSNNYNPSLKN |
| | | G | | | (SEQ ID NO: 473) |
| | | (SEQ ID NO: 269) | | HCDR3 | SYYGMDD |
| | HCDR3 | GTGGY | | | (SEQ ID NO: 474) |
| | | (SEQ ID NO: 270) | | | |
| F3.7.3 VL | LCDR1 | RASESVSIVGTNVIH | F8-7.5 VL | LCDR1 | RTSGDIHNYLA |
| | | (SEQ ID NO: 271) | | | (SEQ ID NO: 475) |
| | LCDR2 | HASNLET | | LCDR2 | KAKTLEN |
| | | (SEQ ID NO: 272) | | | (SEQ ID NO: 476) |
| | LCDR3 | LQSRKIPWT | | LCDR3 | QHFWTTPYT |
| | | (SEQ ID NO: 273) | | | (SEQ ID NO: 477) |
| F3.16.1 VL | LCDR1 | RASESVSIIGTD VIH | F8-8.22 VH | HCDR1 | DYIIH |
| | | (SEQ ID NO: 274) | | | (SEQ ID NO: 478) |
| | LCDR2 | HASNLET | | HCDR2 | YIFPNNGGTGYNQKFRG |
| | | (SEQ ID NO: 275) | | | (SEQ ID NO: 479) |
| | LCDR3 | LQSRKIPWT | | HCDR3 | WRLRQYFDV |
| | | (SEQ ID NO: 276) | | | (SEQ ID NO: 480) |
| F3.23.1 VH | HCDR1 | DYEMH | F8-9.16 VH | HCDR1 | DYFMN |
| | | (SEQ ID NO: 277) | | | (SEQ ID NO: 481) |
| | HCDR2 | AIDPETGDTVYNQNFKG | | HCDR2 | VINPHNGYVNYNQKFQG |
| | | (SEQ ID NO: 278) | | | (SEQ ID NO: 482) |
| | HCDR3 | YGYDWG | | HCDR3 | SAESAWFAY |
| | | (SEQ ID NO: 279) | | | (SEQ ID NO: 483) |
| F3.23.1 VL | LCDR1 | KSSQSLLYSNGKTYLN | F8-9.16 VL | LCDR1 | RSSQSIVHVKGNTYLE |
| | | (SEQ ID NO: 280) | | | (SEQ ID NO: 484) |
| | LCDR2 | LVSKLDS | | LCDR2 | KVSNRFS |
| | | (SEQ ID NO: 281) | | | (SEQ ID NO: 485) |
| | LCDR3 | VQGTHFPWT | | LCDR3 | FQGSHLPYT |
| | | (SEQ ID NO: 282) | | | (SEQ ID NO: 486) |
| F3.38.10 VH | HCDR1 | DYYMN | F8-12.13 VH | HCDR1 | SYWMH |
| | | (SEQ ID NO: 283) | | | (SEQ ID NO: 487) |
| | HCDR2 | VINPKNGVISHNQKFKG | | HCDR2 | MIHPNSGSTNYNEKFKS |
| | | (SEQ ID NO: 284) | | | (SEQ ID NO: 488) |
| | HCDR3 | YGSRFYAMDY | | HCDR3 | RTPIITLVGHWYFDV |
| | | (SEQ ID NO: 285) | | | (SEQ ID NO: 489) |
| F3.38.10 VL | LCDR1 | RSSQSLIHSDGNTYLQ | F8-12.13 VL | LCDR1 | KASQDVGTAVA |
| | | (SEQ ID NO: 286) | | | (SEQ ID NO: 490) |
| | LCDR2 | KVSNRFS | | LCDR2 | WASTRHT |
| | | (SEQ ID NO: 287) | | | (SEQ ID NO: 491) |
| | LCDR3 | SQTTHVPFT | | LCDR3 | QQYSSYPLT |
| | | (SEQ ID NO: 288) | | | (SEQ ID NO: 492) |
| F3.45.21 VH | HCDR1 | SGYYWN | F8-13.8 VH | HCDR1 | DYNMN |
| | | (SEQ ID NO: 289) | | | (SEQ ID NO: 493) |
| | HCDR2 | FIRFDGTNNYNPSLKN | | HCDR2 | IINPNYGTSSYNQKFKG |
| | | (SEQ ID NO: 290) | | | (SEQ ID NO: 494) |
| | HCDR3 | EGSYAPAWFAY | | HCDR3 | VGRDNSGFDY |
| | | (SEQ ID NO: 291) | | | (SEQ ID NO: 495) |
| F3.45.21 VL | LCDR1 | SASSSLSYMY | F8-13.8 VL | LCDR1 | SASSSVSYMY |
| | | (SEQ ID NO: 292) | | | (SEQ ID NO: 496) |
| | LCDR2 | DTSNLAS | | LCDR2 | DTSNLAS |
| | | (SEQ ID NO: 293) | | | (SEQ ID NO: 497) |
| | LCDR3 | QQWSSYPPT | | LCDR3 | QQWSSYPPT |
| | | (SEQ ID NO: 294) | | | (SEQ ID NO: 498) |
| F3.51.8 VH | HCDR1 | DYEMH | F8-15.19 VH | HCDR1 | DYNMN |
| | | (SEQ ID NO: 295) | | | (SEQ ID NO: 499) |
| | HCDR2 | GFDPETGGTAHNQKFKG | | HCDR2 | IINPYYGTTSNNQNFKG |
| | | (SEQ ID NO: 296) | | | (SEQ ID NO: 500) |
| | HCDR3 | YGSIWGDC | | HCDR3 | WLRRDAMDY |
| | | (SEQ ID NO: 297) | | | (SEQ ID NO: 501) |
| F3.51.8 VL | LCDR1 | KSSQSLLYSDGKTYLN | F8-15.19 VL | LCDR1 | KASQDVGSSVA |
| | | (SEQ ID NO: 298) | | | (SEQ ID NO: 502) |
| | LCDR2 | LVSKLDS | | LCDR2 | WTSTRHT |
| | | (SEQ ID NO: 299) | | | (SEQ ID NO: 503) |
| | LCDR3 | WQGTHFPWT | | LCDR3 | QQYSSYPWT |
| | | (SEQ ID NO: 300) | | | (SEQ ID NO: 504) |
| F3-63.5 VH | HCDR1 | DYEMH | F8-18.9 VH | HCDR1 | DYNMN |
| | | (SEQ ID NO: 301) | | | (SEQ ID NO: 505) |
| | HCDR2 | GIDPETGDTAYNQQFKG | | HCDR2 | IINPNYGTTSYNQKFKG |
| | | (SEQ ID NO: 302) | | | (SEQ ID NO: 506) |
| | HCDR3 | YASSREDY | | HCDR3 | GGYDNEAMDY |
| | | (SEQ ID NO: 303) | | | (SEQ ID NO: 507) |
| F3-63.5 VL | LCDR1 | KSSQSLLYSNGKTYLS | F8-18.9 VL | LCDR1 | SASSSVSYMH |
| | | (SEQ ID NO: 304) | | | (SEQ ID NO: 508) |
| | LCDR2 | QVSKLDS | | LCDR2 | DTSKLAS |
| | | (SEQ ID NO: 305) | | | (SEQ ID NO: 509) |
| | LCDR3 | VQITHFPQT | | LCDR3 | QQWSSNPPT |
| | | (SEQ ID NO: 306) | | | SEQ ID NO: 510) |
| F3.74.20 VH | HCDR1 | DSEMH | F8-19.21 VH | HCDR1 | DYNMN |
| | | (SEQ ID NO: 307) | | | (SEQ ID NO: 511) |
| | HCDR2 | AIDPEIDGTAYNQNFRD | | HCDR2 | VINPNYGTTGYNQKFKG |
| | | (SEQ ID NO: 308) | | | (SEQ ID NO: 512) |
| | HCDR3 | YFGSGYGYFDV | | HCDR3 | GGYDGEAMDY |
| | | (SEQ ID NO: 309) | | | (SEQ ID NO: 513) |
| F3.74.20 VL | LCDR1 | RSSQSLVYSNGNTYLH | F8-19.21 VL | LCDR1 | SASSSVNYMH |
| | | (SEQ ID NO: 310) | | | (SEQ ID NO: 514) |
| | LCDR2 | KVSNRFS | | LCDR2 | DTSKLAS |
| | | (SEQ ID NO: 311) | | | (SEQ ID NO: 515) |
| | LCDR3 | SQSTHIPFT | | LCDR3 | QQWSSYPPT |
| | | (SEQ ID NO: 312) | | | (SEQ ID NO: 516) |
| F3.80.22 VH | HCDR1 | DYEIH | F8-22.23 VH | HCDR1 | SYGVH |
| | | (SEQ ID NO: 313) | | | (SEQ ID NO: 517) |
| | HCDR2 | AFDPEIGGSAYNQKFKD | | HCDR2 | VIWSGGSTDYNAAFIS |
| | | (SEQ ID NO: 314) | | | (SEQ ID NO: 518) |
| | HCDR3 | YYGSSSGYFDV | | HCDR3 | NGGSMITTLYYAMDY |
| | | (SEQ ID NO: 315) | | | (SEQ ID NO: 519) |
| F3.80.22 VL | LCDR1 | RSSQSLEHNNGNTYLH | F8-22.23 VL | LCDR1 | RSSQSLVHSYGNTYLH |
| | | (SEQ ID NO: 316) | | | (SEQ ID NO: 520) |
| | LCDR2 | KVSNRFS | | LCDR2 | KVSNRFS |
| | | (SEQ ID NO: 317) | | | (SEQ ID NO: 521) |
| | LCDR3 | SQSTHVPFT | | LCDR3 | SQSTHIPFT |
| | | (SEQ ID NO: 318) | | | (SEQ ID NO: 522) |
| F4-94.15 VH | HCDR1 | SYWVH | F8-24.14 VH | HCDR1 | DYNMN |
| | | (SEQ ID NO: 319) | | | (SEQ ID NO: 523) |
| | HCDR2 | NIDPSDSEIHYNQKFKD | | HCDR2 | VIIPNYGTSSYNQKFKG |
| | | (SEQ ID NO: 320) | | | (SEQ ID NO: 524) |
| | HCDR3 | GGVPWFAY | | HCDR3 | WDNYYGSSLDY |
| | | (SEQ ID NO: 321) | | | (SEQ ID NO: 525) |
| F4-94.15 VL | LCDR1 | SASSSVSSTYLY | F8-27.1 VH | HCDR1 | SYAMS |
| | | (SEQ ID NO: 322) | | | (SEQ ID NO: 526) |
| | LCDR2 | STSNLAS | | HCDR2 | TISDGGNYTYYPDNVKG |
| | | (SEQ ID NO: 323) | | | (SEQ ID NO: 527) |
| | LCDR3 | HQWISYPYT | | HCDR3 | DRGYPYAMDS |
| | | (SEQ ID NO: 324) | | | (SEQ ID NO: 528) |
| F4-127.10 VH | HCDR1 | DYYMN | F8-27.1/ | LCDR1 | KSSQSVLYSSNQKNYLA |
| | | (SEQ ID NO: 325) | F8-28.23 VL | | (SEQ ID NO: 529) |
| | HCDR2 | VINPYNDVISYNQKFKG | | LCDR2 | WASTRES |
| | | (SEQ ID NO: 326) | | | (SEQ ID NO: 530) |
| | HCDR3 | YGSSYYAMDY | | LCDR3 | HQYLSSLT |
| | | (SEQ ID NO: 327) | | | (SEQ ID NO: 531) |
| F4-127.10 VL | LCDR1 | RSSQSLVHSNGNTYLQ | F8-28.23 VH | HCDR1 | SYAMS |
| | | (SEQ ID NO: 328) | | | (SEQ ID NO: 532) |
| | LCDR2 | KVSNRFS | | HCDR2 | TISDGGSYTYYPDNVEG |
| | | (SEQ ID NO: 329) | | | (SEQ ID NO: 533) |
| | LCDR3 | SQTTHVPFT | | HCDR3 | DRGYSYALDY |
| | | (SEQ ID NO: 330) | | | (SEQ ID NO: 534) |
| F5-9.16 VH | HCDR1 | DYFMN | F8-29.1 VH | HCDR1 | SYTMH |
| | | (SEQ ID NO: 331) | | | (SEQ ID NO: 535) |
| | HCDR2 | VINPSNGVINYNQRFKG | | HCDR2 | YINPSSGYTKYNQKFKD |
| | | (SEQ ID NO: 332) | | | (SEQ ID NO: 536) |
| | HCDR3 | SYDFAWFAY | | HCDR3 | YYSNPDYYAMDY |
| | | (SEQ ID NO: 333) | | | (SEQ ID NO: 537) |
| F5-9.16 VL | LCDR1 | RSSQNIVHSNGNTYLE | F8-29.1 VL | LCDR1 | SASSSVSYMH |
| | | (SEQ ID NO: 334) | | | (SEQ ID NO: 538) |
| | LCDR2 | TVSNRFS | | LCDR2 | STSNLAS |
| | | (SEQ ID NO: 335) | | | (SEQ ID NO: 539) |
| | LCDR3 | FQGSHVPYT | | LCDR3 | QQRSSYPLT |
| | | (SEQ ID NO: 336) | | | (SEQ ID NO: 540) |
| F6-62.5 VH | HCDR1 | AYWMH | F8-31.22 VH | HCDR1 | TYGMS |
| | | (SEQ ID NO: 337) | | | (SEQ ID NO: 541) |
| | HCDR2 | NINPSNGGTDFNEKFKS | | HCDR2 | TISSGGSYTYYPDSLKG |
| | | (SEQ ID NO: 338) | | | (SEQ ID NO: 542) |
| | HCDR3 | GRGYFDV | | HCDR3 | HEGYYYGKDY |
| | | (SEQ ID NO: 339) | | | (SEQ ID NO: 543) |
| F6-62.5 VL | LCDR1 | RASKNIGTSIH | F8-31.22 VL | LCDR1 | RASHSISNNLH |
| | | (SEQ ID NO: 340) | | | (SEQ ID NO: 544) |
| | LCDR2 | YASESIS | | LCDR2 | YASQSIS |
| | | (SEQ ID NO: 341) | | | (SEQ ID NO: 545) |
| | LCDR3 | QQSNSWPTLT | | LCDR3 | QQTNSWPLT |
| | | (SEQ ID NO: 342) | | | (SEQ ID NO: 546) |
| F6-76.1 VH | HCDR1 | DYYIN | F8-32.3 VH | HCDR1 | SYAIS |
| | | (SEQ ID NO: 343) | | | (SEQ ID NO: 547) |
| | HCDR2 | WIFPGRGSTFYYEKFKG | | HCDR2 | VIWTGGGTNYNSALKS |
| | | (SEQ ID NO: 344) | | | (SEQ ID NO: 548) |
| | HCDR3 | RGDSAGYEDLDY | | HCDR3 | NNYGRLDYAMDY |
| | | (SEQ ID NO: 345) | | | (SEQ ID NO: 549) |
| F6-76.1 VL | LCDR1 | KASENVVTYVS | F8-32.3 VL | LCDR1 | RSSQSLVHGNGNTYLH |
| | | (SEQ ID NO: 346) | | | (SEQ ID NO: 550) |
| | LCDR2 | GASKRYT | | LCDR2 | KVSNRFS |
| | | (SEQ ID NO: 347) | | | (SEQ ID NO: 551) |
| | LCDR3 | GQSYSYPYT | | LCDR3 | SQSTHVPFT |
| | | (SEQ ID NO: 348) | | | (SEQ ID NO: 552) |
| F7.2.3 VH | HCDR1 | SNGMS | F8-33.12 VH | HCDR1 | DYYMH |
| | | (SEQ ID NO: 349) | | | (SEQ ID NO: 553) |
| | HCDR2 | TISSGGRYTYYPDSVKG | | HCDR2 | RIDPEDGETKYAPKFQG |
| | | (SEQ ID NO: 350) | | | (SEQ ID NO: 554) |
| | HCDR3 | RGIYDYFDC | | HCDR3 | GSGTGAMDY |
| | | (SEQ ID NO: 351) | | | (SEQ ID NO: 555) |
| F7.2.3 VL | LCDR1 | RASENIHSSLA | F8-33.12 VL | LCDR1 | KASQNVRTAVG |
| | | (SEQ ID NO: 352) | | | (SEQ ID NO: 556) |
| | LCDR2 | AATNLAD | | LCDR2 | LASNRHT |
| | | (SEQ ID NO: 353) | | | (SEQ ID NO: 557) |
| | LCDR3 | QHFWGTPWT | | LCDR3 | LQHWNSPYT |
| | | (SEQ ID NO: 354) | | | (SEQ ID NO: 558) |
| F7.6.17 VH | HCDR1 | TYTMH | F8-36.12 VH | HCDR1 | DYYMN |
| | | (SEQ ID NO: 355) | | | (SEQ ID NO: 559) |
| | HCDR2 | YISPTGDFTKYNQKFKD | | HCDR2 | DINPNNGGTSYNQKFKG |
| | | (SEQ ID NO: 356) | | | (SEQ ID NO: 560) |
| | HCDR3 | WNGTVWFAY | | HCDR3 | WGYGSSSRGYFDY |
| | | (SEQ ID NO: 357) | | | (SEQ ID NO: 561) |
| F7.6.17 VL | LCDR1 | KASQALGTNVA | F8-36.12 VL | LCDR1 | RASSSVSYMH |
| | | (SEQ ID NO: 358) | | | (SEQ ID NO: 562) |
| | LCDR2 | SASYRYS | | LCDR2 | ATSNLAS |
| | | (SEQ ID NO: 359) | | | (SEQ ID NO: 563) |
| | LCDR3 | QQYNNYPLT | | LCDR3 | QQWSSNPWT |
| | | (SEQ ID NO: 360) | | | (SEQ ID NO: 564) |
| | HCDR1 | DYNMD | | | |
| | | (SEQ ID NO: 361) | | | |
| F7.11.11 VH | HCDR1 | DYNMD | | | |
| | | (SEQ ID NO: 361) | | | |
| | HCDR2 | DVFPYNGDSIYNQKFEG | | | |
| | | (SEQ ID NO: 362) | | | |
| | HCDR3 | RKTGTGYFDV | | | |
| | | (SEQ ID NO: 363) | | | |

### EXAMPLE 4. IDENTIFICATION OF MSLN HUMAN-MURINE CHIMERIC ANTIBODIES

### 4.1. Assay on Binding of Chimeric Antibodies to Human MSLN Proteins by Enzyme-Linked Immunosorbent Assay (ELISA)

To assay the binding activity of the MSLN human-murine chimeric antibodies to the human MSLN full-length protein, MSLN-R1 protein, MSLN-R2 protein, MSLN-R3 protein, and MSLN-R3-3 protein, the purified proteins obtained in Example 2 were diluted to a final concentration of 2 μg/mL with PBS and then added to a 96-well ELISA plate at 50 μL/well. The plate was sealed with a plastic film and incubated at 4° C. overnight. The next day, the plate was washed twice with PBST, and a blocking buffer [PBS+2% (w/w) BSA] was added for blocking at room temperature for 2 h. The blocking buffer was discarded, and a chimeric antibody or negative control antibody serially diluted from 100 nM was added at 50 μL/well. After incubation at 37° C. for 2 h, the plate was washed 3 times with PBST. A horseradish peroxidase (HRP)-labeled secondary antibody (purchased from Jackson, Cat. No. 109-035-088) was added. After incubation at 37° C. for 2 h, the plate was washed 5 times with PBST. A TMB substrate was added at 50 μL/well for incubation at room temperature for 5-10 min, and then a stop solution (1.0 N HCl) was added at 50 μL/well. OD_{450 nm} values were read using an ELISA plate reader (Multimode Plate Reader, EnSight, purchased from Perkin Elmer), and assay results for the binding activity of the chimeric antibodies to the human MSLN full-length protein, MSLN-R1 protein, MSLN-R2 protein, MSLN-R3 protein, and MSLN-R3-3 protein by ELISA are shown in Tables 24-51 and FIGS. 19-24. Tables 24-51 show that the purified chimeric antibodies had different degrees of binding activity to the human MSLN full-length protein, MSLN-R1 protein, MSLN-R2 protein, MSLN-R3 protein, and MSLN-R3-3 protein at the ELISA level. Based on the difference in antigen-antibody binding in the in vitro ELISA assays, F1-F8 chimeric antibodies can be divided into the following five types: (1) binding only to human MSLN full-length protein, e.g., S009-F8.4.5; (2) binding only to human MSLN-R3 protein, e.g., S009-F3.7.3 and F3.38.10; (3) binding to both human MSLN full-length protein and human MSLN-R1 protein, e.g., S009-F2.16.10; (4) binding to both human MSLN full-length protein and human MSLN-R2 protein, e.g., S009-F7.2.3; and (5) binding to both human MSLN full-length protein and human MSLN-R3 protein, e.g., S009-F7.33.24, F7.44.20 and F3.80.22 (also binding to R3-3); weakly binding to R3, F2.23.12 and F2.39.3.

The negative control antibody hIgG1 is an antibody against hen egg lysozyme, anti-hel-hIgG1 (purchased from Biointron, Cat. No. B117901), and the data in the table are OD_{450 nm} values.

TABLE 24
Assay results for binding reactions of F1 chimeric antibodies
with human MSLN full-length protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F1.2.12 | 2.74 | 2.31 | 2.27 | 1.75 | 0.49 | 0.11 | 0.06 | 0.06 |
| S009-F1.7.14 | 2.59 | 2.05 | 1.43 | 0.30 | 0.08 | 0.06 | 0.06 | 0.06 |
| S009-F1.25.10 | 2.57 | 2.44 | 2.30 | 1.67 | 0.43 | 0.10 | 0.06 | 0.06 |
| S009-F1.35.24 | 2.48 | 2.27 | 2.22 | 1.62 | 0.41 | 0.09 | 0.06 | 0.06 |
| S009-F1.56.1 | 2.74 | 2.44 | 2.30 | 1.83 | 0.55 | 0.11 | 0.05 | 0.05 |
| S009-F1.57.1 | 2.51 | 2.42 | 2.03 | 0.82 | 0.15 | 0.06 | 0.05 | 0.05 |
| S009-F1.59.1 | 2.65 | 2.38 | 2.31 | 1.76 | 0.47 | 0.10 | 0.06 | 0.05 |
| S009-F1.62.9 | 2.61 | 2.59 | 2.35 | 1.79 | 0.48 | 0.11 | 0.06 | 0.05 |
| Tab108 | 2.22 | 1.92 | 1.64 | 1.00 | 0.19 | 0.06 | 0.05 | 0.05 |
| Tab142 | 2.50 | 2.50 | 2.36 | 1.76 | 0.48 | 0.10 | 0.08 | 0.06 |
| Tab131 | 0.20 | 0.07 | 0.05 | 0.05 | 0.05 | 0.05 | 0.05 | 0.05 |
| anti-hel-hIgG1 | 0.08 | 0.06 | 0.05 | 0.05 | 0.04 | 0.06 | 0.05 | 0.06 |

TABLE 25
Assay results for binding reactions of F1 chimeric
antibodies with human MSLN-R1 protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F1.2.12 | 0.05 | 0.05 | 0.04 | 0.05 | 0.04 | 0.05 | 0.04 | 0.04 |
| S009-F1.7.14 | 0.27 | 0.07 | 0.05 | 0.05 | 0.04 | 0.04 | 0.05 | 0.05 |
| S009-F1.25.10 | 0.05 | 0.04 | 0.03 | 0.03 | 0.04 | 0.05 | 0.04 | 0.05 |
| S009-F1.35.24 | 0.06 | 0.04 | 0.04 | 0.04 | 0.04 | 0.04 | 0.04 | 0.04 |
| S009-F1.56.1 | 0.12 | 0.06 | 0.05 | 0.04 | 0.03 | 0.05 | 0.04 | 0.05 |
| S009-F1.57.1 | 0.07 | 0.04 | 0.03 | 0.03 | 0.05 | 0.04 | 0.04 | 0.05 |
| S009-F1.59.1 | 0.06 | 0.04 | 0.03 | 0.03 | 0.05 | 0.04 | 0.03 | 0.05 |
| S009-F1.62.9 | 0.06 | 0.04 | 0.04 | 0.04 | 0.05 | 0.04 | 0.04 | 0.05 |
| Tab108 | 0.08 | 0.06 | 0.04 | 0.04 | 0.04 | 0.04 | 0.04 | 0.05 |
| Tab142 | 2.22 | 2.18 | 1.93 | 1.02 | 0.18 | 0.06 | 0.05 | 0.05 |
| Tab131 | 0.10 | 0.05 | 0.05 | 0.05 | 0.04 | 0.04 | 0.04 | 0.04 |
| anti-hel-hIgG1 | 0.06 | 0.05 | 0.04 | 0.05 | 0.05 | 0.05 | 0.04 | 0.05 |

TABLE 26
Assay results for binding reactions of F1 chimeric
antibodies with human MSLN-R2 protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F1.2.12 | 1.44 | 0.30 | 0.10 | 0.06 | 0.05 | 0.05 | 0.05 | 0.05 |
| S009-F1.7.14 | 0.11 | 0.05 | 0.05 | 0.04 | 0.05 | 0.04 | 0.04 | 0.05 |
| S009-F1.25.10 | 0.05 | 0.05 | 0.04 | 0.04 | 0.04 | 0.04 | 0.04 | 0.05 |
| S009-F1.35.24 | 0.06 | 0.05 | 0.05 | 0.04 | 0.05 | 0.05 | 0.04 | 0.05 |
| S009-F1.56.1 | 2.62 | 2.31 | 2.04 | 0.88 | 0.19 | 0.05 | 0.04 | 0.05 |
| S009-F1.57.1 | 0.11 | 0.05 | 0.04 | 0.07 | 0.04 | 0.04 | 0.05 | 0.05 |
| S009-F1.59.1 | 0.06 | 0.05 | 0.04 | 0.05 | 0.06 | 0.04 | 0.03 | 0.04 |
| S009-F1.62.9 | 0.06 | 0.05 | 0.04 | 0.06 | 0.04 | 0.05 | 0.03 | 0.04 |
| Tab108 | 0.08 | 0.05 | 0.04 | 0.04 | 0.04 | 0.04 | 0.03 | 0.04 |
| Tab142 | 0.13 | 0.12 | 0.08 | 0.05 | 0.05 | 0.05 | 0.04 | 0.05 |
| Tab131 | 0.08 | 0.05 | 0.04 | 0.04 | 0.04 | 0.04 | 0.04 | 0.05 |
| anti-hel-hIgG1 | 0.05 | 0.05 | 0.05 | 0.05 | 0.05 | 0.05 | 0.05 | 0.05 |

TABLE 27
Assay results for binding reactions of F1 chimeric
antibodies with human MSLN-R3 protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F1.2.12 | 0.06 | 0.06 | 0.05 | 0.05 | 0.05 | 0.06 | 0.05 | 0.06 |
| S009-F1.7.14 | 0.10 | 0.07 | 0.05 | 0.05 | 0.04 | 0.05 | 0.05 | 0.05 |
| S009-F1.25.10 | 2.81 | 2.07 | 1.44 | 0.34 | 0.09 | 0.05 | 0.05 | 0.05 |
| S009-F1.35.24 | 0.06 | 0.05 | 0.05 | 0.05 | 0.05 | 0.05 | 0.05 | 0.05 |
| S009-F1.56.1 | 0.06 | 0.05 | 0.04 | 0.05 | 0.04 | 0.04 | 0.05 | 0.06 |
| S009-F1.57.1 | 2.25 | 1.81 | 0.86 | 0.19 | 0.06 | 0.05 | 0.05 | 0.05 |
| S009-F1.59.1 | 2.51 | 2.05 | 1.62 | 1.00 | 0.17 | 0.06 | 0.05 | 0.05 |
| S009-F1.62.9 | 2.32 | 2.00 | 1.06 | 0.26 | 0.07 | 0.06 | 0.04 | 0.05 |
| Tab108 | 2.04 | 1.32 | 0.15 | 0.05 | 0.04 | 0.05 | 0.05 | 0.05 |
| Tab142 | 0.06 | 0.05 | 0.05 | 0.05 | 0.05 | 0.05 | 0.05 | 0.05 |
| Tab131 | 0.08 | 0.05 | 0.05 | 0.05 | 0.05 | 0.05 | 0.05 | 0.07 |
| anti-hel-hIgG1 | 0.06 | 0.05 | 0.05 | 0.05 | 0.05 | 0.05 | 0.05 | 0.05 |

TABLE 28
Assay results for binding reactions of F1 chimeric
antibodies with human MSLN-R3-3 protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F1.2.12 | 0.16 | 0.08 | 0.07 | 0.07 | 0.06 | 0.08 | 0.06 | 0.08 |
| S009-F1.7.14 | 0.72 | 0.13 | 0.07 | 0.07 | 0.07 | 0.07 | 0.07 | 0.07 |
| S009-F1.25.10 | 2.78 | 2.41 | 2.17 | 1.82 | 0.59 | 0.13 | 0.07 | 0.07 |
| S009-F1.35.24 | 0.15 | 0.07 | 0.07 | 0.08 | 0.07 | 0.07 | 0.07 | 0.07 |
| S009-F1.56.1 | 0.12 | 0.08 | 0.07 | 0.06 | 0.08 | 0.07 | 0.07 | 0.07 |
| S009-F1.57.1 | 0.11 | 0.07 | 0.07 | 0.07 | 0.07 | 0.07 | 0.06 | 0.07 |
| S009-F1.59.1 | 0.18 | 0.07 | 0.07 | 0.06 | 0.06 | 0.06 | 0.06 | 0.07 |
| S009-F1.62.9 | 0.13 | 0.07 | 0.06 | 0.07 | 0.07 | 0.07 | 0.06 | 0.07 |
| Tab108 | 0.41 | 0.11 | 0.07 | 0.06 | 0.06 | 0.06 | 0.07 | 0.07 |
| Tab142 | 0.15 | 0.08 | 0.06 | 0.06 | 0.07 | 0.07 | 0.07 | 0.07 |
| Tab131 | 0.64 | 0.10 | 0.07 | 0.06 | 0.06 | 0.06 | 0.07 | 0.07 |
| anti-hel-hIgG1 | 0.13 | 0.07 | 0.06 | 0.06 | 0.06 | 0.07 | 0.07 | 0.08 |

TABLE 29
Assay results for binding reactions of F2 chimeric antibodies
with human MSLN full-length protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F2.13.3 | 2.77 | 2.37 | 2.08 | 1.30 | 0.26 | 0.08 | 0.06 | 0.06 |
| S009-F2.16.10 | 2.92 | 2.40 | 2.22 | 1.71 | 0.41 | 0.09 | 0.05 | 0.06 |
| S009-F2.17.3 | 2.81 | 2.48 | 1.93 | 0.64 | 0.13 | 0.06 | 0.05 | 0.06 |
| S009-F2.21.4 | 2.95 | 2.56 | 2.46 | 1.93 | 0.41 | 0.09 | 0.05 | 0.06 |
| S009-F2.23.12 | 2.72 | 2.50 | 2.21 | 1.27 | 0.24 | 0.06 | 0.05 | 0.06 |
| S009-F2.38.12 | 2.57 | 2.43 | 2.56 | 1.85 | 0.46 | 0.09 | 0.06 | 0.06 |
| S009-F2.39.3 | 2.53 | 2.37 | 1.57 | 0.42 | 0.08 | 0.05 | 0.05 | 0.06 |
| S009-F2.47.1 | 2.57 | 2.40 | 2.31 | 1.78 | 0.45 | 0.10 | 0.06 | 0.06 |
| S009-F2-56.12 | 0.40 | 0.37 | 0.41 | 0.37 | 0.16 | 0.07 | 0.06 | 0.07 |
| S009-F2.58.8 | 2.87 | 2.47 | 1.56 | 0.40 | 0.09 | 0.06 | 0.05 | 0.06 |
| Tab142 | 2.41 | 2.47 | 2.02 | 0.59 | 0.11 | 0.05 | 0.05 | 0.06 |
| Tab108 | 2.38 | 2.31 | 1.14 | 0.24 | 0.06 | 0.05 | 0.05 | 0.06 |
| Tab131 | 0.07 | 0.05 | 0.05 | 0.05 | 0.04 | 0.05 | 0.05 | 0.06 |
| anti-hel-hIgG1 | 0.07 | 0.05 | 0.05 | 0.05 | 0.05 | 0.05 | 0.05 | 0.06 |

TABLE 30
Assay results for binding reactions of F2 chimeric
antibodies with human MSLN-R1 protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F2.13.3 | 0.06 | 0.05 | 0.05 | 0.05 | 0.05 | 0.05 | 0.05 | 0.06 |
| S009-F2.16.10 | 2.03 | 1.62 | 0.94 | 0.44 | 0.09 | 0.06 | 0.05 | 0.06 |
| S009-F2.17.3 | 0.07 | 0.06 | 0.04 | 0.05 | 0.04 | 0.05 | 0.05 | 0.06 |
| S009-F2.21.4 | 0.06 | 0.05 | 0.05 | 0.05 | 0.05 | 0.05 | 0.05 | 0.06 |
| S009-F2.23.12 | 0.06 | 0.05 | 0.05 | 0.05 | 0.06 | 0.05 | 0.05 | 0.06 |
| S009-F2.38.12 | 0.17 | 0.06 | 0.05 | 0.05 | 0.05 | 0.05 | 0.05 | 0.06 |
| S009-F2.39.3 | 0.07 | 0.06 | 0.05 | 0.05 | 0.05 | 0.05 | 0.05 | 0.06 |
| S009-F2.47.1 | 0.36 | 0.23 | 0.09 | 0.05 | 0.05 | 0.05 | 0.05 | 0.06 |
| S009-F2-56.12 | 0.16 | 0.07 | 0.06 | 0.06 | 0.06 | 0.06 | 0.06 | 0.06 |
| S009-F2.58.8 | 0.83 | 0.43 | 0.13 | 0.06 | 0.05 | 0.05 | 0.05 | 0.06 |
| Tab142 | 2.30 | 2.35 | 2.02 | 0.87 | 0.15 | 0.06 | 0.05 | 0.06 |
| Tab108 | 0.11 | 0.06 | 0.05 | 0.05 | 0.05 | 0.05 | 0.05 | 0.06 |
| Tab131 | 0.06 | 0.05 | 0.05 | 0.05 | 0.05 | 0.05 | 0.05 | 0.06 |
| anti-hel-hIgG1 | 0.07 | 0.06 | 0.05 | 0.05 | 0.05 | 0.05 | 0.05 | 0.06 |

TABLE 31
Assay results for binding reactions of F2 chimeric
antibodies with human MSLN-R2 protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F2.13.3 | 0.07 | 0.06 | 0.05 | 0.05 | 0.05 | 0.05 | 0.06 | 0.06 |
| S009-F2.16.10 | 0.05 | 0.05 | 0.05 | 0.05 | 0.05 | 0.05 | 0.05 | 0.06 |
| S009-F2.17.3 | 0.06 | 0.05 | 0.05 | 0.05 | 0.05 | 0.05 | 0.05 | 0.06 |
| S009-F2.21.4 | 0.06 | 0.05 | 0.05 | 0.05 | 0.05 | 0.05 | 0.05 | 0.06 |
| S009-F2.23.12 | 0.06 | 0.05 | 0.05 | 0.05 | 0.05 | 0.05 | 0.05 | 0.06 |
| S009-F2.38.12 | 0.06 | 0.05 | 0.05 | 0.05 | 0.05 | 0.05 | 0.05 | 0.06 |
| S009-F2.39.3 | 0.06 | 0.05 | 0.05 | 0.05 | 0.05 | 0.05 | 0.05 | 0.06 |
| S009-F2.47.1 | 0.06 | 0.05 | 0.05 | 0.05 | 0.05 | 0.05 | 0.05 | 0.06 |
| S009-F2-56.12 | 0.17 | 0.07 | 0.06 | 0.06 | 0.06 | 0.06 | 0.06 | 0.07 |
| S009-F2.58.8 | 2.63 | 2.43 | 2.04 | 0.63 | 0.12 | 0.06 | 0.05 | 0.06 |
| Tab142 | 0.10 | 0.08 | 0.06 | 0.05 | 0.05 | 0.05 | 0.05 | 0.06 |
| Tab108 | 0.09 | 0.05 | 0.05 | 0.05 | 0.05 | 0.05 | 0.06 | 0.06 |
| Tab131 | 0.07 | 0.05 | 0.05 | 0.05 | 0.05 | 0.05 | 0.05 | 0.06 |
| anti-hel-hIgG1 | 0.09 | 0.06 | 0.05 | 0.05 | 0.05 | 0.05 | 0.05 | 0.06 |

TABLE 32
Assay results for binding reactions of F2 chimeric
antibodies with human MSLN-R3 protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F2.13.3 | 0.06 | 0.06 | 0.05 | 0.05 | 0.05 | 0.06 | 0.05 | 0.06 |
| S009-F2.16.10 | 0.06 | 0.05 | 0.05 | 0.05 | 0.05 | 0.05 | 0.05 | 0.06 |
| S009-F2.17.3 | 2.30 | 1.19 | 0.29 | 0.07 | 0.05 | 0.05 | 0.05 | 0.06 |
| S009-F2.21.4 | 0.06 | 0.05 | 0.05 | 0.05 | 0.05 | 0.05 | 0.05 | 0.06 |
| S009-F2.23.12 | 2.08 | 1.40 | 0.30 | 0.09 | 0.05 | 0.05 | 0.05 | 0.06 |
| S009-F2.38.12 | 0.06 | 0.05 | 0.05 | 0.05 | 0.04 | 0.05 | 0.05 | 0.05 |
| S009-F2.39.3 | 1.84 | 0.98 | 0.25 | 0.07 | 0.05 | 0.05 | 0.05 | 0.05 |
| S009-F2.47.1 | 0.06 | 0.05 | 0.05 | 0.05 | 0.05 | 0.05 | 0.05 | 0.05 |
| S009-F2-56.12 | 0.13 | 0.07 | 0.06 | 0.05 | 0.05 | 0.05 | 0.05 | 0.06 |
| S009-F2.58.8 | 0.07 | 0.05 | 0.05 | 0.05 | 0.05 | 0.05 | 0.05 | 0.05 |
| Tab142 | 0.07 | 0.05 | 0.05 | 0.05 | 0.05 | 0.05 | 0.05 | 0.06 |
| Tab108 | 1.93 | 1.56 | 0.18 | 0.07 | 0.05 | 0.05 | 0.05 | 0.06 |
| Tab131 | 0.07 | 0.05 | 0.05 | 0.05 | 0.05 | 0.05 | 0.05 | 0.06 |
| anti-hel-hIgG1 | 0.08 | 0.06 | 0.05 | 0.05 | 0.05 | 0.05 | 0.05 | 0.06 |

TABLE 33
Assay results for binding reactions of F2 chimeric
antibodies with human MSLN-R3-3 protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F2.13.3 | 0.06 | 0.07 | 0.05 | 0.05 | 0.05 | 0.05 | 0.05 | 0.05 |
| S009-F2.16.10 | 0.05 | 0.05 | 0.05 | 0.05 | 0.05 | 0.05 | 0.05 | 0.05 |
| S009-F2.17.3 | 0.05 | 0.05 | 0.05 | 0.05 | 0.05 | 0.05 | 0.05 | 0.05 |
| S009-F2.21.4 | 0.05 | 0.05 | 0.05 | 0.05 | 0.05 | 0.05 | 0.05 | 0.05 |
| S009-F2.23.12 | 0.06 | 0.05 | 0.05 | 0.05 | 0.05 | 0.05 | 0.05 | 0.05 |
| S009-F2.38.12 | 0.06 | 0.05 | 0.05 | 0.05 | 0.05 | 0.05 | 0.05 | 0.05 |
| S009-F2.39.3 | 0.05 | 0.05 | 0.05 | 0.05 | 0.05 | 0.05 | 0.05 | 0.05 |
| S009-F2.47.1 | 0.06 | 0.06 | 0.05 | 0.06 | 0.05 | 0.06 | 0.05 | 0.06 |
| S009-F2-56.12 | 0.14 | 0.07 | 0.06 | 0.06 | 0.05 | 0.06 | 0.06 | 0.07 |
| S009-F2.58.8 | 0.07 | 0.06 | 0.06 | 0.06 | 0.06 | 0.06 | 0.05 | 0.06 |
| Tab142 | 0.06 | 0.05 | 0.05 | 0.05 | 0.05 | 0.05 | 0.05 | 0.05 |
| Tab108 | 0.07 | 0.05 | 0.05 | 0.05 | 0.05 | 0.05 | 0.05 | 0.05 |
| Tab131 | 0.06 | 0.05 | 0.05 | 0.05 | 0.05 | 0.05 | 0.05 | 0.05 |
| anti-hel-hIgG1 | 0.06 | 0.05 | 0.05 | 0.05 | 0.05 | 0.05 | 0.05 | 0.05 |

TABLE 34
Assay results for binding reactions of F3 chimeric antibodies
with human MSLN full-length protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F3.7.3 | 0.13 | 0.08 | 0.07 | 0.07 | 0.07 | 0.06 | 0.06 | 0.08 |
| S009-F3.16.1 | 0.12 | 0.11 | 0.08 | 0.08 | 0.07 | 0.06 | 0.06 | 0.08 |
| S009-F3.23.1 | 2.39 | 2.11 | 1.98 | 1.76 | 0.70 | 0.15 | 0.07 | 0.07 |
| S009-F3.38.10 | 0.10 | 0.06 | 0.07 | 0.07 | 0.07 | 0.06 | 0.06 | 0.07 |
| S009-F3.45.21 | 2.23 | 1.80 | 1.75 | 1.63 | 0.69 | 0.35 | 0.10 | 0.08 |
| S009-F3.51.8 | 2.58 | 2.06 | 1.93 | 1.68 | 0.63 | 0.14 | 0.07 | 0.08 |
| S009-F3-63.5 | 2.76 | 2.15 | 1.99 | 1.72 | 0.62 | 0.14 | 0.06 | 0.06 |
| S009-F3.74.20 | 2.17 | 1.97 | 1.88 | 1.59 | 0.42 | 0.11 | 0.07 | 0.08 |
| S009-F3.80.22 | 2.31 | 1.99 | 1.97 | 1.71 | 0.69 | 0.14 | 0.07 | 0.07 |
| Tab108 | 1.83 | 1.93 | 1.72 | 1.38 | 0.49 | 0.10 | 0.07 | 0.07 |
| Tab142 | 2.29 | 2.01 | 1.84 | 1.65 | 0.59 | 0.13 | 0.07 | 0.07 |
| Tab131 | 0.14 | 0.08 | 0.06 | 0.07 | 0.07 | 0.07 | 0.06 | 0.08 |
| anti-hel-hIgG1 | 0.10 | 0.08 | 0.07 | 0.06 | 0.07 | 0.07 | 0.07 | 0.07 |

TABLE 35
Assay results for binding reactions of F3 chimeric
antibodies with human MSLN-R1 protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F3.7.3 | 0.12 | 0.08 | 0.07 | 0.07 | 0.06 | 0.07 | 0.07 | 0.08 |
| S009-F3.16.1 | 0.10 | 0.08 | 0.06 | 0.07 | 0.06 | 0.07 | 0.07 | 0.08 |
| S009-F3.23.1 | 0.08 | 0.06 | 0.06 | 0.06 | 0.06 | 0.06 | 0.07 | 0.07 |
| S009-F3.38.10 | 0.11 | 0.07 | 0.06 | 0.06 | 0.06 | 0.07 | 0.07 | 0.07 |
| S009-F3.45.21 | 0.65 | 0.11 | 0.06 | 0.05 | 0.06 | 0.06 | 0.06 | 0.07 |
| S009-F3.51.8 | 0.07 | 0.06 | 0.06 | 0.06 | 0.07 | 0.07 | 0.07 | 0.07 |
| S009-F3-63.5 | 0.09 | 0.07 | 0.06 | 0.05 | 0.05 | 0.05 | 0.05 | 0.06 |
| S009-F3.74.20 | 0.11 | 0.08 | 0.07 | 0.06 | 0.06 | 0.06 | 0.06 | 0.07 |
| S009-F3.80.22 | 0.07 | 0.06 | 0.05 | 0.06 | 0.06 | 0.06 | 0.06 | 0.06 |
| Tab108 | 0.29 | 0.09 | 0.06 | 0.06 | 0.05 | 0.06 | 0.06 | 0.07 |
| Tab142 | 2.12 | 2.00 | 1.93 | 1.56 | 0.59 | 0.12 | 0.07 | 0.07 |
| Tab131 | 0.10 | 0.07 | 0.06 | 0.06 | 0.06 | 0.07 | 0.08 | 0.08 |
| anti-hel-hlgG1 | 0.09 | 0.07 | 0.05 | 0.06 | 0.06 | 0.07 | 0.06 | 0.07 |

TABLE 36
Assay results for binding reactions of F3 chimeric
antibodies with human MSLN-R2 protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F3.7.3 | 0.12 | 0.09 | 0.07 | 0.07 | 0.06 | 0.07 | 0.07 | 0.08 |
| S009-F3.16.1 | 0.12 | 0.08 | 0.06 | 0.18 | 0.06 | 0.06 | 0.06 | 0.08 |
| S009-F3.23.1 | 0.09 | 0.17 | 0.20 | 0.22 | 0.10 | 0.06 | 0.06 | 0.08 |
| S009-F3.38.10 | 0.13 | 0.18 | 0.21 | 0.18 | 0.11 | 0.06 | 0.07 | 0.08 |
| S009-F3.45.21 | 0.11 | 0.15 | 0.20 | 0.23 | 0.16 | 0.06 | 0.07 | 0.07 |
| S009-F3.51.8 | 0.08 | 0.15 | 0.20 | 0.06 | 0.06 | 0.06 | 0.06 | 0.07 |
| S009-F3-63.5 | 0.09 | 0.07 | 0.05 | 0.05 | 0.05 | 0.05 | 0.06 | 0.07 |
| S009-F3.74.20 | 0.09 | 0.17 | 0.09 | 0.06 | 0.06 | 0.06 | 0.06 | 0.07 |
| S009-F3.80.22 | 0.06 | 0.17 | 0.06 | 0.06 | 0.07 | 0.06 | 0.07 | 0.07 |
| Tab108 | 0.53 | 0.22 | 0.22 | 0.06 | 0.14 | 0.07 | 0.07 | 0.07 |
| Tab142 | 0.16 | 0.23 | 0.23 | 0.10 | 0.09 | 0.06 | 0.07 | 0.08 |
| Tab131 | 0.14 | 0.09 | 0.09 | 0.19 | 0.06 | 0.07 | 0.07 | 0.08 |
| anti-hel-hIgG1 | 0.09 | 0.08 | 0.06 | 0.06 | 0.06 | 0.07 | 0.07 | 0.07 |

TABLE 37
Assay results for binding reactions of F3 chimeric
antibodies with human MSLN-R3 protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F3.7.3 | 2.16 | 1.74 | 1.63 | 1.13 | 0.43 | 0.11 | 0.07 | 0.08 |
| S009-F3.16.1 | 1.92 | 1.91 | 1.80 | 1.61 | 0.65 | 0.15 | 0.08 | 0.09 |
| S009-F3.23.1 | 2.31 | 1.95 | 1.77 | 1.51 | 0.48 | 0.12 | 0.08 | 0.10 |
| S009-F3.38.10 | 2.05 | 1.85 | 1.71 | 1.47 | 0.41 | 0.12 | 0.08 | 0.09 |
| S009-F3.45.21 | 0.09 | 0.07 | 0.05 | 0.06 | 0.07 | 0.24 | 0.09 | 0.09 |
| S009-F3.51.8 | 2.18 | 1.91 | 1.83 | 1.55 | 0.41 | 0.12 | 0.09 | 0.09 |
| S009-F3-63.5 | 2.36 | 1.98 | 1.78 | 1.43 | 0.41 | 0.10 | 0.06 | 0.06 |
| S009-F3.74.20 | 2.06 | 1.83 | 1.88 | 1.07 | 0.29 | 0.10 | 0.07 | 0.09 |
| S009-F3.80.22 | 2.02 | 1.88 | 1.91 | 1.55 | 0.51 | 0.12 | 0.08 | 0.08 |
| Tab108 | 1.16 | 1.61 | 1.30 | 0.52 | 0.11 | 0.07 | 0.07 | 0.07 |
| Tab142 | 0.10 | 0.07 | 0.05 | 0.06 | 0.05 | 0.06 | 0.07 | 0.08 |
| Tab131 | 0.12 | 0.07 | 0.06 | 0.05 | 0.05 | 0.07 | 0.07 | 0.08 |
| anti-hel-hIgG1 | 0.10 | 0.07 | 0.06 | 0.06 | 0.05 | 0.07 | 0.07 | 0.07 |

TABLE 38
Assay results for binding reactions of F3 chimeric
antibodies with human MSLN-R3-3 protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F3.7.3 | 0.11 | 0.08 | 0.07 | 0.07 | 0.07 | 0.07 | 0.07 | 0.08 |
| S009-F3.16.1 | 0.11 | 0.08 | 0.07 | 0.08 | 0.07 | 0.08 | 0.07 | 0.08 |
| S009-F3.23.1 | 2.33 | 2.01 | 1.87 | 1.54 | 0.57 | 0.18 | 0.09 | 0.09 |
| S009-F3.38.10 | 0.32 | 0.10 | 0.08 | 0.08 | 0.08 | 0.08 | 0.07 | 0.07 |
| S009-F3.45.21 | 0.11 | 0.08 | 0.07 | 0.08 | 0.07 | 0.25 | 0.10 | 0.07 |
| S009-F3.51.8 | 2.04 | 1.92 | 1.80 | 1.42 | 0.51 | 0.13 | 0.09 | 0.07 |
| S009-F3-63.5 | 2.30 | 1.95 | 1.75 | 1.46 | 0.40 | 0.10 | 0.07 | 0.07 |
| S009-F3.74.20 | 2.07 | 1.90 | 1.83 | 0.96 | 0.21 | 0.09 | 0.07 | 0.07 |
| S009-F3.80.22 | 1.93 | 1.91 | 1.84 | 1.47 | 0.55 | 0.15 | 0.08 | 0.08 |
| Tab108 | 0.40 | 0.12 | 0.07 | 0.08 | 0.06 | 0.08 | 0.07 | 0.07 |
| Tab142 | 0.13 | 0.08 | 0.07 | 0.07 | 0.07 | 0.07 | 0.07 | 0.08 |
| Tab131 | 0.12 | 0.07 | 0.07 | 0.07 | 0.07 | 0.08 | 0.08 | 0.07 |
| anti-hel-hIgG1 | 0.09 | 0.07 | 0.06 | 0.06 | 0.06 | 0.08 | 0.07 | 0.07 |

TABLE 39
Assay results for binding reactions of F4, F5, and F6 chimeric
antibodies with human MSLN full-length protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F4-94.15 | 1.90 | 1.84 | 1.79 | 1.57 | 0.57 | 0.13 | 0.06 | 0.06 |
| S009-F4-127.10 | 0.21 | 0.09 | 0.06 | 0.06 | 0.06 | 0.06 | 0.06 | 0.06 |
| S009-F5-9.16 | 1.12 | 0.19 | 0.06 | 0.04 | 0.04 | 0.04 | 0.04 | 0.05 |
| S009-F6-62.5 | 3.22 | 3.11 | 3.13 | 2.45 | 0.94 | 0.27 | 0.05 | 0.05 |
| S009-F6-76.1 | 2.83 | 2.87 | 2.87 | 2.40 | 1.09 | 0.29 | 0.10 | 0.05 |
| Tab142 | 1.98 | 1.91 | 1.81 | 1.55 | 0.52 | 0.12 | 0.06 | 0.06 |
| Tab108 | 1.33 | 1.68 | 1.70 | 1.29 | 0.38 | 0.09 | 0.06 | 0.06 |
| Tab106 | 2.00 | 1.90 | 1.81 | 1.33 | 0.37 | 0.09 | 0.06 | 0.06 |
| anti-hel-hIgG1 | 0.16 | 0.08 | 0.06 | 0.06 | 0.06 | 0.06 | 0.06 | 0.07 |

TABLE 40
Assay results for binding reactions of F4, F5, and F6
chimeric antibodies with human MSLN-R1 protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F4-94.15 | 0.07 | 0.06 | 0.05 | 0.05 | 0.05 | 0.05 | 0.05 | 0.07 |
| S009-F4-127.10 | 0.08 | 0.06 | 0.05 | 0.05 | 0.05 | 0.05 | 0.05 | 0.06 |
| S009-F5-9.16 | 0.09 | 0.05 | 0.04 | 0.04 | 0.04 | 0.04 | 0.05 | 0.04 |
| S009-F6-62.5 | 0.15 | 0.06 | 0.04 | 0.04 | 0.04 | 0.04 | 0.06 | 0.05 |
| S009-F6-76.1 | 0.06 | 0.04 | 0.04 | 0.04 | 0.04 | 0.04 | 0.04 | 0.04 |
| Tab142 | 1.86 | 1.80 | 1.69 | 1.40 | 0.44 | 0.11 | 0.06 | 0.06 |
| Tab108 | 0.17 | 0.07 | 0.06 | 0.05 | 0.05 | 0.05 | 0.05 | 0.06 |
| Tab106 | 1.92 | 1.28 | 0.20 | 0.07 | 0.05 | 0.06 | 0.05 | 0.07 |
| anti-hel-hIgG1 | 0.07 | 0.06 | 0.05 | 0.06 | 0.06 | 0.06 | 0.06 | 0.07 |

TABLE 41
Assay results for binding reactions of F4, F5, and F6
chimeric antibodies with human MSLN-R2 protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F4-94.15 | 0.07 | 0.06 | 0.05 | 0.05 | 0.05 | 0.05 | 0.06 | 0.07 |
| S009-F4-127.10 | 0.09 | 0.06 | 0.05 | 0.05 | 0.05 | 0.05 | 0.06 | 0.06 |
| S009-F5-9.16 | 0.13 | 0.06 | 0.04 | 0.04 | 0.04 | 0.04 | 0.04 | 0.05 |
| S009-F6-62.5 | 0.20 | 0.09 | 0.06 | 0.04 | 0.04 | 0.04 | 0.04 | 0.05 |
| S009-F6-76.1 | 0.33 | 0.20 | 0.12 | 0.07 | 0.04 | 0.04 | 0.05 | 0.05 |
| Tab142 | 0.12 | 0.08 | 0.07 | 0.06 | 0.05 | 0.06 | 0.06 | 0.07 |
| Tab108 | 0.21 | 0.08 | 0.06 | 0.06 | 0.06 | 0.06 | 0.06 | 0.07 |
| Tab106 | 2.07 | 1.39 | 0.30 | 0.08 | 0.06 | 0.06 | 0.06 | 0.07 |
| anti-hel-hIgG1 | 0.08 | 0.06 | 0.06 | 0.06 | 0.06 | 0.06 | 0.06 | 0.07 |

TABLE 42
Assay results for binding reactions of F4, F5, and F6
chimeric antibodies with human MSLN-R3 protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F4-94.15 | 0.10 | 0.07 | 0.11 | 0.17 | 0.07 | 0.05 | 0.05 | 0.07 |
| S009-F4-127.10 | 2.10 | 1.93 | 1.82 | 1.31 | 0.41 | 0.09 | 0.06 | 0.07 |
| S009-F5-9.16 | 2.28 | 2.36 | 2.32 | 1.82 | 1.11 | 0.26 | 0.12 | 0.05 |
| S009-F6-62.5 | 0.32 | 0.15 | 0.06 | 0.04 | 0.04 | 0.04 | 0.04 | 0.04 |
| S009-F6-76.1 | 0.08 | 0.05 | 0.04 | 0.04 | 0.04 | 0.05 | 0.04 | 0.05 |
| Tab142 | 0.16 | 0.19 | 0.23 | 0.15 | 0.05 | 0.05 | 0.05 | 0.06 |
| Tab108 | 1.08 | 1.64 | 1.48 | 0.58 | 0.16 | 0.05 | 0.06 | 0.06 |
| Tab106 | 1.79 | 1.69 | 1.57 | 0.88 | 0.18 | 0.06 | 0.05 | 0.07 |
| anti-hel-hIgG1 | 0.11 | 0.07 | 0.05 | 0.07 | 0.06 | 0.06 | 0.06 | 0.07 |

TABLE 43
Assay results for binding reactions of F4, F5, and F6 chimeric
antibodies with human MSLN-R3-3 protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F4-94.15 | 0.09 | 0.06 | 0.05 | 0.05 | 0.05 | 0.05 | 0.06 | 0.07 |
| S009-F4-127.10 | 0.14 | 0.06 | 0.05 | 0.05 | 0.05 | 0.05 | 0.06 | 0.07 |
| S009-F5-9.16 | 1.99 | 0.67 | 0.13 | 0.05 | 0.04 | 0.04 | 0.05 | 0.05 |
| S009-F6-62.5 | 0.09 | 0.05 | 0.05 | 0.04 | 0.04 | 0.04 | 0.04 | 0.05 |
| S009-F6-76.1 | 0.07 | 0.04 | 0.04 | 0.05 | 0.05 | 0.04 | 0.05 | 0.04 |
| Tab142 | 0.08 | 0.05 | 0.05 | 0.05 | 0.05 | 0.05 | 0.06 | 0.07 |
| Tab108 | 0.23 | 0.08 | 0.06 | 0.05 | 0.05 | 0.05 | 0.06 | 0.07 |
| Tab106 | 1.94 | 1.35 | 0.33 | 0.08 | 0.05 | 0.05 | 0.06 | 0.07 |
| anti-hel-hIgG1 | 0.08 | 0.06 | 0.06 | 0.06 | 0.06 | 0.06 | 0.07 | 0.07 |

TABLE 44
Assay results for binding reactions of F7 chimeric antibodies
with human MSLN full-length protein by ELISA

| OD450 | Antibody concentration (nM) | | | | | | | |
|---|---|---|---|---|---|---|---|---|
| Antibody name | 200 | 20 | 2 | 0.2 | 0.02 | 0.002 | 0.0002 | none |
| S009-F7.2.3 | 2.91 | 2.50 | 2.50 | 2.30 | 1.28 | 0.41 | 0.15 | 0.06 |
| S009-F7.6.17 | 2.75 | 2.49 | 2.34 | 2.40 | 1.78 | 0.84 | 0.22 | 0.08 |
| S009-F7.11.11 | 2.62 | 2.35 | 2.13 | 1.54 | 0.41 | 0.16 | 0.08 | 0.08 |
| S009-F7.12.13 | 2.65 | 2.19 | 2.21 | 2.14 | 1.44 | 0.60 | 0.15 | 0.06 |
| S009-F7.18.10 | 2.40 | 2.29 | 2.28 | 2.33 | 1.91 | 0.97 | 0.31 | 0.07 |
| S009-F7.21.16 | 2.42 | 2.46 | 2.31 | 2.15 | 1.39 | 0.82 | 0.35 | 0.08 |
| S009-F7.23.19 | 2.50 | 2.46 | 2.40 | 2.22 | 1.32 | 0.41 | 0.11 | 0.06 |
| S009-F7.25.19 | 2.32 | 2.48 | 2.39 | 2.40 | 1.90 | 1.25 | 0.52 | 0.06 |
| S009-F7.26.15 | 2.39 | 2.52 | 2.43 | 2.33 | 1.76 | 0.91 | 0.25 | 0.06 |
| S009-F7.30.5 | 2.36 | 2.43 | 2.41 | 2.39 | 1.77 | 0.96 | 0.36 | 0.06 |
| S009-F7.33.24 | 2.48 | 2.53 | 2.54 | 2.60 | 2.01 | 1.06 | 0.26 | 0.06 |
| S009-F7.41.18 | 2.30 | 2.24 | 2.26 | 2.04 | 1.00 | 0.25 | 0.08 | 0.06 |
| S009-F7.44.20 | 2.36 | 2.21 | 2.24 | 2.26 | 1.82 | 0.93 | 0.26 | 0.06 |
| S009-F7.48.1 | 2.62 | 2.21 | 2.12 | 1.87 | 0.86 | 0.24 | 0.09 | 0.06 |
| S009-F7.53.2 | 2.60 | 2.20 | 2.10 | 1.53 | 0.47 | 0.12 | 0.06 | 0.06 |
| S009-F7.61.21 | 2.15 | 2.14 | 2.14 | 1.96 | 0.95 | 0.23 | 0.09 | 0.06 |
| S009-F7.65.13 | 2.13 | 2.12 | 2.11 | 2.10 | 1.35 | 0.52 | 0.24 | 0.06 |
| S009-F7.66.12 | 2.10 | 2.20 | 2.23 | 2.24 | 1.77 | 1.19 | 0.77 | 0.07 |
| S009-F7.67.12 | 1.96 | 2.23 | 2.07 | 1.78 | 0.75 | 0.23 | 0.13 | 0.05 |
| S009-F7.69.8 | 2.10 | 2.26 | 2.29 | 2.15 | 1.42 | 0.43 | 0.13 | 0.06 |
| Tab142 | 2.63 | 2.48 | 2.32 | 1.68 | 0.48 | 0.10 | 0.07 | 0.06 |
| Tab106 | 2.58 | 2.42 | 2.41 | 1.94 | 0.98 | 0.47 | 0.31 | 0.06 |
| anti-hel-hIgG1 | 0.17 | 0.06 | 0.05 | 0.04 | 0.04 | 0.04 | 0.08 | 0.06 |

TABLE 45
Assay results for binding reactions of F7 chimeric
antibodies with human MSLN-R1 protein by ELISA

| OD450 | Antibody concentration (nM) | | | | | | | |
|---|---|---|---|---|---|---|---|---|
| Antibody name | 200 | 20 | 2 | 0.2 | 0.02 | 0.002 | 0.0002 | none |
| S009-F7.2.3 | 0.10 | 0.08 | 0.08 | 0.06 | 0.06 | 0.06 | 0.06 | 0.07 |
| S009-F7.6.17 | 0.08 | 0.06 | 0.05 | 0.05 | 0.05 | 0.05 | 0.05 | 0.07 |
| S009-F7.11.11 | 0.06 | 0.06 | 0.05 | 0.05 | 0.05 | 0.05 | 0.05 | 0.07 |
| S009-F7.12.13 | 0.06 | 0.06 | 0.05 | 0.05 | 0.05 | 0.05 | 0.05 | 0.06 |
| S009-F7.18.10 | 2.77 | 2.45 | 2.45 | 2.46 | 2.05 | 1.17 | 0.38 | 0.08 |
| S009-F7.21.16 | 0.17 | 0.06 | 0.05 | 0.05 | 0.05 | 0.05 | 0.04 | 0.07 |
| S009-F7.23.19 | 0.08 | 0.05 | 0.05 | 0.05 | 0.05 | 0.05 | 0.05 | 0.06 |
| S009-F7.25.19 | 0.19 | 0.06 | 0.05 | 0.05 | 0.05 | 0.05 | 0.04 | 0.06 |
| S009-F7.26.15 | 0.12 | 0.06 | 0.05 | 0.05 | 0.05 | 0.05 | 0.04 | 0.06 |
| S009-F7.30.5 | 0.32 | 0.10 | 0.06 | 0.05 | 0.05 | 0.05 | 0.05 | 0.06 |
| S009-F7.33.24 | 0.15 | 0.08 | 0.06 | 0.05 | 0.06 | 0.06 | 0.06 | 0.06 |
| S009-F7.41.18 | 0.09 | 0.07 | 0.06 | 0.06 | 0.05 | 0.06 | 0.06 | 0.06 |
| S009-F7.44.20 | 0.07 | 0.05 | 0.04 | 0.04 | 0.04 | 0.04 | 0.04 | 0.06 |
| S009-F7.48.1 | 0.07 | 0.05 | 0.04 | 0.04 | 0.04 | 0.04 | 0.04 | 0.06 |
| S009-F7.53.2 | 0.07 | 0.05 | 0.05 | 0.04 | 0.04 | 0.05 | 0.05 | 0.06 |
| S009-F7.61.21 | 0.07 | 0.05 | 0.05 | 0.05 | 0.05 | 0.05 | 0.05 | 0.07 |
| S009-F7.65.13 | 0.08 | 0.05 | 0.05 | 0.05 | 0.05 | 0.05 | 0.05 | 0.07 |
| S009-F7.66.12 | 0.13 | 0.06 | 0.05 | 0.04 | 0.04 | 0.04 | 0.06 | 0.06 |
| S009-F7.67.12 | 0.07 | 0.05 | 0.05 | 0.05 | 0.05 | 0.05 | 0.04 | 0.07 |
| S009-F7.69.8 | 0.11 | 0.06 | 0.04 | 0.05 | 0.04 | 0.05 | 0.05 | 0.07 |
| Tab142 | 2.72 | 2.27 | 2.27 | 2.00 | 1.01 | 0.25 | 0.09 | 0.06 |
| Tab106 | 2.30 | 2.13 | 0.95 | 0.25 | 0.11 | 0.07 | 0.07 | 0.07 |
| anti-hel-hIgG1 | 0.08 | 0.05 | 0.04 | 0.04 | 0.04 | 0.05 | 0.07 | 0.06 |

TABLE 46
Assay results for binding reactions of F7 chimeric
antibodies with human MSLN-R2 protein by ELISA

| OD450 | Antibody concentration (nM) | | | | | | | |
|---|---|---|---|---|---|---|---|---|
| Antibody name | 200 | 20 | 2 | 0.2 | 0.02 | 0.002 | 0.0002 | none |
| S009-F7.2.3 | 2.74 | 2.38 | 2.37 | 2.12 | 1.22 | 0.41 | 0.14 | 0.06 |
| S009-F7.6.17 | 0.13 | 0.07 | 0.05 | 0.05 | 0.05 | 0.05 | 0.05 | 0.06 |
| S009-F7.11.11 | 0.07 | 0.06 | 0.05 | 0.05 | 0.05 | 0.05 | 0.05 | 0.06 |
| S009-F7.12.13 | 0.08 | 0.06 | 0.05 | 0.05 | 0.05 | 0.05 | 0.05 | 0.06 |
| S009-F7.18.10 | 0.14 | 0.09 | 0.09 | 0.06 | 0.05 | 0.05 | 0.05 | 0.06 |
| S009-F7.21.16 | 0.20 | 0.07 | 0.06 | 0.05 | 0.05 | 0.05 | 0.05 | 0.06 |
| S009-F7.23.19 | 0.09 | 0.05 | 0.05 | 0.05 | 0.05 | 0.05 | 0.14 | 0.06 |
| S009-F7.25.19 | 0.31 | 0.09 | 0.06 | 0.05 | 0.05 | 0.05 | 0.05 | 0.06 |
| S009-F7.26.15 | 0.18 | 0.08 | 0.05 | 0.05 | 0.05 | 0.05 | 0.05 | 0.06 |
| S009-F7.30.5 | 0.50 | 0.15 | 0.06 | 0.06 | 0.05 | 0.05 | 0.05 | 0.06 |
| S009-F7.33.24 | 0.08 | 0.04 | 0.06 | 0.06 | 0.06 | 0.06 | 0.06 | 0.07 |
| S009-F7.41.18 | 0.15 | 0.12 | 0.11 | 0.07 | 0.07 | 0.07 | 0.07 | 0.08 |
| S009-F7.44.20 | 0.10 | 0.06 | 0.05 | 0.05 | 0.05 | 0.05 | 0.05 | 0.06 |
| S009-F7.48.1 | 0.09 | 0.07 | 0.05 | 0.05 | 0.05 | 0.05 | 0.05 | 0.06 |
| S009-F7.53.2 | 0.08 | 0.06 | 0.05 | 0.05 | 0.05 | 0.05 | 0.05 | 0.07 |
| S009-F7.61.21 | 0.09 | 0.06 | 0.05 | 0.05 | 0.05 | 0.05 | 0.05 | 0.07 |
| S009-F7.65.13 | 0.10 | 0.05 | 0.05 | 0.05 | 0.05 | 0.05 | 0.05 | 0.07 |
| S009-F7.66.12 | 0.28 | 0.09 | 0.06 | 0.05 | 0.05 | 0.05 | 0.27 | 0.07 |
| S009-F7.67.12 | 0.10 | 0.07 | 0.06 | 0.06 | 0.05 | 0.05 | 0.05 | 0.06 |
| S009-F7.69.8 | 0.20 | 0.07 | 0.06 | 0.05 | 0.06 | 0.06 | 0.05 | 0.07 |
| Tab142 | 0.48 | 0.24 | 0.21 | 0.14 | 0.08 | 0.06 | 0.06 | 0.06 |
| Tab106 | 2.54 | 2.42 | 1.42 | 0.47 | 0.17 | 0.11 | 0.12 | 0.07 |
| anti-hel-hIgG1 | 0.09 | 0.07 | 0.06 | 0.06 | 0.05 | 0.05 | 0.52 | 0.06 |

TABLE 47
Assay results for binding reactions of F7 chimeric
antibodies with human MSLN-R3 protein by ELISA

| OD450 | Antibody concentration (nM) | | | | | | | |
|---|---|---|---|---|---|---|---|---|
| Antibody name | 200 | 20 | 2 | 0.2 | 0.02 | 0.002 | 0.0002 | none |
| S009-F7.2.3 | 0.20 | 0.10 | 0.07 | 0.06 | 0.06 | 0.06 | 0.06 | 0.07 |
| S009-F7.6.17 | 3.10 | 2.81 | 2.86 | 2.64 | 1.55 | 0.56 | 0.13 | 0.08 |
| S009-F7.11.11 | 3.12 | 2.58 | 2.41 | 1.37 | 0.32 | 0.09 | 0.06 | 0.06 |
| S009-F7.12.13 | 2.96 | 2.67 | 2.48 | 1.67 | 0.36 | 0.15 | 0.06 | 0.06 |
| S009-F7.18.10 | 0.29 | 0.14 | 0.07 | 0.06 | 0.05 | 0.05 | 0.05 | 0.07 |
| S009-F7.21.16 | 2.92 | 2.52 | 2.75 | 2.50 | 1.32 | 0.71 | 0.31 | 0.08 |
| S009-F7.23.19 | 2.77 | 2.63 | 2.52 | 1.90 | 0.88 | 0.25 | 0.08 | 0.09 |
| S009-F7.25.19 | 2.83 | 2.71 | 2.76 | 2.60 | 1.81 | 0.90 | 0.30 | 0.07 |
| S009-F7.26.15 | 2.74 | 2.73 | 2.79 | 2.45 | 1.63 | 0.60 | 0.16 | 0.07 |
| S009-F7.30.5 | 2.71 | 2.84 | 2.75 | 2.66 | 1.70 | 0.74 | 0.25 | 0.09 |
| S009-F7.33.24 | 2.84 | 2.67 | 2.85 | 2.70 | 2.13 | 1.04 | 0.23 | 0.08 |
| S009-F7.41.18 | 3.00 | 2.46 | 2.43 | 1.92 | 0.69 | 0.12 | 0.07 | 0.06 |
| S009-F7.44.20 | 2.91 | 2.48 | 2.46 | 2.20 | 0.90 | 0.27 | 0.10 | 0.06 |
| S009-F7.48.1 | 2.69 | 2.43 | 2.05 | 0.94 | 0.26 | 0.08 | 0.05 | 0.06 |
| S009-F7.53.2 | 2.69 | 2.42 | 1.71 | 0.50 | 0.14 | 0.06 | 0.05 | 0.06 |
| S009-F7.61.21 | 2.61 | 2.39 | 2.17 | 0.80 | 0.17 | 0.06 | 0.05 | 0.06 |
| S009-F7.65.13 | 2.63 | 2.34 | 2.39 | 1.30 | 0.35 | 0.14 | 0.07 | 0.07 |
| S009-F7.66.12 | 2.60 | 2.37 | 2.25 | 1.95 | 0.04 | 0.23 | 0.14 | 0.06 |
| S009-F7.67.12 | 2.75 | 2.49 | 2.45 | 1.93 | 0.69 | 0.24 | 0.11 | 0.07 |
| S009-F7.69.8 | 2.56 | 2.55 | 2.54 | 1.96 | 0.38 | 0.11 | 0.06 | 0.07 |
| Tab142 | 0.28 | 0.11 | 0.06 | 0.05 | 0.05 | 0.05 | 0.05 | 0.06 |
| Tab106 | 2.12 | 2.26 | 2.33 | 1.90 | 0.97 | 0.65 | 0.35 | 0.06 |
| anti-hel-hIgG1 | 0.12 | 0.07 | 0.05 | 0.05 | 0.05 | 0.05 | 0.33 | 0.06 |

TABLE 48
Assay results for binding reactions of F8 chimeric antibodies
with human MSLN full-length protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F8-4.5 | 2.94 | 2.79 | 2.80 | 1.31 | 0.18 | 0.10 | 0.07 | 0.06 |
| S009-F8-5.15 | 3.16 | 2.75 | 2.51 | 1.53 | 0.34 | 0.08 | 0.05 | 0.05 |
| S009-F8-7.5 | 3.22 | 2.73 | 2.55 | 1.88 | 0.49 | 0.12 | 0.06 | 0.05 |
| S009-F8-8.22 | 3.15 | 3.15 | 2.75 | 1.97 | 0.91 | 0.23 | 0.10 | 0.05 |
| S009-F8-9.16 | 1.07 | 0.26 | 0.06 | 0.04 | 0.04 | 0.05 | 0.07 | 0.04 |
| S009-F8-12.13 | 2.94 | 2.53 | 2.42 | 2.22 | 1.26 | 0.49 | 0.32 | 0.05 |
| S009-F8-13.8 | 2.69 | 2.34 | 2.05 | 1.18 | 0.24 | 0.07 | 0.05 | 0.05 |
| S009-F8-15.19 | 2.64 | 2.47 | 2.23 | 1.11 | 0.24 | 0.08 | 0.05 | 0.05 |
| S009-F8-18.9 | 2.42 | 2.39 | 2.29 | 1.93 | 0.71 | 0.15 | 0.06 | 0.05 |
| S009-F8-19.21 | 2.37 | 2.32 | 2.05 | 1.61 | 0.40 | 0.10 | 0.06 | 0.06 |
| S009-F8-22.23 | 2.90 | 2.88 | 2.49 | 1.71 | 0.92 | 0.44 | 0.29 | 0.04 |
| S009-F8-24.14 | 2.91 | 2.98 | 2.77 | 2.02 | 0.75 | 0.18 | 0.10 | 0.06 |
| S009-F8-27.1 | 2.50 | 2.37 | 2.23 | 1.34 | 0.31 | 0.08 | 0.05 | 0.04 |
| S009-F8-28.23 | 3.00 | 3.13 | 2.63 | 1.04 | 0.24 | 0.08 | 0.05 | 0.05 |
| S009-F8-29.1 | 3.07 | 3.20 | 2.90 | 1.71 | 0.48 | 0.13 | 0.07 | 0.18 |
| S009-F8-31.22 | 2.66 | 2.56 | 2.53 | 2.19 | 0.85 | 0.20 | 0.08 | 0.08 |
| S009-F8-32.3 | 3.03 | 2.85 | 2.64 | 1.63 | 0.48 | 0.11 | 0.07 | 0.04 |
| S009-F8-33.12 | 2.95 | 2.92 | 2.57 | 1.61 | 0.43 | 0.11 | 0.05 | 0.06 |
| S009-F8-36.12 | 2.71 | 2.69 | 2.21 | 0.73 | 0.18 | 0.06 | 0.05 | 0.04 |
| Tab106 | 2.81 | 2.80 | 2.52 | 1.81 | 0.64 | 0.18 | 0.12 | 0.04 |
| Tab142 | 2.84 | 2.80 | 2.58 | 1.37 | 0.39 | 0.10 | 0.06 | 0.04 |
| anti-hel-hIgG1 | 0.06 | 0.05 | 0.04 | 0.04 | 0.04 | 0.05 | 0.05 | 0.04 |

TABLE 49
Assay results for binding reactions of F8 chimeric
antibodies with human MSLN-R1 protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F8-4.5 | 0.24 | 0.09 | 0.05 | 0.05 | 0.05 | 0.05 | 0.05 | 0.05 |
| S009-F8-5.15 | 2.29 | 1.65 | 0.68 | 0.12 | 0.05 | 0.05 | 0.04 | 0.05 |
| S009-F8-7.5 | 0.12 | 0.06 | 0.05 | 0.05 | 0.04 | 0.05 | 0.04 | 0.05 |
| S009-F8-8.22 | 2.63 | 2.63 | 2.46 | 1.78 | 0.61 | 0.16 | 0.08 | 0.06 |
| S009-F8-9.16 | 0.09 | 0.05 | 0.04 | 0.04 | 0.04 | 0.04 | 0.04 | 0.04 |
| S009-F8-12.13 | 2.17 | 2.16 | 2.06 | 1.97 | 1.18 | 0.52 | 0.32 | 0.05 |
| S009-F8-13.8 | 2.35 | 2.10 | 1.80 | 1.26 | 0.28 | 0.08 | 0.05 | 0.05 |
| S009-F8-15.19 | 2.07 | 1.55 | 0.54 | 0.10 | 0.05 | 0.05 | 0.04 | 0.05 |
| S009-F8-18.9 | 2.15 | 2.12 | 1.96 | 1.73 | 0.62 | 0.15 | 0.06 | 0.05 |
| S009-F8-19.21 | 2.13 | 2.11 | 1.82 | 1.46 | 0.39 | 0.10 | 0.06 | 0.05 |
| S009-F8-22.23 | 2.55 | 2.51 | 2.34 | 1.72 | 0.78 | 0.36 | 0.26 | 0.04 |
| S009-F8-24.14 | 2.34 | 2.24 | 1.63 | 0.64 | 0.13 | 0.07 | 0.05 | 0.04 |
| S009-F8-27.1 | 0.35 | 0.08 | 0.05 | 0.05 | 0.04 | 0.04 | 0.04 | 0.05 |
| S009-F8-28.23 | 0.05 | 0.04 | 0.04 | 0.04 | 0.04 | 0.05 | 0.04 | 0.04 |
| S009-F8-29.1 | 0.05 | 0.04 | 0.04 | 0.04 | 0.04 | 0.04 | 0.04 | 0.04 |
| S009-F8-31.22 | 2.12 | 2.17 | 2.05 | 1.95 | 0.80 | 0.19 | 0.08 | 0.05 |
| S009-F8-32.3 | 2.81 | 2.79 | 2.60 | 1.64 | 0.42 | 0.11 | 0.06 | 0.04 |
| S009-F8-33.12 | 2.64 | 2.66 | 2.45 | 1.60 | 0.43 | 0.11 | 0.05 | 0.04 |
| S009-F8-36.12 | 0.05 | 0.04 | 0.04 | 0.04 | 0.04 | 0.04 | 0.04 | 0.03 |
| Tab106 | 0.30 | 0.07 | 0.04 | 0.04 | 0.04 | 0.04 | 0.04 | 0.04 |
| Tab142 | 2.66 | 2.71 | 2.45 | 1.75 | 0.53 | 0.13 | 0.06 | 0.05 |
| anti-hel-hIgG1 | 0.06 | 0.04 | 0.04 | 0.04 | 0.04 | 0.04 | 0.04 | 0.04 |

TABLE 50
Assay results for binding reactions of F8 chimeric
antibodies with human MSLN-R2 protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F8-4.5 | 0.08 | 0.06 | 0.05 | 0.05 | 0.05 | 0.05 | 0.05 | 0.05 |
| S009-F8-5.15 | 0.05 | 0.05 | 0.04 | 0.04 | 0.04 | 0.05 | 0.05 | 0.05 |
| S009-F8-7.5 | 2.32 | 2.18 | 1.85 | 1.18 | 0.31 | 0.09 | 0.06 | 0.05 |
| S009-F8-8.22 | 0.09 | 0.05 | 0.04 | 0.04 | 0.04 | 0.05 | 0.05 | 0.04 |
| S009-F8-9.16 | 0.07 | 0.05 | 0.04 | 0.04 | 0.04 | 0.04 | 0.04 | 0.05 |
| S009-F8-12.13 | 0.15 | 0.07 | 0.05 | 0.05 | 0.05 | 0.04 | 0.04 | 0.05 |
| S009-F8-13.8 | 0.06 | 0.05 | 0.04 | 0.05 | 0.04 | 0.05 | 0.05 | 0.05 |
| S009-F8-15.19 | 0.05 | 0.05 | 0.04 | 0.04 | 0.04 | 0.04 | 0.05 | 0.05 |
| S009-F8-18.9 | 0.08 | 0.06 | 0.05 | 0.04 | 0.04 | 0.04 | 0.04 | 0.05 |
| S009-F8-19.21 | 0.06 | 0.06 | 0.05 | 0.04 | 0.04 | 0.04 | 0.05 | 0.05 |
| S009-F8-22.23 | 0.17 | 0.11 | 0.08 | 0.05 | 0.04 | 0.05 | 0.04 | 0.04 |
| S009-F8-24.14 | 0.05 | 0.04 | 0.04 | 0.04 | 0.04 | 0.04 | 0.04 | 0.04 |
| S009-F8-27.1 | 0.05 | 0.05 | 0.05 | 0.04 | 0.04 | 0.05 | 0.04 | 0.05 |
| S009-F8-28.23 | 0.05 | 0.04 | 0.04 | 0.04 | 0.04 | 0.04 | 0.04 | 0.04 |
| S009-F8-29.1 | 0.06 | 0.04 | 0.04 | 0.04 | 0.04 | 0.04 | 0.05 | 0.04 |
| S009-F8-31.22 | 0.08 | 0.05 | 0.04 | 0.05 | 0.04 | 0.04 | 0.05 | 0.05 |
| S009-F8-32.3 | 0.07 | 0.05 | 0.04 | 0.04 | 0.04 | 0.04 | 0.04 | 0.04 |
| S009-F8-33.12 | 0.05 | 0.04 | 0.04 | 0.04 | 0.04 | 0.04 | 0.04 | 0.04 |
| S009-F8-36.12 | 0.05 | 0.04 | 0.04 | 0.03 | 0.04 | 0.04 | 0.04 | 0.04 |
| Tab106 | 0.22 | 0.06 | 0.04 | 0.04 | 0.04 | 0.04 | 0.04 | 0.04 |
| Tab142 | 0.13 | 0.11 | 0.09 | 0.06 | 0.05 | 0.04 | 0.04 | 0.04 |
| anti-hel-hIgG1 | 0.05 | 0.04 | 0.04 | 0.03 | 0.04 | 0.04 | 0.04 | 0.04 |

TABLE 51
Assay results for binding reactions of F8 chimeric
antibodies with human MSLN-R3 protein by ELISA

| OD450 | Antibody concentration (nM) | | | | | | | |
|---|---|---|---|---|---|---|---|---|
| Antibody name | 100 | 10 | 1 | 0.1 | 0.01 | 0.001 | 0.0001 | none |
| S009-F8-4.5 | 0.12 | 0.07 | 0.05 | 0.05 | 0.04 | 0.05 | 0.05 | 0.06 |
| S009-F8-5.15 | 0.06 | 0.05 | 0.04 | 0.04 | 0.04 | 0.05 | 0.04 | 0.05 |
| S009-F8-7.5 | 0.25 | 0.10 | 0.05 | 0.05 | 0.04 | 0.04 | 0.04 | 0.05 |
| S009-F8-8.22 | 0.15 | 0.08 | 0.05 | 0.04 | 0.04 | 0.04 | 0.04 | 0.04 |
| S009-F8-9.16 | 2.40 | 2.03 | 2.23 | 1.38 | 0.50 | 0.11 | 0.06 | 0.03 |
| S009-F8-12.13 | 0.20 | 0.10 | 0.06 | 0.05 | 0.04 | 0.05 | 0.04 | 0.05 |
| S009-F8-13.8 | 0.06 | 0.05 | 0.04 | 0.04 | 0.04 | 0.05 | 0.04 | 0.05 |
| S009-F8-15.19 | 0.08 | 0.05 | 0.04 | 0.04 | 0.04 | 0.04 | 0.04 | 0.05 |
| S009-F8-18.9 | 0.10 | 0.06 | 0.05 | 0.04 | 0.04 | 0.05 | 0.04 | 0.05 |
| S009-F8-19.21 | 0.09 | 0.05 | 0.04 | 0.04 | 0.04 | 0.04 | 0.04 | 0.05 |
| S009-F8-22.23 | 0.10 | 0.05 | 0.04 | 0.04 | 0.04 | 0.04 | 0.04 | 0.04 |
| S009-F8-24.14 | 0.06 | 0.05 | 0.04 | 0.04 | 0.04 | 0.04 | 0.04 | 0.04 |
| S009-F8-27.1 | 0.06 | 0.05 | 0.04 | 0.07 | 0.04 | 0.05 | 0.04 | 0.05 |
| S009-F8-28.23 | 0.07 | 0.05 | 0.04 | 0.04 | 0.07 | 0.04 | 0.04 | 0.04 |
| S009-F8-29.1 | 0.10 | 0.06 | 0.06 | 0.04 | 0.04 | 0.15 | 0.06 | 0.04 |
| S009-F8-31.22 | 0.13 | 0.07 | 0.05 | 0.04 | 0.04 | 0.04 | 0.04 | 0.05 |
| S009-F8-32.3 | 0.07 | 0.04 | 0.04 | 0.04 | 0.04 | 0.04 | 0.04 | 0.04 |
| S009-F8-33.12 | 0.06 | 0.05 | 0.04 | 0.03 | 0.04 | 0.04 | 0.04 | 0.03 |
| S009-F8-36.12 | 1.19 | 0.14 | 0.05 | 0.04 | 0.04 | 0.04 | 0.04 | 0.04 |
| Tab106 | 2.28 | 1.81 | 1.99 | 1.38 | 0.52 | 0.14 | 0.09 | 0.04 |
| Tab142 | 0.09 | 0.05 | 0.04 | 0.04 | 0.04 | 0.05 | 0.04 | 0.04 |
| anti-hel-hIgG1 | 0.06 | 0.04 | 0.04 | 0.03 | 0.04 | 0.03 | 0.04 | 0.03 |

### 4.2 Assay on Binding of Chimeric Antibodies to Different MSLN-Expressing Cells by Flow Cytometry Assay (FACS)

The desired cells were expanded to the logarithmic growth phase in a T-75 cell culture flask. For adherent cells OVCAR3, A431, HEK293T-hMSLN-B8, HEK293T-hMSLN-R3, HEK293T-monkey MSLN, and 293T, the medium was removed by pipetting, and the cells were washed twice with a PBS buffer, digested with trypsin, and washed twice with a PBS buffer again after the digestion was stopped. After the cells from the previous step were subjected to cell counting, the cell pellet was resuspended to 2×10⁶ cells/mL in a blocking buffer of [PBS+2% (w/w) BSA] and added into a 96-well FACS reaction plate at 50 μL/well. A chimeric antibody (a sample to be tested) was added at 50 μL/well, and the plate was incubated on ice for 2 h. After the plate was centrifuged and washed 3 times with a PBS buffer, a goat anti-human IgG H+L antibody (Jackson, Cat. No. 109605088) was added at 50 μL/well for incubation on ice for 1 h. After the plate was centrifuged and washed 5 times with a PBST buffer, assay and analysis were performed by FACS (FACS CantoII, purchased from BD). Data analysis was performed by software (Flowjo) to obtain the mean fluorescence intensity (MFI) of the cells. Then, analysis was performed by software (GraphPad Prism8), the data were analyzed, and the analysis results are shown in Tables 52-57 and FIGS. 25-30, indicating that (1) all of the chimeric antibodies could bind to the 293T-hMSLN.B8 cells; (2) most of the chimeric antibodies could bind to OVCAR3; (3) part of the chimeric antibodies bound to HEK293T-hMSLN-R3; (4) the chimeric antibody showed binding activity to most of the 293T cells over-expressing monkey MSLN; the binding of the chimeric antibodies to endogenous A431 and 293T cells was simultaneously assayed using the same method, and (5) all of the chimeric antibodies did not bind to A431 cells and 293T cells, showing good specificity.

TABLE 52
Assay results for binding reactions of F1 chimeric antibodies with cells by FACS

| | Cell | | | | | | | | | |
|---|---|---|---|---|---|---|---|---|---|---|
| | | | | | | | HEK293T-monkey | | | |
| | OVCAR3 | | HEK293T-hMSLN-B8 | | HEK293T-hMSLN-R3 | | MSLN | | 293T | A431 |
| | Maximum | | Maximum | | Maximum | | Maximum | | Maximum | Maximum |
| | mean | | mean | | mean | | mean | | mean | mean |
| | fluores- | | fluores- | | fluores- | | fluores- | | fluores- | fluores- |
| Antibody | cence | | cence | | cence | | cence | | cence | cence |
| name | intensity | EC50 (nM) | intensity | EC50 (nM) | intensity | EC50(nM) | intensity | EC50(nM) | intensity | intensity |
| S009-F1.2.12 | 9615 | 1.87 | 87220 | ~0.86 | 239 | NB¹ | 29204 | 1.39 | 75 | 155 |
| S009-F1.7.14 | 2247 | WB² | 70321 | 3.40 | 239 | NB | 47456 | 5.69 | 227 | 888 |
| S009-F1.25.10 | 429 | NB | 41889 | WB | 6263 | 1.44 | 11451 | WB | 113 | 166 |
| S009-F1.35.24 | 10492 | 2.08 | 108575 | 2.93 | 680 | NB | 52067 | 4.75 | 62 | 133 |
| S009-F1.56.1 | 11692 | 0.67 | 96272 | 1.59 | 363 | NB | 43063 | 0.79 | 127 | 947 |
| S009-F1.57.1 | 948 | WB | 8746 | WB | 6655 | 3.90 | 4490 | WB | 142 | 165 |
| S009-F1.59.1 | 445 | NB | 32812 | WB | 7071 | 0.31 | 1415 | NB | 205 | 192 |
| S009-F1.62.9 | 425 | NB | 9553 | WB | 6929 | 0.95 | 965 | NB | 77 | 235 |
| Tab108 | 10202 | 0.95 | 62206 | 2.98 | 6361 | 0.61 | 31924 | 0.94 | 112 | 446 |
| Tab142 | 15862 | 3.43 | 107196 | 1.97 | 833 | NB | 53324 | 0.67 | 84 | 390 |
| Tab131 | 2389 | WB | 45559 | 0.96 | 1182 | WB | 5583 | WB | 107 | 168 |
| hIgG1 | 122 | NB | 267 | NB | 102 | NB | 102 | NB | 96 | 169 |

¹NB: no binding;
²WB: worse binding

TABLE 53
Assay results for binding reactions of F2 chimeric antibodies with cells by FACS

| | | | | | | | HEK293T-monkey | | | |
|---|---|---|---|---|---|---|---|---|---|---|
| | OVCAR3 | | HEK293T-hMSLN-B8 | | HEK293T-hMSLN-R3 | | MSLN | | 293T | A431 |
| | Maximum | | Maximum | | Maximum | | Maximum | | Maximum | Maximum |
| Cell | mean | | mean | | mean | | mean | | mean | mean |
| Antibody | fluorescence | EC50 | fluorescence | EC50 | fluorescence | EC50 | fluorescence | EC50 | fluorescence | fluorescence |
| name | intensity | (nM) | intensity | (nM) | intensity | (nM) | intensity | (nM) | intensity | intensity |
| S009-F2.13.3 | 14823 | 0.57 | 112410 | 1.15 | 112 | NB | 138 | NB | 56 | 241 |
| S009-F2.16.10 | 14378 | 2.96 | 109166 | 0.88 | 114 | NB | 18381 | 0.30 | 55 | 252 |
| S009-F2.17.3 | 17570 | 2.92 | 148143 | 3.95 | 3836 | 1.48 | 24254 | 1.63 | 70 | 471 |
| S009-F2.21.4 | 5261 | WB | 91756 | ~0.82 | 348 | NB | 20074 | 0.44 | 55 | 193 |
| S009-F2.23.12 | 14645 | 1.00 | 143882 | 2.39 | 3877 | 0.50 | 20390 | ~0.83 | 70 | 130 |
| S009-F2.38.12 | 4514 | WB | 97733 | ~0.82 | 136 | NB | 6744 | WB | 56 | 158 |
| S009-F2.39.3 | 14617 | 5.52 | 138287 | 5.79 | 3287 | 4.19 | 20841 | 4.18 | 55 | 627 |
| S009-F2.47.1 | 15364 | 10.43 | 98209 | ~0.82 | 4579 | WB | 17052 | 0.32 | 2422 | 1218 |
| S009-F2-56.12 | 6136 | 0.54 | 67021 | ~0.79 | 94 | NB | 95 | NB | 95 | 145 |
| S009-F2.58.8 | 13106 | 1.82 | 140750 | 3.50 | 366 | NB | 1000 | NB | 106 | 221 |
| Tab108 | 11813 | 0.63 | 78365 | 2.66 | 2692 | 0.45 | 14061 | 0.81 | 72 | 446 |
| Tab131 | 1523 | WB | 78217 | 5.31 | 371 | 0.98 | 891 | WB | 60 | 390 |
| Tab142 | 18364 | 4.22 | 150390 | 3.20 | 289 | NB | 21015 | 0.49 | 58 | 168 |
| hIgG1 | 122 | NB | 267 | NB | 102 | NB | 102 | NB | 96 | 169 |

TABLE 54
Assay results for binding reactions of F3 chimeric antibodies with cells by FACS

| | | | | | | | HEK293T-monkey | | | |
|---|---|---|---|---|---|---|---|---|---|---|
| | OVCAR3 | | HEK293T-hMSLN-B8 | | HEK293T-hMSLN-R3 | | MSLN | | 293T | A431 |
| | Maximum | | Maximum | | Maximum | | Maximum | | Maximum | Maximum |
| Cell | mean | | mean | | mean | | mean | | mean | mean |
| Antibody | fluorescence | EC50 | fluorescence | EC50 | fluorescence | EC50 | fluorescence | EC50 | fluorescence | fluorescence |
| name | intensity | (nM) | intensity | (nM) | intensity | (nM) | intensity | (nM) | intensity | intensity |
| S009-F3.7.3 | 3699 | 1.94 | 88817 | 2.25 | 7915 | 0.72 | 7098 | WB | 94 | 253 |
| S009-F3.16.1 | 4189 | 1.61 | 89833 | 1.46 | 8065 | 0.43 | 24761 | 1.40 | 79 | 455 |
| S009-F3.23.1 | 288 | WB | 26430 | 33.67 | 4236 | 0.84 | 88 | NB | 64 | 201 |
| S009-F3.38.10 | 2625 | WB | 80621 | 6.84 | 6766 | 3.41 | 22343 | 4.63 | 62 | 124 |
| S009-F3.45.21 | 9714 | 5.61 | 97578 | 1.41 | 81 | NB | 41070 | 0.56 | 60 | 162 |
| S009-F3.51.8 | 337 | WE | 27906 | 21.76 | 4467 | 0.75 | 88 | NB | 109 | 237 |
| S009-F3-63.5 | 227 | NB | 35401 | WB | 8266 | 1.18 | 105 | NB | 89 | 134 |
| S009-F3.74.20 | 613 | WB | 36364 | 8.68 | 5067 | 0.62 | 188 | NB | 60 | 205 |
| S009-F3.80.22 | 263 | WB | 24678 | 36.40 | 4801 | 0.99 | 57 | NB | 58 | 103 |
| Tab108 | 7762 | 1.04 | 51738 | 1.85 | 3283 | 0.59 | 23870 | 0.84 | 81 | 446 |
| Tab131 | 638 | WE | 43894 | 3.92 | 205 | WB | 96 | NB | 66 | 390 |
| Tab142 | 11637 | 3.43 | 105314 | 2.28 | 97 | NB | 43336 | 0.43 | 82 | 168 |
| hIgG1 | 122 | NB | 267 | NB | 102 | NB | 102 | NB | 96 | 169 |

TABLE 55
Assay results for binding reactions of F4, F5, and F6 chimeric antibodies with cells by FACS

| | | | | | | | HEK293T-monkey | | | |
|---|---|---|---|---|---|---|---|---|---|---|
| | OVCAR3 | | HEK293T-hMSLN-B8 | | HEK293T-hMSLN-R3 | | MSLN | | 293T | A431 |
| | Maximum | | Maximum | | Maximum | | Maximum | | Maximum | Maximum |
| Cell | mean | | mean | | mean | | mean | | mean | mean |
| Antibody | fluorescence | EC50 | fluorescence | EC50 | fluorescence | EC50 | fluorescence | EC50 | fluorescence | fluorescence |
| name | intensity | (nM) | intensity | (nM) | intensity | (nM) | intensity | (nM) | intensity | intensity |
| S009-F4-94.15 | 7059 | 0.65 | 113125 | 3.06 | 84 | NB | 29930 | 0.37 | 97 | 298 |
| S009-F4- | 3365 | WB | 101584 | 6.07 | 14236 | 1.97 | 41051 | 3.42 | 95 | 362 |
| 127.10 | | | | | | | | | | |
| S009-F5-9.16 | 3506 | 0.82 | 240329 | 0.76 | 63930 | 0.35 | 365 | NB | 185 | 379 |
| S009-F6-62.5 | 2599 | 0.31 | 201872 | 0.41 | 186 | NB | 202 | NB | 168 | 166 |
| S009-F6-76.1 | 6810 | 4.13 | 230670 | 1.39 | 135 | NB | 131 | NB | 91 | 163 |
| Tab106 | 18448 | 0.84 | 171873 | 2.17 | 25392 | 0.28 | 32811 | 0.26 | 363 | 875 |
| Tab108 | 6698 | 0.61 | 193371 | 3.37 | 14920 | 0.69 | 44229 | 0.35 | 189 | 446 |
| Tab142 | 8068 | 1.04 | 243022 | 3.65 | 98 | NB | 63356 | 0.35 | 106 | 168 |
| hIgG1 | 122 | NB | 267 | NB | 102 | NB | 102 | NB | 96 | 169 |

TABLE 56
Assay results for binding reactions of F7 chimeric antibodies with cells by FACS

| | OVCAR3 | | HEK293T-hMSLN-R3 | | HEK293T-monkey MSLN | | 293T | A431 |
|---|---|---|---|---|---|---|---|---|
| | Maximum | | Maximum | | Maximum | | Maximum | Maximum |
| Cell | mean | | mean | | mean | | mean | mean |
| Antibody | fluorescence | EC50 | fluorescence | EC50 | fluorescence | EC50 | fluorescence | fluorescence |
| name | intensity | (nM) | intensity | (nM) | intensity | (nM) | intensity | intensity |
| S009-F7.2.3 | 22757 | 1.74 | 33 | NB | 2320 | WB | 144 | 150 |
| S009-F7.6.17 | 31795 | 0.99 | 6419 | 0.31 | 56055 | 0.70 | 265 | 142 |
| S009-F7.11.11 | 31753 | 0.86 | 6264 | 0.28 | 49481 | 0.41 | 112 | 108 |
| S009-F7.12.13 | 21351 | 0.32 | 6611 | 0.12 | 7628 | 6.34 | 115 | 156 |
| S009-F7.18.10 | 29853 | 3.29 | 39 | NB | 50563 | 0.47 | 123 | 115 |
| S009-F7.21.16 | 32560 | 0.52 | 6817 | 0.16 | 50945 | 0.30 | 129 | 191 |
| S009-F7.23.19 | 29289 | 0.15 | 6567 | 0.05 | 54256 | 0.13 | 116 | 183 |
| S009-F7.25.19 | 24158 | 0.18 | 5350 | 0.06 | 43225 | ~0.16 | 171 | 123 |
| S009-F7.26.15 | 16203 | 0.20 | 6943 | 0.09 | 108 | NB | 285 | 330 |
| S009-F7.30.5 | 31755 | 0.66 | 6161 | 0.24 | 47357 | 0.34 | 158 | 136 |
| S009-F7.33.24 | 31180 | 0.15 | 6476 | 0.06 | 47251 | 0.13 | 139 | 148 |
| S009-F7.41.18 | 25915 | 0.22 | 7463 | 0.35 | 64203 | 0.54 | 235 | 248 |
| S009-F7.44.20 | 25666 | 0.05 | 7473 | 0.04 | 45619 | 0.04 | 220 | 135 |
| S009-F7.48.1 | 23970 | ~4.00 | 5153 | 1.87 | 43181 | 2.88 | 173 | 138 |
| S009-F7.53.2 | 23611 | 1.09 | 6481 | 1.30 | 47358 | 1.13 | 115 | 97 |
| S009-F7.61.21 | 23680 | 1.08 | 6246 | 0.48 | 39833 | 0.75 | 117 | 98 |
| S009-F7.65.13 | 26487 | 0.87 | 6489 | 0.91 | 50228 | 0.87 | 130 | 193 |
| S009-F7.66.12 | 21888 | 0.87 | 5904 | 0.38 | 60 | NB | 127 | 154 |
| S009-F7.67.12 | 29077 | 0.37 | 6929 | 0.14 | 59343 | 0.36 | 136 | 120 |
| S009-F7.69.8 | 16634 | 0.36 | 6593 | 0.14 | 9231 | 12.48 | 289 | 388 |
| Tab142 | 39024 | 5.34 | 42 | NB | 55948 | 0.28 | 154 | 125 |
| Tab106 | 38118 | 1.14 | 7460 | 0.65 | 36456 | 0.82 | 3872 | 4220 |

TABLE 57
Assay results for binding reactions of F8 chimeric antibodies with cells by FACS

| | OVCAR3 | | HEK293T-hMSLN-R3 | | HEK293T-monkey MSLN | | 293T | A431 |
|---|---|---|---|---|---|---|---|---|
| | Maximum | | Maximum | | Maximum | | Maximum | Maximum |
| Cell | mean | | mean | | mean | | mean | mean |
| Antibody | fluorescence | EC50 | fluorescence | EC50 | fluorescence | EC50 | fluorescence | fluorescence |
| name | intensity | (nM) | intensity | (nM) | intensity | (nM) | intensity | intensity |
| S009-F8-4.5 | 13973 | 4.93 | 192 | NB | 35016 | 0.64 | 101 | 277 |
| S009-F8-5.15 | 4918 | 4.08 | 98 | NB | 20171 | ~0.18 | 79 | 162 |
| S009-F8-7.5 | 8991 | 0.71 | 279 | NB | 100 | NB | 154 | 150 |
| S009-F8-8.22 | 4267 | 9.90 | 122 | NB | 112313 | 0.36 | 70 | 94 |
| S009-F8-9.16 | 5375 | 0.86 | 127882 | 0.53 | 348 | WB | 278 | 432 |
| S009-F8-12.13 | 9834 | 2.24 | 135 | NE | 21538 | 0.08 | 105 | 133 |
| S009-F8-13.8 | 13005 | 1.42 | 95 | NB | 20525 | ~0.15 | 85 | 106 |
| S009-F8-15.19 | 8140 | 3.16 | 165 | NB | 28193 | 0.39 | 78 | 182 |
| S009-F8-18.9 | 10815 | 24.06 | 131 | NB | 24920 | 0.81 | 74 | 102 |
| S009-F8-19.21 | 13035 | 5.13 | 98 | NB | 24860 | 0.30 | 70 | 126 |
| S009-F8-22.23 | 5499 | 12.79 | 404 | WB | 114476 | 1.09 | 91 | 132 |
| S009-F8-24.14 | 4700 | 11.46 | 82 | NB | 105243 | 0.52 | 87 | 61 |
| S009-F8-27.1 | 7625 | 77.86 | 120 | NB | 31317 | 2.05 | 83 | 99 |
| S009-F8-28.23 | 1291 | 59.75 | 51 | NB | 90925 | 3.36 | 63 | 50 |
| S009-F8-29.1 | 5433 | 33.36 | 69 | NB | 80 | NB | 55 | 57 |
| S009-F8-31.22 | 7754 | 0.64 | 189 | NB | 3391 | WB | 96 | 260 |
| S009-F8-32.3 | 8274 | 14.67 | 50 | NB | 112003 | 0.69 | 52 | 69 |
| S009-F8-33.12 | 2414 | 28.44 | 64 | NB | 106417 | 0.64 | 49 | 56 |
| S009-F8-36.12 | 2509 | 15.19 | 7968 | 11.00 | 173 | NB | 51 | 65 |
| Tab106 | 14159 | 3 | 24534 | 1 | 12390 | 0 | 260 | 560 |
| Tab142 | 14765 | 9 | 127 | NB | 39177 | 1 | 86 | 137 |

### EXAMPLE 5. ASSAY ON AFFINITY OF MSLN ANTIBODIES

### 5.1. Assay on Affinity of Chimeric Antibodies for Human MSLN-FL-his Protein

Anti-human MSLN chimeric antibodies were captured using a Protein A chip (GE Healthcare; 29-127-558). The sample and running buffer was HBS-EP+ (10 mM HEPES, 150 mM NaCl, 3 mM EDTA, 0.05% surfactant P20) (GE Healthcare; BR-1006-69). The flow cell was set at 25° C. The sample block was set at 16° C. Both were pretreated with the running buffer. In each cycle, first, the antibody to be tested was captured using the Protein A chip, and then a single concentration of human MSLN-FL-his protein was injected. The association and dissociation processes of the antibody with the antigen protein were recorded, and finally, the chip was regenerated using Glycine pH 1.5 (GE Healthcare; BR-1003-54). The association was determined by injecting different concentrations of human MSLN-FL-his in solution for 240 s on end, wherein the flow rate was 30 μL/min, and the protein was diluted in a 1:1 dilution ratio from 200 nM (see detailed results for actual concentrations tested) to obtain 5 concentrations in total. The dissociation phase was monitored for up to 600 s and triggered by switching from the sample solution to the running buffer. The surface was regenerated by washing with 10 mM glycine solution (pH 1.5) at a flow rate of 30 μL/min for 30 s. The difference in bulk refractive index was corrected by subtracting the responses obtained from the goat anti-human Fc surface. Blank injections were also subtracted (=double reference). To calculate the apparent KD and other kinetic parameters, the Langmuir 1:1 model was used. The association rate (Ka), dissociation rate (Kd) and binding affinity (KD) of the human MSLN chimeric antibodies with the human MSLN-FL-his protein are shown in Tables 58-59, wherein the antibodies Tab 106, Tab 108, and Tab 142 were used as controls. The results show that most of the human MSLN chimeric antibodies had an affinity of not less than 7.45E-7 M for the human MSLN protein, wherein S009-F3.7.3, S009-F3.16.1, S009-F3.38.10, and S009-F8-9.16 bound to the human MSLN-R3 protein, and did not show any data from SPR assays.

TABLE 58
Assay results for affinity of F1, F2, F3, F4, F5, and
F6 chimeric antibodies for human MSLN by SPR (biacore)

| Antibody name | ka (1/Ms) | kd (1/s) | KD (M) |
|---|---|---|---|
| S009-F1.2.12 | 9.06E+04 | 1.43E−03 | 1.58E−08 |
| S009-F1.7.14 | 5.53E+05 | 7.09E−03 | 1.28E−08 |
| S009-F1.25.10 | 3.60E+03 | 6.70E−04 | 1.86E−07 |
| S009-F1.35.24 | 9.31E+04 | 8.51E−05 | 9.14E−10 |
| S009-F1.56.1 | 7.99E+05 | 7.10E−04 | 8.89E−10 |
| S009-F1.59.1 | 1.64E+03 | 9.22E−04 | 5.63E−07 |
| S009-F1.62.9 | 4.67E+02 | 3.48E−04 | 7.45E−07 |
| S009-F2.13.3 | 2.19E+05 | 1.89E−03 | 8.65E−09 |
| S009-F2.16.10 | 1.09E+06 | 1.64E−03 | 1.51E−09 |
| S009-F2.17.3 | 6.41E+04 | 1.74E−04 | 2.72E−09 |
| S009-F2.21.4 | 7.80E+05 | 2.27E−02 | 2.91E−08 |
| S009-F2.23.12 | 8.19E+04 | 7.14E−04 | 8.71E−09 |
| S009-F2.38.12 | 3.54E+05 | 1.09E−02 | 3.08E−08 |
| S009-F2.39.3 | 4.00E+04 | 1.22E−04 | 3.06E−09 |
| S009-F2.47.1 | 2.01E+06 | 1.03E−02 | 5.13E−09 |
| S009-F2-56.12 | 7.24E+04 | 3.12E−03 | 4.31E−08 |
| S009-F2.58.8 | 2.36E+05 | 1.23E−04 | 5.24E−10 |
| S009-F3.23.1 | 1.02E+03 | 7.03E−04 | 6.89E−07 |
| S009-F3.45.21 | 5.52E+05 | 8.30E−04 | 1.50E−09 |
| S009-F3.51.8 | 2.47E+03 | 3.50E−04 | 1.42E−07 |
| S009-F3-63.5 | 4.56E+03 | 4.78E−04 | 1.05E−07 |
| S009-F3.74.20 | 1.03E+04 | 5.04E−05 | 4.92E−09 |
| S009-F3.80.22 | 2.20E+03 | 3.32E−04 | 1.51E−07 |
| S009-F4-94.15 | 1.54E+05 | 1.85E−04 | 1.20E−09 |
| S009-F4-127.10 | 2.53E+03 | 4.33E−04 | 1.71E−07 |
| S009-F6-62.5 | 1.44E+05 | 1.73E−02 | 1.20E−07 |
| S009-F6-76.1 | 3.67E+04 | 3.69E−04 | 1.01E−08 |
| Tab108 | 3.77E+05 | 1.26E−04 | 3.33E−10 |

TABLE 59
Assay results for affinity of F7 and F8 chimeric
antibodies for human MSLN by SPR (biacore)

| Antibody name | ka (1/Ms) | kd (1/s) | KD (M) |
|---|---|---|---|
| S009-F7.2.3 | 6.43E+05 | 8.51E−03 | 1.32E−08 |
| S009-F7.6.17 | 3.29E+05 | 3.89E−04 | 1.18E−09 |
| S009-F7.11.11 | 1.33E+06 | 2.40E−04 | 1.81E−10 |
| S009-F7.12.13 | 2.20E+05 | 8.92E−03 | 4.05E−08 |
| S009-F7.18.10 | 1.83E+06 | 1.17E−03 | 6.40E−10 |
| S009-F7.21.16 | 4.58E+05 | 7.73E−04 | 1.69E−09 |
| S009-F7.23.19 | 5.11E+05 | 6.95E−03 | 1.36E−08 |
| S009-F7.25.19 | 4.17E+05 | 1.13E−03 | 2.71E−09 |
| S009-F7.26.15 | 4.88E+06 | 9.75E−04 | 2.00E−10 |
| S009-F7.30.5 | 1.84E+05 | 7.05E−04 | 3.83E−09 |
| S009-F7.33.24 | 4.98E+05 | 5.06E−03 | 1.02E−08 |
| S009-F7.41.18 | 2.00E+05 | 8.35E−05 | 4.18E−10 |
| S009-F7.44.20 | 3.51E+05 | 1.06E−03 | 3.01E−09 |
| S009-F7.48.1 | 2.31E+04 | 4.13E−04 | 1.78E−08 |
| S009-F7.53.2 | 1.99E+05 | 6.50E−04 | 3.27E−09 |
| S009-F7.61.21 | 3.06E+05 | 1.54E−03 | 5.03E−09 |
| S009-F7.65.13 | 3.74E+05 | 5.38E−04 | 1.44E−09 |
| S009-F7.66.12 | 1.80E+05 | 2.36E−03 | 1.31E−08 |
| S009-F7.67.12 | 1.08E+06 | 6.50E−04 | 6.02E−10 |
| S009-F7.69.8 | 6.87E+05 | 4.47E−03 | 6.51E−09 |
| S009-F8-4.5 | 7.27E+05 | 5.99E−04 | 8.24E−10 |
| S009-F8-5.15 | 6.20E+05 | 1.70E−02 | 2.74E−08 |
| S009-F8-7.5 | 2.32E+05 | 2.15E−02 | 9.26E−08 |
| S009-F8-8.22 | 7.32E+05 | 9.37E−03 | 1.28E−08 |
| S009-F8-12.13 | 5.39E+05 | 2.96E−03 | 5.50E−09 |
| S009-F8-13.8 | 1.31E+06 | 1.25E−03 | 9.50E−10 |
| S009-F8-15.19 | 1.35E+06 | 7.24E−03 | 5.37E−09 |
| S009-F8-18.9 | 2.76E+05 | 8.76E−04 | 3.17E−09 |
| S009-F8-19.21 | 8.71E+05 | 1.72E−03 | 1.98E−09 |
| S009-F8-22.23 | 4.33E+05 | 2.75E−03 | 6.35E−09 |
| S009-F8-24.14 | 5.49E+05 | 5.01E−03 | 9.13E−09 |
| S009-F8-27.1 | 1.20E+05 | 2.34E−04 | 1.95E−09 |
| S009-F8-28.23 | 1.92E+05 | 8.43E−03 | 4.40E−08 |
| S009-F8-29.1 | 7.28E+05 | 1.99E−02 | 2.74E−08 |
| S009-F8-31.22 | 3.15E+05 | 2.76E−02 | 8.76E−08 |
| S009-F8-32.3 | 5.10E+05 | 3.31E−03 | 6.50E−09 |
| S009-F8-33.12 | 6.27E+06 | 3.61E−02 | 5.77E−09 |
| S009-F8-36.12 | 1.18E+05 | 1.22E−02 | 1.03E−07 |
| Tab106 | 4.94E+05 | 1.87E−04 | 3.78E−10 |
| Tab142 | 9.33E+05 | 2.17E−04 | 2.33E−10 |

### EXAMPLE 6. ANTIBODY-ANTIGEN BINDING EPITOPE BINNING

### 6.1. Competitive ELISA

In order to identify antigen-binding sites of antibodies, MSLN chimeric antibodies were grouped using competitive ELISA. Referring to the method described in Example 5 (5.1), ELISA plates were coated with 2 μg/mL chimeric antibodies; the human MSLN protein was subjected to a gradient dilution from 30 μg/mL, and EC₈₀ was calculated as the concentration in competitive ELISA.

The chimeric antibodies were diluted to 2 μg/mL with PBS and allowed to coat 96-well high-adsorption ELISA plates at 50 μL/well. After the plates were coated at 4° C. overnight, 250 μL of a blocking buffer (PBS containing 2% (w/w) BSA) was added for two hours of blocking at room temperature. 40 μg/mL of the antibodies to be tested were added, and then the human MSLN-FL-His protein with an EC₈₀ concentration corresponding to each of the antibodies to be tested was added for incubation for 2 h. The plates were washed 5 times with PBS, an HRP-labeled anti-His secondary antibody (purchased from Genescript, Cat. No. A00612) was then added for incubation for 1 h, and the plates were washed 5 times again. A TMB substrate was added at 50 μL/well, and after 10 minutes of incubation at room temperature, a stop solution (1.0 M HCl) was added at 50 μL/well. OD₄₅₀ nm values were read using an ELISA plate reader (Insight, purchased from PerkinElmer), and the competition rate between the antibodies was calculated according to the OD₄₅₀ nm values using a formula. The results are shown in FIGS. 31-37: the higher the value of the competition rate, the closer the epitopes to which two antibodies bind.

### EXAMPLE 7. HUMANIZATION OF MSLN ANTIBODIES

Chimeric antibodies F2.23.12, F2.39.3, F7.44.20, F7.33.24, F3.80.22, and F3.38.10 were humanized.

By alignment with the IMGT database (website: imgt.cines.fr) for germline genes from heavy and light chain variable regions of human antibodies, germline genes, with high homology with the murine antibodies, from heavy and light chain variable regions were selected as templates, and CDRs of the murine antibodies were separately grafted into corresponding humanized templates to form variable region sequences in the order of FR1-CDR1-FR2-CDR2-FR3-CDR3-FR4. Based on the three-dimensional structure of the antibody, embedded residues, residues directly interacting with the CDRs, and residues in framework regions that had an important influence on the conformation of VL and VH were back-mutated, thus giving humanized monoclonal antibodies. The amino acid residues of CDRs of the antibodies were identified and annotated by the Kabat numbering scheme.

### 7.1. Humanization of S009-F2.39.3

For antibody S009-F2.39.3, humanized light chain templates were IGKV4-1*01/IGKV2-29*02 and IGKJ4*01, and humanized heavy chain templates were IGHV1-46*01 and IGHJ1*01. CDRs of the murine antibody S009-F2.39.3 were separately grafted into their humanized templates, so as to obtain the corresponding humanized versions. Key amino acids in FR sequences of the humanized antibodies of S009-F2.39.3 were back-mutated to amino acids corresponding to the murine antibody as needed to ensure the original affinity. Detailed back mutation design is shown in Table 60.

TABLE 60
Back mutation design for humanized antibodies of S009-F2.39.3

| VL | | VH | |
|---|---|---|---|
| F2.39.3.VL1 | Graft(IGKV4-1*01) + | F2.39.3.VH1 | Graft(IGHV1-46*01) + |
| | P49S | | R72V, T74K |
| F2.39.3.VL2 | Graft(IGKV4-1*01) + | F2.39.3.VH2 | Graft(IGHV1-46*01) + |
| | P49S, K51T | | R72V, T74K, V79A |
| F2.39.3.VL3 | Graft(IGKV2-29*02) + | F2.39.3.VH3 | Graft(IGHV1-46*01) + |
| | I54V | | R72V, T74K, V79A, |
| F2.39.3.VL4 | Graft(IGKV2-29*02) + | | Q109P |
| | L43Q, Q51T, I54V | | |

Note: Graft denotes that the CDRs of the murine antibody are grafted into the human germline template FR sequences; P49S denotes that P at position 49 of Graft is mutated to S, and so on for others. The back-mutated amino acids are numbered in the natural order.

Specific sequences of the variable regions of the S009-F2.39.3 humanized antibodies are as follows:

S009-F2.39.3. VL1 has an amino acid sequence set
forth in SEQ ID NO: 565:
DIVMTQSPDSLAVSLGERATINCKSSQTLLNSVSQNNYLAWYQQKPGQSP
KLLIYFASTRESGVPDRFSGSGSGTDFTLTISSLQAEDVAVYYCQQHYRT
PYTFGGGTKVEIK.
S009-F2.39.3. VL2 has an amino acid sequence set
forth in SEQ ID NO: 566:
DIVMTQSPDSLAVSLGERATINCKSSQTLLNSVSQNNYLAWYQQKPGQSP
TLLIYFASTRESGVPDRFSGSGSGTDFTLTISSLQAEDVAVYYCQQHYRT
PYTFGGGTKVEIK.
S009-F2.39.3.VL3 has an amino acid sequence set
forth in SEQ ID NO: 567:
DIVMTQTPLSLSVTPGQPASISCKSSQTLLNSVSQNNYLAWYLQKPGQSP
QLLVYFASTRESGVPDRFSGSGSGTDFTLKISRVEAEDVGVYYCQQHYRT
PYTFGGGTKVEIK.
S009-F2.39.3.VL4 has an amino acid sequence set
forth in SEQ ID NO: 568:
DIVMTQTPLSLSVTPGQPASISCKSSQTLLNSVSQNNYLAWYQQKPGQSP
TLLVYFASTRESGVPDRFSGSGSGTDFTLKISRVEAEDVGVYYCQQHYRT
PYTFGGGTKVEIK.
S009-F2.39.3. VH1 has an amino acid sequence set
forth in SEQ ID NO: 569:
EVQLVQSGAEVKKPGASVKVSCKASGYTFTNYWMHWVRQAPGQGLEWMGN
INPSSGDSYYNERFMSRVTMTVDKSTSTVYMELSSLRSEDTAVYYCARSG
GLWLAFWGQGTLVTVSS.
S009-F2.39.3. VH2 has an amino acid sequence set
forth in SEQ ID NO: 570:
EVQLVQSGAEVKKPGASVKVSCKASGYTFTNYWMHWVRQAPGQGLEWMGN
INPSSGDSYYNERFMSRVTMTVDKSTSTAYMELSSLRSEDTAVYYCARSG
GLWLAFWGQGTLVTVSS.
S009-F2.39.3. VH3 has an amino acid sequence set
forth in SEQ ID NO: 571:
EVQLVQSGAEVKKPGASVKVSCKASGYTFTNYWMHWVRQAPGQGLEWMGN
INPSSGDSYYNERFMSRVTMTVDKSTSTAYMELSSLRSEDTAVYYCARSG
GLWLAFWGPGTLVTVSS.
The humanized light chain template IGKV4-1*01 has
an amino acid sequence set forth in SEQ ID NO:
572:
DIVMTQSPDSLAVSLGERATINCKSSQSVLYSSNNKNYLAWYQQKPGQPP
KLLIYWASTRESGVPDRFSGSGSGTDFTLTISSLQAEDVAVYYCQQYYST
P.
The humanized light chain template IGKV2-29*02 has
an amino acid sequence set forth in SEQ ID NO:
573:
DIVMTQTPLSLSVTPGQPASISCKSSQSLLHSDGKTYLYWYLQKPGQSPQ
LLIYEVSSRFSGVPDRFSGSGSGTDFTLKISRVEAEDVGVYYCMQGIHL
P.
The humanized light chain template IGKJ4*01 has an
amino acid sequence set forth in SEQ ID NO: 574:
FGGGTKVEIK.
The humanized heavy chain template IGHV1-46*01 has
an amino acid sequence set forth in SEQ ID NO:
575:
QVQLVQSGAEVKKPGASVKVSCKASGYTFTSYYMHWVRQAPGQGLEWMGI
INPSGGSTSYAQKFQGRVTMTRDTSTSTVYMELSSLRSEDTAVYYCAR.
The humanized heavy chain template IGHJ1*01 has an
amino acid sequence set forth in SEQ ID NO: 576:
WGQGTLVTVSS.

According to the Kabat numbering scheme, the analysis results for VH and VL sequences of the humanized antibodies described above are shown in Table 61.

TABLE 61
Kabat analysis results for VH and VL sequences of S009-F2.39.3 humanized
antibodies

| Variable region | | | |
|---|---|---|---|
| No. | CDR1 | CDR2 | CDR3 |
| F2.39.3.VL1/2/3/ | KSSQTLLNSVSQNNYLA | FASTRES | QQHYRTPYT |
| 4 | SEQ ID NO: 247 | SEQ ID NO: 248 | SEQ ID NO: 249 |
| F2.39.3.VH1/2/3 | NYWMH | NINPSSGDSYYNERFMS | SGGLWLAF |
| | SEQ ID NO: 244 | SEQ ID NO: 245 | SEQ ID NO: 246 |

### 7.2. Humanization of S009-F2.23.12

For antibody S009-F2.23.12, humanized light chain templates were IGKV1-33*01 and IGKJ4*01, and humanized heavy chain templates were IGHV1-46*01 and IGHJ6*01. CDRs of the murine antibody S009-F2.23.12 were separately grafted into their humanized templates, so as to obtain the corresponding humanized versions. Key amino acids in FR sequences of the humanized antibodies of S009-F2.23.12 were back-mutated to amino acids corresponding to the murine antibody as needed to ensure the original affinity (there are sites in the antibody that are susceptible to chemical modification, and we performed point mutations at these sites to eliminate modification risks). Detailed back mutation design is shown in Table 62.

TABLE 62
Back mutation design for humanized antibodies of S009-F2.23.12

| VL | | VH | |
|---|---|---|---|
| S009- | Graft(IGKV1-33*01) + | S009- | Graft(IGHV1-46*01) + |
| F2.23.12.VL1 | Y49H | F2.23.12.VH1 | R72V, T74K |
| S009- | Graft(IGKV1-33*01) + | S009- | Graft(IGHV1-46*01) + |
| F2.23.12.VL2 | Q38H, Y49H | F2.23.12.VH2 | R72V, T74K, V79A |
| S009- | Graft(IGKV1-33*01) + | S009- | Graft(IGHV1-46*01) + |
| F2.23.12.VL3 | Q38H, Y49H, T69R | F2.23.12.VH3 | R72V, T74K, V79A + N55D |
| S009- | Graft(IGKV1-33*01) + | S009- | Graft(IGHV1-46*01) + |
| F2.23.12.VL4 | Q38H, Y49H, T69R, F71Y | F2.23.12.VH4 | R72V, T74K, V79A + N55Q |

Note: Graft denotes that the CDRs of the murine antibody are grafted into the human germline template FR sequences; Y49H denotes that Y at position 49 of Graft is mutated to H, and so on for others. The back-mutated amino acids are numbered in the natural order.

Specific sequences of the variable regions of the S009-F2.23.12 humanized antibodies are as follows:

S009-F2.23.12.VL1 has an amino acid sequence set
forth in SEQ ID NO: 577:
DIQMTQSPSSLSASVGDRVTITCKASQDINKYIAWYQQKPGKAPKLLIHY
TSELQPGVPSRFSGSGSGTDFTFTISSLQPEDIATYYCLQYANPLRTFGG
GTKVEIK.
S009-F2.23.12. VL2 has an amino acid sequence set
forth in SEQ ID NO: 578:
DIQMTQSPSSLSASVGDRVTITCKASQDINKYIAWYQHKPGKAPKLLIHY
TSELQPGVPSRFSGSGSGTDFTFTISSLQPEDIATYYCLQYANPLRTFGG
GTKVEIK.
S009-F2.23.12. VL3 has an amino acid sequence set
forth in SEQ ID NO: 579:
DIQMTQSPSSLSASVGDRVTITCKASQDINKYIAWYQHKPGKAPKLLIHY
TSELQPGVPSRFSGSGSGRDFTFTISSLQPEDIATYYCLQYANPLRTFGG
GTKVEIK.
S009-F2.23.12. VL4 has an amino acid sequence set
forth in SEQ ID NO: 580:
DIQMTQSPSSLSASVGDRVTITCKASQDINKYIAWYQHKPGKAPKLLIHY
TSELQPGVPSRFSGSGSGRDYTFTISSLQPEDIATYYCLQYANPLRTFGG
GTKVEIK.
S009-F2.23.12. VH1 has an amino acid sequence set
forth in SEQ ID NO: 581:
EVQLVQSGAEVKKPGASVKVSCKASGYTFTNYWMHWVRQAPGQGLEWMGN
INPSNGGPYYNERFRSRVTMTVDKSTSTVYMELSSLRSEDTAVYYCARPY
YGSSYGYFDYWGQGTTVTVSS.
S009-F2.23.12. VH2 has an amino acid sequence set
forth in SEQ ID NO: 582:
EVQLVQSGAEVKKPGASVKVSCKASGYTFTNYWMHWVRQAPGQGLEWMGN
INPSNGGPYYNERFRSRVTMTVDKSTSTAYMELSSLRSEDTAVYYCARPY
YGSSYGYFDYWGQGTTVTVSS.
S009-F2.23.12. VH3 has an amino acid sequence set
forth in SEQ ID NO: 583:
EVQLVQSGAEVKKPGASVKVSCKASGYTFTNYWMHWVRQAPGQGLEWMGN
INPSDGGPYYNERFRSRVTMTVDKSTSTAYMELSSLRSEDTAVYYCARPY
YGSSYGYFDYWGQGTTVTVSS.
S009-F2.23.12. VH4 has an amino acid sequence set
forth in SEQ ID NO: 584:
EVQLVQSGAEVKKPGASVKVSCKASGYTFTNYWMHWVRQAPGQGLEWMGN
INPSQGGPYYNERFRSRVTMTVDKSTSTAYMELSSLRSEDTAVYYCARPY
YGSSYGYFDYWGQGTTVTVSS.
The humanized light chain template IGKV1-33*01 has
an amino acid sequence set forth in SEQ ID NO:
585:
DIQMTQSPSSLSASVGDRVTITCQASQDISNYLNWYQQKPGKAPKLLIYD
ASNLETGVPSRFSGSGSGTDFTFTISSLQPEDIATYYCQQYDNLP.
The humanized light chain template IGKJ4*01 has an
amino acid sequence set forth in SEQ ID NO: 574:
FGGGTKVEIK.
The humanized heavy chain template IGHV1-46*01 has
an amino acid sequence set forth in SEQ ID NO:
575:
QVQLVQSGAEVKKPGASVKVSCKASGYTFTSYYMHWVRQAPGQGLEWMGI
INPSGGSTSYAQKFQGRVTMTRDTSTSTVYMELSSLRSEDTAVYYCAR.
The humanized heavy chain template IGHJ6*01 has an
amino acid sequence set forth in SEQ ID NO: 586:
WGQGTTVTVSS.

According to the Kabat numbering scheme, the analysis results for VH and VL sequences of the humanized antibodies described above are shown in Table 63.

TABLE 63
Kabat analysis results for VH and VL sequences of S009-F2.23.12 humanized
antibodies

| Variable region No. | CDR1 | CDR2 | CDR3 |
|---|---|---|---|
| F2.23.12.VL1/2/3/4 | KASQDINKYIA | YTSELQP | LQYANPLRT |
| | SEQ ID NO: 235 | SEQ ID NO: 236 | SEQ ID NO: 237 |
| F2.23.12. VH1/2 | NYWMH | NINPSNGGPYYNERFRS | PYYGSSYGYFDY |
| | SEQ ID NO: 232 | SEQ ID NO: 233 | SEQ ID NO: 234 |
| F2.23.12. VH3 | NYWMH | NINPSDGGPYYNERFRS | PYYGSSYGYFDY |
| | SEQ ID NO: 232 | SEQ ID NO: 587 | SEQ ID NO: 234 |
| F2.23.12. VH4 | NYWMH | NINPSQGGPYYNERFRS | PYYGSSYGYFDY |
| | SEQ ID NO: 232 | SEQ ID NO: 588 | SEQ ID NO: 234 |

### 7.3. Humanization of S009-F7.44.20

For antibody S009-F7.44.20, humanized light chain templates were IGKV3-11*01/IGKV6-21*01 and IGKJ4*01, and humanized heavy chain templates were IGHV1-46*01 and IGHJ6*01. CDRs of the murine antibody S009-F7.44.20 were separately grafted into their humanized templates, so as to obtain the corresponding humanized versions. Key amino acids in FR sequences of the humanized antibodies of S009-F7.44.20 were back-mutated to amino acids corresponding to the murine antibody as needed to ensure the original affinity (there are sites in the antibody that are susceptible to chemical modification, and we performed point mutations at these sites to eliminate modification risks). Detailed back mutation design is shown in Table 64.

TABLE 64
Back mutation design for humanized antibodies of S009-F7.44.20

| VL | | VH | |
|---|---|---|---|
| S009- | Graft(IGKV6-21*01) + | S009- | Graft(IGHV1-46*01) + |
| F7.44.20.VL1 | K50Y | F7.44.20.VH1 | R72E, T74K |
| S009- | Graft(IGKV6-21*01) + | S009- | Graft(IGHV1-46*01) + |
| F7.44.20.VL2 | K50Y, F72Y | F7.44.20.VH1a | R72E, T74K + N65Q |
| S009- | Graft(IGKV3-11*01) + | S009- | Graft(IGHV1-46*01) + |
| F7.44.20.VL3 | A44S, F72Y | F7.44.20.VH2 | R72E, T74K, T76A |
| S009- | Graft(IGKV3-11*01) + | S009- | Graft(IGHV1-46*01) + R72E, |
| F7.44.20.VL4 | Q43S, A44S, F72Y | F7.44.20.VH2a | T74K, T76A + N65S |

Note: Graft denotes that the CDRs of the murine antibody are grafted into the human germline template FR sequences; K50Y denotes that K at position 50 of Graft is mutated to Y, and so on for others. The back-mutated amino acids are numbered in the natural order.

Specific sequences of the variable regions of the S009-F7.44.20 humanized antibodies are as follows:

S009-F7.44.20. VL1 has an amino acid sequence set
forth in SEQ ID NO: 589:
EIVLTQSPDFQSVTPKEKVTITCSASSSVISSYLSWYQQKPDQSPKLLIY
RTSNLASGVPSRFSGSGSGTDFTLTINSLEAEDAATYYCHQWSSFPYTFG
GGTKVEIK.
S009-F7.44.20. VL2 has an amino acid sequence set
forth in SEQ ID NO: 590:
EIVLTQSPDFQSVTPKEKVTITCSASSSVISSYLSWYQQKPDQSPKLLIY
RTSNLASGVPSRFSGSGSGTDYTLTINSLEAEDAATYYCHQWSSFPYTFG
GGTKVEIK.
S009-F7.44.20. VL3 has an amino acid sequence set
forth in SEQ ID NO: 591:
EIVLTQSPATLSLSPGERATLSCSASSSVISSYLSWYQQKPGQSPRLLIY
RTSNLASGIPARFSGSGSGTDYTLTISSLEPEDFAVYYCHQWSSFPYTFG
GGTKVEIK.
S009-F7.44.20. VL4 has an amino acid sequence set
forth in SEQ ID NO: 592:
EIVLTQSPATLSLSPGERATLSCSASSSVISSYLSWYQQKPGSSPRLLIY
RTSNLASGIPARFSGSGSGTDYTLTISSLEPEDFAVYYCHQWSSFPYTFG
GGTKVEIK.
S009-F7.44.20. VH1 has an amino acid sequence set
forth in SEQ ID NO: 593:
EVQLVQSGAEVKKPGASVKVSCKASGYTFTDYNMDWVRQAPGQGLEWMGD
INPSTGGTIYNQKFNGRVTMTEDKSTSTVYMELSSLRSEDTAVYYCARRR
IGTGYFDVWGQGTTVTVSS.
S009-F7.44.20. VH1a has an amino acid sequence set
forth in SEQ ID NO: 594:
EVQLVQSGAEVKKPGASVKVSCKASGYTFTDYNMDWVRQAPGQGLEWMGD
INPSTGGTIYNQKFQGRVTMTEDKSTSTVYMELSSLRSEDTAVYYCARRR
IGTGYFDVWGQGTTVTVSS.
S009-F7.44.20. VH2 has an amino acid sequence set
forth in SEQ ID NO: 595:
EVQLVQSGAEVKKPGASVKVSCKASGYTFTDYNMDWVRQAPGQGLEWMGD
INPSTGGTIYNQKFNGRVTMTEDKSASTVYMELSSLRSEDTAVYYCARRR
IGTGYFDVWGQGTTVTVSS.
S009-F7.44.20. VH2a has an amino acid sequence set
forth in SEQ ID NO: 596:
EVQLVQSGAEVKKPGASVKVSCKASGYTFTDYNMDWVRQAPGQGLEWMGD
INPSTGGTIYNQKFSGRVTMTEDKSASTVYMELSSLRSEDTAVYYCARRR
IGTGYFDVWGQGTTVTVSS.
The humanized light chain template IGKV3-11*01 has
an amino acid sequence set forth in SEQ ID NO:
597:
EIVLTQSPATLSLSPGERATLSCRASQSVSSYLAWYQQKPGQAPRLLIYD
ASNRATGIPARFSGSGSGTDFTLTISSLEPEDFAVYYCQQRSNWP.
The humanized light chain template IGKV6-21*01 has
an amino acid sequence set forth in SEQ ID NO:
598:
EIVLTQSPDFQSVTPKEKVTITCRASQSIGSSLHWYQQKPDQSPKLLIKY
ASQSFSGVPSRFSGSGSGTDFTLTINSLEAEDAATYYCHQSSSLP.
The humanized light chain template IGKJ4*01 has an
amino acid sequence set forth in SEQ ID NO: 574:
FGGGTKVEIK.
The humanized heavy chain template IGHV1-46*01 has
an amino acid sequence set forth in SEQ ID NO:
575:
QVQLVQSGAEVKKPGASVKVSCKASGYTFTSYYMHWVRQAPGQGLEWMGI
INPSGGSTSYAQKFQGRVTMTRDTSTSTVYMELSSLRSEDTAVYYCAR.

The humanized heavy chain template IGHJ6*01 has an amino acid sequence set forth in SEQ ID NO: 586: WGQGTTVTVSS.

According to the Kabat numbering scheme, the analysis results for VH and VL sequences of the humanized antibodies described above are shown in Table 65.

TABLE 65
Kabat analysis results for VH and VL sequences of S009-F7.44.20 humanized
antibodies

| Variable region No. | CDR1 | CDR2 | CDR3 |
|---|---|---|---|
| F7.44.20. VL1/2/3/4 | SASSSVISSYLS | RTSNLAS | HQWSSFPYT |
| | SEQ ID NO: 418 | SEQ ID NO: 419 | SEQ ID NO: 420 |
| F7.44.20.VH1/2 | DYNMD | DINPSTGGTIYNQKENG | RRIGTGYFDV |
| | SEQ ID NO: 415 | SEQ ID NO: 416 | SEQ ID NO: 417 |
| F7.44.20.VH1a | DYNMD | DINPSTGGTIYNQKFQG | RRIGTGYFDV |
| | SEQ ID NO: 415 | SEQ ID NO: 599 | SEQ ID NO: 417 |
| F7.44.20. VH2a | DYNMD | DINPSTGGTIYNQKFSG | RRIGTGYFDV |
| | SEQ ID NO: 415 | SEQ ID NO: 600 | SEQ ID NO: 417 |

### 7.4. Humanization of S009-F7.33.24

For antibody S009-F7.33.24, humanized light chain templates were IGKV2-29*02/IGKV4-1*01 and IGKJ4*01, and humanized heavy chain templates were IGHV1-3*01 and IGHJ1*01. CDRs of the murine antibody S009-F7.33.24 were separately grafted into their humanized templates, so as to obtain the corresponding humanized versions. Key amino acids in FR sequences of the humanized antibodies of S009-F7.33.24 were back-mutated to amino acids corresponding to the murine antibody as needed to ensure the original affinity (there are sites in the antibody that are susceptible to chemical modification, and we performed point mutations at these sites to eliminate modification risks). Detailed back mutation design is shown in Table 66.

TABLE 66
Back mutation design for humanized antibodies of S009-F7.33.24

| VL | | VH | |
|---|---|---|---|
| S009- | Graft(IGKV4-1*01) + | S009- | Graft(IGHV1-3*01) + |
| F7.33.24.VL1 | P43S, L46A | F7.33.24.VH1 | R72V, T74K, R98P |
| S009- | Graft(IGKV2-29*02) + | S009- | Graft(IGHV1-3*01) + |
| F7.33.24.VL2 | L46A | F7.33.24.VH1a | R72V, T74K, R98P + N55S |
| S009- | Graft(IGKV2-29*02) + | S009- | Graft(IGHV1-3*01) + R44G, |
| F7.33.24.VL3 | L37Q, L46A | F7.33.24.VH2 | R72V, T74K, R98P |
| | | S009- | Graft(IGHV1-3*01) + R44G, |
| | | F7.33.24.VH2a | R72V, T74K, R98P + G56A |

Note: Graft denotes that the CDRs of the murine antibody are grafted into the human germline template FR sequences; P43S denotes that P at position 43 of Graft is mutated to S, and so on for others. The back-mutated amino acids are numbered in the natural order.

Specific sequences of the variable regions of the S009-F7.33.24 humanized antibodies are as follows:

S009-F7.33.24. VL1 has an amino acid sequence set
forth in SEQ ID NO: 601:
DIVMTQSPDSLAVSLGERATINCKASQNVGTNIAWYQQKPGQSPKALIYS
ASYRYSGVPDRFSGSGSGTDFTLTISSLQAEDVAVYYCQQYNSYPLTFGG
GTKVEIK.
S009-F7.33.24. VL2 has an amino acid sequence set
forth in SEQ ID NO: 602:
DIVMTQTPLSLSVTPGQPASISCKASQNVGTNIAWYLQKPGQSPQALIYS
ASYRYSGVPDRFSGSGSGTDFTLKISRVEAEDVGVYYCQQYNSYPLTFGG
GTKVEIK.
S009-F7.33.24. VL3 has an amino acid sequence set
forth in SEQ ID NO: 603:
DIVMTQTPLSLSVTPGQPASISCKASQNVGTNIAWYQQKPGQSPQALIYS
ASYRYSGVPDRFSGSGSGTDFTLKISRVEAEDVGVYYCQQYNSYPLTFGG
GTKVEIK.
S009-F7.33.24. VH1 has an amino acid sequence set
forth in SEQ ID NO: 604:
EVQLVQSGAEVKKPGASVKVSCKASGYTFTSYWMHWVRQAPGQRLEWMGN
VNPSNGGSNYNEKFKNRVTITVDKSASTAYMELSSLRSEDTAVYYCAPHY
IGSRPGFAYWGQGTLVTVSS.
S009-F7.33.24. VH1a has an amino acid sequence set
forth in SEQ ID NO: 605:
EVQLVQSGAEVKKPGASVKVSCKASGYTFTSYWMHWVRQAPGQRLEWMGN
VNPSSGGSNYNEKFKNRVTITVDKSASTAYMELSSLRSEDTAVYYCAPHY
IGSRPGFAYWGQGTLVTVSS.
S009-F7.33.24. VH2 has an amino acid sequence set
forth in SEQ ID NO: 606:
EVQLVQSGAEVKKPGASVKVSCKASGYTFTSYWMHWVRQAPGQGLEWMGN
VNPSNGGSNYNEKFKNRVTITVDKSASTAYMELSSLRSEDTAVYYCAPHY
IGSRPGFAYWGQGTLVTVSS.
S009-F7.33.24. VH2a has an amino acid sequence set
forth in SEQ ID NO: 607:
EVQLVQSGAEVKKPGASVKVSCKASGYTFTSYWMHWVRQAPGQGLEWMGN
VNPSNAGSNYNEKFKNRVTITVDKSASTAYMELSSLRSEDTAVYYCAPHY
IGSRPGFAYWGQGTLVTVSS.
The humanized light chain template IGKV2-29*02 has
an amino acid sequence set forth in SEQ ID NO:
573:
DIVMTQTPLSLSVTPGQPASISCKSSQSLLHSDGKTYLYWYLQKPGQSPQ
LLIYEVSSRFSGVPDRFSGSGSGTDFTLKISRVEAEDVGVYYCMQGIHL
P.
The humanized light chain template IGKV4-1*01 has
an amino acid sequence set forth in SEQ ID NO:
572:
DIVMTQSPDSLAVSLGERATINCKSSQSVLYSSNNKNYLAWYQQKPGQPP
KLLIYWASTRESGVPDRFSGSGSGTDFTLTISSLQAEDVAVYYCQQYYST
P.
The humanized light chain template IGKJ4*01 has an
amino acid sequence set forth in SEQ ID NO: 574:
FGGGTKVEIK.
The humanized heavy chain template IGHV1-3*01 has
an amino acid sequence set forth in SEQ ID NO:
608:
QVQLVQSGAEVKKPGASVKVSCKASGYTFTSYAMHWVRQAPGQRLEWMGW
INAGNGNTKYSQKFQGRVTITRDTSASTAYMELSSLRSEDTAVYYCAR.
The humanized heavy chain template IGHJ1*01 has an
amino acid sequence set forth in SEQ ID NO: 576:
WGQGTLVTVSS.

According to the Kabat numbering scheme, the analysis results for VH and VL sequences of the humanized antibodies described above are shown in Table 67.

TABLE 67
Kabat analysis results for VH and VL sequences of S009-F7.33.24 humanized
antibodies

| Variable region No. | CDR1 | CDR2 | CDR3 |
|---|---|---|---|
| F7.33.24.VL1/2/3 | KASQNVGTNIA | SASYRYS | QQYNSYPLT |
| | SEQ ID NO: 406 | SEQ ID NO: 407 | SEQ ID NO: 408 |
| F7.33.24.VH1/2 | SYWMH | NVNPSNGGSNYNEKFKN | HYIGSRPGFAY |
| | SEQ ID NO: 403 | SEQ ID NO: 404 | SEQ ID NO: 405 |
| F7.33.24. VH1a | SYWMH | NVNPSSGGSNYNEKFKN | HYIGSRPGFAY |
| | SEQ ID NO: 403 | SEQ ID NO: 609 | SEQ ID NO: 405 |
| F7.33.24.VH2a | SYWMH | NVNPSNAGSNYNEKFKN | HYIGSRPGFAY |
| | SEQ ID NO: 403 | SEQ ID NO: 610 | SEQ ID NO: 405 |

### 7.5. Humanization of S009-F3.80.22

For antibody S009-F3.80.22, humanized light chain templates were IGKV2-40*01 and IGKJ2*01, and humanized heavy chain templates were IGHV1-69-2*01 and IGHJ6*01. CDRs of the murine antibody S009-F3.80.22 were separately grafted into their humanized templates, so as to obtain the corresponding humanized versions. Key amino acids in FR sequences of the humanized antibodies of S009-F3.80.22 were back-mutated to amino acids corresponding to the murine antibody as needed to ensure the original affinity (there are sites in the antibody that are susceptible to chemical modification, and we performed point mutations at these sites to eliminate modification risks). Detailed back mutation design is shown in Table 68.

TABLE 68
Back mutation design for humanized antibodies of S009-F3.80.22

| VL | | VH | |
|---|---|---|---|
| S009- | Graft(IGKV2-40*01) + | S009- | Graft(IGHV1-69-2*01) + T74K, |
| F3.80.22.VL1 | Y41F | F3.80.22.VH1 | A97T, T98D |
| S009- | Graft(IGKV2-40*01) + | S009- | Graft(IGHV1-69-2*01) + V24A, |
| F3.80.22.VL1a | Y41F + N33D | F3.80.22.VH2 | T74K, D77S, A97T, T98D |
| S009- | Graft(IGKV2-40*01) + | S009- | Graft(IGHV1-69-2*01) + V24A, |
| F3.80.22.VL2 | Y41F, Q43H | F3.80.22.VH3 | T74K, D77S, A97T, T98D, Q112T |
| S009- | Graft(IGKV2-40*01) + | S009- | Graft(IGHV1-69-2*01) + V24A, |
| F3.80.22.VL2a | Y41F, Q43H + G34A | F3.80.22.VH4 | T74K, T76S, D77S, A97T, T98D, Q112T |

Note: Graft denotes that the CDRs of the murine antibody are grafted into the human germline template FR sequences; Y41F denotes that Y at position 41 of Graft is mutated to F, and so on for others. The back-mutated amino acids are numbered in the natural order.

Specific sequences of the variable regions of the S009-F3.80.22 humanized antibodies are as follows:

S009-F3.80.22. VL1 has an amino acid sequence set
forth in SEQ ID NO: 611:
DIVMTQTPLSLPVTPGEPASISCRSSQSLEHNNGNTYLHWFLQKPGQSPQ
LLIYKVSNRFSGVPDRFSGSGSGTDFTLKISRVEAEDVGVYYCSQSTHVP
FTFGQGTKLEIK.
S009-F3.80.22. VLla has an amino acid sequence set
forth in SEQ ID NO: 612:
DIVMTQTPLSLPVTPGEPASISCRSSQSLEHNDGNTYLHWFLQKPGQSPQ
LLIYKVSNRFSGVPDRFSGSGSGTDFTLKISRVEAEDVGVYYCSQSTHVP
FTFGQGTKLEIK.
S009-F3.80.22. VL2 has an amino acid sequence set
forth in SEQ ID NO: 613:
DIVMTQTPLSLPVTPGEPASISCRSSQSLEHNNGNTYLHWFLHKPGQSPQ
LLIYKVSNRFSGVPDRFSGSGSGTDFTLKISRVEAEDVGVYYCSQSTHVP
FTFGQGTKLEIK.
S009-F3.80.22. VL2a has an amino acid sequence set
forth in SEQ ID NO: 614:
DIVMTQTPLSLPVTPGEPASISCRSSQSLEHNNANTYLHWFLHKPGQSPQ
LLIYKVSNRFSGVPDRFSGSGSGTDFTLKISRVEAEDVGVYYCSQSTHVP
FTFGQGTKLEIK.
S009-F3.80.22. VH1 has an amino acid sequence set
forth in SEQ ID NO: 615:
EVQLVQSGAEVKKPGATVKISCKVSGYTFTDYEIHWVQQAPGKGLEWMGA
FDPEIGGSAYNQKFKDRVTITADKSTDTAYMELSSLRSEDTAVYYCTDYY
GSSSGYFDVWGQGTTVTVSS.
S009-F3.80.22. VH2 has an amino acid sequence set
forth in SEQ ID NO: 616:
EVQLVQSGAEVKKPGATVKISCKASGYTFTDYEIHWVQQAPGKGLEWMGA
FDPEIGGSAYNQKFKDRVTITADKSTSTAYMELSSLRSEDTAVYYCTDYY
GSSSGYFDVWGQGTTVTVSS.
S009-F3.80.22. VH3 has an amino acid sequence set
forth in SEQ ID NO: 617:
EVQLVQSGAEVKKPGATVKISCKASGYTFTDYEIHWVQQAPGKGLEWMGA
FDPEIGGSAYNQKFKDRVTITADKSTSTAYMELSSLRSEDTAVYYCTDYY
GSSSGYFDVWGTGTTVTVSS.
S009-F3.80.22. VH4 has an amino acid sequence set
forth in SEQ ID NO: 618:
EVQLVQSGAEVKKPGATVKISCKASGYTFTDYEIHWVQQAPGKGLEWMGA
FDPEIGGSAYNQKFKDRVTITADKSSSTAYMELSSLRSEDTAVYYCTDYY
GSSSGYFDVWGTGTTVTVSS.
The humanized light chain template IGKV2-40*01 has
an amino acid sequence set forth in SEQ ID NO:
619:
DIVMTQTPLSLPVTPGEPASISCRSSQSLLDSDDGNTYLDWYLQKPGQSP
QLLIYTLSYRASGVPDRFSGSGSGTDFTLKISRVEAEDVGVYYCMQRIEF
P.
The humanized light chain template IGKJ2*01 has an
amino acid sequence set forth in SEQ ID NO: 620:
FGQGTKLEIK.
The humanized heavy chain template IGHV1-69-2*01
has an amino acid sequence set forth in SEQ ID NO:
621:
EVQLVQSGAEVKKPGATVKISCKVSGYTFTDYYMHWVQQAPGKGLEWMGL
VDPEDGETIYAEKFQGRVTITADTSTDTAYMELSSLRSEDTAVYYCAT.
The humanized heavy chain template IGHJ6*01 has an
amino acid sequence set forth in SEQ ID NO: 586:
WGQGTTVTVSS.

According to the Kabat numbering scheme, the analysis results for VH and VL sequences of the humanized antibodies described above are shown in Table 69.

TABLE 69
Kabat analysis results for VH and VL sequences of S009-F3.80.22 humanized
antibodies

| Variable region No. | CDR1 | CDR2 | CDR3 |
|---|---|---|---|
| F3.80.22.VL1/2 | RSSQSLEHNNGNTYLH | KVSNRFS | SQSTHVPFT |
| | SEQ ID NO: 316 | SEQ ID NO: 317 | SEQ ID NO: 318 |
| F3.80.22. VLla | RSSQSLEHNDGNTYLH | KVSNRFS | SQSTHVPFT |
| | SEQ ID NO: 622 | SEQ ID NO: 317 | SEQ ID NO: 318 |
| F3.80.22. VL2a | RSSQSLEHNNANTYLH | KVSNRFS | SQSTHVPFT |
| | SEQ ID NO: 623 | SEQ ID NO: 317 | SEQ ID NO: 318 |
| F3.80.22. VH1/2/3/4 | DYEIH | AFDPEIGGSAYNQKFKD | YYGSSSGYFDV |
| | SEQ ID NO: 313 | SEQ ID NO: 314 | SEQ ID NO: 315 |

### 7.6. Humanization of S009-F3.38.10

For antibody S009-F3.38.10, humanized light chain templates were IGKV4-1*01/IGKV2-40*01 and IGKJ2*01, and humanized heavy chain templates were IGHV1-3*01 and IGHJ6*01. CDRs of the murine antibody S009-F3.38.10 were separately grafted into their humanized templates, so as to obtain the corresponding humanized versions. Key amino acids in FR sequences of the humanized antibodies of S009-F3.38.10 were back-mutated to amino acids corresponding to the murine antibody as needed to ensure the original affinity (there are sites in the antibody that are susceptible to chemical modification, and we performed point mutations at these sites to eliminate modification risks). Detailed back mutation design is shown in Table 70.

TABLE 70
Back mutation design for humanized antibodies of S009-F3.38.10

| VL | | VH | |
|---|---|---|---|
| S009- | Graft(IGKV2-40*01) | S009- | Graft(IGHV1-3*01) + T28I, |
| F3.38.10.VL1 | | F3.38.10.VH1 | R98N |
| S009- | Graft(IGKV4-1*01) + | S009- | Graft(IGHV1-3*01) + T28I, |
| F3.38.10.VL2 | P48S | F3.38.10.VH2 | R72V, T74K, R98N |
| | | S009- | Graft(IGHV1-3*01) + T28I, |
| | | F3.38.10.VH3 | R72V, T74K, S77N, R98N |
| | | S009- | Graft(IGHV1-3*01) + T28I, |
| | | F3.38.10.VH3a | R72V, T74K, S77N, R98N + N55Q |
| | | S009- | Graft(IGHV1-3*01) + T28I, |
| | | F3.38.10.VH4 | R44S, R72V, T74K, S77N, R98N |
| | | S009- | Graft(IGHV1-3*01) + T28I, |
| | | F3.38.10.VH5 | P41H, R72V, T74K, S77N, R98N |

Note: Graft denotes that the CDRs of the murine antibody are grafted into the human germline template FR sequences; P48S denotes that P at position 48 of Graft is mutated to S, and so on for others. The back-mutated amino acids are numbered in the natural order.

Specific sequences of the variable regions of the S009-F3.38.10 humanized antibodies are as follows:

S009-F3.38.10.VL1 has an amino acid sequence set
forth in SEQ ID NO: 624:
DIVMTQTPLSLPVTPGEPASISCRSSQSLIHSDGNTYLQWYLQKPGQSPQ
LLIYKVSNRFSGVPDRFSGSGSGTDFTLKISRVEAEDVGVYYCSQTTHVP
FTFGQGTKLEIK.
S009-F3.38.10. VL2 has an amino acid sequence set
forth in SEQ ID NO: 625:
DIVMTQSPDSLAVSLGERATINCRSSQSLIHSDGNTYLQWYQQKPGQSPK
LLIYKVSNRFSGVPDRFSGSGSGTDFTLTISSLQAEDVAVYYCSQTTHVP
FTFGQGTKLEIK.
S009-F3.38.10.VH1 has an amino acid sequence set
forth in SEQ ID NO: 626:
EVQLVQSGAEVKKPGASVKVSCKASGYIFTDYYMNWVRQAPGQRLEWMGV
INPKNGVISHNQKFKGRVTITRDTSASTAYMELSSLRSEDTAVYYCANYG
SRFYAMDYWGQGTTVTVSS.
S009-F3.38.10. VH2 has an amino acid sequence set
forth in SEQ ID NO: 627:
EVQLVQSGAEVKKPGASVKVSCKASGYIFTDYYMNWVRQAPGQRLEWMGV
INPKNGVISHNQKFKGRVTITVDKSASTAYMELSSLRSEDTAVYYCANYG
SRFYAMDYWGQGTTVTVSS.
S009-F3.38.10.VH3 has an amino acid sequence set
forth in SEQ ID NO: 628:
EVQLVQSGAEVKKPGASVKVSCKASGYIFTDYYMNWVRQAPGQRLEWMGV
INPKNGVISHNQKFKGRVTITVDKSANTAYMELSSLRSEDTAVYYCANYG
SRFYAMDYWGQGTTVTVSS.
S009-F3.38.10.VH3a has an amino acid sequence set
forth in SEQ ID NO: 629:
EVQLVQSGAEVKKPGASVKVSCKASGYIFTDYYMNWVRQAPGQRLEWMGV
INPKQGVISHNQKFKGRVTITVDKSANTAYMELSSLRSEDTAVYYCANYG
SRFYAMDYWGQGTTVTVSS.
S009-F3.38.10. VH4 has an amino acid sequence set
forth in SEQ ID NO: 630:
EVQLVQSGAEVKKPGASVKVSCKASGYIFTDYYMNWVRQAPGQSLEWMGV
INPKNGVISHNQKFKGRVTITVDKSANTAYMELSSLRSEDTAVYYCANYG
SRFYAMDYWGQGTTVTVSS.
S009-F3.38.10. VH5 has an amino acid sequence set
forth in SEQ ID NO: 631:
EVQLVQSGAEVKKPGASVKVSCKASGYIFTDYYMNWVRQAHGQRLEWMGV
INPKNGVISHNQKFKGRVTITVDKSANTAYMELSSLRSEDTAVYYCANYG
SRFYAMDYWGQGTTVTVSS.
The humanized light chain template IGKV4-1*01 has
an amino acid sequence set forth in SEQ ID NO:
572:
DIVMTQSPDSLAVSLGERATINCKSSQSVLYSSNNKNYLAWYQQKPGQPP
KLLIYWASTRESGVPDRFSGSGSGTDFTLTISSLQAEDVAVYYCQQYYST
P.
The humanized light chain template IGKV2-40*01 has
an amino acid sequence set forth in SEQ ID NO:
619:
DIVMTQTPLSLPVTPGEPASISCRSSQSLLDSDDGNTYLDWYLQKPGQSP
QLLIYTLSYRASGVPDRFSGSGSGTDFTLKISRVEAEDVGVYYCMQRIEF
P.
The humanized light chain template IGKJ2*01 has an
amino acid sequence set forth in SEQ ID NO: 620:
FGQGTKLEIK.
The humanized heavy chain template IGHV1-3*01 has
an amino acid sequence set forth in SEQ ID NO:
608:
QVQLVQSGAEVKKPGASVKVSCKASGYTFTSYAMHWVRQAPGQRLEWMGW
INAGNGNTKYSQKFQGRVTITRDTSASTAYMELSSLRSEDTAVYYCAR.
The humanized heavy chain template IGHJ6*01 has an
amino acid sequence set forth in SEQ ID NO: 586:
WGQGTTVTVSS.

According to the Kabat numbering scheme, the analysis results for VH and VL sequences of the humanized antibodies described above are shown in Table 71.

TABLE 71
Kabat analysis results for VH and VL sequences of S009-F3.38.10 humanized
antibodies

| Variable region No. | CDR1 | CDR2 | CDR3 |
|---|---|---|---|
| F3.38.10.VL1/2 | RSSQSLIHSDGNTYLQ | KVSNRFS | SQTTHVPFT |
| | SEQ ID NO: 286 | SEQ ID NO: 287 | SEQ ID NO: 288 |
| F3.38.10.VH1/2/3/4/ | DYYMN | VINPKNGVISHNQKFKG | YGSRFYAMDY |
| 5 | SEQ ID NO: 283 | SEQ ID NO: 284 | SEQ ID NO: 285 |
| F3.38.10.VH3a | DYYMN | VINPKQGVISHNQKFKG | YGSRFYAMDY |
| | SEQ ID NO: 283 | SEQ ID NO: 632 | SEQ ID NO: 285 |

### 7.7. Preparation of Control Antibodies

Both the plasmid construction and the production and purification of positive and negative control antibodies were completed by Biointron Biological Inc.

Positive controls: YP218 sequences from the patent US2015252118A1 (VH-SEQ ID NO: 13; VL-SEQ ID NO: 15) were constructed as a VH-(G4S)3-VL-his antibody and a VL-(G4S)3-VH-his antibody, designated as Tab 110 and Tab111, respectively. Negative control: The isotype negative control for the MSLN humanized antibodies was an irrelevant antibody m971 that does not bind to the MSLN protein, which has heavy and light chain variable region sequences from the patent U.S. Pat. No. 8,591,889B (VH-SEQ ID NO: 3, positions 1-124; VL-SEQ ID NO: 4, positions 1-107), was constructed as VH-(G4S)3-VL-his, and was designated as Tab084 (NC).

### 7.8. Preparation of Anti-MSLN Humanized Antibodies

Anti-MSLN humanized antibodies were expressed in the form of VH-(G4S)3-VL-his or VL-(G4S)3-VH-his. The expression and purification of the antibodies were performed by Biointron Biological Inc. The purified humanized antibodies were assayed and analyzed for protein concentration, purity, endotoxin (Lonza kit), and the like. The purity of humanized antibodies varies greatly, so antibodies with >50% purity were selected for further activity verification.

### EXAMPLE 8. IDENTIFICATION OF BINDING CAPACITY OF ANTI-MSLN HUMANIZED ANTIBODIES TO MSLN PROTEINS

According to the present invention, different light and heavy chain sequences were selected for cross combination from the above back mutation design for the light and heavy chain variable regions of the anti-MSLN humanized antibodies, respectively, and expressed in the form of VH-(G4S)3-VL-his or VL-(G4S)3-VH-his, and finally, the following anti-MSLN humanized antibodies were obtained.

TABLE 72
Variable region numbering corresponding
to anti-MSLN humanized antibodies

| Antibody | Corresponding | Antibody | Corresponding |
|---|---|---|---|
| name | variable region | name | variable region |
| F2.39.3-H1 | VH1 + VL1 | F7.44.20-H15 | VH2a + VL3 |
| F2.39.3-H2 | VH1 + VL2 | F7.44.20-H16 | VH2a + VL4 |
| F2.39.3-H5 | VH2 + VL1 | F7.33.24-H1 | VH1 + VL1 |
| F2.39.3-H6 | VH2 + VL2 | F7.33.24-H2 | VH1 + VL2 |
| F2.39.3-H8 | VH2 + VL4 | F7.33.24-H3 | VH1 + VL3 |
| F2.39.3-H9 | VH3 + VL1 | F7.33.24-H4 | VH1a + VL1 |
| F2.23.12-H1 | VH1 + VL1 | F7.33.24-H5 | VH1a + VL2 |
| F2.23.12-H4 | VH1 + VL4 | F7.33.24-H6 | VH1a + VL3 |
| F2.23.12-H5 | VH2 + VL1 | F7.33.24-H7 | VH2 + VL1 |
| F2.23.12-H8 | VH2 + VL4 | F7.33.24-H8 | VH2 + VL2 |
| F2.23.12-H13 | VH4 + VL1 | F7.33.24-H9 | VH2 + VL3 |
| F2.23.12-H16 | VH4 + VL4 | F7.33.24-H10 | VH2a + VL1 |
| F7.44.20-H1 | VH1 + VL1 | F7.33.24-H11 | VH2a + VL2 |
| F7.44.20-H2 | VH1 + VL2 | F7.33.24-H12 | VH2a + VL3 |
| F7.44.20-H3 | VH1 + VL3 | F3.80.22-H1 | VL1 + VH1 |
| F7.44.20-H4 | VH1 + VL4 | F3.80.22-H2 | VL1 + VH2 |
| F7.44.20-H5 | VH1a + VL1 | F3.80.22-H3 | VL1 + VH3 |
| F7.44.20-H6 | VH1a + VL2 | F3.80.22-H4 | VL1 + VH4 |
| F7.44.20-H7 | VH1a + VL3 | F3.80.22-H5 | VL1a + VH1 |
| F7.44.20-H8 | VH1a + VL4 | F3.80.22-H6 | VL1a + VH2 |
| F7.44.20-H9 | VH2 + VL1 | F3.80.22-H7 | VL1a + VH3 |
| F7.44.20-H10 | VH2 + VL2 | F3.80.22-H8 | VL1a + VH4 |
| F7.44.20-H11 | VH2 + VL3 | F3.80.22-H10 | VL2 + VH2 |
| F7.44.20-H12 | VH2 + VL4 | F3.80.22-H17 | VH1 + VL1a |
| F7.44.20-H13 | VH2a + VL1 | F3.38.10-L1H5 | VL1 + VH5 |
| F7.44.20-H14 | VH2a + VL2 | F3.38.10-L2H3a | VL2 + VH3a |

### 8.1. Assay on Binding of Antibodies to Human MSLN Proteins by Enzyme-Linked Immunosorbent Assay (ELISA)

To assay the binding activity of the anti-MSLN humanized antibodies to human MSLN proteins, a human MSLN-hFc protein (Acro, Cat. No. MSN-H5253) was diluted with PBS to a final concentration of 2 μg/mL and then added to a 96-well ELISA plate at 50 μL/well. The plate was sealed with a plastic film and incubated at 4° C. overnight. The next day, the plate was washed twice with PBST, and a blocking buffer [PBS+2% (w/w) BSA] was added for blocking at room temperature for 2 h. The blocking buffer was discarded, and the plate was washed twice with PBST. An antibody to be tested or a control antibody diluted in a 1:10 gradient from a starting concentration of 100 nM was added at 50 μL/well. After incubation at 37° C. for 2 h, the plate was washed 3 times with PBST. A horseradish peroxidase (HRP)-labeled Anti-his secondary antibody (purchased from Genscript, Cat. No. A00612) was added. After incubation at 37° C. for half an hour, the plate was washed 5 times with PBST. A TMB substrate was added at 50 μL/well for incubation at room temperature for 10-15 min, and then a stop solution (1.0 N HCl) was added at 50 μL/well. OD_{450 nm} values were read using an ELISA plate reader (Multimode Plate Reader, EnSight, purchased from Perkin Elmer). The experimental results are shown in A-H in FIG. 38, A-F in FIG. 39, and Tables 73-80. The negative control was an irrelevant antibody m971 that does not bind to the MSLN protein, which has heavy chain and light chain variable region sequences from the patent U.S. Pat. No. 8,591,889B, was constructed as VH-(G4S)3-VL-his, and was designated as Tab084 (NC); the positive controls were YP218 antibodies constructed as VH-(G4S)3-VL-his and VL-(G4S)3-VH-his, designated as Tab 110 and Tab 111, respectively. The data in the table are OD_{450 nm} values. The results indicate that the Anti-MSLN humanized antibodies tested had binding activity to both the human MSLN full-length protein and the human MSLN-R3 protein, except for F3.38.10, which had no binding activity to the human MSLN full-length protein.

TABLE 73
Binding reactions of F2.39.3 humanized antibodies
with human MSLN full-length protein at ELISA level

| Antibody | Antibody concentration (nM) | | | | | | |
|---|---|---|---|---|---|---|---|
| name | 100 | 10 | 1 | 0.1 | 0.01 | 0.001 | 0.0001 |
| F2.39.3-H1 | 1.68 | 1.23 | 0.55 | 0.17 | 0.07 | 0.06 | 0.06 |
| F2.39.3-H2 | 1.82 | 1.19 | 0.30 | 0.10 | 0.06 | 0.06 | 0.06 |
| F2.39.3-H5 | 1.87 | 1.21 | 0.39 | 0.11 | 0.06 | 0.06 | 0.06 |
| F2.39.3-H6 | 1.69 | 1.18 | 0.31 | 0.10 | 0.07 | 0.06 | 0.06 |
| F2.39.3-H8 | 1.95 | 1.17 | 0.31 | 0.10 | 0.06 | 0.06 | 0.05 |
| F2.39.3-H9 | 1.81 | 1.22 | 0.39 | 0.11 | 0.06 | 0.06 | 0.05 |
| Tab111 | 1.86 | 1.54 | 0.56 | 0.16 | 0.07 | 0.06 | 0.05 |
| NC | 0.05 | 0.05 | 0.05 | 0.05 | 0.05 | 0.05 | 0.05 |

TABLE 74
Binding reactions of F2.23.12 humanized antibodies
with human MSLN full-length protein at ELISA level

| Antibody | Antibody concentration (nM) | | | | | | |
|---|---|---|---|---|---|---|---|
| name | 100 | 10 | 1 | 0.1 | 0.01 | 0.001 | 0.0001 |
| F2.23.12-H1 | 1.22 | 0.82 | 0.26 | 0.09 | 0.07 | 0.06 | 0.07 |
| F2.23.12-H4 | 1.22 | 0.92 | 0.57 | 0.18 | 0.09 | 0.08 | 0.07 |
| F2.23.12-H5 | 1.37 | 0.83 | 0.22 | 0.09 | 0.07 | 0.06 | 0.06 |
| F2.23.12-H8 | 1.22 | 0.62 | 0.17 | 0.09 | 0.07 | 0.07 | 0.05 |
| F2.23.12-H13 | 1.38 | 0.73 | 0.16 | 0.08 | 0.07 | 0.07 | 0.05 |
| F2.23.12-H16 | 1.20 | 0.64 | 0.13 | 0.07 | 0.06 | 0.07 | 0.05 |
| Tab111 | 2.40 | 1.61 | 0.44 | 0.11 | 0.06 | 0.05 | 0.06 |
| NC | 0.07 | 0.07 | 0.06 | 0.07 | 0.06 | 0.06 | 0.05 |

TABLE 75
Binding reactions of F7.44.20 humanized antibodies
with human MSLN full-length protein at ELISA level

| Antibody | Antibody concentration (nM) | | | | | | |
|---|---|---|---|---|---|---|---|
| name | 100 | 10 | 1 | 0.1 | 0.01 | 0.001 | 0.0001 |
| F7.44.20-H1 | 3.04 | 2.94 | 1.46 | 0.22 | 0.06 | 0.05 | 0.05 |
| F7.44.20-H2 | 3.06 | 2.71 | 1.26 | 0.16 | 0.05 | 0.04 | 0.04 |
| F7.44.20-H3 | 2.96 | 2.74 | 1.22 | 0.15 | 0.06 | 0.04 | 0.04 |
| F7.44.20-H4 | 3.20 | 2.99 | 1.14 | 0.19 | 0.06 | 0.04 | 0.05 |
| F7.44.20-H5 | 2.94 | 3.01 | 1.30 | 0.19 | 0.05 | 0.04 | 0.04 |
| F7.44.20-H6 | 3.04 | 2.91 | 1.95 | 0.37 | 0.07 | 0.05 | 0.05 |
| F7.44.20-H7 | 3.05 | 2.99 | 1.74 | 0.30 | 0.06 | 0.04 | 0.04 |
| F7.44.20-H8 | 3.09 | 2.55 | 1.13 | 0.15 | 0.05 | 0.04 | 0.05 |
| F7.44.20-H9 | 3.25 | 2.97 | 1.30 | 0.19 | 0.06 | 0.05 | 0.05 |
| F7.44.20-H10 | 3.25 | 2.93 | 1.44 | 0.24 | 0.06 | 0.04 | 0.05 |
| F7.44.20-H11 | 3.28 | 2.91 | 1.69 | 0.27 | 0.06 | 0.05 | 0.05 |
| F7.44.20-H12 | 3.17 | 2.92 | 1.02 | 0.16 | 0.06 | 0.05 | 0.05 |
| F7.44.20-H13 | 3.12 | 2.81 | 0.98 | 0.16 | 0.06 | 0.06 | 0.06 |
| F7.44.20-H14 | 3.13 | 2.89 | 1.20 | 0.19 | 0.07 | 0.05 | 0.05 |
| F7.44.20-H15 | 3.26 | 2.78 | 1.24 | 0.19 | 0.06 | 0.05 | 0.05 |
| F7.44.20-H16 | 2.97 | 2.40 | 1.12 | 0.16 | 0.05 | 0.05 | 0.05 |
| Tab111 | 2.96 | 2.74 | 0.94 | 0.16 | 0.06 | 0.05 | 0.05 |
| Tab110 | 2.31 | 1.62 | 0.18 | 0.04 | 0.04 | 0.04 | 0.10 |
| F7.44.20 | 2.97 | 2.48 | 0.83 | 0.22 | 0.05 | 0.17 | 0.04 |
| F7.44.20-VL-VH | 2.98 | 2.14 | 1.06 | 0.11 | 0.05 | 0.20 | 0.04 |
| NC | 0.04 | 0.04 | 0.06 | 0.13 | 0.04 | 0.11 | 0.05 |

TABLE 76
Binding reactions of F7.33.24 humanized antibodies
with human MSLN full-length protein at ELISA level

| Antibody | Antibody concentration (nM) | | | | | | |
|---|---|---|---|---|---|---|---|
| name | 100 | 10 | 1 | 0.1 | 0.01 | 0.001 | 0.0001 |
| F7.33.24-H1 | 2.90 | 2.53 | 0.59 | 0.10 | 0.05 | 0.04 | 0.05 |
| F7.33.24-H2 | 2.91 | 2.07 | 0.40 | 0.08 | 0.05 | 0.05 | 0.05 |
| F7.33.24-H3 | 2.93 | 2.58 | 0.91 | 0.13 | 0.06 | 0.05 | 0.05 |
| F7.33.24-H4 | 2.95 | 2.21 | 0.45 | 0.08 | 0.07 | 0.05 | 0.05 |
| F7.33.24-H5 | 2.85 | 1.91 | 0.33 | 0.07 | 0.04 | 0.05 | 0.05 |
| F7.33.24-H6 | 2.96 | 2.36 | 0.45 | 0.08 | 0.05 | 0.05 | 0.05 |
| F7.33.24-H7 | 2.79 | 2.32 | 0.48 | 0.10 | 0.05 | 0.06 | 0.05 |
| F7.33.24-H8 | 2.70 | 2.04 | 0.48 | 0.09 | 0.05 | 0.05 | 0.05 |
| F7.33.24-H9 | 2.78 | 2.49 | 0.66 | 0.10 | 0.05 | 0.04 | 0.05 |
| F7.33.24-H10 | 3.03 | 2.28 | 0.51 | 0.09 | 0.05 | 0.04 | 0.04 |
| F7.33.24-H11 | 2.65 | 2.18 | 0.57 | 0.09 | 0.04 | 0.04 | 0.04 |
| F7.33.24-H12 | 2.65 | 2.28 | 0.58 | 0.10 | 0.04 | 0.04 | 0.05 |
| Tab111 | 2.96 | 2.74 | 0.94 | 0.16 | 0.06 | 0.05 | 0.05 |
| Tab110 | 2.31 | 1.62 | 0.18 | 0.04 | 0.04 | 0.04 | 0.10 |
| F7.33.24 | 2.58 | 1.63 | 0.27 | 0.06 | 0.21 | 0.26 | 0.04 |
| NC | 0.04 | 0.04 | 0.06 | 0.13 | 0.04 | 0.11 | 0.05 |

TABLE 77
Binding reactions of F3.38.10 humanized antibodies
with human MSLN full-length protein at ELISA level

| Antibody | Antibody concentration (nM) | | | | | | |
|---|---|---|---|---|---|---|---|
| name | 100 | 10 | 1 | 0.1 | 0.01 | 0.001 | 0.0001 |
| F3.38.10-L1H5 | 0.08 | 0.06 | 0.08 | 0.07 | 0.06 | 0.07 | 0.07 |
| F3.38.10-L2H3a | 0.07 | 0.06 | 0.06 | 0.06 | 0.06 | 0.07 | 0.07 |
| Tab111 | 1.31 | 1.05 | 0.19 | 0.08 | 0.06 | 0.06 | 0.06 |
| NC | 0.07 | 0.07 | 0.07 | 0.06 | 0.07 | 0.07 | 0.06 |

TABLE 78
Binding reactions of F7.44.20 humanized antibodies
with human MSLN-R3 protein at ELISA level

| Antibody | Antibody concentration (nM) | | | | | | |
|---|---|---|---|---|---|---|---|
| name | 100 | 10 | 1 | 0.1 | 0.01 | 0.001 | 0.0001 |
| F7.44.20-H1 | 2.30 | 2.27 | 1.16 | 0.16 | 0.06 | 0.05 | 0.05 |
| F7.44.20-H2 | 2.35 | 2.22 | 0.95 | 0.11 | 0.05 | 0.05 | 0.05 |
| F7.44.20-H3 | 2.21 | 2.12 | 0.83 | 0.10 | 0.04 | 0.04 | 0.05 |
| F7.44.20-H4 | 2.32 | 2.11 | 0.74 | 0.10 | 0.04 | 0.04 | 0.05 |
| F7.44.20-H5 | 2.27 | 2.05 | 0.80 | 0.11 | 0.04 | 0.04 | 0.05 |
| F7.44.20-H6 | 2.12 | 2.15 | 1.31 | 0.21 | 0.05 | 0.05 | 0.05 |
| F7.44.20-H7 | 2.10 | 2.23 | 1.18 | 0.16 | 0.05 | 0.04 | 0.05 |
| F7.44.20-H8 | 2.15 | 2.07 | 0.76 | 0.09 | 0.04 | 0.04 | 0.05 |
| F7.44.20-H9 | 2.52 | 2.10 | 0.72 | 0.11 | 0.04 | 0.04 | 0.04 |
| F7.44.20-H10 | 2.44 | 2.25 | 0.81 | 0.11 | 0.04 | 0.04 | 0.04 |
| F7.44.20-H11 | 2.43 | 2.41 | 1.10 | 0.16 | 0.05 | 0.04 | 0.05 |
| F7.44.20-H12 | 2.44 | 2.12 | 0.60 | 0.09 | 0.05 | 0.05 | 0.05 |
| F7.44.20-H13 | 1.94 | 1.57 | 0.45 | 0.08 | 0.05 | 0.05 | 0.05 |
| F7.44.20-H14 | 1.90 | 1.69 | 0.66 | 0.10 | 0.04 | 0.04 | 0.04 |
| F7.44.20-H15 | 1.86 | 1.85 | 0.77 | 0.11 | 0.04 | 0.04 | 0.05 |
| F7.44.20-H16 | 1.80 | 1.60 | 0.72 | 0.10 | 0.06 | 0.04 | 0.04 |
| Tab111 | 2.10 | 1.88 | 0.51 | 0.09 | 0.05 | 0.05 | 0.05 |
| Tab110 | 0.98 | 0.78 | 0.06 | 0.04 | 0.04 | 0.04 | 0.05 |
| F7.44.20 | 1.96 | 1.68 | 0.39 | 0.07 | 0.04 | 0.04 | 0.04 |
| F7.44.20-VL-VH | 1.80 | 1.68 | 0.55 | 0.08 | 0.05 | 0.04 | 0.05 |
| NC | 0.05 | 0.04 | 0.04 | 0.04 | 0.04 | 0.04 | 0.05 |

TABLE 79
Binding reactions of F7.33.24 humanized antibodies
with human MSLN-R3 protein at ELISA level

| Antibody | Antibody concentration (nM) | | | | | | |
|---|---|---|---|---|---|---|---|
| name | 100 | 10 | 1 | 0.1 | 0.01 | 0.001 | 0.0001 |
| F7.33.24-H1 | 1.93 | 1.61 | 0.52 | 0.08 | 0.04 | 0.04 | 0.04 |
| F7.33.24-H2 | 1.90 | 1.62 | 0.42 | 0.06 | 0.04 | 0.04 | 0.04 |
| F7.33.24-H3 | 1.91 | 1.86 | 0.89 | 0.13 | 0.04 | 0.04 | 0.04 |
| F7.33.24-H4 | 1.95 | 1.64 | 0.45 | 0.07 | 0.04 | 0.04 | 0.04 |
| F7.33.24-H5 | 1.92 | 1.53 | 0.39 | 0.06 | 0.04 | 0.04 | 0.04 |
| F7.33.24-H6 | 1.98 | 1.74 | 0.53 | 0.07 | 0.04 | 0.04 | 0.04 |
| F7.33.24-H7 | 1.95 | 1.52 | 0.33 | 0.06 | 0.04 | 0.04 | 0.04 |
| F7.33.24-H8 | 2.16 | 1.92 | 0.63 | 0.09 | 0.05 | 0.04 | 0.05 |
| F7.33.24-H9 | 2.25 | 2.08 | 0.82 | 0.11 | 0.05 | 0.04 | 0.04 |
| F7.33.24-H10 | 2.26 | 1.85 | 0.55 | 0.08 | 0.04 | 0.05 | 0.05 |
| F7.33.24-H11 | 2.10 | 1.94 | 0.68 | 0.09 | 0.05 | 0.04 | 0.05 |
| F7.33.24-H12 | 2.04 | 1.95 | 0.65 | 0.09 | 0.04 | 0.04 | 0.04 |
| Tab111 | 2.10 | 1.88 | 0.51 | 0.09 | 0.05 | 0.05 | 0.05 |
| Tab110 | 0.98 | 0.78 | 0.06 | 0.04 | 0.04 | 0.04 | 0.05 |
| F7.33.24 | 1.92 | 1.78 | 0.71 | 0.09 | 0.04 | 0.04 | 0.04 |
| NC | 0.05 | 0.04 | 0.04 | 0.04 | 0.04 | 0.04 | 0.05 |

TABLE 80
Binding reactions of F3.38.10 humanized antibodies
with human MSLN-R3 protein at ELISA level

| Antibody | Antibody concentration (nM) | | | | | | |
|---|---|---|---|---|---|---|---|
| name | 100 | 10 | 1 | 0.1 | 0.01 | 0.001 | 0.0001 |
| F3.38.10-L1H5 | 2.36 | 1.19 | 0.27 | 0.08 | 0.07 | 0.07 | 0.07 |
| F3.38.10-L2H3a | 1.88 | 0.72 | 0.12 | 0.06 | 0.05 | 0.05 | 0.05 |
| Tab111 | 1.89 | 1.05 | 0.25 | 0.07 | 0.05 | 0.05 | 0.05 |
| NC | 0.06 | 0.05 | 0.04 | 0.07 | 0.05 | 0.05 | 0.05 |

### 8.2. Assay on Binding of Antibodies to HEK293T Recombinant Cells Expressing Human MSLN-R3 by Flow Cytometry Assay (FACS)

The desired cells were expanded to the logarithmic growth phase in a T-175 cell culture flask, the medium was removed by pipetting, and the cells were washed twice with a PBS buffer and digested with trypsin. Then a complete medium was added to stop the digestion, and the cells were blown into a single cell suspension. After counting, the cells were centrifuged, and the cell pellet was washed twice with PBS, resuspended to 2×10⁶ cells/mL in an FACS buffer (PBS+2% fetal bovine serum), and added to a 96-well FACS reaction plate at 50 μL/well. An antibody to be tested or a control antibody (diluted in a 5-fold gradient from a starting concentration of 200 nM) was added at 50 μL/well and uniformly mixed with the cell suspension, and the resulting mixture was incubated at 4° C. for 1 h. The plate was centrifuged and washed 3 times with a PBS buffer, and an iFluor 647-labeled Anti-His secondary antibody (purchased from Genescript, Cat. No. A01802-100) was added at 50 μL/well for incubation at 4° C. for 1 h. After the plate was centrifuged and washed 3 times again with a PBS buffer and resuspended in 100 μL of PBS, assay and analysis were performed by FACS (FACS Canto™, purchased from BD). Data analysis was performed by software (CellQuest) to obtain the mean fluorescence intensity (MFI) of the cells. Then, analysis was performed by software (GraphPad Prism8), data were fitted, and EC₅₀ values were calculated. The analysis results are shown in A-J in FIG. 40A and FIG. 40B, A-J in FIG. 41A and FIG. 41B, and Tables 81-85, with Tab084 used as a negative control (NC), and Tab110 and Tab111 used as positive controls. As can be seen from the results, all of the anti-MSLN humanized antibodies had binding activity to the HEK293T recombinant cells expressing human MSLN-R3 protein and did not bind to the HEK293T null cells, indicating that the anti-MSLN humanized antibodies specifically bound to the human MSLN-R3 membrane protein.

TABLE 81
Binding reactions of F2.39.3 and F2.23.12
humanized antibodies with HEK293T-hMSLN-R3

| | HEK293T-hMSLN-R3 | | HEK293T | |
|---|---|---|---|---|
| Antibody | Maxi- | EC50 | Maxi- | EC50 |
| name | mum_MFI | (nM) | mum_MFI | (nM) |
| F2.39.3-H1 | 5817 | 4.91 | 100 | No binding |
| F2.39.3-H2 | 5969 | 6.33 | 260 | No binding |
| F2.39.3-H5 | 5650 | 4.65 | 338 | No binding |
| F2.39.3-H6 | 5782 | 6.26 | 224 | No binding |
| F2.39.3-H8 | 5601 | 6.91 | 230 | No binding |
| F2.39.3-H9 | 5565 | 5.66 | 211 | No binding |
| F2.23.12-H1 | 4715 | 4.70 | 203 | No binding |
| F2.23.12-H4 | 4762 | 2.67 | 227 | No binding |
| F2.23.12-H5 | 4734 | 4.16 | 215 | No binding |
| F2.23.12-H8 | 4594 | 5.57 | 254 | No binding |
| F2.23.12-H13 | 4709 | 8.30 | 302 | No binding |
| F2.23.12-H16 | 4007 | 9.39 | 268 | No binding |
| Tab111 | 4966 | 1.22 | 277 | No binding |
| NC | 439 | NB | 436 | No binding |

TABLE 82
Binding reactions of F7.44.20 humanized antibodies with HEK293T-hMSLN-R3

| | HEK293T-hMSLN-R3 | | HEK293T | |
|---|---|---|---|---|
| Antibody name | Maximum_MFI | EC50 (nM) | Maximum_MFI | EC50 (nM) |
| F7.44.20-H1 | 1753 | 1.72 | 185 | No binding |
| F7.44.20-H2 | 1689 | 1.71 | 209 | No binding |
| F7.44.20-H3 | 1486 | 1.57 | 194 | No binding |
| F7.44.20-H4 | 1468 | 2.64 | 235 | No binding |
| F7.44.20-H5 | 1700 | 0.94 | 173 | No binding |
| F7.44.20-H6 | 1535 | 3.44 | 169 | No binding |
| F7.44.20-H7 | 1506 | 2.12 | 172 | No binding |
| F7.44.20-H8 | 1654 | 1.95 | 159 | No binding |
| F7.44.20-H9 | 1559 | 1.67 | 206 | No binding |
| F7.44.20-H10 | 1473 | 0.88 | 213 | No binding |
| F7.44.20-H11 | 1681 | 1.14 | 180 | No binding |
| F7.44.20-H12 | 1408 | 2.88 | 148 | No binding |
| F7.44.20-H13 | 1598 | 0.63 | 159 | No binding |
| F7.44.20-H14 | 1437 | 1.52 | 240 | No binding |
| F7.44.20-H15 | 1500 | 0.91 | 327 | No binding |
| F7.44.20-H16 | 1436 | 2.23 | 192 | No binding |
| Tab111 | 2116 | 0.76 | 230 | No binding |
| Tab110 | 461 | Weak binding | 171 | No binding |
| F7.44.20 | 1450 | 1.27 | 224 | No binding |
| F7.44.20-VL-VH | 1853 | 0.48 | 183 | No binding |
| NC | 92 | No binding | 144 | No binding |

TABLE 83
Binding reactions of F7.33.24 humanized antibodies with HEK293T-hMSLN-R3

| | HEK293T-hMSLN-R3 | | HEK293T | |
|---|---|---|---|---|
| Antibody name | Maximum_MFI | EC50 (nM) | Maximum_MFI | EC50 (nM) |
| F7.33.24-H1 | 2190 | 2.55 | 1179 | No binding |
| F7.33.24-H2 | 1518 | 1.21 | 499 | No binding |
| F7.33.24-H3 | 1972 | 2.89 | 1086 | No binding |
| F7.33.24-H4 | 1962 | 2.15 | 405 | No binding |
| F7.33.24-H5 | 1468 | 1.35 | 577 | No binding |
| F7.33.24-H6 | 1537 | 1.01 | 194 | No binding |
| F7.33.24-H7 | 1650 | 1.61 | 335 | No binding |
| F7.33.24-H8 | 1405 | 1.52 | 216 | No binding |
| F7.33.24-H9 | 1471 | 1.29 | 417 | No binding |
| F7.33.24-H10 | 1563 | 1.10 | 683 | No binding |
| F7.33.24-H11 | 1589 | 0.42 | 541 | No binding |
| F7.33.24-H12 | 1330 | 1.27 | 298 | No binding |
| Tab111 | 2116 | 0.76 | 230 | No binding |
| Tab110 | 461 | Weak binding | 171 | No binding |
| F7.33.24 | 1494 | 1.63 | 167 | No binding |
| NC | 92 | No binding | 144 | No binding |

TABLE 84
Binding reactions of F3.80.22 humanized antibodies with HEK293T-hMSLN-R3

| | HEK293T-hMSLN-R3 | | HEK293T | |
|---|---|---|---|---|
| Antibody name | Maximum_MFI | EC50 (nM) | Maximum_MFI | EC50 (nM) |
| F3.80.22-H1 | 1699 | 2.14 | 360 | No binding |
| F3.80.22-H2 | 1791 | 0.67 | 261 | No binding |
| F3.80.22-H3 | 1747 | 4.41 | 283 | No binding |
| F3.80.22-H4 | 1409 | WB | 300 | No binding |
| F3.80.22-H5 | 1371 | 5.29 | 339 | No binding |
| F3.80.22-H6 | 1443 | 8.39 | 245 | No binding |
| F3.80.22-H7 | 1527 | 3.91 | 330 | No binding |
| F3.80.22-H8 | 1400 | 10.19 | 260 | No binding |
| F3.80.22-H10 | 1805 | 1.99 | 274 | No binding |
| F3.80.22-H17 | 1791 | 4.41 | 323 | No binding |
| Tab111 | 3068 | 1.60 | 467 | No binding |
| NC | 304 | No binding | 295 | No binding |

TABLE 85
Binding reactions of F3.38.10 humanized antibodies with HEK293T-hMSLN-R3

| | HEK293T-hMSLN-R3 | | HEK293T | |
|---|---|---|---|---|
| Antibody name | Maximum_MFI | EC50 (nM) | Maximum_MFI | EC50 (nM) |
| F3.38.10-L1H5 | 1283 | 11.99 | 127 | No binding |
| F3.38.10-L2H3a | 795 | 166.00 | 134 | No binding |
| Tab111 | 777 | 30.21 | 133 | No binding |
| NC | 156 | No binding | 128 | No binding |

### 8.3. Assay on Binding of Antibodies to OVCAR3 Tumor Cells Expressing Human MSLN by Flow Cytometry Assay (FACS)

The preparation of the assay cells and the antibodies to be tested and the assay were performed with reference to Example 2.2. The analysis results are shown in A-J in FIG. 42A and FIG. 42B, A-J in FIG. 43A and FIG. 43B, and Tables 86-90, with Tab084 used as a negative control (NC), and Tab 110 and Tab111 used as positive controls. As can be seen from the results, the F3.80.22 humanized antibodies had no binding activity, F3.38.10 humanized antibodies had weak (no) binding activity, and the remaining Anti-MSLN humanized antibodies tested had binding activity to the OVCAR3 tumor cells expressing human MSLN protein and did not bind to the A431 cells not expressing human MSLN protein, indicating that the anti-MSLN humanized antibodies can better specifically bind to the OVCAR3 cells.

TABLE 86
Binding reactions of F2.39.3 and F2.23.12 humanized antibodies with OVCAR3

| | OVCAR3 | | A431 | |
|---|---|---|---|---|
| Antibody name | Maximum_MFI | EC50 (nM) | Maximum_MFI | EC50 (nM) |
| F2.39.3-H1 | 4004 | 10.99 | 269 | No binding |
| F2.39.3-H2 | 4215 | 54.05 | 216 | No binding |
| F2.39.3-H5 | 3135 | 6.37 | 219 | No binding |
| F2.39.3-H6 | 4158 | 40.26 | 183 | No binding |
| F2.39.3-H8 | 4121 | 18.64 | 241 | No binding |
| F2.39.3-H9 | 4304 | 17.23 | 223 | No binding |
| F2.23.12-H1 | 3248 | 9.56 | 151 | No binding |
| F2.23.12-H4 | 3626 | 9.90 | 157 | No binding |
| F2.23.12-H5 | 3335 | 11.26 | 192 | No binding |
| F2.23.12-H8 | 3039 | 14.48 | 172 | No binding |
| F2.23.12-H13 | 3108 | 38.64 | 134 | No binding |
| F2.23.12-H16 | 2923 | 80.42 | 163 | No binding |
| Tab111 | 4303 | 0.64 | 214 | No binding |
| NC | 336 | No binding | 334 | No binding |

TABLE 87
Binding reactions of F7.44.20 humanized antibodies with OVCAR3

| | OVCAR3 | | A431 | |
|---|---|---|---|---|
| Antibody name | Maximum_MFI | EC50 (nM) | Maximum_MFI | EC50 (nM) |
| F7.44.20-H1 | 1819 | 0.40 | 261 | No binding |
| F7.44.20-H2 | 1819 | 0.99 | 189 | No binding |
| F7.44.20-H3 | 1809 | 0.69 | 217 | No binding |
| F7.44.20-H4 | 1675 | 1.26 | 218 | No binding |
| F7.44.20-H5 | 1963 | 1.00 | 259 | No binding |
| F7.44.20-H6 | 1828 | 1.60 | 249 | No binding |
| F7.44.20-H7 | 1838 | 1.11 | 312 | No binding |
| F7.44.20-H8 | 1880 | 1.04 | 280 | No binding |
| F7.44.20-H9 | 2215 | 1.61 | 283 | No binding |
| F7.44.20-H10 | 2321 | 1.21 | 406 | No binding |
| F7.44.20-H11 | 1952 | 0.61 | 312 | No binding |
| F7.44.20-H12 | 2095 | 1.50 | 332 | No binding |
| F7.44.20-H13 | 2154 | 1.33 | 258 | No binding |
| F7.44.20-H14 | 2141 | 1.83 | 308 | No binding |
| F7.44.20-H15 | 1930 | 1.77 | 333 | No binding |
| F7.44.20-H16 | 1646 | 1.66 | 224 | No binding |
| Tab111 | 2709 | 0.97 | 343 | No binding |
| Tab110 | 999 | 1.70 | 231 | No binding |
| F7.44.20 | 1597 | 2.42 | 201 | No binding |
| F7.44.20-VL-VH | 2917 | 1.09 | 290 | No binding |
| NC | 209 | No binding | 183 | No binding |

TABLE 88
Binding reactions of F7.33.24 humanized antibodies with OVCAR3

| | OVCAR3 | | A431 | |
|---|---|---|---|---|
| Antibody name | Maximum_MFI | EC50 (nM) | Maximum_MFI | EC50 (nM) |
| F7.33.24-H1 | 2183 | 1.05 | 791 | No binding |
| F7.33.24-H2 | 2189 | 2.07 | 299 | No binding |
| F7.33.24-H3 | 2371 | 2.06 | 354 | No binding |
| F7.33.24-H4 | 2228 | 3.04 | 325 | No binding |
| F7.33.24-H5 | 2424 | 2.78 | 295 | No binding |
| F7.33.24-H6 | 2298 | 1.08 | 300 | No binding |
| F7.33.24-H7 | 2186 | 2.00 | 329 | No binding |
| F7.33.24-H8 | 2248 | 1.80 | 291 | No binding |
| F7.33.24-H9 | 2194 | 2.60 | 362 | No binding |
| F7.33.24-H10 | 2087 | 3.04 | 526 | No binding |
| F7.33.24-H11 | 2130 | 2.25 | 273 | No binding |
| F7.33.24-H12 | 2202 | 0.92 | 302 | No binding |
| Tab111 | 2709 | 0.97 | 343 | No binding |
| Tab110 | 999 | 1.70 | 231 | No binding |
| F7.33.24 | 2263 | 4.75 | 285 | No binding |
| NC | 209 | No binding | 183 | No binding |

TABLE 89
Binding reactions of F3.80.22 humanized antibodies with OVCAR3

| | OVCAR3 | | A431 | |
|---|---|---|---|---|
| Antibody name | Maximum_MFI | EC50 (nM) | Maximum_MFI | EC50 (nM) |
| F3.80.22-H1 | 471 | No binding | 273 | No binding |
| F3.80.22-H2 | 512 | No binding | 363 | No binding |
| F3.80.22-H3 | 502 | No binding | 319 | No binding |
| F3.80.22-H4 | 505 | No binding | 320 | No binding |
| F3.80.22-H5 | 508 | No binding | 309 | No binding |
| F3.80.22-H6 | 473 | No binding | 319 | No binding |
| F3.80.22-H7 | 479 | No binding | 294 | No binding |
| F3.80.22-H8 | 502 | No binding | 294 | No binding |
| F3.80.22-H10 | 462 | No binding | 287 | No binding |
| F3.80.22-H17 | 456 | No binding | 283 | No binding |
| Tab111 | 11203 | 2.33 | 704 | No binding |
| NC | 469 | No binding | 255 | No binding |

TABLE 90
Binding reactions of F3.38.10 humanized antibodies with OVCAR3

| | OVCAR3 | | A431 | |
|---|---|---|---|---|
| Antibody name | Maximum_MFI | EC50 (nM) | Maximum_MFI | EC50 (nM) |
| F3.38.10-L1H5 | 795 | 99.75 | 435 | No binding |
| F3.38.10-L2H3a | 499 | No binding | 339 | No binding |
| Tab111 | 9077 | 2.52 | 556 | No binding |
| NC | 454 | No binding | 382 | No binding |

### EXAMPLE 9. IDENTIFICATION OF CROSS-BINDING ACTIVITY OF ANTI-MSLN HUMANIZED ANTIBODIES TO HEK293T-MONKEY MSLN CELLS

HEK293T-monkey MSLN cells were collected and subjected to FACS assay and data analysis according to the methods described in Example 2.2. The analysis results are shown in A-J in FIG. 44A and FIG. 44B and Tables 91-95, with Tab084 used as a negative control (NC), and Tab 110 and Tab111 used as positive controls. As can be seen from the results, the F3.80.22 humanized antibodies had no binding activity, the F3.38.10 humanized antibodies had weak binding activity, and the remaining anti-MSLN humanized antibodies tested had better binding activity to HEK293T-monkey MSLN cells.

TABLE 91
Binding reactions of F2.39.3 and F2.23.12 humanized
antibodies with HEK293-monkey MSLN

HEK293T-monkey MSLN

| Antibody name | Maximum_MFI | EC50 (nM) |
|---|---|---|
| F2.39.3-H1 | 24444 | 5.31 |
| F2.39.3-H2 | 24909 | 8.74 |
| F2.39.3-H5 | 22458 | 5.64 |
| F2.39.3-H6 | 24080 | 8.90 |
| F2.39.3-H8 | 25965 | 12.82 |
| F2.39.3-H9 | 23346 | 7.73 |
| F2.23.12-H1 | 16032 | 6.72 |
| F2.23.12-H4 | 14476 | 2.50 |
| F2.23.12-H5 | 17676 | 5.86 |
| F2.23.12-H8 | 14724 | 5.22 |
| F2.23.12-H13 | 17566 | 10.95 |
| F2.23.12-H16 | 16124 | 12.27 |
| Tab111 | 21262 | 4.40 |
| NC | 279 | No binding |

TABLE 92
Binding reactions of F7.44.20 humanized
antibodies with HEK293-monkey MSLN

HEK293-monkey MSLN

| Antibody name | Maximum_MFI | EC50 (nM) |
|---|---|---|
| F7.44.20-H1 | 6547 | 6.05 |
| F7.44.20-H2 | 6952 | 5.50 |
| F7.44.20-H3 | 7002 | 7.34 |
| F7.44.20-H4 | 6451 | 10.37 |
| F7.44.20-H5 | 7779 | 4.55 |
| F7.44.20-H6 | 6021 | 14.97 |
| F7.44.20-H7 | 6133 | 9.24 |
| F7.44.20-H8 | 6325 | 7.09 |
| F7.44.20-H9 | 7460 | 8.05 |
| F7.44.20-H10 | 9021 | 4.25 |
| F7.44.20-H11 | 7213 | 3.74 |
| F7.44.20-H12 | 6669 | 10.26 |
| F7.44.20-H13 | 9316 | 2.35 |
| F7.44.20-H14 | 8133 | 7.37 |
| F7.44.20-H15 | 8087 | 4.47 |
| F7.44.20-H16 | 5978 | 12.23 |
| Tab111 | 10748 | 2.11 |
| Tab110 | 1334 | Weak binding |
| F7.44.20 | 7038 | 8.38 |
| F7.44.20-VL-VH | 11080 | 2.97 |
| NC | 104 | No binding |

TABLE 93
Binding reactions of F7.33.24 humanized
antibodies with HEK293-monkey MSLN

HEK293-monkey MSLN

| Antibody name | Maximum_MFI | EC50 (nM) |
|---|---|---|
| F7.33.24-H1 | 9054 | 4.16 |
| F7.33.24-H2 | 8173 | 5.11 |
| F7.33.24-H3 | 8307 | 5.33 |
| F7.33.24-H4 | 9385 | 5.23 |
| F7.33.24-H5 | 8429 | 4.98 |
| F7.33.24-H6 | 8444 | 3.54 |
| F7.33.24-H7 | 8817 | 5.00 |
| F7.33.24-H8 | 8001 | 4.60 |
| F7.33.24-H9 | 8077 | 4.08 |
| F7.33.24-H10 | 8623 | 3.94 |
| F7.33.24-H11 | 8583 | 1.46 |
| F7.33.24-H12 | 7938 | 5.07 |
| Tab111 | 10748 | 2.11 |
| Tab110 | 1334 | Weak binding |
| F7.33.24 | 8913 | 7.97 |
| NC | 104 | No binding |

TABLE 94
Binding reactions of F3.80.22 humanized
antibodies with HEK293-monkey MSLN

HEK293-monkey MSLN

| Antibody name | Maximum_MFI | EC50 (nM) |
|---|---|---|
| F3.80.22-H1 | 278 | No binding |
| F3.80.22-H2 | 303 | No binding |
| F3.80.22-H3 | 330 | No binding |
| F3.80.22-H4 | 357 | No binding |
| F3.80.22-H5 | 302 | No binding |
| F3.80.22-H6 | 338 | No binding |
| F3.80.22-H7 | 329 | No binding |
| F3.80.22-H8 | 317 | No binding |
| F3.80.22-H10 | 381 | No binding |
| F3.80.22-H17 | 356 | No binding |
| Tab111 | 24260 | 5.79 |
| NC | 231 | No binding |

TABLE 95
Binding reactions of F3.38.10 humanized
antibodies with HEK293-monkey MSLN

HEK293-monkey MSLN

| Antibody name | Maximum_MFI | EC50 (nM) |
|---|---|---|
| F3.38.10-L1H5 | 1019 | 90.08 |
| F3.38.10-L2H3a | 362 | Weak binding |
| Tab111 | 5147 | 3.44 |
| NC | 172 | No binding |

### EXAMPLE 10. ASSAY ON AFFINITY OF ANTI-MSLN HUMANIZED ANTIBODIES

The strength of antibody-antigen binding was assayed with a BIAcore 8K instrument using an anti-human antibody capture method. First, an anti-Human IgG antibody was immobilized onto a CM5 chip (Cytiva; 29-1496-03) using an amino coupling method according to the instruction of the Human Antibody Capture Kit (Cytiva; 29-2346-00); NHS and EDC were mixed with HBS-EP+pH 7.4 (10 mM HEPES, 150 mM NaCl, 3 mM EDTA, 0.05% surfactant P20) (Cytiva; BR-1006-69) as a mobile phase to activate the chip for about 600 s; the anti-Human IgG antibody was diluted to 15 μg/mL with 10 mM sodium acetate (pH 5.0) and injected for 420 s, and finally, the remaining activated sites were blocked with ethanolamine. Then, the affinity of the antibody for the antigen was assayed using a multi-cycle kinetic method; in each cycle, firstly, an MSLN-hFc or CD3e-hFc recombinant protein was captured using an anti-human antibody, and then a single concentration of the antibody to be tested was injected; the association and dissociation processes of the antibody with the antigen protein were recorded, and finally, the chip was regenerated using 3 M MgCl₂, wherein the mobile phase was HBS-EP+pH 7.4, the flow rate was 30 μL/min, the regeneration time was 30 s, and the assay temperature was 25° C. Finally, according to a 1:1 binding model, the data were analyzed, and the antibody-antigen binding kinetic parameters, including the association rate constant ka, the dissociation rate constant kd, the equilibrium dissociation constant KD, and the maximum binding signal Rmax, were fitted.

The results showed that the binding signal of F3.38.10-L1H5 to the human MSLN protein was not detected, and the affinity of the remaining anti-MSLN humanized molecules for the human MSLN protein was not less than 3.11E-8.

TABLE 96
Assay results for affinity of Anti-MSLN humanized
antibodies for human MSLN protein by SPR (biacore)

| Antibody name | ka (1/Ms) | kd (1/s) | KD (M) |
|---|---|---|---|
| F2.39.3-H2 | 3.44E+04 | 3.39E−05 | 9.85E−10 |
| F2.39.3-H9 | 4.35E+04 | 3.19E−05 | 7.34E−10 |
| F2.23.12-H5 | 6.12E+04 | 4.73E−04 | 7.74E−09 |
| F2.23.12-H13 | 1.55E+04 | 4.82E−04 | 3.11E−08 |
| F7.44.20-H5 | 4.42E+05 | 3.13E−04 | 7.09E−10 |
| F7.44.20-H9 | 4.49E+05 | 2.08E−04 | 4.64E−10 |
| F7.44.20-H16 | 3.67E+05 | 6.79E−04 | 1.85E−09 |
| F7.33.24-H2 | 2.54E+05 | 4.66E−04 | 1.83E−09 |
| F7.33.24-H5 | 4.09E+05 | 4.35E−04 | 1.06E−09 |
| F7.33.24-H8 | 4.02E+05 | 5.29E−04 | 1.32E−09 |
| F3.80.22-H5 | 3.56E+03 | 5.65E−04 | 1.59E−07 |
| F3.80.22-H10 | 8.47E+03 | 1.77E−04 | 2.09E−08 |

| | |
|---|---|
| F3.38.10-L1H5 | No binding |

### REFERENCE TO A “SEQUENCE LISTING,” A TABLE, OR A COMPUTER PROGRAM LISTING APPENDIX SUBMITTED AS AN ASCII TEXT FILE

The material in the ASCII text file, named “L-TN-OF220356SPUS-SR0421-sequence listing-US,” created Nov. 16, 2023, file size of 335,872 bytes, is hereby incorporated by reference.

1-27. (canceled)

28. An antibody or an antigen-binding fragment specifically binding to MSLN, wherein the antibody or the antigen-binding fragment comprises:
(a) HCDR1 with an amino acid sequence set forth in SEQ ID NO: 415, HCDR2 with an amino acid sequence set forth in SEQ ID NO: 416 or 599, and HCDR3 with an amino acid sequence set forth in SEQ ID NO: 417; and
(b) amino acid sequence set forth in SEQ ID NO: 418 for LCDR1, amino acid sequence set forth in SEQ ID NO: 419 for LCDR2, and amino acid sequence set forth in SEQ ID NO: 420 for LCDR3,
wherein the HCDR1-3 and the LCDR1-3 are determined according to the Kabat numbering scheme.

29. The antibody or the antigen-binding fragment according to claim 28, wherein the antibody or the antigen-binding fragment comprises:
(1) a VH set forth in any one of SEQ ID NOs: 594 or 104; and
(2) a VL set forth in any one of SEQ ID NOs: 589 or 105.

30. The antibody or the antigen-binding fragment according to claim 28, wherein:
the antibody or the antigen-binding fragment comprises or does not comprise an antibody heavy chain constant region and/or light chain constant region;
optionally, the antibody heavy chain constant region may be selected from human, or mouse;
optionally, the antibody heavy chain constant region may be selected from IgG, IgM, IgA, IgE, or IgD, and the IgG may be selected from IgG1, IgG2, IgG3, or IgG4;
optionally, the heavy chain constant region may be selected from an Fc region, a CH3 region, a heavy chain constant region without a CH1 fragment, or an intact heavy chain constant region;
preferably, the heavy chain constant region has an amino acid sequence set forth in SEQ ID NO: 158; and/or
preferably, the light chain constant region has an amino acid sequence set forth in SEQ ID NO: 159.

31. The antibody or the antigen-binding fragment according to claim 28, wherein the antibody or the antigen-binding fragment specifically binds to a human MSLN protein; and/or the antibody or the antigen-binding fragment binds to human MSLN with a dissociation constant (KD) of not greater than 8.00E-7 M.

32. The antibody or the antigen-binding fragment according to claim 28, wherein the antibody or the antigen-binding fragment is:
(1) a chimeric antibody or a fragment thereof;
(2) a humanized antibody or a fragment thereof; or
(3) a fully human antibody or a fragment thereof.

33. The antibody or the antigen-binding fragment according to claim 28, wherein the antibody or the antigen-binding fragment is further conjugated to a therapeutic agent or a tracer.

34. The antibody or the antigen-binding fragment according to claim 33, wherein the therapeutic agent is selected from a drug, a toxin, a radioisotope, a chemotherapeutic agent, or an immunomodulator, and the tracer is selected from a radiocontrast medium, a paramagnetic ion, a metal, a fluorescent label, a chemiluminescent label, an ultrasound contrast agent, or a photosensitizer.

35. A multispecific molecule, wherein the multispecific molecule comprises the antibody or the antigen-binding fragment according to claim 28; preferably, the multispecific molecule further comprises another antibody or another antigen-binding fragment specifically binding to an antigen other than MSLN or binding to an epitope of MSLN different from that of the antibody or the antigen-binding fragment.

36. The multispecific molecule according to claim 35, wherein the antigen other than MSLN is an antigen on the surface of a T cell, a B cell, a natural killer cell, a dendritic cell, a macrophage, a monocyte, or a neutrophil; and the antigen other than MSLN is selected from: CD3, CD3γ, CD3δ, CD3ε, CD3ζ, CD16, CD16A, CD32B, PD-1, PD-2, PD-L1, VEGF, NKG2D, CD19, CD20, CD40, CD47, 4-1BB, CD137, EGFR, EGFRvIII, TNF-alpha, CD33, HER2, HER3, HAS, CD5, CD27, EphA2, EpCAM, MUC1, MUC16, CEA, Claudin18.2, folate receptor, Claudin6, WT1, NY-ESO-1, MAGE3, ASGPR1, or CDH16.

37. The multispecific molecule according to claim 35, wherein the multispecific molecule is a tandem scFv, a bifunctional antibody (Db), a single chain bifunctional antibody (scDb), a dual affinity retargeting (DART) antibody, an F(ab′)₂, a dual variable domain (DVD) antibody, a knobs-into-holes (KiH) antibody, a dock-and-lock (DNL) antibody, a chemically cross-linked antibody, a hetero-poly-antibody, or a hetero-conjugate antibody.

38. A chimeric antigen receptor (CAR), wherein the chimeric antigen receptor at least comprises an extracellular antigen-binding domain, a transmembrane domain, and an intracellular signaling domain; and the extracellular antigen-binding domain comprises the antibody or the antigen-binding fragment according to claim 28.

39. An immune effector cell, wherein the immune effector cell expresses the chimeric antigen receptor, wherein the chimeric antigen receptor comprises the antibody or the antigen-binding fragment according to claim 28; preferably, the immune effector cell is selected from a T cell, a natural killer (NK) cell, a natural killer T (NKT) cell, a double negative T (DNT) cell, a monocyte, a macrophage, a dendritic cell, or a mast cell; the T cell is preferably selected from a cytotoxic T cell, a regulatory T cell, or a helper T cell; and/or preferably, the immune effector cell is an auto-immune effector cell or an allogeneic immune effector cell.

40. An isolated nucleic acid fragment, wherein the nucleic acid fragment encodes the antibody or the antigen-binding fragment according to claim 28.

41. A pharmaceutical composition, wherein the pharmaceutical composition comprises the antibody or the antigen-binding fragment according to claim 28, optionally, the pharmaceutical composition further comprises a pharmaceutically acceptable carrier, diluent or adjuvant; and/or optionally, the pharmaceutical composition further comprises an additional antineoplastic agent.

42. A method for preventing and/or treating a tumor, comprising: administering to a patient in need thereof an effective amount of the antibody or the antigen-binding fragment according to claim 28, wherein the tumor is selected from mesothelioma, lung cancer, breast cancer, esophageal cancer, pancreatic cancer, ovarian cancer, or pleural cancer, more preferably epithelioid malignant pleural mesothelioma or lung adenocarcinoma.

43. A method for detecting MSLN expression, comprising: contacting a sample to be tested with the antibody or the antigen-binding fragment according to claim 28 in a condition allowing formation of a complex by the antibody or the antigen-binding fragment and MSLN.


SEQUENCE LISTING (632 sequences)

SEQ ID NO: 1 - protein, 122 aa, Artificial Sequence
  Synthetic sequence
QEQLEESGGDLVQPEGSLTLTCKASGLDFSSSYWICWVRQAPGKGLEWIGCRHTFTANTWSASWVNGRFTISRSTSLGTVDLKMTSLTAADTATYFCARDESNNDGWDFKLWGPGTLVTVSS

SEQ ID NO: 2 - protein, 109 aa, Artificial Sequence
  Synthetic sequence
AYDMTQTPASVSAAVGGTVTIKCQASQSISNYLAWYQQKPGQPPKLLIYQASTLAPGVSSRFKGSGSGTEFTLTISGVECADAATYYCQQGYTSSNVENVFGGGTGVVV

SEQ ID NO: 3 - protein, 123 aa, Artificial Sequence
  Synthetic sequence
QQQLEESGGGLVKPEGSLTLTCKASGFDLGFYFYACWVRQAPGKGLEWIACIYTAGSGSTYYASWAKGRFTISKASSTTVTLQMTSLAAADTATYFCARSTANTRSTYYLNLWGPGTLVTVSS

SEQ ID NO: 4 - protein, 119 aa, Artificial Sequence
  Synthetic sequence
QEQLVESGGGLVQPGASLTLTCTASGIDFSRYYMCWVRQAPGKGLEGIACIYIGGSGSTYYASWAKGRFTISKASSTTVTLQMTSLTAADTATYFCARGTNLNYIFRLWGPGTLVTVSS

SEQ ID NO: 5 - protein, 109 aa, Artificial Sequence
  Synthetic sequence
DVVMTQTPSPVSAAVGGTVTIKCQASQSINNGLAWYQQKPGQPPRLLIYSASNLESGVPSRFKGSGSGTEFTLTISDLECDDAATYYCQCIWDGNSYVNAFGGGTEVVV

SEQ ID NO: 6 - protein, 110 aa, Artificial Sequence
  Synthetic sequence
DVVMTQTPASVSEPVGGTVTIKCQASQRISSYLSWYQQKPGQRPKLLIFGASTLASGVPSRFKGSGSGTEYTLTISDLECADAATYYCQSYAYFDSNNWHAFGGGTEVVV

SEQ ID NO: 7 - protein, 241 aa, Artificial Sequence
  Synthetic sequence
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSGGGGSGGGGSGGGGSDIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIK

SEQ ID NO: 8 - protein, 248 aa, Artificial Sequence
  Synthetic sequence
QQQLEESGGGLVKPEGSLTLTCKASGFDLGFYFYACWVRQAPGKGLEWIACIYTAGSGSTYYASWAKGRFTISKASSTTVTLQMTSLAAADTATYFCARSTANTRSTYYLNLWGPGTLVTVSSGGGGSGGGGSGGGGSDVVMTQTPASVSEPVGGTVTIKCQASQRISSYLSWYQQKPGQRPKLLIFGASTLASGVPSRFKGSGSGTEYTLTISDLECADAATYYCQSYAYFDSNNWHAFGGGTEVVV

SEQ ID NO: 9 - protein, 449 aa, Artificial Sequence
  Synthetic sequence
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 213 aa, Artificial Sequence
  Synthetic sequence
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 232 aa, Artificial Sequence
  Synthetic sequence
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 330 aa, Artificial Sequence
  Synthetic sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 107 aa, Artificial Sequence [sequence omitted]

SEQ ID NO: 14 - protein, 323 aa, Artificial Sequence
  Synthetic sequence
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 15 - protein, 104 aa, Artificial Sequence
  Synthetic sequence
GDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 16 - protein, 117 aa, Artificial Sequence
  Synthetic sequence
QVQLKQTGPGLVQPSQSLSITCTVSGFSLTNYAVHWVRQSPGKGLEWLGVIWSGGATDYNTVFISRLSISKDNSKSQVFFKVNSLQVDDTAIYYCARTGSGYAMDYWGQGTSVTVSS

SEQ ID NO: 17 - protein, 107 aa, Artificial Sequence
  Synthetic sequence
DIVLTQSPATLSVTPGDRVSLSCRTSHNVNTYLHWYQQKSHESPRLLIKYASQSISEIPSRFSGSGSGTNFTLSINSVETEDFGMYFCHQTNRWPLTFGAGTKLELK

SEQ ID NO: 18 - protein, 120 aa, Artificial Sequence
  Synthetic sequence
DVQLQESGPGLVKPSQSLSLTCTVTAYSITSDYAWNWIRQFPGNKLEWMGCIRYSGGTTYNPSLKSRISITRDTSKNQFFLQLNSVTTEDTATYHCARSRQLGDAGFDYWGQGTTLTVSS

SEQ ID NO: 19 - protein, 106 aa, Artificial Sequence
  Synthetic sequence
QIVLTQSPAIMSASPGEKVTISCSASSSVSYMYWYQQKPGSSPKPWISRTSNLASGVPARFSGSGSGTSYSLTISSMEAEDAATYYCQQYHSYPPTLGAGTKLELK

SEQ ID NO: 20 - protein, 120 aa, Artificial Sequence
  Synthetic sequence
EVQLQQSGPELVKPGASLKISCKASGYSFTDYTMNWVKQSHGKNLEWIGLFNPYNGGISYNQKFKGKATLTVDKSSNTAYMELLSLTSDDSAVYFCARDGRGGFYAMDYWGQGTSVTVSS

SEQ ID NO: 21 - protein, 108 aa, Artificial Sequence
  Synthetic sequence
DIQMTQTTSSLSASLGDRVTISCRASQDISIYLNWYQQKPDGPVKLLIYYTSRLHSGVPSRFSGSGSGTDFSLTISNLEQEDIATYFCQQGYTLPPWTFGGGTKLEIK

SEQ ID NO: 22 - protein, 122 aa, Artificial Sequence
  Synthetic sequence
EVQLQQSEPELVKPGASVRISCKASGYSFTDYYMHWVKQSPENRLEWIGEINPSTGGTSYNPKFKDKATLTVDKSSSTAYMQLKSLTSEESAVYYCTRYHYYGSSSYVMDYWGQGTSVTVSS

SEQ ID NO: 23 - protein, 106 aa, Artificial Sequence
  Synthetic sequence
QIVLTQSPAILSASPGERVTMTCSASSSVYHMHWFQQKSGTSPKRWVYDTSKLASGVPARFSGSGSGTSYSLTISSMEAEDAATYYCQHWRTNPLTFGAGTKLELK

SEQ ID NO: 24 - protein, 116 aa, Artificial Sequence
  Synthetic sequence
EVQLQQSGAEVVKPGASVKLSCTASGFNIRHTYMHWVKQRPEQGLEWIGRIDPANGNTEYDPKFQGKATITADTSSNTAYLQLSSLTSEDTAVYYCARDGWYIDVWGAGTTVTVSS

SEQ ID NO: 25 - protein, 113 aa, Artificial Sequence
  Synthetic sequence
DIVMSQSPSSLAVSVGEKVTMSCKSSQSLLYSNNQKNYLAWYQQKPGQSPKLLIYWASTRESGVPDRFTGSGSGTDFTLTISSVKAEDLAVYYCQRYYSYPWTFGGGTKLEIK

SEQ ID NO: 26 - protein, 112 aa, Artificial Sequence
  Synthetic sequence
EAQLQQSGAVFVKPGASVKLSCTASGFSSKDIFLHWVKQRPEQGLEWIGRIDPASDNTIYDPKFQDRATITSDRSSNTAYLQFSSLTSEDTAVYFCLYFDFWGQGTTLTVSS

SEQ ID NO: 27 - protein, 112 aa, Artificial Sequence
  Synthetic sequence
DIVMTQATPSISVTPGESVSISCRSSESLLHSNGNTYLYWFLQRPGQSPQLLIYRMSNLASGVPDRFSVSGSGTAFTLRISRVEAEDVGVYYCMQHLEYPLTFGAGTKLELK

SEQ ID NO: 28 - protein, 114 aa, Artificial Sequence
  Synthetic sequence
EVKLEGSGGGLVQPGGSMMLSCVATGFTFSSYWMNWVRQSPDRGLEWVAEIRLKSNGFAAFYAESVKGRFTISRDDSKSSVYLQMNNLRTEDTGIYYCTYFVHWGQGILVTVSE

SEQ ID NO: 29 - protein, 111 aa, Artificial Sequence
  Synthetic sequence
DIVLTQSPTSLAVSLGQRATISCRTSESVEYFGTILIQWYQQKPGQPPKLLIFGASNVESGVPARFSGSGSGTDFSLNIHPVEEEDIAMYFCQQNRKVPYTFGGGTTLELK

SEQ ID NO: 30 - protein, 112 aa, Artificial Sequence
  Synthetic sequence
EVQLQQSGADLVKPGASVKLSCTVFGLNFKDTFMHWVRQRPEQGLEWIGRIDPANDNSIYGPKFQDKATITADTSSNTAYLHLSSLTSEDTAVYYCLYFDNWGHGTTLTVSS

SEQ ID NO: 31 - protein, 112 aa, Artificial Sequence
  Synthetic sequence
DIVMTQAASSEPVTPGESVSISCRSDKSLLHSNGHNYLYWFLQRPGQSPHLLIYRMSNLASGVPDRFSGSGSGTAFTLRISRVEAEDVGVYYCMQHLEYPLTFGAGTKLELK

SEQ ID NO: 32 - protein, 116 aa, Artificial Sequence
  Synthetic sequence
QVQLQQSGAELMKPGASVKLSCKATGYTFTGYWIEWVKQRPGHGLEWIGEILPGSGSTNYNEKFKGKATFTADTSSNTAYMQLSSLTTEDSAIYYCARAGAWFAYWGQGTLVTVSA

SEQ ID NO: 33 - protein, 108 aa, Artificial Sequence
  Synthetic sequence
QIVLTQSPAIMSASLGERVTMTCTASSSVSSSYLHWYQQKPGSSPKLWTYSTSNLASGVPARFSGSGSGTSYSLTISSMEAEDAATYYCHQYHRSPWTFGGGTKLEIK

SEQ ID NO: 34 - protein, 121 aa, Artificial Sequence
  Synthetic sequence
QVQLQQPGAELIKPGASVKLSCKASGYTFTSYWMHWVKQRPGQGLEWIGIIHPNIGSTNYNERFKSKATLTVDKSSSTAYMQLSSLTSEDSAVYYCARRSSNYGDWYFDVWGTGTTVTVSS

SEQ ID NO: 35 - protein, 107 aa, Artificial Sequence
  Synthetic sequence
DIVMTQSHRFMSTSVGDRVSITCKASQDVGTSVAWYQQKPGQSPKLLIYWASTQHTGVPDRFTGSGSGTDFTLTINNVQSEDLLDYFCQQYSSYPLTFGAGTKLELK

SEQ ID NO: 36 - protein, 115 aa, Artificial Sequence
  Synthetic sequence
QVQLQQPGTELVTPGASVKLSCKASGYSFTSYWMHWVKQRPGQGLEWIGNINPSNGDTFYNEKFKNKATLTVDKSSSTAYMQLSSLTSEDSAVYYCARGWLRDYWGQGTTLTVSS

SEQ ID NO: 37 - protein, 113 aa, Artificial Sequence
  Synthetic sequence
DIVMTQSPSSLAVSVGQKVTMSCKSSQSLLNSSSQKNYLAWYQQKPGQSPKLLVYFASTKDSGVPDRFIGSGSGTDFTLTINSVQAEDLADYFCQQHYTTPYTFGGGTKLEIK

SEQ ID NO: 38 - protein, 120 aa, Artificial Sequence
  Synthetic sequence
EVQLQQSGPELVKPGASVKIPCKASGYTFTDYNMDWVKQSHGKSLEWIGDINPNNGGSIYNQRFKGKATLTVDKSSSTAYMELRSLTSEDSAVYYCARRAYYSTGYFDVWGTGTTVTVSS

SEQ ID NO: 39 - protein, 106 aa, Artificial Sequence
  Synthetic sequence
QIVLTQSPALMSASPGEKVTITCSASSSISYMHWFQQKPGTSPKLWIYSTSTLASGVPARFSGSGSGTSYSLTISRMEAEDTATYYCQQRSSYPPTFGGGTKLEIK

SEQ ID NO: 40 - protein, 121 aa, Artificial Sequence
  Synthetic sequence
QVQLQQPGTELVKPGASVKLSCKASGYTFTNYWMHWVKQRPGQGLEWIGNINPSNGGPYYNERFRSKATLTVDKSSSTAYMQLSSLTSEDSAVYYCARPYYGSSYGYFDYWGQGTTLTVSS

SEQ ID NO: 41 - protein, 107 aa, Artificial Sequence
  Synthetic sequence
DIQMTQSPSSLSASLGGKVTITCKASQDINKYIAWYQHKPGKGPRLLIHYTSELQPGIPSRFSGNGSGRDYSFSISNLEPEDIATYYCLQYANPLRTFGGGTKLEIK

SEQ ID NO: 42 - protein, 120 aa, Artificial Sequence
  Synthetic sequence
QVQLQQPGTELVKPGASVKLSCKASGYTFTSYWMHWVKQRPGQGLEWIGNINPSNGGTNYNEKIKNKATLTVDKSSSTAYMQLSSLTSEDSAVYYCARWNYYGNYPFDYWGQGTTLTVSS

SEQ ID NO: 43 - protein, 107 aa, Artificial Sequence
  Synthetic sequence
DIVMTQSQKFMSTTVGDRVSITCKASQNVGTAVAWYQQKPGQSPKLLIYSASNRYTGVPDRFTGSGSGTDFTLTISNMQSEDLADYFCQQYSSYPLTFGAGTKLELK

SEQ ID NO: 44 - protein, 117 aa, Artificial Sequence
  Synthetic sequence
QVQLQQPGTELVKPGASVKLSCKASGYTFTNYWMHWVKQRPGQGLEWIGNINPSSGDSYYNERFMSKAKMTVDKSSSTAYMQLSSLTSEDSAVYYCARSGGLWLAFWGPGTLVTVSA

SEQ ID NO: 45 - protein, 113 aa, Artificial Sequence
  Synthetic sequence
DIVMTQSPSSLAMSVGQKVTMSCKSSQTLLNSVSQNNYLAWYQQKPGQSPTLLVYFASTRESGVPDRFIGGGSGTDFTLTISSVQAEDLADYFCQQHYRTPYTFGGGTNLEIK

SEQ ID NO: 46 - protein, 119 aa, Artificial Sequence
  Synthetic sequence
QVQLQQPGAELVKPGASVKLSCKASGYTFTSYWMHWVKQRPGQGLEWIGMIHPNSGSTNYNEKFKSKATLTVDKSSSTAYMQLSSLTSEDSAVYYCARPVVPYWYFDVWGTGTTVTVSS

SEQ ID NO: 47 - protein, 107 aa, Artificial Sequence
  Synthetic sequence
DIVMTQSQKFMSTTVGDRVSITCKASQNVGTAVAWYQQKPGQSPKLLIYSASNRYTGVPDRFTGSGSGTDFTLTISNMQSEDLADYFCQQSSSYPLTFGAGTKLELK

SEQ ID NO: 48 - protein, 115 aa, Artificial Sequence
  Synthetic sequence
DVQLQESGPGMVKPSQSLSLTCTVTGYSITSGYDWHWIRHFPGNKLEWMGYISYSGSTNYNPSLKSRISITHDTSKNHFFLKLKSVTTEDTATYYCARGTGPDYWGQGTTLTVSS

SEQ ID NO: 49 - protein, 107 aa, Artificial Sequence
  Synthetic sequence
DIVMTQSQKFMSTSVGDRVSITCKASQNVRTAVAWYQQKPGQSPKALIHLPSNRHTGVPDRFTGSGSGTDFTLTISNVQSEDLADYFCLQHWNYPLTFGGGTKLEIK

SEQ ID NO: 50 - protein, 121 aa, Artificial Sequence
  Synthetic sequence
QVQLLQSGAELAKPGASVKLSCKASGYTFTSYWVHWVKQRPGQGLEWIGYINPNSGYTKYNQKFKDKATLTADKSSSTAYMQLSSLTYEDSAVYYCADHYYGSSRDYFDYWGQGTTLTVSS

SEQ ID NO: 51 - protein, 106 aa, Artificial Sequence
  Synthetic sequence
QIVLTQSPAIMSASPGEKVTMTCSASSSVSYMYWYQQRPGSSPRLLIYDTSNLASGVPVRFSGSGSGTSYSLTISRMEAEDAATYYCQQWNSYPYTFGGGTKLEIK

SEQ ID NO: 52 - protein, 116 aa, Artificial Sequence
  Synthetic sequence
EVNFEESGGGLVQPGGSMKLSCVASGFTFSNYWMNWVRQSPEKGLEWVAQIRLKSDNYATHYAESVKGRFTISRDDFKSSVYLQMNNLRAEDTGIYYCTVGTGGYWGQGTTLTVSS

SEQ ID NO: 53 - protein, 111 aa, Artificial Sequence
  Synthetic sequence
DIVLTQSPASLAMSLGKRATISCRASESVSIVGTNVIHWFQQKPGQPPKLLIYHASNLETGVPARFSGSGSGTDFTLTIDPVEEDDVAIYHCLQSRKIPWTFGGGTKLEIK

SEQ ID NO: 54 - protein, 116 aa, Artificial Sequence
  Synthetic sequence
EVKFEESGGGLVQPGGSMKLSCVASGFTFSNYWMNWVRQSPEKGLEWVAQIRLKSDNYATHYAESVKGRFSISRDDSKSSVYLQMNNLRAEDTGIYYCSVGTGGYWGQGTTLTVSS

SEQ ID NO: 55 - protein, 111 aa, Artificial Sequence
  Synthetic sequence
DIVLTQSPASLALSPGKRATISCRASESVSIIGTDVIHWFQQKPGQPPKLLIYHASNLETGVPARFSGSGSRTDFTLTIDPVEEDDVAIYYCLQSRKIPWTFGGGTKLEIK

SEQ ID NO: 56 - protein, 115 aa, Artificial Sequence
  Synthetic sequence
QVQLQQSGADLVRPGASVTLSCKASGYTFTDYEMHWVKQTPVHGLEWIGAIDPETGDTVYNQNFKGKAILTADKSSSTAYMELRSLTSEDSAVYYCTRYGYDWGWGQGTTLTVSS

SEQ ID NO: 57 - protein, 112 aa, Artificial Sequence
  Synthetic sequence
DVVMTQTPLTLSVTIGQPASISCKSSQSLLYSNGKTYLNWLLQRPGQSPKRLIYLVSKLDSGVPDRFTGSASGTDFTLKISRVEADDLGVYYCVQGTHFPWTFGGGTKLEIK

SEQ ID NO: 58 - protein, 119 aa, Artificial Sequence
  Synthetic sequence
EVQLQQSGPVLVKPGASVKMSCEASGYIFTDYYMNWVKKSHGKSLEWIGVINPKNGVISHNQKFKGKATLTVDKSSNTAYMELSSLTSEDSAVYYCANYGSRFYAMDYWGQGTSVTVSS

SEQ ID NO: 59 - protein, 112 aa, Artificial Sequence
  Synthetic sequence
DVVMTQTPLSLPVSLGDQASISCRSSQSLIHSDGNTYLQWYLQKSGQSPKLLIYKVSNRFSGVPDRFSGSGSGTDFTLKISRVETEDLGVYFCSQTTHVPFTFGSGTKLEIK

SEQ ID NO: 60 - protein, 120 aa, Artificial Sequence
  Synthetic sequence
DVQLQESGPGLVKPSQSLSLTCSVTGDSITSGYYWNWIRQFPGNKLEWMGFIRFDGTNNYNPSLKNRISITRDTSKNQFFLKLNSVTTEDTATYYCAREGSYAPAWFAYWGQGTLVTVSA

SEQ ID NO: 61 - protein, 106 aa, Artificial Sequence
  Synthetic sequence
QIVLTQSPAIMSASPGEKVTMTCSASSSLSYMYWYQQKPGSSPRLLIYDTSNLASGVPVRFSGSGSGTSYSLTISRMEAEDAATYYCQQWSSYPPTFGGGTKLEIK

SEQ ID NO: 62 - protein, 117 aa, Artificial Sequence
  Synthetic sequence
QVQLQQSGAELVRPGASVTLSCKASGYTFTDYEMHWVKQTPVHGLEWIGGFDPETGGTAHNQKFKGKAILTADKSSSTAYMELRSLTSEDSAVYYCSNYGSIWGDCWGQGTSVTVSS

SEQ ID NO: 63 - protein, 112 aa, Artificial Sequence
  Synthetic sequence
DVVMTQTPLTLSVTVGQPASISCKSSQSLLYSDGKTYLNWLLQRPGQSPKRLIYLVSKLDSGVPDRFTASGSGTDFTLKISRVEAEDLGVYYCWQGTHFPWTFGGGTKLEIK

SEQ ID NO: 64 - protein, 117 aa, Artificial Sequence
  Synthetic sequence
QVQLQQSGAELVRPGASVTLSCKASGYTFTDYEMHWVKQTPVHGLEWIGGIDPETGDTAYNQQFKGKAILTADRSSSTAYMELRSLTSEDSAVYYCTNYASSREDYWGQGTTLTVSS

SEQ ID NO: 65 - protein, 112 aa, Artificial Sequence
  Synthetic sequence
DVVMTQTPLTLSVTIGQPASISCKSSQSLLYSNGKTYLSWLLQRPGQSPKRLIYQVSKLDSGVPDRFTGSGSGTEFTLKISTVEAEDLGVYYCVQITHFPQTFGGGTKLEIK

SEQ ID NO: 66 - protein, 120 aa, Artificial Sequence
  Synthetic sequence
RVQLQQSGAELVRPGASVTLSCKASGYTFTDSEMHWVKQTPVHGLEWIGAIDPEIDGTAYNQNFRDKAILTADKSSSTAYMELRSLTSEDSAVYYCTTYFGSGYGYFDVWGTGTTVTVSS

SEQ ID NO: 67 - protein, 112 aa, Artificial Sequence
  Synthetic sequence
DVVMTQTPLSLSVSLGDQASISCRSSQSLVYSNGNTYLHWFLQKPGQSPKLLIYKVSNRFSGVPDRFSGSGSGTDFTLKINRVEAEDLGVYFCSQSTHIPFTFGAGTKLELK

SEQ ID NO: 68 - protein, 120 aa, Artificial Sequence
  Synthetic sequence
QVQLQQSGAELVRPGASVTLSCKASGYTFTDYEIHWVKQTPVHGLEWIGAFDPEIGGSAYNQKFKDRATLTADKSSSTAYMELHSLTAEDSAVYYCTDYYGSSSGYFDVWGTGTTVTVSS

SEQ ID NO: 69 - protein, 112 aa, Artificial Sequence
  Synthetic sequence
DVVMTQTPLSLPVSLGDQASISCRSSQSLEHNNGNTYLHWFLHKPGQSPKLLIYKVSNRFSGVPDRFSGSGSGTDFTLKISRVEAEDLGVYFCSQSTHVPFTFGSGTKLEIK

SEQ ID NO: 70 - protein, 117 aa, Artificial Sequence
  Synthetic sequence
QVQLHQPGAELVRPGSSVKLSCKASGYTFTSYWVHWVQQRPRQGLEWIGNIDPSDSEIHYNQKFKDKATLTVDKSSSTAYIHLSRLTSEDSAIYYCVRGGVPWFAYWGQGTLVTVSA

SEQ ID NO: 71 - protein, 108 aa, Artificial Sequence
  Synthetic sequence
QIVLTQSPAIMSASPGEKVTLTCSASSSVSSTYLYWFQQKPGSSPKVWIYSTSNLASGVPARFSGSGSGTSYSLTISSMEAEDAASYFCHQWISYPYTFGGGTKLEIK

SEQ ID NO: 72 - protein, 119 aa, Artificial Sequence
  Synthetic sequence
EVQLQQSGPVLVKPGASVKMSCKASGYTFTDYYMNWVKQSHGKSPEWIGVINPYNDVISYNQKFKGKATLTVDKSSSTAYMELNSLTSEDSAVYYCANYGSSYYAMDYWGQGTSVTVSS

SEQ ID NO: 73 - protein, 112 aa, Artificial Sequence
  Synthetic sequence
DVVMTQTPLSLSVSLGDQASISCRSSQSLVHSNGNTYLQWSLQKPGQSPNLLIYKVSNRFSGVPDRFSGSGSGTDFTLKISRVEAEDLGVYFCSQTTHVPFTFGGGTKLEIR

SEQ ID NO: 74 - protein, 118 aa, Artificial Sequence
  Synthetic sequence
EVQLRQSGPVLVKPGASVKMSCKASGYTFTDYFMNWVKPSHGKSLEWIGVINPSNGVINYNQRFKGKATLTVDKSSSTAYMELNSLTSEDSAVYYCARSYDFAWFAYWGQGTLVTVSA

SEQ ID NO: 75 - protein, 112 aa, Artificial Sequence
  Synthetic sequence
DVLMTQTPLSLPVSLGDQASISCRSSQNIVHSNGNTYLEWYLQKPGQSPKLLIYTVSNRFSGVPDRFSGSGSGTDFTLKISRVEAEDLGVYYCFQGSHVPYTFGGGTKLEIK

SEQ ID NO: 76 - protein, 116 aa, Artificial Sequence
  Synthetic sequence
QVQLQQPGTELVKPGASVKLSCRASGYTFTAYWMHWVKQRPGQGLEWIGNINPSNGGTDFNEKFKSKATLTVDKSSSTAYMQLSSLTSEDSAVYYCARGRGYFDVWGTGTTVTVSS

SEQ ID NO: 77 - protein, 108 aa, Artificial Sequence
  Synthetic sequence
DILLTQSPAILSVSPGERVSFSCRASKNIGTSIHWYQQRTNGSPRLLIKYASESISGIPSRFSGSGSGTDFTLNINSVESEDIADYYCQQSNSWPTLTFGAGTKLELK

SEQ ID NO: 78 - protein, 121 aa, Artificial Sequence
  Synthetic sequence
QVQLQQSGPELVKPGASVKISCKASGYTFTDYYINWVKQRPGQGLEWIGWIFPGRGSTFYYEKFKGKATLTVDKSSSTAYMLLSSLTSEDSAVYFCARRGDSAGYEDLDYWGQGTSVTVSS

SEQ ID NO: 79 - protein, 107 aa, Artificial Sequence
  Synthetic sequence
NIVMTQSPKFMSMSVGERVTLSCKASENVVTYVSWYQQKPKQSPKLLIYGASKRYTGVPDRFTGSGSATDFTLTISSVQAEDLGDYHCGQSYSYPYTFGGGTKLEIK

SEQ ID NO: 80 - protein, 118 aa, Artificial Sequence
  Synthetic sequence
ELQLVESGGDLVKPGGSLKLSCAASGFTFSSNGMSWLRQTPDKRLEWVATISSGGRYTYYPDSVKGRFTISRDNAKNTLYLQMSSLKSEDTAMYYCARRGIYDYFDCWGQGTTLTVSS

SEQ ID NO: 81 - protein, 107 aa, Artificial Sequence
  Synthetic sequence
DIQMTQSPASLSISVGETVTITCRASENIHSSLAWYQQKQGKSPQLLVYAATNLADGVPSRFSGSGSGSQYSLKINSLQSEDFGTYYCQHFWGTPWTFGGGTKLDIK

SEQ ID NO: 82 - protein, 118 aa, Artificial Sequence
  Synthetic sequence
QVQLQQSGAELARPGASVKMSCKASGYTFTTYTMHWVKQRPGQGLEWIGYISPTGDFTKYNQKFKDKATLTTDKSSNTAFMQLSSLTSEDSAVYYCARWNGTVWFAYWGQGTLVTVSA

SEQ ID NO: 83 - protein, 107 aa, Artificial Sequence
  Synthetic sequence
DIVMTQSQKFMSTSVGDRVTVTCKASQALGTNVAWYQQKPGQSPKVLIYSASYRYSGVPYRFAGGGSGTDFTLTITNVQSEDLAEYFCQQYNNYPLTFGAGTKLELK

SEQ ID NO: 84 - protein, 119 aa, Artificial Sequence
  Synthetic sequence
EVQLQQSGPELVKPGASVKIPCKASGYTITDYNMDWVKQSRGKSLEWIGDVFPYNGDSIYNQKFEGRATLTVDKYSSTAYMELRGLTSEDTAVYFCARRKTGTGYFDVWGTGTTVTVSS

SEQ ID NO: 85 - protein, 108 aa, Artificial Sequence
  Synthetic sequence
QLVLTQSPAIMSASPGEKVTLTCSASSSVSSTYLSWYLQKPGSSPKLWIYSTSNLASGVPARFSGSGSGTSYSLTISSMEAEDAASYFCHQWSSYPYTFGGGTKLEIK

SEQ ID NO: 86 - protein, 119 aa, Artificial Sequence
  Synthetic sequence
EVQLQQSGPELVKPGASVKIPCKASGYTFTDYNMDWVKQSHGKGLEWIGDINPNTDDTIYNQKFNGKATLTVDKSSSTAYMELRSLTSEDTAVYYCSRRLIGTGYFDVWGTGTTVTVSS

SEQ ID NO: 87 - protein, 108 aa, Artificial Sequence
  Synthetic sequence
QIVLTQSPAIMSASPGEKVTLTCSASSSVISTYLCWYRQKPGSSPELWIYSTSNLASGVPARFSGSGSGTSYSLTISSMEAEDAASYFCHQWSNYPYTFGGGTKLGMK

SEQ ID NO: 88 - protein, 118 aa, Artificial Sequence
  Synthetic sequence
QVQLQQSGAELVRPGTSVKVSCKASGYAFTNYLIEWVKQRPGQGLEWIGVINPGSGGTKYNEKFKGKATLTADKSSSTAYMQLSSLTSEDSAVYFCASDYDYDPFAYWGQGTLVTVSA

SEQ ID NO: 89 - protein, 107 aa, Artificial Sequence
  Synthetic sequence
DIVMTQSHKFMSTSVGDRVSITCKASQDVSTAVAWYQRKPGQSPKLLIYWASTRHTGVPDRFTGSGSGTDYTLTISYMQAEDLALYYCQQHYSTPYTFGGGTKLEIK

SEQ ID NO: 90 - protein, 120 aa, Artificial Sequence
  Synthetic sequence
QVQLQQPGTELVKPGASVKLSCKASGYTFTTFWMHWVKQRPGQGLEWIGGVNPTNGGTNYNEKFRTKATLTVDKSSTTADMQLSSLTSEDSAVYYCAPHYISSRPGFAYWGQGTLVTVSG

SEQ ID NO: 91 - protein, 107 aa, Artificial Sequence
  Synthetic sequence
DIVMTQSQKFMSTSGGDRVSVTCKASQNVGTNVAWYQQKPGQSPKALIYSASYRYSGVPDRFTGSGSGTDFILTISNVQSEDLAEYFCQQYNSYPLTFGGGTKLEIK

SEQ ID NO: 92 - protein, 125 aa, Artificial Sequence
  Synthetic sequence
EVKLVESGGGLVQPGGSLSLSCAASGFTFTDYYMSWVRQPPGKALEWLGFIRSKANAYTTEYSASVKGRFTISRDNSQSILYLQMNDLRAEDSATYYCARYYYYGSGYVWYFDVWGTGTTVTVSS

SEQ ID NO: 93 - protein, 107 aa, Artificial Sequence
  Synthetic sequence
DIVMTQSQKFMSTSVGDRVSVTCKASQSVGTNVAWYQQKPGQFPKALIYSASYRYSGVPDRFTGSGSGTDFTLTIINVQSEDLAEYFCQQYNSYPLTFGAGTKLELK

SEQ ID NO: 94 - protein, 120 aa, Artificial Sequence
  Synthetic sequence
QVQLQQPGTELVKPGASVKLSCKASGYTFTSYWMHWVKQRPGQGLEWIGNIIPSNGGTKYNEKFKSKATLTVDKSSSTAYMQLSSLTSEDSAVYYCSPHYYGGSPGFAYWGQGTLITVSA

SEQ ID NO: 95 - protein, 107 aa, Artificial Sequence
  Synthetic sequence
DIVMTQSQKFMSTSVGDRVSVICKASQNVGTNVAWYQQKPGQSPKALIYSASYRYSGVPDRFTGSGSGTDFTLTISNVQSEDLAEYFCQQYNSYPLTFGGGTKLEIK

SEQ ID NO: 96 - protein, 120 aa, Artificial Sequence
  Synthetic sequence
DVQLQESGPGLVKPSQTVFLTCTVTGISITTGNYRWSWIRQFPGNKLEWIGYIYYTGFITYNPSLTSRTTITRDTPKNQFFLEMKSLTAEDTATYYCARDDYDYDVFAYWGQGTLVTVSA

SEQ ID NO: 97 - protein, 107 aa, Artificial Sequence
  Synthetic sequence
DIQMTQSPASLSASVGETVTITCRASGNIHNYLAWYHQKQGKSPQFLVYKAKTLADGVPSRFSGSGSGTQYSLKINRLQPEDFGSYYCQHFLSIPLTFGAGTKLELK

SEQ ID NO: 98 - protein, 120 aa, Artificial Sequence
  Synthetic sequence
QVQLQQPKTELVKPGASVKLSCKASGYIFTTYWMHWVKQRPGQGLEWIGNVNPSNGGTMYNEKFERRATLTVDKSSSTADMQLSSLTSEDSAVYYCVPHYIGSRPGFAYWGQGTLVTVSG

SEQ ID NO: 99 - protein, 107 aa, Artificial Sequence
  Synthetic sequence
DIVMTQSQKFMSTSVGDRVSVTCKASQNVGTNVAWYQHKPGQSPKALLYSASYRYSGVPDRFTGSGSGTDFTLTITNVQSEDLAEYFCQQYNSYPLTFGGGTKLEIK

SEQ ID NO: 100 - protein, 120 aa, Artificial Sequence
  Synthetic sequence
QVQLQQPGTELVKPGASVKLSCKASGYTFTSYWMHWVKQRPGQGLEWIGNVNPSNGGSNYNEKFKNKATLTVDKSSSTADMQLNSLTSEDSAVYYCAPHYIGSRPGFAYWGQGTLVTVSG

SEQ ID NO: 101 - protein, 107 aa, Artificial Sequence
  Synthetic sequence
DIVMTQSQKFMSTSVGDRVSVTCKASQNVGTNIAWYQQKPGQSPKALIYSASYRYSGVPDRFTGSGSGTDFTLTITNVQSEDLAEYFCQQYNSYPLTFGGGTKVEIK

SEQ ID NO: 102 - protein, 115 aa, Artificial Sequence
  Synthetic sequence
QVQLQQPGTELVKPGASVKLSCKASDYTFTSYWMHWVKQRPGQGLEWIGSINPSNGDTYYNEKFKNKATLTVDKSSSTAYMQLSSLTSDDSAVYYCARGWYFDVWGTGTTVTVSS

SEQ ID NO: 103 - protein, 113 aa, Artificial Sequence
  Synthetic sequence
DIVMTQSPSSLTVSVGQKVTMSCKSSQSLLNSSSQKNYLAWYQQKPGQSPKLLVYFASTRESGVPDRFIGSGSGTDFTLIISSVQAEDLADYFCQQHYSTPRTFGGGTKLEII

SEQ ID NO: 104 - protein, 119 aa, Artificial Sequence
  Synthetic sequence
EVQLQQSGPELVKPGASVKIPCKASGYTFTDYNMDWVKQSHGKSLEWIGDINPSTGGTIYNQKFNGKATLTEDKSASTVYMEFRSLTSDDTAVYYCARRRIGTGYFDVWGTGTTVTVSS

SEQ ID NO: 105 - protein, 108 aa, Artificial Sequence
  Synthetic sequence
QIVLTQSPALMSASPGEKVTLTCSASSSVISSYLSWYQQKPGSSPKLLIYRTSNLASGVPARFSGSGSGTSYSLTISSMEAEDAASYFCHQWSSFPYTFGGGTKLEIK

SEQ ID NO: 106 - protein, 120 aa, Artificial Sequence
  Synthetic sequence
QVQLQQPGTELVKPGASVKLSCKASGYTFTSYWMHWVKQRPGQGLEWIGNINPSNGGTNNNENFKSKATLTVDTSSSTAYMQLSSLTSEDSAVYYCVRNGYHGYWYFDVWGTGTTVTVSS

SEQ ID NO: 107 - protein, 107 aa, Artificial Sequence
  Synthetic sequence
DIVMTQSQKFMSTSVGDRVSVTCKASQNVGTNVAWYQQQPGQSPKALIYSASYRYSGVPDRFTGSVSGTDFTLTISNVQSEDLAEYFCQQYNSYPLTFGGGTKLEIK

SEQ ID NO: 108 - protein, 119 aa, Artificial Sequence
  Synthetic sequence
EVQLQQSGPELVKPGASVKIPCKTSGYTFTDYNMDWVKQSHGKSLEWIGDINPNTDGAIYNQKFQNKATLTVDKSSSTAYMELRSLTSEDTAVYYCTRRKLGRKFFDYWGQGTTLTVSS

SEQ ID NO: 109 - protein, 106 aa, Artificial Sequence
  Synthetic sequence
QIVLTQSPAIMSASPGEQVTMTCSASSSVGYMNWYQQKPGSSPRLLIYDTSNLASGVPVHFSGSGSGTSYSLTISRMEAAFAATYYCQQWYVYPYTFGGGTKLEIK

SEQ ID NO: 110 - protein, 119 aa, Artificial Sequence
  Synthetic sequence
DVQLQQSGPELVEPGASVRIPCKAAGHTFTDYNVDWVKQSHGQSLEWIGDVNPNTDGAIYNQKFEGKATLTVDTSSSTAYMELRSLTSEDTAVYFCARRRLGQGGFDSWGQGTTLTVSS

SEQ ID NO: 111 - protein, 108 aa, Artificial Sequence
  Synthetic sequence
QVVLTQSPAIMSASPGEKVTLTCGASSSLISKYLYWYQQKPGSSPKLWIYSTSNLASGVPARFSGSGSGTSYSLTISSMEAEDAASYFCHQWSSYPYTFGGGTKVEIK

SEQ ID NO: 112 - protein, 119 aa, Artificial Sequence
  Synthetic sequence
EVQLQQSGPELVKPGTSVKILCKASGDTFTAYNMDWVKQRHGQSLEWIGDINPNTDSTIYNQKFEGKAILTVDKSSSTAYMELRSLTSEDTAVYYCARRKLGRGYFDYWGQGTTLTVSS

SEQ ID NO: 113 - protein, 106 aa, Artificial Sequence
  Synthetic sequence
QIVLTQSPAIMSASPGEKVTMTCSVNLSVRYIYWYQQKPGSSPRLLIHDTSNLASGVPVRFSGSGSGTSYSLTISRMEAEDAATYYCQQWSSFPYTFGGGTKLEIK

SEQ ID NO: 114 - protein, 123 aa, Artificial Sequence
  Synthetic sequence
QIPLQQSGPELVKPGASVRISCKASDYAFSSSWMNWVKQRPEKGLEWIGRIFVESGNTHYNDKFNGKATLTADKSSRTAYIQLSSLTSEDSAVYFCTRENIFYHGHSSWFAYWGQGTLVTVSA

SEQ ID NO: 115 - protein, 111 aa, Artificial Sequence
  Synthetic sequence
DIVLTQSPASLVVSLGQRATISCRAGESVDDFGISYVHWYQQKPGQPPKLLIYRAANLESGIPARFSGSGSRTDFTLTINPVETDDVAIYYCQQNNKDPFTFGSGTKLEIK

SEQ ID NO: 116 - protein, 114 aa, Artificial Sequence
  Synthetic sequence
QVQLQQPGTELVKPGASVRLSCRASGYSFSSYWMHWVKQRPGQGLEWLGNINPTTGDINYNEKFRNRATLAVDKSSTTAYLQLTSLTSEDSAVYFCVRHDGFLWGQGTTLTVSS

SEQ ID NO: 117 - protein, 107 aa, Artificial Sequence
  Synthetic sequence
DIQMTQSPASLSASVGETVTITCRASENIYSYLAWYQQKQGKSPQLLVYYAKTLAEGVSSRFSGSGSGTQFSLKINSLQPEDFGTYYCQHQFGTPRTFGGGTKLEIR

SEQ ID NO: 118 - protein, 121 aa, Artificial Sequence
  Synthetic sequence
EVHLQQSGPELVKPGASVRIPCKASGYTFTDYNMDWVKQSHGKSLEWIGDINPDNGGTIYTQKFKGKATLTVDKSSSTAYMELRSLTSEDTAVYYCARRLHYYGSSGLDYWGQGTTLTVSS

SEQ ID NO: 119 - protein, 107 aa, Artificial Sequence
  Synthetic sequence
DIQMTQSPASLSAYVGETVTITCRASGNIHNYLAWYQQKQGKSPQLLVYNAKTLADGVPSRFSGSGSGTQYSLKINSLQPEDFGSYYCQHFWSTVWTFGGGTKLEIK

SEQ ID NO: 120 - protein, 116 aa, Artificial Sequence
  Synthetic sequence
QVQLQQPGAELVKPGASVKLSCKASGYTFTIYWMHWVKQRPGQGLEWIGMIHPNSGNTNYNEKFKSKATLTVDKSSSTAYMQLSSLTSEDSAVYYCSLIHWYFDVWGTGTTVTVSS

SEQ ID NO: 121 - protein, 106 aa, Artificial Sequence
  Synthetic sequence
QIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLELK

SEQ ID NO: 122 - protein, 121 aa, Artificial Sequence
  Synthetic sequence
QVQLQQPGTELVKPGASVKLSCKASGYTFTSYWMHWVKQRPGQGLEWIGHSNPSNGGTNYNEKFKSKATLTVDKSSSTAYMQLSSLTSEDSAVYYCARWVRGSNYEYFDVWGTGTTVTVSS

SEQ ID NO: 123 - protein, 107 aa, Artificial Sequence
  Synthetic sequence
DIVMTQSQKFMSTTVGDRVSIICKASQNVGSAVAWCQQKPGQSPKLLIYSASNRYTGVPDRFTGSGSGTDFTLTISNMQSEDLADYFCQQYSSYPLTFAGGTKLEIK

SEQ ID NO: 124 - protein, 116 aa, Artificial Sequence
  Synthetic sequence
DVQLQESGPGLVKPSQSLSLTCSVTGYSITSGYYWNWIRQFPGNKLEWMAYIRYDGSNNYNPSLKNRISIIRDTSKNQFFLRLNSVTTEDTATYYCAKSYYGMDDWGQGISVTVSS

SEQ ID NO: 125 - protein, 107 aa, Artificial Sequence
  Synthetic sequence
DIQMTQSPASLSASVGETVTITCRTSGDIHNYLAWYQQKQGKSPQLLVYKAKTLENGVPSRFTGSGSGTQFSLKIDSLQPEDFGTYYCQHFWTTPYTFGGGTKLEIK

SEQ ID NO: 126 - protein, 118 aa, Artificial Sequence
  Synthetic sequence
EVQLQQSGPELVKPGASVKMSCKASGYTFTDYIIHWVKQSHGKSLEWIAYIFPNNGGTGYNQKFRGKATLTVNKSSSTAYMELRSLTSEDSAVYYCARWRLRQYFDVWGTGTTVTVSS

SEQ ID NO: 127 - protein, 107 aa, Artificial Sequence
  Synthetic sequence
DIVMTQSQKFMSTIVGDRVSITCKASQNVGTAVAWYQQKPGQSPKLLIYSASNRYTGVPDRFTGSGSGTDFTLTISNMQSEDLADYFCQQYSSYPLTFGGGTKLEIK

SEQ ID NO: 128 - protein, 118 aa, Artificial Sequence
  Synthetic sequence
EVHLQQSGPVLVKPGASVKMSCKASGYTFTDYFMNWVKQSHGKSLEWIGVINPHNGYVNYNQKFQGRATLTVDKSSSTVYMELNSLTSEDSAVYHCARSAESAWFAYWGQRTLVTVSA

SEQ ID NO: 129 - protein, 112 aa, Artificial Sequence
  Synthetic sequence
DVLMTQTPLSLPVSLGDQVSISCRSSQSIVHVKGNTYLEWYLQKPGQSPELLIYKVSNRFSGVPDRFSGGGSGTDFTLKISRVEPEDLGVYYCFQGSHLPYTFGGGTKLEIK

SEQ ID NO: 130 - protein, 124 aa, Artificial Sequence
  Synthetic sequence
QVQLQQPGAELVKPGASVKLSCKASGYTFTSYWMHWVKQRPGQGLEWIGMIHPNSGSTNYNEKFKSKATLTVDKSSSTAYMQLSSLTSEDSAVYYCARRTPIITLVGHWYFDVWGTGTTVTVSS

SEQ ID NO: 131 - protein, 107 aa, Artificial Sequence
  Synthetic sequence
DIVMTQSHKFMSTSVGDRVSITCKASQDVGTAVAWYQQKPGQSPKLLIYWASTRHTGVPDRFTGSGSGTDFTLTISNVQSEDLADYFCQQYSSYPLTFGAGTKLELK

SEQ ID NO: 132 - protein, 119 aa, Artificial Sequence
  Synthetic sequence
EFQLQQSGPELVKPGASVKISCKASGYSFTDYNMNWVKQTNGKSLEWIGIINPNYGTSSYNQKFKGKATLTVDQSSSTAYMQLNSLTSEDSAVYYCARVGRDNSGFDYWGQGTTLTVSS

SEQ ID NO: 133 - protein, 106 aa, Artificial Sequence
  Synthetic sequence
QIVLTQSPAIMSASPGEKVTMTCSASSSVSYMYWYQQKPGSSPRLLIYDTSNLASGVPVRFSGSGSGTSYSLTISRMEAEDAATYYCQQWSSYPPTFGSGTELEIK

SEQ ID NO: 134 - protein, 118 aa, Artificial Sequence
  Synthetic sequence
EFQLQQSGPELVKPGASVKISCKASGYSFTDYNMNWVKQSNGKSLEWIGIINPYYGTTSNNQNFKGKATLTVDQSSSTAYMQLNSLTSDDSAVYYCARWLRRDAMDYWGQGTSVTVSS

SEQ ID NO: 135 - protein, 107 aa, Artificial Sequence
  Synthetic sequence
DIVMTPSHKFMSTSVGDRVSIACKASQDVGSSVAWYQQKPGQSPKLLIYWTSTRHTGVPDRFTGSGSGTDFTLTISNVQSEDLADYFCQQYSSYPWTFGGGTKLEIK

SEQ ID NO: 136 - protein, 119 aa, Artificial Sequence
  Synthetic sequence
EFQLQQSGPELVKPGASVKISCKASGYSFTDYNMNWVKQSNGKSLEWIGIINPNYGTTSYNQKFKGRATLTVDQSSSTAYVQLNSLTSEDSAVYYCARGGYDNEAMDYWGQGTSVTVSS

SEQ ID NO: 137 - protein, 106 aa, Artificial Sequence
  Synthetic sequence
QIVLTQSPAIMSASPGEKVTMTCSASSSVSYMHWYQQKSGTSPKRWIYDTSKLASGVPARFSGSGSGTSYSLTISSMEAEDAATYYCQQWSSNPPTFGAGTKLELK

SEQ ID NO: 138 - protein, 119 aa, Artificial Sequence
  Synthetic sequence
EFQLQQSGPELVKPGASVKISCKTSGYSFTDYNMNWVKQSNGKSLEWIGVINPNYGTTGYNQKFKGKATLTVDQSSSTAYMQLNSLTSEDSAVYYCARGGYDGEAMDYWGHGTSVTVSS

SEQ ID NO: 139 - protein, 106 aa, Artificial Sequence
  Synthetic sequence
QIVLTQSPAIMSASPGEKVTMTCSASSSVNYMHWYQQKSGTSPKRWIYDTSKLASGVPARFSGSGSGTSYSLTISSMEAEDAATYYCQQWSSYPPTFGGGTKLEIK

SEQ ID NO: 140 - protein, 123 aa, Artificial Sequence
  Synthetic sequence
QVQLKQSGPGLVQPSQSLSITCTVSGFSLFSYGVHWVRQSPGKGLEWLGVIWSGGSTDYNAAFISRLSISKDNSKSQVFFKMNSLQADDTAMYYCARNGGSMITTLYYAMDYWGQGTSVTVSS

SEQ ID NO: 141 - protein, 112 aa, Artificial Sequence
  Synthetic sequence
DVVMTQTPLSLPVSLGDQASISCRSSQSLVHSYGNTYLHWYLQKPGQSPKLLIYKVSNRFSGVPDRFSGSGSGTDFTLKISRVEAEDLGVYFCSQSTHIPFTFGGGTKLEIK

SEQ ID NO: 142 - protein, 120 aa, Artificial Sequence
  Synthetic sequence
EFQLQQSGPELVKPGASVKISCKASGYSFTDYNMNWVKLSNGKSLEWIGVIIPNYGTSSYNQKFKGKATLTVDQSSSTAYMQLNSLTSEDSAVYYCARWDNYYGSSLDYWGQGTTLTVSS

SEQ ID NO: 143 - protein, 107 aa, Artificial Sequence
  Synthetic sequence
DIVMTQSQKFMSTTVGDRVSITCKASQNVGTAVAWYQQKPGQSPKLLIYSASNRYTGVPDRFTGSGSGTDFILTISNMQSEDLADYFCQQYSSYPLTFGAGTKLELK

SEQ ID NO: 144 - protein, 119 aa, Artificial Sequence
  Synthetic sequence
EVQLVESGGGLVKPGGSLKLSCAASGFTFSSYAMSWVRQTPEKRLEWVATISDGGNYTYYPDNVKGRFTISRDNAKNNLYLQMSHLKSEDTAMYYCAGDRGYPYAMDSWGQGTSVTVSS

SEQ ID NO: 145 - protein, 112 aa, Artificial Sequence
  Synthetic sequence
NIMMTQSPSSLAVSAGEKVTMSCKSSQSVLYSSNQKNYLAWYQQKPGQSPKLLIYWASTRESGVPDRFTGSGSGTDFTLTISNVHAEDLAVYYCHQYLSSLTFGAGTKLELK

SEQ ID NO: 146 - protein, 119 aa, Artificial Sequence
  Synthetic sequence
EVQLVESGGDLVKPGGSLKLSCAASGFTFSSYAMSWVRQTPEKRLEWVATISDGGSYTYYPDNVEGRFTISRDNAKNNLYLQMSHLKSEDTAMYYCAGDRGYSYALDYWGQGTSVTVSS

SEQ ID NO: 147 - protein, 112 aa, Artificial Sequence
  Synthetic sequence
NIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSLTFGAGTKLELK

SEQ ID NO: 148 - protein, 121 aa, Artificial Sequence
  Synthetic sequence
QVQLQQSGAELARPGASVKMSCKASGYTFISYTMHWVKQRPGQGLEWIGYINPSSGYTKYNQKFKDKATLTADKSSSTAYMQLNSLTSEDSAVYYCARYYSNPDYYAMDYWGQGTSVTVSS

SEQ ID NO: 149 - protein, 106 aa, Artificial Sequence
  Synthetic sequence
QIVLTQSPAIMSASPGEKVTITCSASSSVSYMHWFQQKPGTSPKLWIYSTSNLASGVPARFSGSGSGTSYSLTISRMEAEDAATYYCQQRSSYPLTFGGGTKLELK

SEQ ID NO: 150 - protein, 119 aa, Artificial Sequence
  Synthetic sequence
EVQLVESGGDLVKPGGSLKLSCAASGFTFSTYGMSWVRQTPDKRLEWVATISSGGSYTYYPDSLKGRFTISRDNAKNTLYLQMSSLKSEDTAMYYCARHEGYYYGKDYWGQGTTLTVSS

SEQ ID NO: 151 - protein, 107 aa, Artificial Sequence
  Synthetic sequence
DIVLTQSPATLSVTPGDSVSLSCRASHSISNNLHWYQQKSHESPRLLIKYASQSISGIPSKFSGSGSGTDFTLSINSVETEDFGMYFCQQTNSWPLTFGAGTKLELK

SEQ ID NO: 152 - protein, 120 aa, Artificial Sequence
  Synthetic sequence
QVQLKESGPGLVAPSQSLSITCTVSGFSLSSYAISWVRQPPGKGLEWLGVIWTGGGTNYNSALKSRLSISKDNSKSQVFLKMNSLQTDDTARFYCARNNYGRLDYAMDYWGQGTSVTVSS

SEQ ID NO: 153 - protein, 112 aa, Artificial Sequence
  Synthetic sequence
DVVMTQTPLSLPVSLGDQASISCRSSQSLVHGNGNTYLHWYLQKPGQSPKLLIYKVSNRFSGVPDRFSGSGSGTDFTLKISRVETEDLGVYFCSQSTHVPFTFGSGTKLEIK

SEQ ID NO: 154 - protein, 118 aa, Artificial Sequence
  Synthetic sequence
EVQLQQSGAELVKPGASVKLSCTASAFNIKDYYMHWVKQRTEQGLEWIGRIDPEDGETKYAPKFQGKATITADTSSNTAYLQLSSLTSEDTAVYYCASGSGTGAMDYWGQGTSVTVSS

SEQ ID NO: 155 - protein, 107 aa, Artificial Sequence
  Synthetic sequence
DIVMTQSQKFMSTSVGDRVSITCKASQNVRTAVGWYQQKPGQSPKALIYLASNRHTGVPDRFTGSGSGTDFTLTISNVQSEDLADYFCLQHWNSPYTFGGGTKLVIK

SEQ ID NO: 156 - protein, 122 aa, Artificial Sequence
  Synthetic sequence
EVQLQQSGPELVKPGASVKISCKASGYTFTDYYMNWVKQSHGKSLEWIGDINPNNGGTSYNQKFKGKATLTVDKSSSTAYMELRSLTSEDSAVYYCASWGYGSSSRGYFDYWGQGTTLTVSS

SEQ ID NO: 157 - protein, 106 aa, Artificial Sequence
  Synthetic sequence
QIVLSQSPAILSASPGEKVTMTCRASSSVSYMHWYQQKPGSSPKPWIYATSNLASGVPARFSGSGSGTSYSLTISRVEAEDAATYYCQQWSSNPWTFGGGTKLEIK

SEQ ID NO: 158 - protein, 330 aa, Artificial Sequence; identical to SEQ ID NO: 12
  Synthetic sequence

SEQ ID NO: 159 - protein, 107 aa, Artificial Sequence [sequence omitted]

SEQ ID NO: 160 - protein, 5 aa, Artificial Sequence
  Synthetic sequence
NYAVH

SEQ ID NO: 161 - protein, 16 aa, Artificial Sequence
  Synthetic sequence
VIWSGGATDYNTVFIS

SEQ ID NO: 162 - protein, 9 aa, Artificial Sequence
  Synthetic sequence
TGSGYAMDY

SEQ ID NO: 163 - protein, 11 aa, Artificial Sequence
  Synthetic sequence
RTSHNVNTYLH

SEQ ID NO: 164 - protein, 7 aa, Artificial Sequence
  Synthetic sequence
YASQSIS

SEQ ID NO: 165 - protein, 9 aa, Artificial Sequence
  Synthetic sequence
HQTNRWPLT

SEQ ID NO: 166 - protein, 6 aa, Artificial Sequence
  Synthetic sequence
SDYAWN

SEQ ID NO: 167 - protein, 16 aa, Artificial Sequence
  Synthetic sequence
CIRYSGGTTYNPSLKS

SEQ ID NO: 168 - protein, 11 aa, Artificial Sequence
  Synthetic sequence
SRQLGDAGFDY

SEQ ID NO: 169 - protein, 10 aa, Artificial Sequence
  Synthetic sequence
SASSSVSYMY

SEQ ID NO: 170 - protein, 7 aa, Artificial Sequence
  Synthetic sequence
RTSNLAS

SEQ ID NO: 171 - protein, 9 aa, Artificial Sequence
  Synthetic sequence
QQYHSYPPT

SEQ ID NO: 172 - protein, 5 aa, Artificial Sequence
  Synthetic sequence
DYTMN

SEQ ID NO: 173 - protein, 17 aa, Artificial Sequence
  Synthetic sequence
LFNPYNGGISYNQKFKG

SEQ ID NO: 174 - protein, 11 aa, Artificial Sequence
  Synthetic sequence
DGRGGFYAMDY

SEQ ID NO: 175 - protein, 11 aa, Artificial Sequence
  Synthetic sequence
RASQDISIYLN

SEQ ID NO: 176 - protein, 7 aa, Artificial Sequence
  Synthetic sequence
YTSRLHS

SEQ ID NO: 177 - protein, 10 aa, Artificial Sequence
  Synthetic sequence
QQGYTLPPWT

SEQ ID NO: 178 - protein, 5 aa, Artificial Sequence
  Synthetic sequence
DYYMH

SEQ ID NO: 179 - protein, 17 aa, Artificial Sequence
  Synthetic sequence
EINPSTGGTSYNPKFKD

SEQ ID NO: 180 - protein, 13 aa, Artificial Sequence
  Synthetic sequence
YHYYGSSSYVMDY

SEQ ID NO: 181 - protein, 10 aa, Artificial Sequence
  Synthetic sequence
SASSSVYHMH

SEQ ID NO: 182 - protein, 7 aa, Artificial Sequence
  Synthetic sequence
DTSKLAS

SEQ ID NO: 183 - protein, 9 aa, Artificial Sequence
  Synthetic sequence
QHWRTNPLT

SEQ ID NO: 184 - protein, 5 aa, Artificial Sequence
  Synthetic sequence
HTYMH

SEQ ID NO: 185 - protein, 17 aa, Artificial Sequence
  Synthetic sequence
RIDPANGNTEYDPKFQG

SEQ ID NO: 186 - protein, 7 aa, Artificial Sequence
  Synthetic sequence
DGWYIDV

SEQ ID NO: 187 - protein, 17 aa, Artificial Sequence
  Synthetic sequence
KSSQSLLYSNNQKNYLA

SEQ ID NO: 188 - protein, 7 aa, Artificial Sequence
  Synthetic sequence
WASTRES

SEQ ID NO: 189 - protein, 9 aa, Artificial Sequence
  Synthetic sequence
QRYYSYPWT

SEQ ID NO: 190 - protein, 5 aa, Artificial Sequence
  Synthetic sequence
DIFLH

SEQ ID NO: 191 - protein, 17 aa, Artificial Sequence
  Synthetic sequence
RIDPASDNTIYDPKFQD

SEQ ID NO: 192 - protein, 3 aa, Artificial Sequence
  Synthetic sequence
FDF

SEQ ID NO: 193 - protein, 16 aa, Artificial Sequence
  Synthetic sequence
RSSESLLHSNGNTYLY

SEQ ID NO: 194 - protein, 7 aa, Artificial Sequence
  Synthetic sequence
RMSNLAS

SEQ ID NO: 195 - protein, 9 aa, Artificial Sequence
  Synthetic sequence
MQHLEYPLT

SEQ ID NO: 196 - protein, 5 aa, Artificial Sequence
  Synthetic sequence
SYWMN

SEQ ID NO: 197 - protein, 19 aa, Artificial Sequence
  Synthetic sequence
EIRLKSNGFAAFYAESVKG

SEQ ID NO: 198 - protein, 3 aa, Artificial Sequence
  Synthetic sequence
FVH

SEQ ID NO: 199 - protein, 15 aa, Artificial Sequence
  Synthetic sequence
RTSESVEYFGTILIQ

SEQ ID NO: 200 - protein, 7 aa, Artificial Sequence
  Synthetic sequence
GASNVES

SEQ ID NO: 201 - protein, 9 aa, Artificial Sequence
  Synthetic sequence
QQNRKVPYT

SEQ ID NO: 202 - protein, 5 aa, Artificial Sequence
  Synthetic sequence
DTFMH

SEQ ID NO: 203 - protein, 17 aa, Artificial Sequence
  Synthetic sequence
RIDPANDNSIYGPKFQD

SEQ ID NO: 204 - protein, 3 aa, Artificial Sequence
  Synthetic sequence
FDN

SEQ ID NO: 205 - protein, 16 aa, Artificial Sequence
  Synthetic sequence
RSDKSLLHSNGHNYLY

SEQ ID NO: 206 - protein, 7 aa, Artificial Sequence
  Synthetic sequence
RMSNLAS

SEQ ID NO: 207 - protein, 9 aa, Artificial Sequence
  Synthetic sequence
MQHLEYPLT

SEQ ID NO: 208 - protein, 5 aa, Artificial Sequence
  Synthetic sequence
GYWIE

SEQ ID NO: 209 - protein, 17 aa, Artificial Sequence
  Synthetic sequence
EILPGSGSTNYNEKFKG

SEQ ID NO: 210 - protein, 7 aa, Artificial Sequence
  Synthetic sequence
AGAWFAY

SEQ ID NO: 211 - protein, 12 aa, Artificial Sequence
  Synthetic sequence
TASSSVSSSYLH

SEQ ID NO: 212 - protein, 7 aa, Artificial Sequence
  Synthetic sequence
STSNLAS

SEQ ID NO: 213 - protein, 9 aa, Artificial Sequence
  Synthetic sequence
HQYHRSPWT

SEQ ID NO: 214 - protein, 5 aa, Artificial Sequence
  Synthetic sequence
SYWMH

SEQ ID NO: 215 - protein, 17 aa, Artificial Sequence
  Synthetic sequence
IIHPNIGSTNYNERFKS

SEQ ID NO: 216 - protein, 12 aa, Artificial Sequence
  Synthetic sequence
RSSNYGDWYFDV

SEQ ID NO: 217 - protein, 11 aa, Artificial Sequence
  Synthetic sequence
KASQDVGTSVA

SEQ ID NO: 218 - protein, 7 aa, Artificial Sequence
  Synthetic sequence
WASTQHT

SEQ ID NO: 219 - protein, 9 aa, Artificial Sequence
  Synthetic sequence
QQYSSYPLT

SEQ ID NO: 220 - protein, 5 aa, Artificial Sequence
  Synthetic sequence
SYWMH

SEQ ID NO: 221 - protein, 17 aa, Artificial Sequence
  Synthetic sequence
NINPSNGDTFYNEKFKN

SEQ ID NO: 222 - protein, 6 aa, Artificial Sequence
  Synthetic sequence
GWLRDY

SEQ ID NO: 223 - protein, 17 aa, Artificial Sequence
  Synthetic sequence
KSSQSLLNSSSQKNYLA

SEQ ID NO: 224 - protein, 7 aa, Artificial Sequence
  Synthetic sequence
FASTKDS

SEQ ID NO: 225 - protein, 9 aa, Artificial Sequence
  Synthetic sequence
QQHYTTPYT

SEQ ID NO: 226 - protein, 5 aa, Artificial Sequence
  Synthetic sequence
DYNMD

SEQ ID NO: 227 - protein, 17 aa, Artificial Sequence
  Synthetic sequence
DINPNNGGSIYNQRFKG

SEQ ID NO: 228 - protein, 11 aa, Artificial Sequence
  Synthetic sequence
RAYYSTGYFDV

SEQ ID NO: 229 - protein, 10 aa, Artificial Sequence
  Synthetic sequence
SASSSISYMH

SEQ ID NO: 230 - protein, 7 aa, Artificial Sequence
  Synthetic sequence
STSTLAS

SEQ ID NO: 231 - protein, 9 aa, Artificial Sequence
  Synthetic sequence
QQRSSYPPT

SEQ ID NO: 232 - protein, 5 aa, Artificial Sequence
  Synthetic sequence
NYWMH

SEQ ID NO: 233 - protein, 17 aa, Artificial Sequence
  Synthetic sequence
NINPSNGGPYYNERFRS

SEQ ID NO: 234 - protein, 12 aa, Artificial Sequence
  Synthetic sequence
PYYGSSYGYFDY

SEQ ID NO: 235 - protein, 11 aa, Artificial Sequence
  Synthetic sequence
KASQDINKYIA

SEQ ID NO: 236 - protein, 7 aa, Artificial Sequence
  Synthetic sequence
YTSELQP

SEQ ID NO: 237 - protein, 9 aa, Artificial Sequence
  Synthetic sequence
LQYANPLRT

SEQ ID NO: 238 - protein, 5 aa, Artificial Sequence
  Synthetic sequence
SYWMH

SEQ ID NO: 239 - protein, 17 aa, Artificial Sequence
  Synthetic sequence
NINPSNGGTNYNEKIKN

SEQ ID NO: 240 - protein, 11 aa, Artificial Sequence
  Synthetic sequence
WNYYGNYPFDY

SEQ ID NO: 241 - protein, 11 aa, Artificial Sequence
  Synthetic sequence
KASQNVGTAVA

SEQ ID NO: 242 - protein, 7 aa, Artificial Sequence
  Synthetic sequence
SASNRYT

SEQ ID NO: 243 - protein, 9 aa, Artificial Sequence
  Synthetic sequence
QQYSSYPLT

SEQ ID NO: 244 - protein, 5 aa, Artificial Sequence
  Synthetic sequence
NYWMH

SEQ ID NO: 245 - protein, 17 aa, Artificial Sequence
  Synthetic sequence
NINPSSGDSYYNERFMS

SEQ ID NO: 246 - protein, 8 aa, Artificial Sequence
  Synthetic sequence
SGGLWLAF

SEQ ID NO: 247 - protein, 17 aa, Artificial Sequence
  Synthetic sequence
KSSQTLLNSVSQNNYLA

SEQ ID NO: 248 - protein, 7 aa, Artificial Sequence
  Synthetic sequence
FASTRES

SEQ ID NO: 249 - protein, 9 aa, Artificial Sequence
  Synthetic sequence
QQHYRTPYT

SEQ ID NO: 250 - protein, 5 aa, Artificial Sequence
  Synthetic sequence
SYWMH

SEQ ID NO: 251 - protein, 17 aa, Artificial Sequence
  Synthetic sequence
MIHPNSGSTNYNEKFKS

SEQ ID NO: 252 - protein, 10 aa, Artificial Sequence
  Synthetic sequence
PVVPYWYFDV

SEQ ID NO: 253 - protein, 11 aa, Artificial Sequence
  Synthetic sequence
KASQNVGTAVA

SEQ ID NO: 254 - protein, 7 aa, Artificial Sequence
  Synthetic sequence
SASNRYT

SEQ ID NO: 255 - protein, 9 aa, Artificial Sequence
  Synthetic sequence
QQSSSYPLT

SEQ ID NO: 256 - protein, 6 aa, Artificial Sequence
  Synthetic sequence
SGYDWH

SEQ ID NO: 257 - protein, 16 aa, Artificial Sequence
  Synthetic sequence
YISYSGSTNYNPSLKS

SEQ ID NO: 258 - protein, 6 aa, Artificial Sequence
  Synthetic sequence
GTGPDY

SEQ ID NO: 259 - protein, 11 aa, Artificial Sequence
  Synthetic sequence
KASQNVRTAVA

SEQ ID NO: 260 - protein, 7 aa, Artificial Sequence
  Synthetic sequence
LPSNRHT

SEQ ID NO: 261 - protein, 9 aa, Artificial Sequence
  Synthetic sequence
LQHWNYPLT

SEQ ID NO: 262 - protein, 5 aa, Artificial Sequence
  Synthetic sequence
SYWVH

SEQ ID NO: 263 - protein, 17 aa, Artificial Sequence
  Synthetic sequence
YINPNSGYTKYNQKFKD

SEQ ID NO: 264 - protein, 12 aa, Artificial Sequence
  Synthetic sequence
HYYGSSRDYFDY

SEQ ID NO: 265 - protein, 10 aa, Artificial Sequence
  Synthetic sequence
SASSSVSYMY

SEQ ID NO: 266 - protein, 7 aa, Artificial Sequence
  Synthetic sequence
DTSNLAS

SEQ ID NO: 267 - protein, 9 aa, Artificial Sequence
  Synthetic sequence
QQWNSYPYT

SEQ ID NO: 268 - protein, 5 aa, Artificial Sequence
  Synthetic sequence
NYWMN

SEQ ID NO: 269 - protein, 19 aa, Artificial Sequence
  Synthetic sequence
QIRLKSDNYATHYAESVKG

SEQ ID NO: 270 - protein, 5 aa, Artificial Sequence
  Synthetic sequence
GTGGY

SEQ ID NO: 271 - protein, 15 aa, Artificial Sequence
  Synthetic sequence
RASESVSIVGTNVIH

SEQ ID NO: 272 - protein, 7 aa, Artificial Sequence
  Synthetic sequence
HASNLET

SEQ ID NO: 273 - protein, 9 aa, Artificial Sequence
  Synthetic sequence
LQSRKIPWT

SEQ ID NO: 274 - protein, 15 aa, Artificial Sequence
  Synthetic sequence
RASESVSIIGTDVIH

SEQ ID NO: 275 - protein, 7 aa, Artificial Sequence
  Synthetic sequence
HASNLET

SEQ ID NO: 276 - protein, 9 aa, Artificial Sequence
  Synthetic sequence
LQSRKIPWT

SEQ ID NO: 277 - protein, 5 aa, Artificial Sequence
  Synthetic sequence
DYEMH

SEQ ID NO: 278 - protein, 17 aa, Artificial Sequence
  Synthetic sequence
AIDPETGDTVYNQNFKG

SEQ ID NO: 279 - protein, 6 aa, Artificial Sequence
  Synthetic sequence
YGYDWG

SEQ ID NO: 280 - protein, 16 aa, Artificial Sequence
  Synthetic sequence
KSSQSLLYSNGKTYLN

SEQ ID NO: 281 - protein, 7 aa, Artificial Sequence
  Synthetic sequence
LVSKLDS

SEQ ID NO: 282 - protein, 9 aa, Artificial Sequence
  Synthetic sequence
VQGTHFPWT

SEQ ID NO: 283 - protein, 5 aa, Artificial Sequence
  Synthetic sequence
DYYMN

SEQ ID NO: 284 - protein, 17 aa, Artificial Sequence
  Synthetic sequence
VINPKNGVISHNQKFKG

SEQ ID NO: 285 - protein, 10 aa, Artificial Sequence
  Synthetic sequence
YGSRFYAMDY

SEQ ID NO: 286 - protein, 16 aa, Artificial Sequence
  Synthetic sequence
RSSQSLIHSDGNTYLQ

SEQ ID NO: 287 - protein, 7 aa, Artificial Sequence
  Synthetic sequence
KVSNRFS

SEQ ID NO: 288 - protein, 9 aa, Artificial Sequence
  Synthetic sequence
SQTTHVPFT

SEQ ID NO: 289 - protein, 6 aa, Artificial Sequence
  Synthetic sequence
SGYYWN

SEQ ID NO: 290 - protein, 16 aa, Artificial Sequence
  Synthetic sequence
FIRFDGTNNYNPSLKN

SEQ ID NO: 291 - protein, 11 aa, Artificial Sequence
  Synthetic sequence
EGSYAPAWFAY

SEQ ID NO: 292 - protein, 10 aa, Artificial Sequence
  Synthetic sequence
SASSSLSYMY

SEQ ID NO: 293 - protein, 7 aa, Artificial Sequence
  Synthetic sequence
DTSNLAS

SEQ ID NO: 294 - protein, 9 aa, Artificial Sequence
  Synthetic sequence
QQWSSYPPT

SEQ ID NO: 295 - protein, 5 aa, Artificial Sequence
  Synthetic sequence
DYEMH

SEQ ID NO: 296 - protein, 17 aa, Artificial Sequence
  Synthetic sequence
GFDPETGGTAHNQKFKG

SEQ ID NO: 297 - protein, 8 aa, Artificial Sequence
  Synthetic sequence
YGSIWGDC

SEQ ID NO: 298 - protein, 16 aa, Artificial Sequence
  Synthetic sequence
KSSQSLLYSDGKTYLN

SEQ ID NO: 299 - protein, 7 aa, Artificial Sequence
  Synthetic sequence
LVSKLDS

SEQ ID NO: 300 - protein, 9 aa, Artificial Sequence
  Synthetic sequence
WQGTHFPWT

SEQ ID NO: 301 - protein, 5 aa, Artificial Sequence
  Synthetic sequence
DYEMH

SEQ ID NO: 302 - protein, 17 aa, Artificial Sequence
  Synthetic sequence
GIDPETGDTAYNQQFKG

SEQ ID NO: 303 - protein, 8 aa, Artificial Sequence
  Synthetic sequence
YASSREDY

SEQ ID NO: 304 - protein, 16 aa, Artificial Sequence
  Synthetic sequence
KSSQSLLYSNGKTYLS

SEQ ID NO: 305 - protein, 7 aa, Artificial Sequence
  Synthetic sequence
QVSKLDS

SEQ ID NO: 306 - protein, 9 aa, Artificial Sequence
  Synthetic sequence
VQITHFPQT

SEQ ID NO: 307 - protein, 5 aa, Artificial Sequence
  Synthetic sequence
DSEMH

SEQ ID NO: 308 - protein, 17 aa, Artificial Sequence
  Synthetic sequence
AIDPEIDGTAYNQNFRD

SEQ ID NO: 309 - protein, 11 aa, Artificial Sequence
  Synthetic sequence
YFGSGYGYFDV

SEQ ID NO: 310 - protein, 16 aa, Artificial Sequence
  Synthetic sequence
RSSQSLVYSNGNTYLH

SEQ ID NO: 311 - protein, 7 aa, Artificial Sequence
  Synthetic sequence
KVSNRFS

SEQ ID NO: 312 - protein, 9 aa, Artificial Sequence
  Synthetic sequence
SQSTHIPFT

SEQ ID NO: 313 - protein, 5 aa, Artificial Sequence
  Synthetic sequence
DYEIH

SEQ ID NO: 314 - protein, 17 aa, Artificial Sequence
  Synthetic sequence
AFDPEIGGSAYNQKFKD

SEQ ID NO: 315 - protein, 11 aa, Artificial Sequence
  Synthetic sequence
YYGSSSGYFDV

SEQ ID NO: 316 - protein, 16 aa, Artificial Sequence
  Synthetic sequence
RSSQSLEHNNGNTYLH

SEQ ID NO: 317 - protein, 7 aa, Artificial Sequence
  Synthetic sequence
KVSNRFS

SEQ ID NO: 318 - protein, 9 aa, Artificial Sequence
  Synthetic sequence
SQSTHVPFT

SEQ ID NO: 319 - protein, 5 aa, Artificial Sequence
  Synthetic sequence
SYWVH

SEQ ID NO: 320 - protein, 17 aa, Artificial Sequence
  Synthetic sequence
NIDPSDSEIHYNQKFKD

SEQ ID NO: 321 - protein, 8 aa, Artificial Sequence
  Synthetic sequence
GGVPWFAY

SEQ ID NO: 322 - protein, 12 aa, Artificial Sequence
  Synthetic sequence
SASSSVSSTYLY

SEQ ID NO: 323 - protein, 7 aa, Artificial Sequence
  Synthetic sequence
STSNLAS

SEQ ID NO: 324 - protein, 9 aa, Artificial Sequence
  Synthetic sequence
HQWISYPYT

SEQ ID NO: 325 - protein, 5 aa, Artificial Sequence
  Synthetic sequence
DYYMN

SEQ ID NO: 326 - protein, 17 aa, Artificial Sequence
  Synthetic sequence
VINPYNDVISYNQKFKG

SEQ ID NO: 327 - protein, 10 aa, Artificial Sequence
  Synthetic sequence
YGSSYYAMDY

SEQ ID NO: 328 - protein, 16 aa, Artificial Sequence
  Synthetic sequence
RSSQSLVHSNGNTYLQ

SEQ ID NO: 329 - protein, 7 aa, Artificial Sequence
  Synthetic sequence
KVSNRFS

SEQ ID NO: 330 - protein, 9 aa, Artificial Sequence
  Synthetic sequence
SQTTHVPFT

SEQ ID NO: 331 - protein, 5 aa, Artificial Sequence
  Synthetic sequence
DYFMN

SEQ ID NO: 332 - protein, 17 aa, Artificial Sequence
  Synthetic sequence
VINPSNGVINYNQRFKG

SEQ ID NO: 333 - protein, 9 aa, Artificial Sequence
  Synthetic sequence
SYDFAWFAY

SEQ ID NO: 334 - protein, 16 aa, Artificial Sequence
  Synthetic sequence
RSSQNIVHSNGNTYLE

SEQ ID NO: 335 - protein, 7 aa, Artificial Sequence
  Synthetic sequence
TVSNRFS

SEQ ID NO: 336 - protein, 9 aa, Artificial Sequence
  Synthetic sequence
FQGSHVPYT

SEQ ID NO: 337 - protein, 5 aa, Artificial Sequence
  Synthetic sequence
AYWMH

SEQ ID NO: 338 - protein, 17 aa, Artificial Sequence
  Synthetic sequence
NINPSNGGTDFNEKFKS

SEQ ID NO: 339 - protein, 7 aa, Artificial Sequence
  Synthetic sequence
GRGYFDV

SEQ ID NO: 340 - protein, 11 aa, Artificial Sequence
  Synthetic sequence
RASKNIGTSIH

SEQ ID NO: 341 - protein, 7 aa, Artificial Sequence
  Synthetic sequence
YASESIS

SEQ ID NO: 342 - protein, 10 aa, Artificial Sequence
  Synthetic sequence
QQSNSWPTLT

SEQ ID NO: 343 - protein, 5 aa, Artificial Sequence
  Synthetic sequence
DYYIN

SEQ ID NO: 344 - protein, 17 aa, Artificial Sequence
  Synthetic sequence
WIFPGRGSTFYYEKFKG

SEQ ID NO: 345 - protein, 12 aa, Artificial Sequence
  Synthetic sequence
RGDSAGYEDLDY

SEQ ID NO: 346 - protein, 11 aa, Artificial Sequence
  Synthetic sequence
KASENVVTYVS

SEQ ID NO: 347 - protein, 7 aa, Artificial Sequence
  Synthetic sequence
GASKRYT

SEQ ID NO: 348 - protein, 9 aa, Artificial Sequence
  Synthetic sequence
GQSYSYPYT

SEQ ID NO: 349 - protein, 5 aa, Artificial Sequence
  Synthetic sequence
SNGMS

SEQ ID NO: 350 - protein, 17 aa, Artificial Sequence
  Synthetic sequence
TISSGGRYTYYPDSVKG

SEQ ID NO: 351 - protein, 9 aa, Artificial Sequence
  Synthetic sequence
RGIYDYFDC

SEQ ID NO: 352 - protein, 11 aa, Artificial Sequence
  Synthetic sequence
RASENIHSSLA

SEQ ID NO: 353 - protein, 7 aa, Artificial Sequence
  Synthetic sequence
AATNLAD

SEQ ID NO: 354 - protein, 9 aa, Artificial Sequence
  Synthetic sequence
QHFWGTPWT

SEQ ID NO: 355 - protein, 5 aa, Artificial Sequence
  Synthetic sequence
TYTMH

SEQ ID NO: 356 - protein, 17 aa, Artificial Sequence
  Synthetic sequence
YISPTGDFTKYNQKFKD

SEQ ID NO: 357 - protein, 9 aa, Artificial Sequence
  Synthetic sequence
WNGTVWFAY

SEQ ID NO: 358 - protein, 11 aa, Artificial Sequence
  Synthetic sequence
KASQALGTNVA

SEQ ID NO: 359 - protein, 7 aa, Artificial Sequence
  Synthetic sequence
SASYRYS

SEQ ID NO: 360 - protein, 9 aa, Artificial Sequence
  Synthetic sequence
QQYNNYPLT

SEQ ID NO: 361 - protein, 5 aa, Artificial Sequence
  Synthetic sequence
DYNMD

SEQ ID NO: 362 - protein, 17 aa, Artificial Sequence
  Synthetic sequence
DVFPYNGDSIYNQKFEG

SEQ ID NO: 363 - protein, 10 aa, Artificial Sequence
  Synthetic sequence
RKTGTGYFDV

SEQ ID NO: 364 - protein, 12 aa, Artificial Sequence
  Synthetic sequence
SASSSVSSTYLS

SEQ ID NO: 365 - protein, 7 aa, Artificial Sequence
  Synthetic sequence
STSNLAS

SEQ ID NO: 366 - protein, 9 aa, Artificial Sequence
  Synthetic sequence
HQWSSYPYT

SEQ ID NO: 367 - protein, 5 aa, Artificial Sequence
  Synthetic sequence
DYNMD

SEQ ID NO: 368 - protein, 17 aa, Artificial Sequence
  Synthetic sequence
DINPNTDDTIYNQKFNG

SEQ ID NO: 369 - protein, 10 aa, Artificial Sequence
  Synthetic sequence
RLIGTGYFDV

SEQ ID NO: 370 - protein, 10 aa, Artificial Sequence
  Synthetic sequence
RLIGTGYFDV

SEQ ID NO: 371 - protein, 7 aa, Artificial Sequence
  Synthetic sequence
STSNLAS

SEQ ID NO: 372 - protein, 9 aa, Artificial Sequence
  Synthetic sequence
HQWSNYPYT

SEQ ID NO: 373 - protein, 5 aa, Artificial Sequence
  Synthetic sequence
NYLIE

SEQ ID NO: 374 - protein, 17 aa, Artificial Sequence
  Synthetic sequence
VINPGSGGTKYNEKFKG

SEQ ID NO: 375 - protein, 9 aa, Artificial Sequence
  Synthetic sequence
DYDYDPFAY

SEQ ID NO: 376 - protein, 11 aa, Artificial Sequence
  Synthetic sequence
KASQDVSTAVA

SEQ ID NO: 377 - protein, 7 aa, Artificial Sequence
  Synthetic sequence
WASTRHT

SEQ ID NO: 378 - protein, 9 aa, Artificial Sequence
  Synthetic sequence
QQHYSTPYT

SEQ ID NO: 379 - protein, 5 aa, Artificial Sequence
  Synthetic sequence
TFWMH

SEQ ID NO: 380 - protein, 17 aa, Artificial Sequence
  Synthetic sequence
GVNPTNGGTNYNEKFRT

SEQ ID NO: 381 - protein, 11 aa, Artificial Sequence
  Synthetic sequence
HYISSRPGFAY

SEQ ID NO: 382 - protein, 11 aa, Artificial Sequence
  Synthetic sequence
KASQNVGTNVA

SEQ ID NO: 383 - protein, 7 aa, Artificial Sequence
  Synthetic sequence
SASYRYS

SEQ ID NO: 384 - protein, 9 aa, Artificial Sequence
  Synthetic sequence
QQYNSYPLT

SEQ ID NO: 385 - protein, 5 aa, Artificial Sequence
  Synthetic sequence
DYYMS

SEQ ID NO: 386 - protein, 19 aa, Artificial Sequence
  Synthetic sequence
FIRSKANAYTTEYSASVKG

SEQ ID NO: 387 - protein, 14 aa, Artificial Sequence
  Synthetic sequence
YYYYGSGYVWYFDV

SEQ ID NO: 388 - protein, 11 aa, Artificial Sequence
  Synthetic sequence
KASQSVGTNVA

SEQ ID NO: 389 - protein, 7 aa, Artificial Sequence
  Synthetic sequence
SASYRYS

SEQ ID NO: 390 - protein, 9 aa, Artificial Sequence
  Synthetic sequence
QQYNSYPLT

SEQ ID NO: 391 - protein, 5 aa, Artificial Sequence
  Synthetic sequence
SYWMH

SEQ ID NO: 392 - protein, 17 aa, Artificial Sequence
  Synthetic sequence
NIIPSNGGTKYNEKFKS

SEQ ID NO: 393 - protein, 11 aa, Artificial Sequence
  Synthetic sequence
HYYGGSPGFAY

SEQ ID NO: 394 - protein, 7 aa, Artificial Sequence
  Synthetic sequence
TGNYRWS

SEQ ID NO: 395 - protein, 16 aa, Artificial Sequence
  Synthetic sequence
YIYYTGFITYNPSLTS

SEQ ID NO: 396 - protein, 10 aa, Artificial Sequence
  Synthetic sequence
DDYDYDVFAY

SEQ ID NO: 397 - protein, 11 aa, Artificial Sequence
  Synthetic sequence
RASGNIHNYLA

SEQ ID NO: 398 - protein, 7 aa, Artificial Sequence
  Synthetic sequence
KAKTLAD

SEQ ID NO: 399 - protein, 9 aa, Artificial Sequence
  Synthetic sequence
QHFLSIPLT

SEQ ID NO: 400 - protein, 5 aa, Artificial Sequence
  Synthetic sequence
TYWMH

SEQ ID NO: 401 - protein, 17 aa, Artificial Sequence
  Synthetic sequence
NVNPSNGGTMYNEKFER

SEQ ID NO: 402 - protein, 11 aa, Artificial Sequence
  Synthetic sequence
HYIGSRPGFAY

SEQ ID NO: 403 - protein, 5 aa, Artificial Sequence
  Synthetic sequence
SYWMH

SEQ ID NO: 404 - protein, 17 aa, Artificial Sequence
  Synthetic sequence
NVNPSNGGSNYNEKFKN

SEQ ID NO: 405 - protein, 11 aa, Artificial Sequence
  Synthetic sequence
HYIGSRPGFAY

SEQ ID NO: 406 - protein, 11 aa, Artificial Sequence
  Synthetic sequence
KASQNVGTNIA

SEQ ID NO: 407 - protein, 7 aa, Artificial Sequence
  Synthetic sequence
SASYRYS

SEQ ID NO: 408 - protein, 9 aa, Artificial Sequence
  Synthetic sequence
QQYNSYPLT

SEQ ID NO: 409 - protein, 5 aa, Artificial Sequence
  Synthetic sequence
SYWMH

SEQ ID NO: 410 - protein, 17 aa, Artificial Sequence
  Synthetic sequence
SINPSNGDTYYNEKFKN

SEQ ID NO: 411 - protein, 6 aa, Artificial Sequence
  Synthetic sequence
GWYFDV

SEQ ID NO: 412 - protein, 17 aa, Artificial Sequence
  Synthetic sequence
KSSQSLLNSSSQKNYLA

SEQ ID NO: 413 - protein, 7 aa, Artificial Sequence
  Synthetic sequence
FASTRES

SEQ ID NO: 414 - protein, 9 aa, Artificial Sequence
  Synthetic sequence
QQHYSTPRT

SEQ ID NO: 415 - protein, 5 aa, Artificial Sequence
  Synthetic sequence
DYNMD

SEQ ID NO: 416 - protein, 17 aa, Artificial Sequence
  Synthetic sequence
DINPSTGGTIYNQKFNG

SEQ ID NO: 417 - protein, 10 aa, Artificial Sequence
  Synthetic sequence
RRIGTGYFDV

SEQ ID NO: 418 - protein, 12 aa, Artificial Sequence
  Synthetic sequence
SASSSVISSYLS

SEQ ID NO: 419 - protein, 7 aa, Artificial Sequence
  Synthetic sequence
RTSNLAS

SEQ ID NO: 420 - protein, 9 aa, Artificial Sequence
  Synthetic sequence
HQWSSFPYT

SEQ ID NO: 421 - protein, 5 aa, Artificial Sequence
  Synthetic sequence
SYWMH

SEQ ID NO: 422 - protein, 17 aa, Artificial Sequence
  Synthetic sequence
NINPSNGGTNNNENFKS

SEQ ID NO: 423 - protein, 11 aa, Artificial Sequence
  Synthetic sequence
NGYHGYWYFDV

SEQ ID NO: 424 - protein, 5 aa, Artificial Sequence
  Synthetic sequence
DYNMD

SEQ ID NO: 425 - protein, 17 aa, Artificial Sequence
  Synthetic sequence
DINPNTDGAIYNQKFQN

SEQ ID NO: 426 - protein, 10 aa, Artificial Sequence
  Synthetic sequence
RKLGRKFFDY

SEQ ID NO: 427 - protein, 10 aa, Artificial Sequence
  Synthetic sequence
SASSSVGYMN

SEQ ID NO: 428 - protein, 7 aa, Artificial Sequence
  Synthetic sequence
DTSNLAS

SEQ ID NO: 429 - protein, 9 aa, Artificial Sequence
  Synthetic sequence
QQWYVYPYT

SEQ ID NO: 430 - protein, 5 aa, Artificial Sequence
  Synthetic sequence
DYNVD

SEQ ID NO: 431 - protein, 17 aa, Artificial Sequence
  Synthetic sequence
DVNPNTDGAIYNQKFEG

SEQ ID NO: 432 - protein, 10 aa, Artificial Sequence
  Synthetic sequence
RRLGQGGFDS

SEQ ID NO: 433 - protein, 12 aa, Artificial Sequence
  Synthetic sequence
GASSSLISKYLY

SEQ ID NO: 434 - protein, 7 aa, Artificial Sequence
  Synthetic sequence
STSNLAS

SEQ ID NO: 435 - protein, 9 aa, Artificial Sequence
  Synthetic sequence
HQWSSYPYT

SEQ ID NO: 436 - protein, 5 aa, Artificial Sequence
  Synthetic sequence
AYNMD

SEQ ID NO: 437 - protein, 17 aa, Artificial Sequence
  Synthetic sequence
DINPNTDSTIYNQKFEG

SEQ ID NO: 438 - protein, 10 aa, Artificial Sequence
  Synthetic sequence
RKLGRGYFDY

SEQ ID NO: 439 - protein, 10 aa, Artificial Sequence
  Synthetic sequence
SVNLSVRYIY

SEQ ID NO: 440 - protein, 7 aa, Artificial Sequence
  Synthetic sequence
DTSNLAS

SEQ ID NO: 441 - protein, 9 aa, Artificial Sequence
  Synthetic sequence
QQWSSFPYT

SEQ ID NO: 442 - protein, 5 aa, Artificial Sequence
  Synthetic sequence
SSWMN

SEQ ID NO: 443 - protein, 17 aa, Artificial Sequence
  Synthetic sequence
RIFVESGNTHYNDKFNG

SEQ ID NO: 444 - protein, 14 aa, Artificial Sequence
  Synthetic sequence
ENIFYHGHSSWFAY

SEQ ID NO: 445 - protein, 15 aa, Artificial Sequence
  Synthetic sequence
RAGESVDDFGISYVH

SEQ ID NO: 446 - protein, 7 aa, Artificial Sequence
  Synthetic sequence
RAANLES

SEQ ID NO: 447 - protein, 9 aa, Artificial Sequence
  Synthetic sequence
QQNNKDPFT

SEQ ID NO: 448 - protein, 5 aa, Artificial Sequence
  Synthetic sequence
SYWMH

SEQ ID NO: 449 - protein, 17 aa, Artificial Sequence
  Synthetic sequence
NINPTTGDINYNEKFRN

SEQ ID NO: 450 - protein, 5 aa, Artificial Sequence
  Synthetic sequence
HDGFL

SEQ ID NO: 451 - protein, 11 aa, Artificial Sequence
  Synthetic sequence
RASENIYSYLA

SEQ ID NO: 452 - protein, 7 aa, Artificial Sequence
  Synthetic sequence
YAKTLAE

SEQ ID NO: 453 - protein, 9 aa, Artificial Sequence
  Synthetic sequence
QHQFGTPRT

SEQ ID NO: 454 - protein, 5 aa, Artificial Sequence
  Synthetic sequence
DYNMD

SEQ ID NO: 455 - protein, 17 aa, Artificial Sequence
  Synthetic sequence
DINPDNGGTIYTQKFKG

SEQ ID NO: 456 - protein, 12 aa, Artificial Sequence
  Synthetic sequence
RLHYYGSSGLDY

SEQ ID NO: 457 - protein, 11 aa, Artificial Sequence
  Synthetic sequence
RASGNIHNYLA

SEQ ID NO: 458 - protein, 7 aa, Artificial Sequence
  Synthetic sequence
NAKTLAD

SEQ ID NO: 459 - protein, 9 aa, Artificial Sequence
  Synthetic sequence
QHFWSTVWT

SEQ ID NO: 460 - protein, 5 aa, Artificial Sequence
  Synthetic sequence
IYWMH

SEQ ID NO: 461 - protein, 17 aa, Artificial Sequence
  Synthetic sequence
MIHPNSGNTNYNEKFKS

SEQ ID NO: 462 - protein, 7 aa, Artificial Sequence
  Synthetic sequence
IHWYFDV

SEQ ID NO: 463 - protein, 10 aa, Artificial Sequence
  Synthetic sequence
SASSSVSYMY

SEQ ID NO: 464 - protein, 7 aa, Artificial Sequence
  Synthetic sequence
DTSNLAS

SEQ ID NO: 465 - protein, 9 aa, Artificial Sequence
  Synthetic sequence
QQWSSYPLT

SEQ ID NO: 466 - protein, 5 aa, Artificial Sequence
  Synthetic sequence
SYWMH

SEQ ID NO: 467 - protein, 17 aa, Artificial Sequence
  Synthetic sequence
HSNPSNGGTNYNEKFKS

SEQ ID NO: 468 - protein, 12 aa, Artificial Sequence
  Synthetic sequence
WVRGSNYEYFDV

SEQ ID NO: 469 - protein, 11 aa, Artificial Sequence
  Synthetic sequence
KASQNVGSAVA

SEQ ID NO: 470 - protein, 7 aa, Artificial Sequence
  Synthetic sequence
SASNRYT

SEQ ID NO: 471 - protein, 9 aa, Artificial Sequence
  Synthetic sequence
QQYSSYPLT

SEQ ID NO: 472 - protein, 6 aa, Artificial Sequence
  Synthetic sequence
SGYYWN

SEQ ID NO: 473 - protein, 16 aa, Artificial Sequence
  Synthetic sequence
YIRYDGSNNYNPSLKN

SEQ ID NO: 474 - protein, 7 aa, Artificial Sequence
  Synthetic sequence
SYYGMDD

SEQ ID NO: 475 - protein, 11 aa, Artificial Sequence
  Synthetic sequence
RTSGDIHNYLA

SEQ ID NO: 476 - protein, 7 aa, Artificial Sequence
  Synthetic sequence
KAKTLEN

SEQ ID NO: 477 - protein, 9 aa, Artificial Sequence
  Synthetic sequence
QHFWTTPYT

SEQ ID NO: 478 - protein, 5 aa, Artificial Sequence
  Synthetic sequence
DYIIH

SEQ ID NO: 479 - protein, 17 aa, Artificial Sequence
  Synthetic sequence
YIFPNNGGTGYNQKFRG

SEQ ID NO: 480 - protein, 9 aa, Artificial Sequence
  Synthetic sequence
WRLRQYFDV

SEQ ID NO: 481 - protein, 5 aa, Artificial Sequence
  Synthetic sequence
DYFMN

SEQ ID NO: 482 - protein, 17 aa, Artificial Sequence
  Synthetic sequence
VINPHNGYVNYNQKFQG

SEQ ID NO: 483 - protein, 9 aa, Artificial Sequence
  Synthetic sequence
SAESAWFAY

SEQ ID NO: 484 - protein, 16 aa, Artificial Sequence
  Synthetic sequence
RSSQSIVHVKGNTYLE

SEQ ID NO: 485 - protein, 7 aa, Artificial Sequence
  Synthetic sequence
KVSNRFS

SEQ ID NO: 486 - protein, 9 aa, Artificial Sequence
  Synthetic sequence
FQGSHLPYT

SEQ ID NO: 487 - protein, 5 aa, Artificial Sequence
  Synthetic sequence
SYWMH

SEQ ID NO: 488 - protein, 17 aa, Artificial Sequence
  Synthetic sequence
MIHPNSGSTNYNEKFKS

SEQ ID NO: 489 - protein, 15 aa, Artificial Sequence
  Synthetic sequence
RTPIITLVGHWYFDV

SEQ ID NO: 490 - protein, 11 aa, Artificial Sequence
  Synthetic sequence
KASQDVGTAVA

SEQ ID NO: 491 - protein, 7 aa, Artificial Sequence
  Synthetic sequence
WASTRHT

SEQ ID NO: 492 - protein, 9 aa, Artificial Sequence
  Synthetic sequence
QQYSSYPLT

SEQ ID NO: 493 - protein, 5 aa, Artificial Sequence
  Synthetic sequence
DYNMN

SEQ ID NO: 494 - protein, 17 aa, Artificial Sequence
  Synthetic sequence
IINPNYGTSSYNQKFKG

SEQ ID NO: 495 - protein, 10 aa, Artificial Sequence
  Synthetic sequence
VGRDNSGFDY

SEQ ID NO: 496 - protein, 10 aa, Artificial Sequence
  Synthetic sequence
SASSSVSYMY

SEQ ID NO: 497 - protein, 7 aa, Artificial Sequence
  Synthetic sequence
DTSNLAS

SEQ ID NO: 498 - protein, 9 aa, Artificial Sequence
  Synthetic sequence
QQWSSYPPT

SEQ ID NO: 499 - protein, 5 aa, Artificial Sequence
  Synthetic sequence
DYNMN

SEQ ID NO: 500 - protein, 17 aa, Artificial Sequence
  Synthetic sequence
IINPYYGTTSNNQNFKG

SEQ ID NO: 501 - protein, 9 aa, Artificial Sequence
  Synthetic sequence
WLRRDAMDY

SEQ ID NO: 502 - protein, 11 aa, Artificial Sequence
  Synthetic sequence
KASQDVGSSVA

SEQ ID NO: 503 - protein, 7 aa, Artificial Sequence
  Synthetic sequence
WTSTRHT

SEQ ID NO: 504 - protein, 9 aa, Artificial Sequence
  Synthetic sequence
QQYSSYPWT

SEQ ID NO: 505 - protein, 5 aa, Artificial Sequence
  Synthetic sequence
DYNMN

SEQ ID NO: 506 - protein, 17 aa, Artificial Sequence
  Synthetic sequence
IINPNYGTTSYNQKFKG

SEQ ID NO: 507 - protein, 10 aa, Artificial Sequence
  Synthetic sequence
GGYDNEAMDY

SEQ ID NO: 508 - protein, 10 aa, Artificial Sequence
  Synthetic sequence
SASSSVSYMH

SEQ ID NO: 509 - protein, 7 aa, Artificial Sequence
  Synthetic sequence
DTSKLAS

SEQ ID NO: 510 - protein, 9 aa, Artificial Sequence
  Synthetic sequence
QQWSSNPPT

SEQ ID NO: 511 - protein, 5 aa, Artificial Sequence
  Synthetic sequence
DYNMN

SEQ ID NO: 512 - protein, 17 aa, Artificial Sequence
  Synthetic sequence
VINPNYGTTGYNQKFKG

SEQ ID NO: 513 - protein, 10 aa, Artificial Sequence
  Synthetic sequence
GGYDGEAMDY

SEQ ID NO: 514 - protein, 10 aa, Artificial Sequence
  Synthetic sequence
SASSSVNYMH

SEQ ID NO: 515 - protein, 7 aa, Artificial Sequence
  Synthetic sequence
DTSKLAS

SEQ ID NO: 516 - protein, 9 aa, Artificial Sequence
  Synthetic sequence
QQWSSYPPT

SEQ ID NO: 517 - protein, 5 aa, Artificial Sequence
  Synthetic sequence
SYGVH

SEQ ID NO: 518 - protein, 16 aa, Artificial Sequence
  Synthetic sequence
VIWSGGSTDYNAAFIS

SEQ ID NO: 519 - protein, 15 aa, Artificial Sequence
  Synthetic sequence
NGGSMITTLYYAMDY

SEQ ID NO: 520 - protein, 16 aa, Artificial Sequence
  Synthetic sequence
RSSQSLVHSYGNTYLH

SEQ ID NO: 521 - protein, 7 aa, Artificial Sequence
  Synthetic sequence
KVSNRFS

SEQ ID NO: 522 - protein, 9 aa, Artificial Sequence
  Synthetic sequence
SQSTHIPFT

SEQ ID NO: 523 - protein, 5 aa, Artificial Sequence
  Synthetic sequence
DYNMN

SEQ ID NO: 524 - protein, 17 aa, Artificial Sequence
  Synthetic sequence
VIIPNYGTSSYNQKFKG

SEQ ID NO: 525 - protein, 11 aa, Artificial Sequence
  Synthetic sequence
WDNYYGSSLDY

SEQ ID NO: 526 - protein, 5 aa, Artificial Sequence
  Synthetic sequence
SYAMS

SEQ ID NO: 527 - protein, 17 aa, Artificial Sequence
  Synthetic sequence
TISDGGNYTYYPDNVKG

SEQ ID NO: 528 - protein, 10 aa, Artificial Sequence
  Synthetic sequence
DRGYPYAMDS

SEQ ID NO: 529 - protein, 17 aa, Artificial Sequence
  Synthetic sequence
KSSQSVLYSSNQKNYLA

SEQ ID NO: 530 - protein, 7 aa, Artificial Sequence
  Synthetic sequence
WASTRES

SEQ ID NO: 531 - protein, 8 aa, Artificial Sequence
  Synthetic sequence
HQYLSSLT

SEQ ID NO: 532 - protein, 5 aa, Artificial Sequence
  Synthetic sequence
SYAMS

SEQ ID NO: 533 - protein, 17 aa, Artificial Sequence
  Synthetic sequence
TISDGGSYTYYPDNVEG

SEQ ID NO: 534 - protein, 10 aa, Artificial Sequence
  Synthetic sequence
DRGYSYALDY

SEQ ID NO: 535 - protein, 5 aa, Artificial Sequence
  Synthetic sequence
SYTMH

SEQ ID NO: 536 - protein, 17 aa, Artificial Sequence
  Synthetic sequence
YINPSSGYTKYNQKFKD

SEQ ID NO: 537 - protein, 12 aa, Artificial Sequence
  Synthetic sequence
YYSNPDYYAMDY

SEQ ID NO: 538 - protein, 10 aa, Artificial Sequence
  Synthetic sequence
SASSSVSYMH

SEQ ID NO: 539 - protein, 7 aa, Artificial Sequence
  Synthetic sequence
STSNLAS

SEQ ID NO: 540 - protein, 9 aa, Artificial Sequence
  Synthetic sequence
QQRSSYPLT

SEQ ID NO: 541 - protein, 5 aa, Artificial Sequence
  Synthetic sequence
TYGMS

SEQ ID NO: 542 - protein, 17 aa, Artificial Sequence
  Synthetic sequence
TISSGGSYTYYPDSLKG

SEQ ID NO: 543 - protein, 10 aa, Artificial Sequence
  Synthetic sequence
HEGYYYGKDY

SEQ ID NO: 544 - protein, 11 aa, Artificial Sequence
  Synthetic sequence
RASHSISNNLH

SEQ ID NO: 545 - protein, 7 aa, Artificial Sequence
  Synthetic sequence
YASQSIS

SEQ ID NO: 546 - protein, 9 aa, Artificial Sequence
  Synthetic sequence
QQTNSWPLT

SEQ ID NO: 547 - protein, 5 aa, Artificial Sequence
  Synthetic sequence
SYAIS

SEQ ID NO: 548 - protein, 16 aa, Artificial Sequence
  Synthetic sequence
VIWTGGGTNYNSALKS

SEQ ID NO: 549 - protein, 12 aa, Artificial Sequence
  Synthetic sequence
NNYGRLDYAMDY

SEQ ID NO: 550 - protein, 16 aa, Artificial Sequence
  Synthetic sequence
RSSQSLVHGNGNTYLH

SEQ ID NO: 551 - protein, 7 aa, Artificial Sequence
  Synthetic sequence
KVSNRFS

SEQ ID NO: 552 - protein, 9 aa, Artificial Sequence
  Synthetic sequence
SQSTHVPFT

SEQ ID NO: 553 - protein, 5 aa, Artificial Sequence
  Synthetic sequence
DYYMH

SEQ ID NO: 554 - protein, 17 aa, Artificial Sequence
  Synthetic sequence
RIDPEDGETKYAPKFQG

SEQ ID NO: 555 - protein, 9 aa, Artificial Sequence
  Synthetic sequence
GSGTGAMDY

SEQ ID NO: 556 - protein, 11 aa, Artificial Sequence
  Synthetic sequence
KASQNVRTAVG

SEQ ID NO: 557 - protein, 7 aa, Artificial Sequence
  Synthetic sequence
LASNRHT

SEQ ID NO: 558 - protein, 9 aa, Artificial Sequence
  Synthetic sequence
LQHWNSPYT

SEQ ID NO: 559 - protein, 5 aa, Artificial Sequence
  Synthetic sequence
DYYMN

SEQ ID NO: 560 - protein, 17 aa, Artificial Sequence
  Synthetic sequence
DINPNNGGTSYNQKFKG

SEQ ID NO: 561 - protein, 13 aa, Artificial Sequence
  Synthetic sequence
WGYGSSSRGYFDY

SEQ ID NO: 562 - protein, 10 aa, Artificial Sequence
  Synthetic sequence
RASSSVSYMH

SEQ ID NO: 563 - protein, 7 aa, Artificial Sequence
  Synthetic sequence
ATSNLAS

SEQ ID NO: 564 - protein, 9 aa, Artificial Sequence
  Synthetic sequence
QQWSSNPWT

SEQ ID NO: 565 - protein, 113 aa, Artificial Sequence
  Synthetic sequence
DIVMTQSPDSLAVSLGERATINCKSSQTLLNSVSQNNYLAWYQQKPGQSPKLLIYFASTRESGVPDRFSGSGSGTDFTLTISSLQAEDVAVYYCQQHYRTPYTFGGGTKVEIK

SEQ ID NO: 566 - protein, 113 aa, Artificial Sequence
  Synthetic sequence
DIVMTQSPDSLAVSLGERATINCKSSQTLLNSVSQNNYLAWYQQKPGQSPTLLIYFASTRESGVPDRFSGSGSGTDFTLTISSLQAEDVAVYYCQQHYRTPYTFGGGTKVEIK

SEQ ID NO: 567 - protein, 113 aa, Artificial Sequence
  Synthetic sequence
DIVMTQTPLSLSVTPGQPASISCKSSQTLLNSVSQNNYLAWYLQKPGQSPQLLVYFASTRESGVPDRFSGSGSGTDFTLKISRVEAEDVGVYYCQQHYRTPYTFGGGTKVEIK

SEQ ID NO: 568 - protein, 113 aa, Artificial Sequence
  Synthetic sequence
DIVMTQTPLSLSVTPGQPASISCKSSQTLLNSVSQNNYLAWYQQKPGQSPTLLVYFASTRESGVPDRFSGSGSGTDFTLKISRVEAEDVGVYYCQQHYRTPYTFGGGTKVEIK

SEQ ID NO: 569 - protein, 117 aa, Artificial Sequence
  Synthetic sequence
EVQLVQSGAEVKKPGASVKVSCKASGYTFTNYWMHWVRQAPGQGLEWMGNINPSSGDSYYNERFMSRVTMTVDKSTSTVYMELSSLRSEDTAVYYCARSGGLWLAFWGQGTLVTVSS

SEQ ID NO: 570 - protein, 117 aa, Artificial Sequence
  Synthetic sequence
EVQLVQSGAEVKKPGASVKVSCKASGYTFTNYWMHWVRQAPGQGLEWMGNINPSSGDSYYNERFMSRVTMTVDKSTSTAYMELSSLRSEDTAVYYCARSGGLWLAFWGQGTLVTVSS

SEQ ID NO: 571 - protein, 117 aa, Artificial Sequence
  Synthetic sequence
EVQLVQSGAEVKKPGASVKVSCKASGYTFTNYWMHWVRQAPGQGLEWMGNINPSSGDSYYNERFMSRVTMTVDKSTSTAYMELSSLRSEDTAVYYCARSGGLWLAFWGPGTLVTVSS

SEQ ID NO: 572 - protein, 101 aa, Artificial Sequence
  Synthetic sequence
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTP

SEQ ID NO: 573 - protein, 100 aa, Artificial Sequence
  Synthetic sequence
DIVMTQTPLSLSVTPGQPASISCKSSQSLLHSDGKTYLYWYLQKPGQSPQLLIYEVSSRFSGVPDRFSGSGSGTDFTLKISRVEAEDVGVYYCMQGIHLP

SEQ ID NO: 574 - protein, 10 aa, Artificial Sequence
  Synthetic sequence
FGGGTKVEIK

SEQ ID NO: 575 - protein, 98 aa, Artificial Sequence
  Synthetic sequence
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 576 - protein, 11 aa, Artificial Sequence
  Synthetic sequence
WGQGTLVTVSS

SEQ ID NO: 577 - protein, 107 aa, Artificial Sequence
  Synthetic sequence
DIQMTQSPSSLSASVGDRVTITCKASQDINKYIAWYQQKPGKAPKLLIHYTSELQPGVPSRFSGSGSGTDFTFTISSLQPEDIATYYCLQYANPLRTFGGGTKVEIK

SEQ ID NO: 578 - protein, 107 aa, Artificial Sequence
  Synthetic sequence
DIQMTQSPSSLSASVGDRVTITCKASQDINKYIAWYQHKPGKAPKLLIHYTSELQPGVPSRFSGSGSGTDFTFTISSLQPEDIATYYCLQYANPLRTFGGGTKVEIK

SEQ ID NO: 579 - protein, 107 aa, Artificial Sequence
  Synthetic sequence
DIQMTQSPSSLSASVGDRVTITCKASQDINKYIAWYQHKPGKAPKLLIHYTSELQPGVPSRFSGSGSGRDFTFTISSLQPEDIATYYCLQYANPLRTFGGGTKVEIK

SEQ ID NO: 580 - protein, 107 aa, Artificial Sequence
  Synthetic sequence
DIQMTQSPSSLSASVGDRVTITCKASQDINKYIAWYQHKPGKAPKLLIHYTSELQPGVPSRFSGSGSGRDYTFTISSLQPEDIATYYCLQYANPLRTFGGGTKVEIK

SEQ ID NO: 581 - protein, 121 aa, Artificial Sequence
  Synthetic sequence
EVQLVQSGAEVKKPGASVKVSCKASGYTFTNYWMHWVRQAPGQGLEWMGNINPSNGGPYYNERFRSRVTMTVDKSTSTVYMELSSLRSEDTAVYYCARPYYGSSYGYFDYWGQGTTVTVSS

SEQ ID NO: 582 - protein, 121 aa, Artificial Sequence
  Synthetic sequence
EVQLVQSGAEVKKPGASVKVSCKASGYTFTNYWMHWVRQAPGQGLEWMGNINPSNGGPYYNERFRSRVTMTVDKSTSTAYMELSSLRSEDTAVYYCARPYYGSSYGYFDYWGQGTTVTVSS

SEQ ID NO: 583 - protein, 121 aa, Artificial Sequence
  Synthetic sequence
EVQLVQSGAEVKKPGASVKVSCKASGYTFTNYWMHWVRQAPGQGLEWMGNINPSDGGPYYNERFRSRVTMTVDKSTSTAYMELSSLRSEDTAVYYCARPYYGSSYGYFDYWGQGTTVTVSS

SEQ ID NO: 584 - protein, 121 aa, Artificial Sequence
  Synthetic sequence
EVQLVQSGAEVKKPGASVKVSCKASGYTFTNYWMHWVRQAPGQGLEWMGNINPSQGGPYYNERFRSRVTMTVDKSTSTAYMELSSLRSEDTAVYYCARPYYGSSYGYFDYWGQGTTVTVSS

SEQ ID NO: 585 - protein, 95 aa, Artificial Sequence
  Synthetic sequence
DIQMTQSPSSLSASVGDRVTITCQASQDISNYLNWYQQKPGKAPKLLIYDASNLETGVPSRFSGSGSGTDFTFTISSLQPEDIATYYCQQYDNLP

SEQ ID NO: 586 - protein, 11 aa, Artificial Sequence
  Synthetic sequence
WGQGTTVTVSS

SEQ ID NO: 587 - protein, 17 aa, Artificial Sequence
  Synthetic sequence
NINPSDGGPYYNERFRS

SEQ ID NO: 588 - protein, 17 aa, Artificial Sequence
  Synthetic sequence
NINPSQGGPYYNERFRS

SEQ ID NO: 589 - protein, 108 aa, Artificial Sequence
  Synthetic sequence
EIVLTQSPDFQSVTPKEKVTITCSASSSVISSYLSWYQQKPDQSPKLLIYRTSNLASGVPSRFSGSGSGTDFTLTINSLEAEDAATYYCHQWSSFPYTFGGGTKVEIK

SEQ ID NO: 590 - protein, 108 aa, Artificial Sequence
  Synthetic sequence
EIVLTQSPDFQSVTPKEKVTITCSASSSVISSYLSWYQQKPDQSPKLLIYRTSNLASGVPSRFSGSGSGTDYTLTINSLEAEDAATYYCHQWSSFPYTFGGGTKVEIK

SEQ ID NO: 591 - protein, 108 aa, Artificial Sequence
  Synthetic sequence
EIVLTQSPATLSLSPGERATLSCSASSSVISSYLSWYQQKPGQSPRLLIYRTSNLASGIPARFSGSGSGTDYTLTISSLEPEDFAVYYCHQWSSFPYTFGGGTKVEIK

SEQ ID NO: 592 - protein, 108 aa, Artificial Sequence
  Synthetic sequence
EIVLTQSPATLSLSPGERATLSCSASSSVISSYLSWYQQKPGSSPRLLIYRTSNLASGIPARFSGSGSGTDYTLTISSLEPEDFAVYYCHQWSSFPYTFGGGTKVEIK

SEQ ID NO: 593 - protein, 119 aa, Artificial Sequence
  Synthetic sequence
EVQLVQSGAEVKKPGASVKVSCKASGYTFTDYNMDWVRQAPGQGLEWMGDINPSTGGTIYNQKFNGRVTMTEDKSTSTVYMELSSLRSEDTAVYYCARRRIGTGYFDVWGQGTTVTVSS

SEQ ID NO: 594 - protein, 119 aa, Artificial Sequence
  Synthetic sequence
EVQLVQSGAEVKKPGASVKVSCKASGYTFTDYNMDWVRQAPGQGLEWMGDINPSTGGTIYNQKFQGRVTMTEDKSTSTVYMELSSLRSEDTAVYYCARRRIGTGYFDVWGQGTTVTVSS

SEQ ID NO: 595 - protein, 119 aa, Artificial Sequence
  Synthetic sequence
EVQLVQSGAEVKKPGASVKVSCKASGYTFTDYNMDWVRQAPGQGLEWMGDINPSTGGTIYNQKFNGRVTMTEDKSASTVYMELSSLRSEDTAVYYCARRRIGTGYFDVWGQGTTVTVSS

SEQ ID NO: 596 - protein, 119 aa, Artificial Sequence
  Synthetic sequence
EVQLVQSGAEVKKPGASVKVSCKASGYTFTDYNMDWVRQAPGQGLEWMGDINPSTGGTIYNQKFSGRVTMTEDKSASTVYMELSSLRSEDTAVYYCARRRIGTGYFDVWGQGTTVTVSS

SEQ ID NO: 597 - protein, 95 aa, Artificial Sequence
  Synthetic sequence
EIVLTQSPATLSLSPGERATLSCRASQSVSSYLAWYQQKPGQAPRLLIYDASNRATGIPARFSGSGSGTDFTLTISSLEPEDFAVYYCQQRSNWP

SEQ ID NO: 598 - protein, 95 aa, Artificial Sequence
  Synthetic sequence
EIVLTQSPDFQSVTPKEKVTITCRASQSIGSSLHWYQQKPDQSPKLLIKYASQSFSGVPSRFSGSGSGTDFTLTINSLEAEDAATYYCHQSSSLP

SEQ ID NO: 599 - protein, 17 aa, Artificial Sequence
  Synthetic sequence
DINPSTGGTIYNQKFQG

SEQ ID NO: 600 - protein, 17 aa, Artificial Sequence
  Synthetic sequence
DINPSTGGTIYNQKFSG

SEQ ID NO: 601 - protein, 107 aa, Artificial Sequence
  Synthetic sequence
DIVMTQSPDSLAVSLGERATINCKASQNVGTNIAWYQQKPGQSPKALIYSASYRYSGVPDRFSGSGSGTDFTLTISSLQAEDVAVYYCQQYNSYPLTFGGGTKVEIK

SEQ ID NO: 602 - protein, 107 aa, Artificial Sequence
  Synthetic sequence
DIVMTQTPLSLSVTPGQPASISCKASQNVGTNIAWYLQKPGQSPQALIYSASYRYSGVPDRFSGSGSGTDFTLKISRVEAEDVGVYYCQQYNSYPLTFGGGTKVEIK

SEQ ID NO: 603 - protein, 107 aa, Artificial Sequence
  Synthetic sequence
DIVMTQTPLSLSVTPGQPASISCKASQNVGTNIAWYQQKPGQSPQALIYSASYRYSGVPDRFSGSGSGTDFTLKISRVEAEDVGVYYCQQYNSYPLTFGGGTKVEIK

SEQ ID NO: 604 - protein, 120 aa, Artificial Sequence
  Synthetic sequence
EVQLVQSGAEVKKPGASVKVSCKASGYTFTSYWMHWVRQAPGQRLEWMGNVNPSNGGSNYNEKFKNRVTITVDKSASTAYMELSSLRSEDTAVYYCAPHYIGSRPGFAYWGQGTLVTVSS

SEQ ID NO: 605 - protein, 120 aa, Artificial Sequence
  Synthetic sequence
EVQLVQSGAEVKKPGASVKVSCKASGYTFTSYWMHWVRQAPGQRLEWMGNVNPSSGGSNYNEKFKNRVTITVDKSASTAYMELSSLRSEDTAVYYCAPHYIGSRPGFAYWGQGTLVTVSS

SEQ ID NO: 606 - protein, 120 aa, Artificial Sequence
  Synthetic sequence
EVQLVQSGAEVKKPGASVKVSCKASGYTFTSYWMHWVRQAPGQGLEWMGNVNPSNGGSNYNEKFKNRVTITVDKSASTAYMELSSLRSEDTAVYYCAPHYIGSRPGFAYWGQGTLVTVSS

SEQ ID NO: 607 - protein, 120 aa, Artificial Sequence
  Synthetic sequence
EVQLVQSGAEVKKPGASVKVSCKASGYTFTSYWMHWVRQAPGQGLEWMGNVNPSNAGSNYNEKFKNRVTITVDKSASTAYMELSSLRSEDTAVYYCAPHYIGSRPGFAYWGQGTLVTVSS

SEQ ID NO: 608 - protein, 98 aa, Artificial Sequence
  Synthetic sequence
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAR

SEQ ID NO: 609 - protein, 17 aa, Artificial Sequence
  Synthetic sequence
NVNPSSGGSNYNEKFKN

SEQ ID NO: 610 - protein, 17 aa, Artificial Sequence
  Synthetic sequence
NVNPSNAGSNYNEKFKN

SEQ ID NO: 611 - protein, 112 aa, Artificial Sequence
  Synthetic sequence
DIVMTQTPLSLPVTPGEPASISCRSSQSLEHNNGNTYLHWFLQKPGQSPQLLIYKVSNRFSGVPDRFSGSGSGTDFTLKISRVEAEDVGVYYCSQSTHVPFTFGQGTKLEIK

SEQ ID NO: 612 - protein, 112 aa, Artificial Sequence
  Synthetic sequence
DIVMTQTPLSLPVTPGEPASISCRSSQSLEHNDGNTYLHWFLQKPGQSPQLLIYKVSNRFSGVPDRFSGSGSGTDFTLKISRVEAEDVGVYYCSQSTHVPFTFGQGTKLEIK

SEQ ID NO: 613 - protein, 112 aa, Artificial Sequence
  Synthetic sequence
DIVMTQTPLSLPVTPGEPASISCRSSQSLEHNNGNTYLHWFLHKPGQSPQLLIYKVSNRFSGVPDRFSGSGSGTDFTLKISRVEAEDVGVYYCSQSTHVPFTFGQGTKLEIK

SEQ ID NO: 614 - protein, 112 aa, Artificial Sequence
  Synthetic sequence
DIVMTQTPLSLPVTPGEPASISCRSSQSLEHNNANTYLHWFLHKPGQSPQLLIYKVSNRFSGVPDRFSGSGSGTDFTLKISRVEAEDVGVYYCSQSTHVPFTFGQGTKLEIK

SEQ ID NO: 615 - protein, 120 aa, Artificial Sequence
  Synthetic sequence
EVQLVQSGAEVKKPGATVKISCKVSGYTFTDYEIHWVQQAPGKGLEWMGAFDPEIGGSAYNQKFKDRVTITADKSTDTAYMELSSLRSEDTAVYYCTDYYGSSSGYFDVWGQGTTVTVSS

SEQ ID NO: 616 - protein, 120 aa, Artificial Sequence
  Synthetic sequence
EVQLVQSGAEVKKPGATVKISCKASGYTFTDYEIHWVQQAPGKGLEWMGAFDPEIGGSAYNQKFKDRVTITADKSTSTAYMELSSLRSEDTAVYYCTDYYGSSSGYFDVWGQGTTVTVSS

SEQ ID NO: 617 - protein, 120 aa, Artificial Sequence
  Synthetic sequence
EVQLVQSGAEVKKPGATVKISCKASGYTFTDYEIHWVQQAPGKGLEWMGAFDPEIGGSAYNQKFKDRVTITADKSTSTAYMELSSLRSEDTAVYYCTDYYGSSSGYFDVWGTGTTVTVSS

SEQ ID NO: 618 - protein, 120 aa, Artificial Sequence
  Synthetic sequence
EVQLVQSGAEVKKPGATVKISCKASGYTFTDYEIHWVQQAPGKGLEWMGAFDPEIGGSAYNQKFKDRVTITADKSSSTAYMELSSLRSEDTAVYYCTDYYGSSSGYFDVWGTGTTVTVSS

SEQ ID NO: 619 - protein, 101 aa, Artificial Sequence
  Synthetic sequence
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQRIEFP

SEQ ID NO: 620 - protein, 10 aa, Artificial Sequence
  Synthetic sequence
FGQGTKLEIK

SEQ ID NO: 621 - protein, 98 aa, Artificial Sequence
  Synthetic sequence
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCAT

SEQ ID NO: 622 - protein, 16 aa, Artificial Sequence
  Synthetic sequence
RSSQSLEHNDGNTYLH

SEQ ID NO: 623 - protein, 16 aa, Artificial Sequence
  Synthetic sequence
RSSQSLEHNNANTYLH

SEQ ID NO: 624 - protein, 112 aa, Artificial Sequence
  Synthetic sequence
DIVMTQTPLSLPVTPGEPASISCRSSQSLIHSDGNTYLQWYLQKPGQSPQLLIYKVSNRFSGVPDRFSGSGSGTDFTLKISRVEAEDVGVYYCSQTTHVPFTFGQGTKLEIK

SEQ ID NO: 625 - protein, 112 aa, Artificial Sequence
  Synthetic sequence
DIVMTQSPDSLAVSLGERATINCRSSQSLIHSDGNTYLQWYQQKPGQSPKLLIYKVSNRFSGVPDRFSGSGSGTDFTLTISSLQAEDVAVYYCSQTTHVPFTFGQGTKLEIK

SEQ ID NO: 626 - protein, 119 aa, Artificial Sequence
  Synthetic sequence
EVQLVQSGAEVKKPGASVKVSCKASGYIFTDYYMNWVRQAPGQRLEWMGVINPKNGVISHNQKFKGRVTITRDTSASTAYMELSSLRSEDTAVYYCANYGSRFYAMDYWGQGTTVTVSS

SEQ ID NO: 627 - protein, 119 aa, Artificial Sequence
  Synthetic sequence
EVQLVQSGAEVKKPGASVKVSCKASGYIFTDYYMNWVRQAPGQRLEWMGVINPKNGVISHNQKFKGRVTITVDKSASTAYMELSSLRSEDTAVYYCANYGSRFYAMDYWGQGTTVTVSS

SEQ ID NO: 628 - protein, 119 aa, Artificial Sequence
  Synthetic sequence
EVQLVQSGAEVKKPGASVKVSCKASGYIFTDYYMNWVRQAPGQRLEWMGVINPKNGVISHNQKFKGRVTITVDKSANTAYMELSSLRSEDTAVYYCANYGSRFYAMDYWGQGTTVTVSS

SEQ ID NO: 629 - protein, 119 aa, Artificial Sequence
  Synthetic sequence
EVQLVQSGAEVKKPGASVKVSCKASGYIFTDYYMNWVRQAPGQRLEWMGVINPKQGVISHNQKFKGRVTITVDKSANTAYMELSSLRSEDTAVYYCANYGSRFYAMDYWGQGTTVTVSS

SEQ ID NO: 630 - protein, 119 aa, Artificial Sequence
  Synthetic sequence
EVQLVQSGAEVKKPGASVKVSCKASGYIFTDYYMNWVRQAPGQSLEWMGVINPKNGVISHNQKFKGRVTITVDKSANTAYMELSSLRSEDTAVYYCANYGSRFYAMDYWGQGTTVTVSS

SEQ ID NO: 631 - protein, 119 aa, Artificial Sequence
  Synthetic sequence
EVQLVQSGAEVKKPGASVKVSCKASGYIFTDYYMNWVRQAHGQRLEWMGVINPKNGVISHNQKFKGRVTITVDKSANTAYMELSSLRSEDTAVYYCANYGSRFYAMDYWGQGTTVTVSS

SEQ ID NO: 632 - protein, 17 aa, Artificial Sequence
  Synthetic sequence
VINPKQGVISHNQKFKG